SEQUENCE LISTING (41 sequences)

SEQ ID NO: 1 - DNA, 21 bp, Artificial Sequence
  Forward primer Twist-1
agatgtcattgtttccagaga

SEQ ID NO: 2 - DNA, 21 bp, Artificial Sequence
  Reverse primer Twist-1
ttagttatccagctccagagt

SEQ ID NO: 3 - DNA, 20 bp, Artificial Sequence
  Forward primer Nanog
agggtctgctatgagatgct

SEQ ID NO: 4 - DNA, 21 bp, Artificial Sequence
  Reverse primer Nanog
caaccactggtttttctgcca

SEQ ID NO: 5 - DNA, 22 bp, Artificial Sequence
  Forward primer Stat3
gccacgttggtgtttcataatc

SEQ ID NO: 6 - DNA, 22 bp, Artificial Sequence
  Reverse primer Stat3
ttcgaaggttgtgctgatagag

SEQ ID NO: 7 - DNA, 20 bp, Artificial Sequence
  Forward primer Tlr4
atggcatggcttacaccacc

SEQ ID NO: 8 - DNA, 21 bp, Artificial Sequence
  Reverse primer Tlr4
gaggccaattttgtctccaca

SEQ ID NO: 9 - DNA, 22 bp, Artificial Sequence
  Forward primer E-cad
ctgctgctcctactgtttctac

SEQ ID NO: 10 - DNA, 21 bp, Artificial Sequence
  Reverse primer E-cad
tcttcttctccacctccttct

SEQ ID NO: 11 - DNA, 20 bp, Artificial Sequence
  Forward primer N-cad
cagggtggacgtcattgtag

SEQ ID NO: 12 - DNA, 20 bp, Artificial Sequence
  Reverse primer N-cad
agggtctccaccactgattc

SEQ ID NO: 13 - DNA, 20 bp, Artificial Sequence
  Forward primer Gapdh
tggatttggacgcattggtc

SEQ ID NO: 14 - DNA, 21 bp, Artificial Sequence
  Reverse primer Gapdh
tttgcactggtacgtgttgat

SEQ ID NO: 15 - DNA, 16 bp, Artificial Sequence
  putative NANOG binding site
cggacgcgcattangc

SEQ ID NO: 16 - DNA, 32 bp, Artificial Sequence
  Forward primer Nanog-mut1 Proximal
gtttgggaggacgaaggagaccccgaggaagg

SEQ ID NO: 17 - DNA, 32 bp, Artificial Sequence
  Reverse primer Nanog-mut1 Proximal
ccttcctcggggtctccttcgtcctcccaaac

SEQ ID NO: 18 - DNA, 29 bp, Artificial Sequence
  Forward primer Nanog-mut2 Distal
aggtcgtttttgcctggtttgggaggacg

SEQ ID NO: 19 - DNA, 29 bp, Artificial Sequence
  Reverse primer Nanog-mut2 Distal
cgtcctcccaaaccaggcaaaaacgacct

SEQ ID NO: 20 - DNA, 37 bp, Artificial Sequence
  Forward primer Stat3-mut1 Proximal
tttcctataaaacatgattacgtccctcctcctcacg

SEQ ID NO: 21 - DNA, 37 bp, Artificial Sequence
  Reverse primer Stat3-mut1 Proximal
cgtgaggaggagggacgtaatcatgttttataggaaa

SEQ ID NO: 22 - DNA, 41 bp, Artificial Sequence
  Forward primer Stat3-mut2 Distal
ctggaaagcggaaactatgattacgaacttcgaaaagtccc

SEQ ID NO: 23 - DNA, 41 bp, Artificial Sequence
  Reverse primer Stat3-mut2 Distal
gggacttttcgaagttcgtaatcatagtttccgctttccag

SEQ ID NO: 24 - DNA, 21 bp, Artificial Sequence
  Forward primer Nanog-Proximal Binding Site
atggtttgggaggacgagtta

SEQ ID NO: 25 - DNA, 21 bp, Artificial Sequence
  Reverse primer Nanog-Proximal Binding Site
aaagtttccgctttccagtcc

SEQ ID NO: 26 - DNA, 19 bp, Artificial Sequence
  Forward primer Stat3-Distal Binding Site
ggactggaaagcggaaact

SEQ ID NO: 27 - DNA, 22 bp, Artificial Sequence
  Reverse primer Stat3-Distal Binding Site
gcagacttggaggctcttatac

SEQ ID NO: 28 - DNA, 20 bp, Artificial Sequence
  Forward primer Stat3-Proximal Binding Site
gccaggtcgtttttgaatgg

SEQ ID NO: 29 - DNA, 20 bp, Artificial Sequence
  Reverse primer Stat3-Proximal Binding Site
cgtgcaggcggaaagtttgg

SEQ ID NO: 30 - DNA, 19 bp, Artificial Sequence
  Forward primer Specificity control-1
cccagcaatcccaaatcgg

SEQ ID NO: 31 - DNA, 21 bp, Artificial Sequence
  Reverse primer Specificity control-1
cagcaatggcaacagcttcta

SEQ ID NO: 32 - DNA, 18 bp, Artificial Sequence
  Forward primer Specificity control-2
ctcacgtcaggccaatga

SEQ ID NO: 33 - DNA, 19 bp, Artificial Sequence
  Reverse primer Specificity control-2
gagagctgcagacttggag

SEQ ID NO: 34 - DNA, 25 bp, Artificial Sequence
  Reverse primer Oct4 binding site
ataaaataacccaaactaaaaaaaa

SEQ ID NO: 35 - DNA, 28 bp, Artificial Sequence
  Forward primer p53 binding site
gttttttgtagaataaaatttaggaaga

SEQ ID NO: 36 - DNA, 25 bp, Artificial Sequence
  Reverse primer p53 binding site
caaacttatctaccaccatacccaa

SEQ ID NO: 37 - DNA, 25 bp, Artificial Sequence
  Forward primer Oct4 binding site
tttaatgtgaagagtaagtaagaaa

SEQ ID NO: 38 - DNA, 22 bp, Artificial Sequence
  Forward primer (-285) Oct4 binding site
agtgaaatgaggtaaagcctct

SEQ ID NO: 39 - DNA, 18 bp, Artificial Sequence
  Reverse primer (-80) Oct4 binding site
tattctcccaggcaccca

SEQ ID NO: 40 - DNA, 22 bp, Artificial Sequence
  Forward primer (-741) p53 binding site
tacagtgagaacttgtctcaaa

SEQ ID NO: 41 - DNA, 18 bp, Artificial Sequence
  Reverse primer (-541) p53 binding site
gagcctgtgtcctgctta

(19) United States
(12) Patent Application Publication
(10) Pub. No.: US 2016/0304970 A1
(43) Pub. Date: Oct. 20, 2016

(54) GENE BIOMARKER TO DIAGNOSE METASTIC LIVER CANCER AND METHODS FOR TARGETING THE SAME

(71) Applicant: Keigo MACHIDA, Los Angeles, CA (US)

(72) Inventor: Keigo MACHIDA, Los Angeles, CA (US)

(73) Assignee: University of Southern California, Los Angeles, CA (US)

(21) Appl. No.: 15/132,152

(22) Filed: Apr. 18, 2016

Related U.S. Application Data
(60) Provisional application No. 62/149,394, filed on Apr. 17, 2015.

(51) Int. Cl. C12Q 1/68; A61K 31/167; A61K 31/203; A61K 31/44

(52) U.S. Cl. CPC C12Q 1/6886; A61K 31/44; A61K 31/167; A61K 31/203; C12Q 2600/158; C12Q 2600/106

(57) ABSTRACT

A method of identifying subjects with metastatic hepatocellular carcinoma (HCC) for tumor-initiating stem-like cell (TIC) targeted therapy is provided. The method includes obtaining whole blood from a subject, retrieving circulating tumor cells (CTCs) and/or TICs from the whole blood, performing quantitative reverse transcriptase-PCR (qRT) PCR on retrieved CTCs and/or TICs, and identifying specific genes that are upregulated and specific genes that are downregulated. The upregulated genes include NANOG, TWIST1, LIN28, MSI2, ACADVL, BIRC5, miR-22, LepR, YAP1 and IGF2BP3. The downregulated genes include COX6A2, COX15, TET1, TET2 and PTEN.

### CROSS-REFERENCE TO RELATED APPLICATIONS

This application claims priority from U.S. Provisional Application No. 62/149,394, filed on Apr. 17, 2015 and entitled “GENE PANEL FOR BIOMARKER TO DIAGNOSE METASTIC LIVER CANCER,” which is incorporated herein by reference in its entirety.

### STATEMENT REGARDING FEDERALLY-SPONSORED RESEARCH

This invention was made with government support under National Institutes of Health grant R01 AA018857. The government has certain rights in the invention.

### BACKGROUND

Major risk factors for the third most deadly cancer, hepatocellular carcinoma (HCC), are hepatitis C virus (HCV), alcoholism and obesity (He et al., 2008; Okuda, 2000; Okuda et al., 2002; Sanyal et al., 2006; Sanyal et al., 2010; Yao and Terrault, 2001). Compelling evidence identified a synergism between obesity/alcohol and HCV infection with the associated risk of developing HCC (Yuan et al., 2004). The risk for HCC, as assessed by odds ratio, increases from 8-12 to 48-54 by co-morbidities such as alcoholism or obesity (Artinyan et al., 2010; Hassan et al., 2002; Tikhanovich et al., 2014; Yuan et al., 2004). Obesity and alcoholism increase the gut permeability leading to endotoxemia, which in turn activates Toll-like receptor 4 (TLR4) with production of cytokines and an inflammatory response, and subsequent development of obesity/alcohol-related liver disease (Hritz et al., 2008). Therefore, an understanding of the underlying molecular mechanisms of obesity/alcohol/HCV-induced hepatocarcinogenesis is essential for the eventual development of improved therapeutic modalities for this synergistic consequence.

By using mice with liver-specific expression of the HCV NS5A protein, it was discovered that feeding these mice alcohol for 12 months results in development of liver tumors in a manner dependent on TLR4 (Chen et al., 2013). We demonstrated that TLR4 is ectopically induced by the HCV viral protein NS5A in hepatocytes/hepatoblasts. These cells upon activation by circulating endotoxin experience induction of the stem cell marker NANOG, generating TLR4/NANOG-dependent, chemoresistant tumor-initiating stem-like cells (TICs), which can induce HCC in mice (Chen et al., 2013).

TICs are rare, highly malignant cells that are present in diverse tumor types and play a central role in tumorigenesis, malignant progression, and resistance to chemotherapy (Machida et al., 2009; Rountree et al., 2008). We previously characterized NANOG-dependent liver TICs from liver tumors resulting from ectopic activation of TLR4-NANOG pathway in alcohol-fed HCV transgenic mice. NANOG is one of core stemness factors, downstream of TLR4, and its pleiotropic contribution to the genesis and maintenance of TICs occurs via both upregulation of other stem cell factors (e.g., Sox2, Oct4, and CD133) and oncoproteins (YAP1, IGFBP3, and TBC1D15) and downregulation of tumor suppressors (p53, TGF-β). We have recently reported that sorafenib treatment made TICs more susceptible to tumor growth retardation to the point that the tumor size was reduced ˜55% when combined with knockdown of NANOG-inducible proto-oncogenes (YAP1: which induces antioxidant gene programs) (Chen et al., 2013). However, the underlying mechanism of chemoresistance and self-renewal of TICs are not fully understood.

Deaths due to metastatic hepatocellular carcinoma (HCC) continue to mount due to a low success rate of clinical intervention. HCC is the third most deadly cancer in the world (660,000 deaths per year). The incidence of HCC continues to rise with an estimated 33,660 new cases and 24,550 deaths in the US for 2015 and HCC remains an incurable malignancy with unmet medical need. One goal of targeted cancer therapy is to eliminate all malignant tumor-initiating cells (TICs) and/or circulating tumor cells (CTCs: a tiny fraction of blood cells, often fewer than one in a million) for the prevention of relapse and metastasis. Clinical evidence, however, reveals eventual chemoresistance to these drugs in HCC patients (Shen et al., 2008; Villanueva et al., 2008). The 3-year survival rates of 13% to 21% without any specific treatment (Barbara et al., 1992; Ebara et al., 1986). At present, only 10% to 23% of patients with HCC may be surgical candidates for curative-intent treatment (Shah et al., 2011; Sonnenday et al., 2007). The major challenge of chemotherapy is to find a means of overcoming recurrence mechanisms (stemness marker NANOG enrichment) and eliciting effective tumor killing responses targeted to TICs/CTCs. Understanding cancer inside out is the best way to fight the disease. However, genetic testing and precision medicine take lots of money, data, effort and time. Less than 5% of the 1.6 million Americans diagnosed with cancer each year can take advantage of genetic testing, which costs approximately $8,000. Around 70% of genetic testing does not have insurance coverage. To truly fight cancer, physicians need to understand it from the inside out, which means decoding its RNA/DNA.

The current standard of care involves using Sorafenib. Sorafenib is used as single FDA-approved chemotherapy agents for HCC (Huynh et al., 2009). Current diagnostic factors include contrast-enhanced studies (CT-scan or MRI) imaging with lesions greater than 1 cm, liver biopsy and alpha-fetoprotein (AFP) levels. AFP levels are insufficiently sensitive or specific for use as a diagnostic assay. If the AFP level is high, it can be used to monitor for recurrence. The current challenges associated with this standard of care are not accurate and it takes one week to diagnose.

### SUMMARY OF THE INVENTION

In one aspect, a method of identifying subjects with metastatic hepatocellular carcinoma (HCC) for tumor-initiating stem-like cell (TIC) or circulating tumor cells (CTCs) targeted therapy is disclose. The method comprises the steps of obtaining whole blood from a subject; retrieving CTCs and/or TICs from the whole blood; performing quantitative reverse transcriptase-PCR (qRT) PCR on retrieved CTCs and/or TICs; and identifying genes selected from the group consisting of NANOG, TWIST1, LIN28, MSI2, ACADVL, BIRC5, miR-22, LepR, YAP1 and IGF2BP3 that are upregulated and/or genes selected from the group consisting of COX6A2, COX15, TET1, TET2 and PTEN that are downregulated. Advantageously, the targeted therapy targets TICs.

In some embodiments, TICs are CD133+, CD49f+, and CD45−. In some embodiments, TICs the CTCs are CD45− and cytokeratins negative.

In some embodiments, upon the identification of one or more of the genes that are upregulated and/or one or more of the genes that are downregulated, a targeted therapy is initiated.

In some embodiments, the targeted therapy comprises inhibiting a NANOG pathway. In another embodiment, the targeted therapy comprises inhibiting a and Stat3 pathway.

In some embodiments, a chemotherapeutic drug is concurrently administered with the targeted therapy. In some embodiments, the chemotherapeutic drug is sorafenib.

In some embodiments, the targeted therapy comprises enhancing regeneration of mitochondrial oxidative phosphorylation (OXPHOS) genes or reactive oxygen species (ROS). In some embodiments, the targeted therapy further comprises concurrently administering a chemotherapeutic drug. In some embodiments, the chemotherapeutic drug is sorafenib.

In some embodiments, targeted therapy comprises inhibiting mitochondrial fatty acid oxidation (FAO). In some embodiments, the targeted therapy further comprises concurrently administering a chemotherapeutic drug. In some embodiments, the chemotherapeutic drug is sorafenib.

In one aspect, disclosed herein is a method for epigenetically modifying and eradicating tumor-initiating stem-like cells (TICs) in a subject in need thereof. The method comprises administering, to the subject, an effective amount of suberoylanilide hydroxamic acid (SAHA). In some embodiments, the method further comprise administering, to the subject, an effective amount of all trans retinoic acid (ATRA).

Other aspects and advantages of the invention will be apparent from the following description and the appended claims.

It will be understood that embodiments disclosed herein can be used in any combination.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. NANOG plays a critical role in liver oncogenesis. (A) Results of gene expression microarray comparing liver cancers arising from feeding of ethanol or Western diet (WD)-fed in HCV NS5A transgenic mice and WD+HCV Core transgenic (Tg) mice. Venn diagram shows genes associated with each etiology and those shared among two or more liver cancer models. (B) Summary of proteomic analysis of three mouse liver cancer models listed in A. All models showed similar metabolomic properties as shown in the Venn diagram. (C) Heat map showing more extensive proteomic signatures in liver cancer models: alcohol+NS5A; alcohol alone, high cholesterol high-fat Western diet (WD); WD+HCV core gene, and alcohol+HCV core gene. Animals used were either wt, transgenic for either NS5A or core, as indicated. (D) Western diet (WD) combined with alcohol increased tumor incidence in NS5A Tg mice compared to control. Upper panel-tumor incidence percentage. Lower panel-immunoblot of Nanog expression. Sh-Nanog Tg indicates animals receiving inducible transgene for NANOG silencing. (E) Liver tumor formation in NANOG and NS5A Tg mice. Upper panel, liver tumors arising from NS5A and Nanog showing contributions of alcohol+Western diet. Knockdown of NANOG (ΔLi), as indicated, reduced tumor incidence in wt control and NS5A mice. Lower panel-liver histology showing pathology is increased following Nanog knockdown in NS5A Tg mice. (F) NANOG ChIP analysis: comparison of promoter fragments from CD133⁻ and CD133⁺ cell populations. (G) Summary of gene ontology families identified by NANOG ChIP-seq analysis.

FIG. 2. The tumor incidence in several HCC mouse models is TLR4-dependent. (A) Effect of alcohol feeding on tumor formation in Core and NS5A transgenic mice. Upper panel summary of liver tumor incidence among experimental animals: wt, HCV-Core (Core) and HCV-Core+NS5A (Core/NS5A) transgenic mice. Lower panel—the effect of ethanol feeding to wt and transgenic mice on liver tumor development. (B) TLR4 is required for tumor development in Western diet fed (WD) HCV Tg mice. Upper panel-tumor incidence among wt and transgenic mice fed WD. Lower panel—the effect of WD on wt and Tg mice tumor development. (C) TLR4-dependent TICs from DEN/Phenobarbital (Pb) and human HCC (non-viral etiology: no HCV) models. Chimeric mice were generated by transplantation of BM from Tlr4+/+ or Tlr4−/− mice into irradiated Tlr4+/+ or Tlr4−/− mice. DEN-Pb-induced tumor incidence is reduced by recipient TLR4 deficiency but not by donor Tlr4 deficiency. (D and E) Effect of alcohol and WD feeding on plasma endotoxin levels in transgenic mouse genotypes. Alcohol WD feeding equally elevated plasma endotoxin levels in all genotype mice. Serum endotoxin levels are elevated equally in both Tlr4+/+ and Tlr4−/− mice fed ethanol or WD (D) or diethylnitrosamine (DEN)/phenobarbital (Pb) treatment (E). Left panel-effect of alcohol feeding on single and double-transgenic animals. Right panel-effect of WD feeding on single and double transgenic animals. Plasma endotoxin levels were measured in wt, HCV-Core, HCV-NS5A and Tlr4−/− double Tg strains of the wt and single Tg animals, as indicated. (F) TLR4 is induced in the liver tumor samples from the DEN/Phenobarbital HCC models. Activated TLR4 signaling is evident only in HCV Core fed Western diet (WD) as demonstrated by co-immunoprecipitation analysis showing TRAF6 interaction with TAK1. Induction of TLR4 and NANOG were detected in HCV Core Tg livers fed WD or DEN/Phenobarbital-treated mice. (G) Tlr4 mutant mice fed Western diet for 12 months have less NANOG protein levels.

FIG. 3. TLR4 signaling transactivates Nanog promoter through E2F1-binding sites. (A) Nanog promoter ChIP assay using anti-E2F1 antibody following qPCR in TICs. E2F1 showed enrichment in Nanog promoter. (B) Truncated promoter luciferase constructs were used to map the region responsive to TLR4 signaling. LPS-mediated Nanog promoter activity is compare with PBS (Vehicle)-treated cells to demonstrate Nanog promoter activity upon TLR4 stimulation (n=3). *: P<0.05, **: P<0.01. (C and D) E2F1-binding sites were required for efficient Nanog transactivation (n=4). Four mutant-luciferase plasmids were constructed by in vitro mutagenesis “M” indicates the sites of mutation. (D) E2F1 (blue oval) regulated Nanog enhancer for LPS-induced activation. (E) Overexpression of E2F1 resulted in NANOG promoter activation. Gfp, E2F1 or c-MYC were overexpressed in Huh7 cells and examined for luciferase reporter activities in response to LPS stimulation. (F) Silencing E2F1 reduced Nanog mRNA and protein levels in response to LPS. (G) Silencing E2F1 reduced tumor growth in NOG mice.

FIG. 4. NANOG reduced mitochondrial OXPHOS preventing mitochondrial ROS production. (A) Effect of NANOG on expression of selected OXPHOS enzymes. A representative model (inset) shows the putative relationship of Nanog silencing to corresponding increased OXPHOS gene expression in TICs. (B) NANOG ChIP-seq analysis identified mitochondrial OXPHOS genes are major NANOG regulated genes. (C) The Etomoxir (ETO: CPT1 inhibitor)-blocked component of oxygen consumption rate (OCR) and glycolysis inhibitor 2-deoxyglucose (2-DG)-blocked the glycolytic component of extracellular acidification rate. ETO inhibits CPT1 to block entry of fatty acid into mitochondria. (D) Seahorse assays demonstrated that NANOG silencing promoted increased oxygen consumption rate (OCR). Effects of oligomycin, FCCP, 2-DG or ETO, and antimycin/rotenone on OCR were evaluated in the Nanog-silenced TICs (sh-Nanog) and scrambled shRNA-transduced TICs. Real-time measurement of OCR showed that ETO but not 2-DG abrogated FCCP-induced OCR. NANOG silencing switched between FAO and glucose utilization (an adult-like metabolic pattern). (E) Silencing Nanog or Tlr4 reduced ECAR, demonstrating that inhibition of Nanog or Tlr4 reduced glycolytic activity. (Left and Right) ECAR of sh-Scr-TICs represented with a dark blue plot cells that were transduced with lenti-sh-Nanog is shown with a dark green line and lenti-sh-Tlr4 transduced cells are indicated by a light blue line (Left) ECAR represents the sum of FAO and glycolysis, respectively. ECAR measurement after ETO inhibition of β-oxidation showed a rapid decrease of glycolysis only in the sh-Nanog-TICs; yet, sh-Scr-TICs, ETO did not affect ECAR (glycolysis). (Right) ETO treatment does not affect ECAR in sh-Scr-TICs while ETO treatment inhibits ECAR in sh-Nanog-TICs, indicating that TICs are dependent on glycolysis, but inhibition of fatty acid import into mitochondria only inhibits glycolytic activity in sh-Nanog-TICs, but not sh-Scr-TICs. (F) ChIP-qPCR of NANOG in Cox6a2 promoter of TICs. (G) Truncation of Nanog promoter identified region responsive to NANOG-mediated inhibition (n=3, *: P<0.05). Promoter activity increased by deletion of the promoter segment containing critical cis-element(s). (H) Mutagenesis in NANOG binding sites (−1078 and −790) promotes Cox6a2 promoter activity (n=3, *: P<0.05).

FIG. 5. NANOG promoted mitochondrial FAO. (A) Hypothetical model of NANOG-mediated metabolic reprogramming. (B) NANOG ChIP-seq analysis identified FAO genes are NANOG regulated genes (i.e., Acadvl). (C) qRT-PCR and immunoblot analysis of NANOG-target FAO genes in sh-Nanog or Nanog-overexpressing TICs. (D) ChIP-qPCR of NANOG in the Acadvl promoter in TICs. (E, left) Acadvl promoter luciferase constructs used to map the region responsive to NANOG-mediated inhibition. (E, right) Protein levels of FAO enzymes (ACADVL) were measured by immunoblot in TICs expressing sh-NANOG. (F) Mutations in NANOG binding sites in ACADVL promoter reduced ACADVL promoter activity. (G) FAO was measured by incubation of extracts from sh-scrambled and sh-Nanog-TICs with [¹⁴C]palmitate; recovery of acid-soluble metabolites (G, left) and captured ¹⁴CO₂ (G, right) (n=5 per genotype, *P<0.05). (H) Overexpression of Nanog induced FAO oxidation rate. FAO was measured by incubation of [³H] palmitate in TICs transfected with NANOG-expression vectors and vector control one week after transfection of vectors expressing GFP or sh-Nanog (acid-soluble metabolites, n=3-4 per category). *P<0.05.

FIG. 6. NANOG inhibited mitochondrial fatty acid elongation and promotes AMP/ATP ratio increase with AMPKa phosphorylation. (A) Schematic diagram of the proposed role of NANOG in mitochondrial metabolic reprogramming. AMPK activation in the increased ratio of AMP/ATP leads to phosphorylation of ACC to reduce malonyl CoA levels and thus increase mitochondrial fatty acid uptake (via de-repression of CPT1). (B) NANOG ChIP-seq analysis identified that FAO elongation genes (i.e., Acly) were NANOG-regulated genes. (C) qRT-PCR analysis of representative genes associated with fatty acid elongation and synthesis. (D) Rate of fatty acid elongation was affected in Nanog silenced TICs using GC-MS with stable isotope ¹⁴C. The relative ratio of C18:1/C16:1 (oleate/palmitoleate) was determined from measured levels. (E) Abnormal reduction of unsaturated long-chain or polyunsaturated fatty acids (PUFA) in TICs compared to those in hepatocytes. Metabolomics analyses were performed on mouse TICs and control hepatocytes transduced with sh-Nanog or scrambled shRNA control (n=5 per group). (F) Adenosine 5′-monophosphate (AMP) levels increased in TICs whereas Nanog silencing reduced AMP level as determined from metabolomics analysis. (G) The sh-Nanog treatment of TICs affected phosphorylation of AMPKa and AMPKβ associated with ACC phosphorylation. (H) Phospho-AMPK level was increased in human tumor tissues.

FIG. 7. NANOG orchestrated TIC oncogenic and therapeutic resistance mechanisms via mitochondrial metabolic reprogramming. (A) Mitochondrial ROS production increased in sh-Nanog TICs, but total mitochondrial levels were unchanged in TICs compared to sh-Nanog TICs. (B) ROS inducer Paraquat (Para), but not ROS scavenger (NAC), inhibited spheroid formation, but minimal cell death induction was observed (C). (D) Restoration of OXPHOS genes in TICs promoted self-renewal ability. (E) Silencing OXPHOS genes and FAO genes inhibited spheroid formation. (F) Mitochondrial cytochrome c release was increased by the combination of sorafenib and ETO treatment or overexpression of Cox6a2 in TICs. Cytochrome c release from mitochondria was analyzed by immunoblotting of the cytosol (soluble fraction) and mitochondria-rich (heavy membrane: HM) fractions of the cell lysates. TICs and CD133(−) cells transduced with sh-Nanog were lysed and fractionated into purified heavy membrane (HM) and cytosolic (S) fractions. The fractions were then probed for cytochrome c (Cyt c), VDAC1 and Cu/Zn SOD. (G) Overexpression of Cox6a2 and ETO treatment abrogated drug-resistance and reduced tumor growth. (H) A summary diagram depicting the proposed roles of TLR4/NANOG for metabolic reprogramming and genesis of TICs in liver oncogenesis due to alcohol and HCV. NANOG-induced chemotherapy-resistance occurred via mitochondrial metabolic reprogramming (suppression of mitochondrial OXPHOS and promotion of FAO).

FIG. 8. Pathway analysis of proteomics of different liver disease models and validation studies of NANOG target genes identified by NANOG ChIP-seq. (A) Canonical pathways by proteomic analysis affected in tumor tissues of three different liver disease models. Note that NANOG-target genes are tightly linked to all three different HCC mouse models. (B) Canonical pathways affected in diseased livers in three different liver disease models. (C) NANOG enrichment proximal to initiation site of gene promoters in CD133(+) TICs, but not in CD133(−) cells. (D) qRT-PCR analysis of NANOG target genes in the presence or absence of NANOG or STAT3. TICs were transduced, as indicated, by lentivirus overexpressing Nanog (OE), shRNA targeting Nanog (sh-Nanog), retrovirus expressing dominant negative form of STAT3 (STAT3D) or constitutively active form of STAT3 (STAT3C). qRT-PCR analysis of NANOG target genes were performed. All experiments were conducted with TICs. P values represent two-tailed Student's t-tests between untransduced and transduced cells. Values for each cell line are means±S.D., n=4, *P<0.01, **P<0.001. Values for each cell line are means±S.D., n=4. (E) De novo Nanog-binding motifs resemble STAT3-binding motifs. (F) Silencing Nanog reduces STAT3-mediated transactivation in TICs. *P<0.01, **P<0.001. n=4.

FIG. 9. Validation of reconstituted bone-marrow-derived cells and TIr4- and Nanog-dependency of mouse TICs isolated from liver tumor model. (A) Immunostaining of liver sections of Western diet-fed mice. Immunofluorescence staining of Albumin (ALB), a-Fetoprotein (AFP) and TLR4 in liver sections from Western diet (WD)-fed mice. (B, left) Liver histology and tumor incidence of WD fed HCV-Core and HCV-Core/TIr4(−/−)Tg mice. Upper panel (left to right)-representative H&E stained liver sections from HCC-Core, typical examples of fatty liver, and TIr4−/− mice fed high fat diet. Nodular lesions differ from the surrounding liver parenchyma with cytological or structural atypia. (B, right) Frequencies of liver dysplastic nodules and HCCs in WT or Core Tg mice fed control diet or Western diet (WD) for 12 months. The histopathology of the tumors (arrows) shown are dysplastic nodules (DNs) or hepatocellular carcinomas (HCCs) based on their hypercellularity. (C) Validation of efficiency of reconstitution of bone marrow transplantation. Efficiency of reconstitution of bone marrow transplantation was confirmed by LPS-induced IL-6 mRNA expression in isolated splenocytes from TLR4-chimeric mice in comparison to that of untreated mice. (D) WT mice were treated with DEN followed by liposomal clodronate injection (n=18) or liposome vehicle injection (n=12) before phenobarbital feeding. Marker of Kupffer cell (liver-resident macrophage: Emrl) depletion and proliferation marker (Pcna) were evaluated by qPCR. (E and F) Tumor number and size of TIr4 WT (+/+) or deficient (−/−) mice with bone marrow transplantation of WT or TIr4 deficient cells injected with DEN and fed phenobarbital (Pb)-containing water. (G) We performed FACS-based isolation of CD133+/CD49f+ cells from liver tumors of alcohol-fed Core Tg mice. CD133+/CD49f+ cells from these models all have higher expression of stemness genes such as Cd133, Nanog, Oct4, and Sox2 compared to CD133− cells, and the inductions are abrogated by TIr4 silencing with lentiviral shRNA. CD133+/CD49f+ cells from the three HCC models, express stemness genes. (H and I) CD133+/CD49f+ cells form tumors in NOG mice in a manner dependent on TLR4 or NANOG. CD133+/CD49f+TICs isolated from human HCC have tumorigenic activities dependent on TLR4 and NANOG. Scr: Scrambled shRNA-transduced cells. Tumor-initiation property is increased in CD133+ cells and suppressed by TIr4 (H) or Nanog (I) silencing, demonstrating they are self-renewing. Subcutaneous transplantation of CD133+ cells but not CD133− cells transduced with a dsRed lentiviral vector, results in tumor formation in immunocompromised NOG mice, and the tumor growth assessed by dsRed imaging is attenuated by TIr4 or Nanog silencing with lentiviral shRNA prior to transplantation. These results are supportive that CD133+/CD49f+ cells are TLR4/NANOG-dependent TICs and that TIr4 is a putative proto-oncogene involved in the genesis of TICs.

FIG. 10. TLR4 stimulation transactivates NANOG through TAM and TBK1-mediated phosphorylation of E2F1 at serines 337 and 332. (A) TLR4 silencing effect was confirmed by immunoblot in Huh7.5.1 cells. (B) Role of TLR4 activation of Nanog promoter in human hepatocytes with HCV infection. HCV infection in Huh7.5.1 cells induced NANOG promoter activity in response to LPS stimulation. LPS transactivates Nanog through TLR4 signaling. All experiments were conducted with Huh7.5.1 cells. P values represent two-tailed Student's t-tests between untransduced and transduced cells. Values for each cell line are means±S.D., n=4, *P<0.01, **P(0.001. (C) Silencing of E2F1, TAB1 (TAK1-binding protein: MAP3K7), TBK1 or TAK1 was confirmed by immunoblots. Silencing E2F1, TAB1 or TBK1 reduces NANOG protein levels in TICs. (D) TLR4-mediated TAK1/TBK1 phosphorylation of E2F1 transactivated NANOG promoter in TICs. (D, right) Densitonnetric analysis of immunoblots bands. (E) TAK1 and TBK1 were required for efficient transactivation via E2F1 phosphorylation. Several shRNAs targeting E2F1-tranduced TICs were transfected with WT E2F1, E2F1(Ser332A1a), E2F1(Ser337A1a) and E2F1(Ser332Ala, Ser337A1a) and stimulated for LPS. (F) Silencing both TAK1 and TBK1 reduced E2F1 phosphorylation at two serine residues (Ser332 and Ser337) and reduced NANOG protein levels after LPS stimulation. (G) Non-phosphorylatable mutant of E2F1 does not induce NANOG expression in TLR-silenced Huh7 cells. (H) Expression of E2F1 (5332D/S337D) in TIr4-silenced TICs promoted NANOG protein expression. (I) Alanine substitution of phosphorylation sites (5332A, 5337A) abrogated phosphorylation of 5332 and 5337 following LPS stimulation. Several shRNAs targeting E2F1-tranduced TICs were transfected with WT E2F1, E2F1(Ser332A1a), E2F1(Ser337A1a) and E2F1(Ser332Ala, Ser337A1a) and stimulated by LPS. (J) Proposed model. Schematic indicates the predicted elements within the Nanog enhancer and promoter. Phosphorylation of E2F1 and/or overexpression of E2F1 may activate Nanog transcription at the enhancer and promoter.

FIG. 11. Silencing of TIr4 or Nanog promotes basal levels of oxygen consumption rate. (A) Maximum respiratory capacity, spare respiratory capacity, ATP production and basal respiration in sh-Nanog, sh-Tlr4 and sh-Scrambled TICs. Cells were treated with 2-deoxyglucose (2-DG) or ETO as indicated. *P<0.05. (B) Protein levels of NANOG and TLR4 were confirmed in sh-Scrambled, sh-Nanog or sh-Tlr4 TICs in the presence or absence of overexpression of NANOG or TLR4. (C) NANOG overexpression restored lower levels of basal respiration in sh-Tlr4-TICs while TLR4 overexpression did not return to basal level of OCR in sh-Nanog-TICs. Seahorse assays using sh-Tlr4 or sh-Nanog lentivirus-transduced TICs in the presence or absence of Nanog or TLR4 over-expression, respectively. NANOG overexpression in sh-77r4-TICs reduced OCR to a similar level as for sh-Scrambled-TICs. In contrast, overexpression of TLR4 in sh-Nanog-TICs did not reduce OCR at the level of sh-Scrambled TICs, indicating that TLR4-mediated NANOG induction mainly reduced OXPHOS (i.e., OCR levels) in TICs. *P<0.05.

FIG. 12. NANOG cooperates with PPARs to promote FAO of TICS. (A) qRT-PCR analysis of peroxisomal and mitochondria' FAO gene expression in mouse ESCs, primary hepatocytes or mouse TICs transduced with scrambled shRNA or sh-Nanog. Peroxisomal FAO gene Acaal (one of Nanog target genes) is not altered by Nanog silencing in TICs. (B-D) Tumor-bearing mice have higher levels of FAO genes, but lower levels of OXPHOS-related genes while mice without tumors have a similar gene expression profile with those of non-tumor parts of livers. (B) qRT-PCR in mouse tissues. n: numbers of liver specimens analyzed by qRT-PCR. (Note: due to lower tumor incidence of non-Tg mice fed Western diet (WO), the sample number was only one. (C) Staining data quantified by blind analysis by two board-certified pathologists. (D) Immunohistochemistry of mouse tissues. (E) qRT-PCR analysis of PPAR mRNA levels as indicated in sh-Scr-TICs or TICs with shRNA targeting of Nanog (sh-Nanog) or overexpression (O.E.) of Nanog. (F) Sequential ChIP-qPCR analysis of Acadvl promoter region. (G-H) PPAR6 physically interacted with NANOG. TICS co-transfected with PPARs and HA-tagged Nanog wild-type or the deletion mutants were lysed, immunoprecipitated by ct-HA antibody and assayed by immunoblotting with indicated antibodies. Western blots of total lysates or proteins immunoprecipitated with an anti-HA (IP HA) antibody were probed with antibodies. (H) Lysates of TICs transfected with HA or the indicated GST-tagged Nanog deletions were incubated for capture with HA-Sepharose or Glutathione-Sepharose. (I) PPARδ promotes FAO. Fatty acid oxidation rate was measured in the presence or absence of NANOG overexpression or PPARδ.

FIG. 13. Silencing Nanog promotes glutaminolysis pathway, ATP production and glucose flux in TICs judged by metabolomics analysis, qRT-PCR and stable isotope experiments. (A) Glutaminolysis pathway in TICs judged by metabolomics analysis. Amino acid levels by metabolomic analysis of media from CD133(−) and TICs transduced with scrambled shRNA or sh-Nanog. (A, right) (C) Representative metabolites of cells following metabolomics analyses. (B) Amino acid levels following metabolomic analysis of CD133(−) cells and TICs transduced with scrambled shRNA or sh-Nanog. (C) Representative metabolites of cells from metabolomics analyses. (0-F) Glutaminolysis pathway in TICs judged by qRT-PCR and FAO analysis. (D) qRT-PCR analysis of Gott and Glutathione reductase (Grs) in TICs transduced with Nanog, scrambled shRNA or sh-Nanog lentivirus. (E) Oxidation rate was measured in TICs transduced with lentivirus sh-Tlr4, sh-Nanog or sh-Scrambled in glucose-deficient (−), glutamine-deficient (−) or normal culture media. (F) NANOG-mediated glutaminolysis induces generation of antioxidant molecules, including glutathione (GSH) through activation of glutaminolysis-related enzymes, including GOT2 and Glutathione reductase (Gsr). (G) C2-C4 fragment relative abundance of glutamate (%) in TICs. M0, unlabeled glutamate; M1-M3, labeled glutamate in TCA cycle; M4, labeled glutamate derived from [U-¹³C₅, 2,5-¹⁵N₂]-glutamine taken up the cells. When [U-¹³C₅, 2,5-¹⁵N₂]-glutamine is taken up by cells, it loses the ¹⁵N on the 5th carbon and is converted to [U-¹³C₅, 2,5-¹⁵N₂]-glutamate, which loses the ¹⁵N on the 2nd carbon and becomes [U-¹³C₅]-glutamate after rapid equilibration with TCA cycle intermediate a-ketoglutarate. When glutamine and glutamate are analyzed by gas chromatography mass spectrometry under electronic impact ionization (E1), their TFA derivatives give rise to a C2-C4 (m/z 152) and C2-C5 (m/z 198) fragments (Lee, 1996). Thus, the [U-¹³C₅, 2-¹⁵N]-glutamate has a C2-C4 fragment of m/z 156 (M4; contains 3×¹³C and a ¹⁵N) and a C2-C5 fragment of m/z 204 (M5; contains 4×¹³C and a ¹⁵N), which represent the relative abundance of glutamine taken up by the TICs. On the other hand, [U-¹³C₅]-glutamate has a C2-C4 fragment of m/z 155 (M3; contains 3×¹³C) and a C2-C5 fragment of m/z 204 (M4; contains 4×¹³C). When the [U-¹³C₅]-glutamate enters TCA cycle metabolism, it will gradually lose the ¹³C carbon after each cycle to generate M2, M1, and MO C2-C4 fragment and M3, M2, M1, and MO C2-C5 fragments, which represent the TCA cycle activity (Lee, 1996). (H) Relative abundance of glutamate (%) in TICs. As shown in the table above sh-TLR4 or sh-Nanog silencing reduced glutamine uptake by the TICs as evident by decreased percentage of M3 and M4 glutamate (C2-C4) and M4 and M5 (C2-05) fragments. However, sh-Tlr4 or sh-Nanog silencing enhances TCA cycle activity as demonstrated by the increased generation of M0 and M1 glutamate in both fragments. Treatment of TICs with the irreversible inhibitor of the key ‘GABA shunt’ enzyme GABA transaminase, vigabatrin, does not reduce the percentage of labeled succinate, suggesting that sh-TIr4 or sh-Nanog silencing promotes glutamine oxidation in mitochondria. (I) TCA cycle of sh-Nanog TICs when compared to sh-Scrambled TICs. (J-M) Carbon flux analysis demonstrates that Nanog silencing induces glucose flux through pyruvate carboxylase, but not PDH pathway. Glucose flux analysis showed there is a slight increase in total glucose flux to TCA after Nanog silencing. (K) Nanog silencing results in >5% flux through PC, comparing to Scrambled TICs (sh-Scr-TICs) group. (L) In both Scrambled shRNA (sh-Scr) and Nanog silencing TICs (sh-Nanog-TI Cs) groups, glucose flux through PDH for oxidation is negligible. (M) Glucose uptake was not significantly changed. (N and O) ATP production is reduced in ETO-treated TICs. Relative ATP levels per cell were plotted in TICs transduced with (N) scrambled shRNA or (O) sh-Nanog. (P) ETO treatment reduced ATP production while glycolysis inhibitor (2-DG) does not significantly lower ATP production in TICs. (Q) Glutamine withdrawal did not significantly change ATP production (Left), but reduced cell growth rate in all TICs in the presence or absence of silencing of TLR4 or NANOG (Right).

FIG. 14. Restoration of OXPHOS andfor suppression of FAO reduce the tumor growth and drug resistance. (A-B) ROS production is increased by glutamine removal in TiCs regardless of silencing of Nanog or TIN. (A) Glutamine withdrawal induced ROS production in at TICs in the presence or absence of silencing of TLR4 or NANOG. (B) Quantified data are plotted. (C) Nanog silencing reduced spheroid formation through ROS production in TICs. (D-H) NANOG orchestrates in TICs the oncogenic and therapeutic resistant mechanisms that result from mitochondria) metabolic reprogramming. (D) Overexpression of COX6A2 or COX15 were confirmed by immunoblots. (E) Restoration of OXPHOS genes (COX6A2 and COX/5) in TICs promoted OCR (E). (F) Silencing effects of shRNA targeting Acadvl or Echs1 were confirmed by immunoblots. (G) Silencing Acadv1 and Echs1 reduced rate of FAO. (H) Correlation of NANOG-mediated suppression of FAO and role in tumor formation. Mouse TICs were transduced with lentiviral Cox6a2 gene and were subcutaneously transplanted into NOG mice. These mouse TICs were very resistant to the growth inhibitory effect of sorafenib. By contrast expression of Cox6a2 abrogated this resistance and reduced tumor growth in comparison to vehicle treatment alone or control vector group. This demonstrated that overexpression of OXPHOS gene (Cox6a2) and inhibition of FAO by blocking entry of fatty acids into mitochondria (ETO) sensitized TICs to sorafenib and inhibited xenogratt tumor growth engrafted with mouse TICs in mouse recipients. (1-M) COX6A2 silencing or ACADVL overexpression promoted self-renewal ability and NANOG expression in human HOC cell lines. (I and J) Silencing of COX6A2 proteins with shRNA lentivirus transduction or overexpression of ACADVL or PPARD was confirmed by immunoblot analysis (I: HepG2 and J: Hep3B). (K and L) Reduction of OXPHOS gene (e.g., COX6A2) or overexpression of FAO gene (e.g., ACADVL) in human non-TIC HCC cell lines (HepG2 and Hep3B) promoted self-renewal ability (K), NANOG expression (L) and viability in response to sorafenib treatment (M), as judged by spheroid formation assays, qRT-PCR analysis of NANOG and MTT staining assays respectively following sorafenib treatment (10 uM, 48 hours).

FIG. 15. Simplified workflow for the 15 RNA profiling-based diagnosis vs. conventional single-cell DNA sequencing-based diagnosis. Results are rapidly obtained while conventional NGS-based diagnosis will need approximately 1 week to confirm the presence of cancer cells by pathologist using a microscopy. Automated diagnosis eliminates accuracy disparities among medical institutions.

FIG. 16. Accentuated expression of TWIST1 and NANOG in human patient samples. (A) Tissue microarray analysis confirmed the correlation of TWIST1 and NANOG in a large number of patient HCC tumor samples (n=116 samples, paired). (B) Multivariate analysis of survival data based on the Cox proportional hazards model indicated. HR, hazard ratio; CI, confidence interval.

FIG. 17. Identification of selective FDA-approved drug combination. (A) Models of NANOG enrichment in tumor immunity. High-throughput screening of FDA-approved drugs. (B-D) A novel FDA-approved drug combination was identified from high-throughput screening of drugs that selectively kills TICs and inhibits Nanog promoter activity. (E) ATRA+SAHA increases survival rate of xenografted NSG mice.

FIG. 18. Stem cell signature and CTC number in blood are prediction for poor prognosis. A computer-assisted method was used to determine the threshold level between positive and negative expression and compared the clinical outcome of HCC patients in the three groups containing 10 patients with stemness signature (A) and another 10 patients with non-stemness signature (B). (C and D) The survival outcomes of human HCC patients was analyzed after stratification into distinct gene-expression subsets, based on the expression of 15 RNA sets. (E) A multivariate analysis indicated that the 15-gene grouping system had the prognostic value and even better if number of CTC is considered together.

FIG. 19. NS5A Tg mice fed high-cholesterol high-fat diet (HCFD) with or without LPS frequently developed tumors (A) Summary of WT and Tlr4^{-/-} HCV-NS5A Tg mice fed control diet or HCFD with or without LPS from 8 weeks of age for 12 months; N, number of experimental mice (WT-HCFD; *, P<0.05**, P<0.01***, P<0.005, green scripts and symbols—statistical analysis in comparison to LFD, purple scripts and symbols—statistical analysis in comparison to HCFD). (B) Plasma endotoxin and leptin levels in mice fed low-fat diet (LFD) or HCFD. (C) Gross images of non-pathological liver from control diet (1, 2) and liver tumor with multiple nodules from HCFD (3-6) and HCFD+LPS (7). Lower panel shows histology of respective groups. The HCFD tumor shown (arrow) is a dysplastic nodule. (D) Frequencies of liver dysplastic nodules and HCCs in LFD- or HCFD-WT or NS5A Tg mice fed LFD or HCFD for 12 months. Representative H&E staining of tumor sections from WT or NS5A Tg mice fed HCFD or LPS+HCFD. The histopathology of the tumors (arrows) shown are dysplastic nodules (DNs) or hepatocellular carcinomas (HCCs) based on their hypercellularity. Nodular lesions differ from the surrounding liver parenchyma with cytological or structural atypia. (E) Normal liver/liver tumor lysates from WT and NS5A Tg mice fed control chow or HCFD were analyzed for LPS-induced TLR4 signaling. Upper panel, TRAF6 interaction with TAK1, was enhanced in NS5A Tg mice fed HCFD. The interaction between TAK1 and TRAF6 were examined by immunoblots post immunoprecipitation (IP) with TAK1 antibody. As a positive control (shown in last three lanes) mice were challenged with LPS; LPS injected (2 mg/kg) 30 mins, 1 or 2 hours, respectively, before liver tissues were collected for analysis. The relative densitometry units and details are available in supplementary FIG. 1A. Bottom panel, LPS-induced phosphorylation of IKK-β in the liver was increased in NS5A Tg mice fed HCFD. Positive controls (last three lanes), as explained previously. (F) Data summary of body weight changes over 12 month feeding period and statistics are available in (A). The scale bar equals 50 μm.

FIG. 20. TLR4-mediated TWIST1 induction. (A) Chief summary of RNA microarray analysis. Twist1, key regulator of EMT signaling was significantly higher in NS5A+HCFD compared to WT+HCFD. (B) Quantitative analysis of Twist1 from liver/liver tumor tissues of all cohorts, as listed in FIG. 1 A. Heightened Twist1 expression (NS5A Tg mice fed HCFD) was abrogated by TLR4 deficiency. Data normalized to GAPDH expression are listed as the fold change (*, P<0.05). (C) LPS induced TWIST1 in Huh7 cells transduced with an NS5A expression vector (*, P<0.05 compared to cells transduced with an empty vector). This was suppressed by lentiviral expression of shRNA for TLR4 and also in cells transduced with the dominant negative TLR4 vector (TLR4ACyt). (D) LPS induced TWIST1 promoter activity. Huh7 cells transfected with TWIST1 promoter-luciferase construct were stimulated with LPS (10 μg/ml) in culture. Other experimental procedures in this figure are the same as described earlier. TLR4 knockdown or mutation abrogated TWIST1 promoter activity, but adding TLR4 rescued it. Relative light units (RLU) values were normalized by the Renilla luciferase activity driven by SV40 promoter which were used a transfection control (*, P<0.05).

FIG. 21. Twist1 is required for mesenchymal morphology of TICs, down regulation of Twist1 reduces TIC cancer-initiating property. (A) CD133+/CD49f+/CD45− cells were isolated from tumors of two different HCFD-fed NS5A Tg mice and examined for stemness gene expression by qRT-PCR. (B) To silence Twist1 expression, lentivirus shRNA Twist1 was transduced in TICs. Immunoblot analysis confirmed decreased TWIST1 expression in TICs and demonstrated unchanged expression of NANOG and TLR4 (n=3). (C) mRNA levels were validated by qRT-PCR. Expression profile of EMT-regulated genes, including mesenchymal markers (Twist1 and N-cad) and epithelial markers (Albumin and E-cad) were analyzed (n=3; *, P<0.05). (D) Light field microscopy demonstrated an altered morphology of TICs post Twist1 knockdown. Scrambled TICs (1) parenchymal cell phenotype drastically changed to a tadpole shape (2) post-Twist1 knockdown (40×; n=10; insets are enlarged images). In vitro oncogenicity was tested via soft agar colony formation assay. Silencing Twist1 in TICs (4) significantly reduced colony forming ability in contrast to control cells (3). The number of colonies formed were normalized and summarized (n=3; *, P<0.05). (E) shRNA knock down of Twist1 diminished the ability of TICs to effectively migrate in contrast to the scrambled shRNA control, as demonstrated by in vitro cell migration assay. The images were captured at 0 hours and 24 hours after scratching the cell layer with a 100 μl pipet tip (n=3; *, P<0.05). (F) Analyses at day 35 post TICs transplantation (subcutaneously injected into NOG mice). Twist1 silencing reduced the overall tumor volume (1) and weight (2). (3) Gross image of subcutaneous tumors. (4) Non-invasive bioluminescence imaging demonstrates the decrease in overall tumor growth (n=4 NOG mice/cohort; *, P<0.05). (5) H&E staining of xenografted tumor in NOG mice shows HCC histology. The scale bar equals 50 μm.

FIG. 22. NANOG and STAT3 influence Twist1 promoter activation in NS5A TICs. (A) LPS-induces Twist1 promoter activity in TICs. Twist1 promoter analysis with various deletion constructs demonstrated the importance of the TSS proximal segment (nt −209/−1). Relative light units (RLU) values were normalized by Renilla luciferase activity driven by a constitutively active SV40 promoter (pTwist1 nts −1 to −700; *, P<0.05; color matched; pTwist1 nts −1 to −209; #, P<0.05; n=3). (B) NANOG and STAT3 activate the Twist1 promoter. NANOG and STAT3 binding elements in Twist1 promoter region (nts −209 to −51) were mutated by in vitro mutagenesis (pTwist1 1-209WT; *, P<0.05; color matched; n=3). (C) Silencing Tlr4 and Nanog using lentivirus expressing shRNA or Stat3 and Stat3D (retrovirus expressing dominant negative Stat3). *, P<0.05; color matched; n=3). (D) Upper panel, schematic representation (SR) of Twist1 promoter region depicting the locations probed for the consensus binding sequences for NANOG (yellow script), STAT3 (Green lettering), and the specificity control (SC) regions analyzed by CUP (white script). Immediately below the SR are NANOG ChIP-qPCR (black bar graphs) and STAT3 ChIP-qPCR (blue bar graphs) analyses which demonstrated the enrichment of NANOG and STAT3 in TICs post LPS (10 μm/ml) and leptin (5 ng/ml) treatment. The Fold enrichment values are relative expression values normalized to the IgG controls (SC3; *, P<0.05; SC2; $, P<0.05; SC1; #, P<0.05; biological replicates 4; n=2). (E) Protein-Protein-DNA interaction demonstrated by sequential-ChIP-qPCR, indicated that NANOG and STAT3 bind each other on the Twist1 promoter region in TICs post LPS (10 μg/ml) and leptin (5 μg/ml) treatment. The Fold enrichment values are relative expression values normalized to the IgG controls (SC3, SC2, SC1; *, P<0.05; color matched; #, P<0.05; biological replicates 4; n=2).

FIG. 23. Induction of NANOG, pSTAT3 and TWIST1 in HCFD and HCFD+LPS NS5A Tg cohorts. Confocal immunofluorescence (IF) microscopy demonstrated co-localization of TWIST1 with (A) NANOG and (B) pSTAT3 (C) Co-localization of pSTAT3 with NANOG in tumors obtained from HCFD and HCFD+LPS NS5A Tg liver specimens; this immunoreactivity is completely absent in low fat diet liver tissues (magnification 40×oil; n=15 samples/cohort; n=3). Quantifications of the IF data was done using Metamorph software. The scale bar equals 50 μm.

FIG. 24. Accentuated TWIST1 co-localization with TLR4, P-STAT3 and NANOG in human patient samples. (A) Confocal immunofluorescence (IF) imaging studies demonstrated TLR4, P-STAT3, and NANOG often colocalized with TWIST1 in HCC patient liver specimens (Tumor), but absent in noncancerous liver tissue (adjacent) (magnification, 40×oil; n=8 samples/cohort; n=3; Red boxed are cropped images). (B) Paired IHC staining performed at USC corroborated with IF, which demonstrated the significant increase in NANOG and TWIST1 expression in HCC tumor samples (100× magnification; n=18 samples, paired; n=3). (C) Tissue microarray analysis confirmed the correlation of TWIST1 and NANOG in a large number of patient HCC tumor samples (100× magnification; n=116 samples, paired). Adjacent=parent non-cancerous liver, Tumor=human HCC. The liver is removed in order to transplant new liver. (D) In silico analysis using Oncomine™ Gene browser, probing for Twist1 correlation with grade, survival and relapse in HCC patients via Guichard libraries. The scale bar equals 50 μm.

FIG. 25. Twist1 overexpression drives tumor growth independently of Tlr4. TICs were transduced with lentivirus expressing shRNA for Tlr4 or scrambled shRNA followed by a second transduction with retrovirus expressing Twist1 overexpression (OE) plasmid vector or empty vector (Emp). These cells were injected subcutaneously into the rear flanks of NOG mice (1 million cells/injection). (A) Tumor volume measured at day 15, 25 and 30 (also whenever an unexpected death occurred) demonstrated an increasing trend in the tumor volume with intact Tlr4 and Twist1 overexpression when compared to their respective controls (sh-Tlr4+OE vs sh-Tlr4+Emp; ***, P<0.001; n=4 NOG mice/cohort; n=2; statistics performed using 2-way ANOVA). (B) Significant increase in the overall tumor weight (***, P<0.001, ****, P<0.0001, n=4 NOG mice/cohort; n=2). (C) Overexpression of Twist1 promotes Liver and Lung metastasis irrespective of the endogenous Tlr4 expression in TICs. (D) A schematic representation of the proposed link between oncogenic TLR4/NANOG signaling, OB-R/pSTAT3 and an effective TWIST1 pathway in generating TICs.

FIG. 26. Fold change in Twist1 gene expression in TICs. Quantification of TRAF6 expression from FIG. 19E using Image-J.

FIG. 27. RNA microarray analysis. Microarray gene expression data showing expression level differences of NS5A+HCFD vs. Non-Tg+HCFD.

FIG. 28. Immunoflurorescence staining on mouse liver sections for TLR4 and NANOG. Note: The TLR4 staining in the low fat diet (LFD) represents non-parenchymal cell (presumably such as Kupffer cells), whereas in liver sections from mice fed HCFD and in mice fed HCFD+LPS, the TLR4 is co-stained with NANOG representing that the origin of TLR4 in these livers is from both TICs and non-parenchymal cells. The scale bar indicates 50 μm.

FIG. 29. Twist1 is required for the mesenchymal phenotypes, cell proliferation, and self-renewal abilities of TICs. (A) Flow cytometry analysis (forward scatter affected by cell size) indicating the change in cell size after Twist1 knockdown in TICs (B) Analysis of cell number and viability post-infection with Lentivirus expressing sh-Twist1 or sh-scrambled into TICS; significant decrease in both cell number and viability was seen with the infection of the former compared to the later. *P<0.05, n=5. (C) Sphere formation assay demonstrated the significant decrease in the number of spheroids formed when the Twist1 gene is silenced in TICs. *P<0.05, n=3.

FIG. 30. TWIST1 promoter activation in Huh7 cells. TWIST1 promoter analysis with deletion constructs demonstrates the importance of the proximal segments (−209 to −51) in LPS-induced TWIST1 promoter activity.

FIG. 31. Induction of TLR4/NANOG/P-STAT3/TWIST1 pathway components in mouse HCC. (A) TLR4, STAT3, P-STAT3, NANOG and, TWIST1 protein levels are increased in HCC specimens from HCFDNS5ATg mice, as compared with cirrhotic or healthy livers, N=5 samples/cohort, n=3 (B) Twist1 (C) Nanog (D) Stat3 mRNA profiling showing significant increase in HCFD NS5A Tg mice Liver tumor, in contrast with the healthy livers, *p<0.05, N=8 samples/cohort, n=3.

FIG. 32. Immunofluorescence staining of mouse liver sections for TLR4, NANOG, TWIST1, AFP, CD133 and CD49F. Note: Staining of TLR4, NANOG and TWIST1 is reduced in liver sections of Tlr4−/−NS5A Tg mice fed HCFD for 12 months in comparison to those of wild type and NS5A Tg mice or mice fed low fat diet (LFD). Parenchymal cells of NS5A Tg mice fed LFD have diffuse staining of TLR4 while hepatocytes of wild type mice fed LFD have TLR4 staining mainly in non-parenchymal areas. Note: Liver sections from NS5A Tg mice fed HCFD have co-expression (yellow) of TLR4 (green) and AFP (red) or NANOG (red) mainly in parenchymal cells (with larger nuclei) or TICs.

FIG. 33. Immunofluorescence analysis of TLR4 and hapatocyte marker in NS5A Tg and wild type mice. The major source of TLR4 in the liver of wild type mice is from non-parenchymal cells, including the Kupffer cells and stellate cells. The low fat diet (LFD)-fed wild type mice have staining shows TLR4 positive cells, which are presumably Kupffer cells and stellate cells. Liver section of HCFD-fed NS5A Tg mice have TLR4 and NANOG-TLR4-double-positive cells, indicating that TLR4 origin is not only from Kupffer cells and stellate cells but rather from the TICs or hepatocytes. In liver of NS5A Tg mice, both parenchymal and non-parenchymal staining of TLR4 are positive while non-parenchymal area of wild type mice fed LFD mainly have positive staining of TLR4, indicating that hepatocytes and TICS of NS5A Tg mice have elevated levels of TLR4.

FIG. 34. Induction of TLR4, pSTAT3 and TWIST1 in human HCC. (A) Quantification of immunoperoxidase staining using Metamorph software showed increased staining intensity for Twist1, TLR4 and pSTAT3 in human liver tumors compared to the adjacent non-tumorous livers from Liver Tissue Cell Distribution System (LTCDS) of University of Minnesota (UMinn) (40× magnification; n=8 sampfes, paired). (B) Analysis of TCGA data of Twist1 mRNA levedifferent stages of HCC grades and patient survival days with levels of TWIST1 expression.

FIG. 35. Summary of exemplary clinical patient dataset used in the study.

FIG. 36. Twist1 by its very nature promotes tumor formation. (A) Twist1 mRNA was analyzed using qRT-PCR in TICs post TIr4 silencing and Twist1 overexpression, n=3. (B) Tlr4 mRNA was analyzed using qRT-PCR in TICs post TIr4 silencing and Twist1 overexpression, n=3.

FIG. 37. Identification of selective inhibitors for cancer stem cell population. (A) Schematic diagram of drug screenings. To find the most effective inhibitors for TICs, three different screening were conducted: CD133 cell viability screening, Nanog-GFP reporter cell screening and combination screening. (B) For CD133 cell viability screening, human HCC cell line, Huh7, were freshly sorted into two populations, CD133 (+) and (−) cells for used in drug screening. Huh7 cell consistently have 50-60% CD133 (+) cells (B, right panel). (C, D) For CD133 cell viability screening, most compounds tested showed similar effects on CD133 (+) and CD133 (−) cells (R²=0.8) while two compounds (red dots) show specific growth inhibition effect on CD133 (+), but not CD133 (−) cells. One is all-trans retinoic acid (ATRA) and the other one is the secondary generation of retinoic acid, acitretin (D) (n=3, *p<0.05). (E) Left panel—For Nanog-GFP screening, a reporter cell line was established by transducing lentiviral Nanog-GFP reporter in TICs. After antibiotic selection, the reporter cells were sorted into high (top 20%) and low (bottom 20%). Right panel—The GFP^{high} population has higher levels of Nanog staining when compare to that of low population (n=3, *p<0.05). Z-score distribution of drug library candidates (not shown). Candidates selected for repression of Nanog must have a z-score<−1.0 (red square). (F) The hits selected from Nanog-GFP screening showed down-regulation of Nanog gene expression (bottom panel).

FIG. 38. Drug combination treatment induces TIC apoptosis and reduces the self-renewal ability of TICs in vitro. (A) To verify the type of cell death induced by drug combination treatment (5 μg/ml of ATRA with 0.5 μg/ml of SAHA), cell apoptosis was detected by using of Annexin V and propidium Iodide double-stainings at each time point assayed (0, 8, 16 and 24 hrs). Annexin V-positive staining was observed as early as 8 hrs after treatment. (B-E) To determine the drug combination treatment which best induced apoptosis pathways, caspase activities of both extrinsic (death receptor; caspase-8) and intrinsic (mitochondrial; caspase-9) pathways were examined and it was found that ATRA induced both and further activated caspase-3 (C). The tumor spheroid assay (D) and anchorage independent colony formation assay were also performed (E) and it was found that the drug combination treatment reduced colony number significantly (n=3, * p<0.05). (C: Control, R: ATRA, S: SAHA, RS: combination treatment)

FIG. 39. Genome-wide transcriptome analysis of drug treated TICs. (A) To comprehensively illustrate the regulation network of drug treatment on TICs, RNA sequencing of TICs treated singly with ATRA or SAHA or in combination was performed. The gene expression heat map (upper panel) and gene expression profile (lower panel). (B) Principal component analysis of RNA sequencing data shows that the gene expression pattern of ATRA treatment only (red) is relatively similar to control (purple). Moreover, the gene expression pattern of SAHA treatment only (green) or combination treatment (blue) is quite different from the control group. (C) Differential gene expression in the three treatment groups presented as a Venn diagram. This shows that there are unique sets of genes in the drug combination groups which may play a critical role in regulating TICs. (D) GSEA analysis shows that the stem cell up-regulated gene set is highly enriched in control group, but not drug combination group, indicating that the combination treatment inhibits the stemness of TICs. (E) GSEA analysis shows the regulation of apoptosis gene set is highly enriched in the drug combination group, indicating the combination treatment induces TIC apoptosis. (F) The Venn diagram shows the drug combination affected 11% of Nanog target genes that is highly associated with regulations of cell cycle and cell survival pathway of TICs (G).

FIG. 40. Drug combination induced the PTEN pathway. (A) Heatmap of pathway comparison among ATRA, SAHA and combination (R+S) groups (top ranking) (left panel). In addition, drug combination treatment suppresses the Toll-like receptor signaling pathway that it was previously shown that the pathway plays a vital role for TICs (right panel). More detail is shown in Fig S4A. (B) Western blot data showing that the drug combination induced PTEN expression, leading to suppression of AKT phosphorylation and induction of FOXO1/3/4. (C) Activation of FOXOs by the drug combination treatment induced cyclin-dependent kinase inhibitors, p15^{INK4b}, p19^{INK4d}, p21^{Cip1} and p27^{Kip1}, leading to suppression of cyclins (Cyclin E and Cyclin D1) and cyclin-dependent kinases (CDK2). (D) Activation of FOXO induced by drug combination activates the BIM apoptosis pathway.

FIG. 41. Analysis of the unique set of gene in drug combination group. (A) Heatmap of gene expression patterns showing unique sets of genes activated in response to individual drug treatments and dual drug combination. (B) Ingenuity Pathway Analysis of the unique set of genes activated in the drug combination group shows that these genes are highly associated with cancer pathways and DNA repair pathways. (C) Among the unique set of genes, miR-22 was down regulated by the drug combination group. (FPKM stands for fragments per kilobase of transcript per million). (D) Silencing by shmiR-22hg significantly reduced TIC growth (n=3, *p<0.05). (E) Silencing by shmiR-22hg reduced the number of sphere significantly (n=3, *p<0.05). (F) Silencing by shmiR-22hg significantly reduced the number of colonies appearing in soft agar assay (n=4, ***p<0.001). (G) Silencing by shmiR-22hg rendered TICs more susceptible to conventional chemotherapy drugs (n=3, *p<0.05). (H) GSEA analysis shows that the up-regulated gene set in response to Rapamycin was similar to genes upregulated by ATRA+SAHA.

FIG. 42. Drug combination altered DNA methylation of Nanog in TICs. (A) Drug combination induces Tet2 gene expression (n=3, *p<0.05). (B) Drug combination treatment activated the TET2 3′UTR luciferase reporter (n=3, *p<0.05). (C) Drug combination down regulated OCT4 and the upstream regulator of p53, SIRT1. In contrast, the drug combination induces p53, TET2 and DNMT3A. (D) Bisulfite sequencing of Nanog promoter in the presence or absence of ATRA and/or SAHA treatment. In TICs, the p53 binding site of Nanog promoter was highly methylated; however, the OCT4 binding site was less methylated. Drug combination treatment reduced the methylation of the p53 binding site of Nanog, but increased the methylation of OCT4 binding site of Nanog (* p<0.05). C: Vehicle control, R: ATRA, S: SAHA, RS: ATRA+SAHA combination treatment. (E) ChIP-qPCR shows that TET2 and p53 were recruited to Nanog promoter but DNMT3A was removed from Nanog promoter after drug combination treatment. In contrast, DNMT3A was recruited to OCT4 binding site but TET2 and OCT4 were removed from the Nanog promoter after drug combination treatment (n=3, * p<0.05). (C: Control, R: ATRA, S: SAHA, RS: combination treatment).

FIG. 43. Drug combination suppressed tumor growth in vivo. (A) Treatment regimen designed to target CD133 (+) TICs. ATRA was encapsulated into nanoparticles displaying a conjugated anti-CD133 antibody. Tumor growth was reduced in the drug combination group (red), but not by single ATRA (blue) or SAHA (green) or control group (black) (C: Control, R: ATRA, S: SAHA, RS: combination treatment). (B) Histological analysis of tumors from each group showed that the drug combination induced extensive cell death in the tumor. (C) TUNEL staining of tumors from each group shows that the drug combination induced cell apoptosis in the tumor. (D) GSEA analysis shows that tumor recurrence-associated gene set is highly enriched in the control group, but not in the combination treatment group. This result indicates the combination treatment suppresses the recrudesce-associated gene set. (E) GSEA analysis shows that the gene set-associated with poor survival is highly enriched in control group, but not in the combination group. This data indicates the drug combination treatment might improve the overall survival rate. (F) Hypothetical Model: Combined drug treatment down-regulates miR-22, leading to activation of PTEN-FOXO apoptosis pathway and TET-mediated demethylation of p53-binding sites within the Nanog promoter. Specifically, TET2 is recruited to p53-binding sites of the Nanog promoter while DNMT3A is recruited for methylation of an OCT4 binding site within the Nanog promoter, leading to repression of Nanog.

FIG. 44. (A) The two hits selected from CD133 screening, ATRA and actretin, showed specific CD133 killing effect in dose dependent manner. (B) The hits selected from Nanog-GFP screening showed down-regulation of Nanog gene expression. (C) One of combination of ATRA with Nanog hits showed the killing effect in various HCC cells.

FIG. 45. The drug combination of ATRA and SAHA show growth inhibition effect in various HCC cells, such as Huh7, Hep3B, HepG2, human CSCs and mouse CSCs. However, The normal adult stem cells (mouse mesenchymal stem cells) were less sensitivity to this drug treatment.

FIG. 46. (A) Gene set enrichment analysis of ATRA and SAHA treatment only, showing the single treatment activated the specific downstream target genes. (B) Ingenuity Pathway Analysis for the unique set of genes in ATRA treatment only group and SAHA treatment only group. (C) Ingenuity Pathway Analysis shows the combination treatment reduced embryonic stem cell pluripotency pathways.

FIG. 47. (A) Heatmap of pathway comparison among ATRA, SAHA and combination (R+S) groups. (B) IPA analysis of comparison of drug combination treatment to control. These data show the combination treatment down-regulated NFkB pathway. (C) IPA analysis show the drug combination induced PTEN-FOXO axis. Oncomine in silica analysis shows PTEN is suppressed in HCC patients (not shown). (D) PTEN promoter luciferase activity assay shows the combination treatment induced PTEN activity (n=3, ***p<0,01)

FIG. 48. Immunofluorescent staining for NANOG, p53, TET2 and DNMT3A. These data shows that the drug combination reduced NANOG expression and induced p53, TET2 and DNMT3A expression.

FIG. 49. (A) Schematic diagram of Nanoparticle production. The ATRA was encapsulated into PLA-PGE-Maleimide. In addition, the surface of particle was conjugated with a specific CD133 antibody. (B) The size of nanoparticle was measured by Particle Analyzer (Delsa™ Nano). (C) Western blot analysis showed the CD133 antibody conjugation with nanoparticles.

### DETAILED DESCRIPTION OF THE INVENTION

Unless otherwise noted, terms are to be understood according to conventional usage by those of ordinary skill in the relevant art.

In one aspect, disclosed herein are methods for identifying subjects with metastatic hepatocellular carcinoma (HCC) for tumor-initiating stem-like cell (TIC) or circulating tumor cells (CTCs) targeted therapy.

The identification is achieved through expression analysis to identify key genetic markers who expression level varied drastically when compared with controls. In some embodiments, a control is a known healthy subject who does not have HCC. In some embodiments, expression levels used as controls are computed averages based on multiple known healthy subjects to even out statistical variations with a population. In some embodiment, a preferred control may be a healthy subject who is genetically related to a diseased subject with HCC. The goal for this approach is to minimizing the effects of genetic variations.

In preferred embodiments, blood samples (e.g., whole blood samples) are collected from a patient. For example, RNA extracts from the blood samples are then analyzed for expression levels and compared with those of one or more controls. The amount of RNA associated with a particular gene can be quantitated, for example, by quantitative reverse transcriptase-PCR (qRT) PCR. In some embodiments, quantities of RNA molecules are determined by image analysis using labelled probes.

In some embodiments, protein expression levels are measured. In some embodiments, tissue sample may be used for expression level analysis. Here, a number of approaches are possible. RNA extract can be purified from the tissue sample. Or alternatively, the tissue sample can be analyzed by in situ method such as fluorescence hybridization to measure RNA expression level.

A gene is considered upregulated when its expression level is significantly more than that of a control sample, for example, above a threshold level that is beyond the extent of statistical variation. In some embodiments, the threshold level is met when an expression level of a gene in a patient is 5% or more, 10% or more, 15% or more, 20% or more, 25% or more, 30% or more, 40% or more, 50% or more, 60% or more, 75% or more, 100% or more, 150% or more, 200% or more, 500% or more, than the expression level of the same gene in a control subject.

A gene is considered upregulated when its expression level is significantly less than that of a control sample, for example, below a threshold level that is beyond the extent of statistical variation. In some embodiments, the threshold level is met when an expression level of a gene in a patient is 5% or less, 10% or less, 15% or less, 20% or less, 25% or less, 30% or less, 40% or less, 50% or less, 60% or less, 75% or less, 100% or less, 150% or less, 200% or less, 500% less more, than the expression level of the same gene in a control subject.

Exemplary genes that are upregulated in a patient with metastatic hepatocellular carcinoma (HCC) for TIC or CTC targeted therapy include but are not limited to NANOG, TWIST1, LIN28, MSI2, ACADVL, BIRC5, miR-22, LepR, YAP1 and IGF2BP3.

Exemplary genes that are downregulated in a patient with metastatic hepatocellular carcinoma (HCC) for TIC or CTC targeted therapy include but are not limited to COX6A2, COX15, TET1, TET2 and PTEN.

An HCC patient is suitable for TIC or CTC targeted therapy when the patient has at least one upregulated gene among the group of upregulated genes disclosed herein. Alternatively, an HCC patient is suitable for TIC or CTC targeted therapy when the patient has at least one downregulated gene among the group of downregulated genes disclosed herein. Alternatively, an HCC patient is suitable for TIC or CTC targeted therapy when the patient has at least one upregulated gene among the group of upregulated genes disclosed herein and at least one downregulated gene among the group of downregulated genes disclosed herein.

In one aspect of this invention, TLR4-NANOG signaling is investigated to see if it reprograms TICs to promote self-renewal and oncogenesis. It is believed that NANOG promotes self-renewal ability, tumor-initiation property and chemoresistance of TICs through metabolic reprogramming. The specific pathways, which were examined: oxidative phosphorylation (OXPHOS) and fatty acid oxidation (FAO) were identified as novel NANOG-mediated oncogenic pathways by NANOG ChIP-seq analysis and metabolomics. Gene profiling, proteomics and metabolomics approaches were combined to identify the pathway(s) altered in resulting tumors.

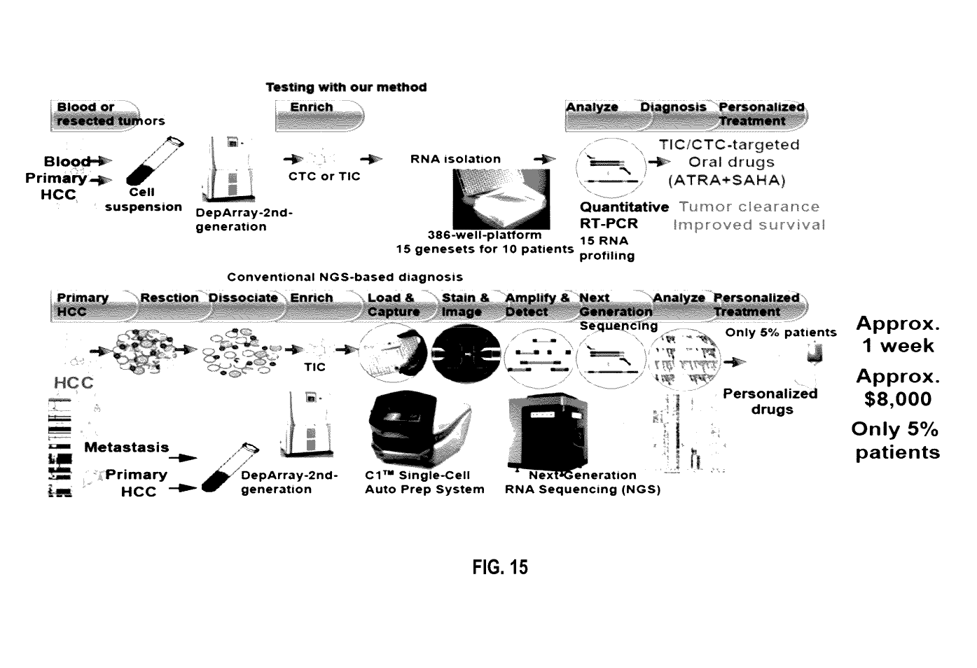

Another aspect of the present invention is to treat HCC patients based on selected RNA profiling by using personalized precision medicine. The method will allow an individual's complete genetic profiling in just a few hours (FIG. 15). In the present method of diagnosing HCC, following a surgery, a pathologist prepares a series of pathological samples from surgically removed HCC tissues. With its rapidness, our method has contributed significantly to the treatment of HCC cancer (FIG. 15). If we do not have to send to next-generation sequence lab, it's quick since a lab can diagnose by regular qRT-PCR.

In one aspect, disclosed herein is a method for epigenetically modifying and eradicating tumor-initiating stem-like cells (TICs) in a subject in need thereof. The method comprises administering, to the subject, an effective amount of suberoylanilide hydroxamic acid (SAHA). In some embodiments, the method further comprise administering, to the subject, an effective amount of all trans retinoic acid (ATRA).

SAHA (Vorinostat®) was first histone deacetylase inhibitor approved by the U.S. Food and Drug Administration (FDA) for the treatment of cutaneous T cell lymphoma (CTCL). It is marketed under the name Zolinza for the treatment of CTCL. Vorinostat has been shown to bind to the active site of histone deacetylases and act as a chelator for Zinc ions also found in the active site of histone deacetylases. Vorinostat's inhibition of histone deacetylases results in the accumulation of acetylated histones and acetylated proteins, including transcription factors crucial for the expression of genes needed to induce cell differentiation.

Vorinostat is a capsule to take orally (by mouth) and should be taken with food. The capsules should be swallowed whole; do not break or chew them. The actual dose you are prescribed is dependent upon tolerance of the medication and kidney function.

In some embodiments, about 200 to 600 mg of SAHA is administered per dose to a patient. In some embodiments, less than 200 mg of SAHA is administered per dose to a patient, such as 150 mg or less, 100 mg or less, or 50 mg or less. In some embodiments, more than 600 mg of SAHA is administered per dose to a patient, such as 700 mg or more, 800 mg or more, 900 mg or more, 1,000 mg or more, 1,200 mg or more, 1,500 mg or more, 2,000 mg or more, or 5,000 mg or more.

In some embodiments, all trans retinoic acid (ATRA) is administered in conjunction with SAHA. ATRA is a pharmaceutical form of the carboxylic acid form of vitamin A and it is marked under the name Tretinoin. In some embodiments, ATRA is administered at a concentration of 2 mg/kg or more, 5 mg/kg or more, 10 mg/kg or more, 12 mg/kg or more, 15 mg/kg or more, 20 mg/kg or more, 25 mg/kg or more, 30 mg/kg or more, 35 mg/kg or more, 40 mg/kg or more, 50 mg/kg or more, 75 mg/kg or more, or 100 mg/kg or more.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### NANOG Reprograms TIC Metabolism: Summary

Stem cell markers such as NANOG have been implicated in various cancers; however, the functional contribution of NANOG to cancer pathogenesis has remained unclear. Here, Toll-like receptor 4 (TLR4) signaling phosphorylates E2F1 is shown to transactivate NANOG. Down-regulation of Nanog reduces tumor progression. NANOG ChIP-seq identified genes associated with NANOG-dependent mitochondrial metabolic pathways to maintain tumor-initiating stem-like cells (TICs). The causal roles of NANOG in mitochondrial metabolic reprogramming occurred through the inhibition of oxidative phosphorylation (OXPHOS) with decreased production of mitochondrial ROS and activation of fatty acid oxidation (FAO), which was required for self-renewal and drug resistance. Restoration of OXPHOS activity and inhibition of FAO rendered TICs susceptible to a standard care chemotherapy drug, sorafenib. This study provides insights into the mechanisms of NANOG-mediated generation of TICs, tumorigenesis and chemo-resistance due to metabolic reprograming of mitochondrial functions.

### Example 2

### NANOG Reprograms TIC Metabolism: Experimental Procedures

Mice:

HCV NS5A Tg mice (Majumder et al., 2002) were generated by Dr. Ratna B. Ray (St. Louis Univ.) on a C57BL/6 background. NS5A transgenic (Tg) and Tlr4 deficient mice (Jackson Lab) were intercrossed at least six times. HCV Core transgenic mice were generated in University of Southern California (USC) Transgenic Core facility (Machida et al., 2010). Mice were fed a Lieber-DeCarli diet containing 3.5% ethanol or isocaloric dextrin (Bioserv, Frenchtown, N.J.) and/or high-cholesterol high-saturated fat diet, as indicated. To test the role of Nanog in hepatocytes/hepatoblasts in liver oncogenesis in alcohol-fed HCV mice, shRNA was overexpressed against Nanog in mice harboring CMV-loxP-Gfp-stop-loxP/U6-sh-Nanog and Albumin-Cre. In these mice the shRNA is conditionally expressed to knockdown Nanog (KD) in albumin-expressing cells (Yamaguchi et al., 2009).

Cell Lines:

TICs were grown in DMEM/F12 or Kubota medium for all experiments. HEK293T and Huh7 cells were cultured in DMEM (Cellgro) with 10% FBS and essential amino acid supplements.

Vector:

PPARδ expression and mutant (1-299 aa truncation form) constructs were gifts from Dr. Carlo V. Catapano at the Oncology Institute of Southern Switzerland.

Endotoxin Measurement:

For endotoxin measurements, blood was collected from inferior vena cava with pyrogen-free heparin as previously described (Mathurin et al., 2000). Extreme care was taken to eliminate pyrogen and endotoxin contamination of all surgical instruments and laboratory supplies. Blood samples were transferred to appropriate glass tubes made pyrogen-free by heating at 180° C. for 24 hr. Pyrogen-free water was supplied by the manufacturer. Immediately before assay, plasma samples were diluted and heated to 75° C. for 10 minutes to denature endotoxin-binding proteins that can mask endotoxin detection. Levels of endotoxin were measured using the Limulus amebocyte lysate pyrogen test and a kinetic assay program (Kinetic test, Kinetic-QCL, Santa Clara, Calif.; BioWhittaker). The threshold of endotoxin detection was 0.1 pg/mL.

dsRed Imaging Analysis:

Tumor progression and metastases (in lungs and spleens) were monitored by whole-body dsRed bioluminescence imaging (IVIS system, Xenogen) every 8 days over 90 days, as previously described. Images were captured directly to a microcomputer (Xenogen). Imaging at lower magnification that visualizes the entire animal were carried out in a light box illuminated by blue light fiber optics (Xenogen, Inc.), and images were recorded with a thermoelectrically cooled color CCD camera.

Tumor Collection and Analysis:

Harvested tumors were measured for the actual volume and weight. The tumor tissues were divided for snap-freezing for mRNA and protein analysis of targeted OXPHOS/FAO genes and histological fixation with 3% paraformaldehyde followed by sucrose treatment for subsequent immune-staining of target gene products.

Gene Array Analysis of Liver:

Systematic gene microarray analyses were performed for dysplastic and normal tumors, to identify changes in known or unknown signaling pathways that are tightly associated with synergistic induction of liver tumor by Western diet (WD) or alcohol. For microarray analysis, livers isolated from five mice were subjected to RNA isolation, later pooled to achieve collection of sufficient amounts of samples for hybridization to the mouse microarray (Affymetrix Inc.). The Affymetrix mouse gene chip (Mouse genome 430.2 array) was used and hybridization and scanning was in the Genome Core Facility Children's Hospital of Los Angeles. Genes were categorized by related functions for assessment of pathophysiological effects of alcohol in liver. 83 gene transcripts of those positive showed increased expression using 4.0 fold (balanced differential expression) as a cutoff. To identify changes associated with synergism by alcohol or WD, comparative analysis was done in the cells isolated from non-Tg mice vs. Tg mice fed WD. Briefly, data were background-corrected, normalized by RMA (Robust multi-array average) and transformed to median of control samples. Probe level data were summarized to gene level. To find differentially expressed genes a t-test (p<0.05) was used and genes were further ranked by a fold change. The data have been deposited in GEO of NCBI under GSE.

Proteomics:

Proteomic analysis was performed at the Proteomic Exploration Laboratory at California Institute of Technology, Pasadena, Calif. In brief, the livers were lysed for protein extraction and extracted proteins were subjected to one-dimensional SDS gel electrophoresis, and stained protein bands were used for in-gel trypsin digestion and MS sequencing.

Chemicals and Reagents.

Trypsin (modified sequencing grade) was from Promega (Madison, Wis.). Acetonitrile and water (Chromasolv LC-MS quality), iodoacetamide (99+%), trifluoroacetic acid (99+%), dithiothreitol (DTT 99%) and glacial acetic acid (99%) were supplied by Sigma-Aldrich (St. Louis, Mo.).

Isolation and Preparation of Proteins from Mouse Liver.

Animal handling followed AALAC and National Institutes of Health guidelines, and experimental procedures were approved by the IACUC. Tissues were homogenized in 1 ml of sodium phosphate buffer (pH 7.4) using a Polytron homogenizer at 4° C. Low speed centrifugation (800 rpm) was used to remove non-homgenized tissues and debris. Supernatants were re-centrifuged and 0.136 ml of 80% sucrose was added to 1 ml of sample. Sodium phosphate buffer (pH 7.4 with protease inhibitor) was added, centrifuged for 1 hr at 4° C. at 35,000 rpm and washed three times with Tris-EDTA (10 mM Tris, 1 mM EDTA) buffer. The pellet fraction was subjected to chloroform, methanol and water extraction. The interfacial material (proteins) was collected and collected by centrifugation for 15 min at 10,000 rpm. The pellet was washed using Tris-EDTA buffer.

Separation of Proteins by 1D PAGE.

The protein (10-50 m in 20 μL of SDS sample buffer) were separated using 1D SDS PAGE on a 10% BisTris NuPAGE gel using NuPAGE MES SDS running buffer (20×) at a voltage of 120V for the first half hour after which the voltage was reduced to 80V The separated proteins on the gel were stained using colloidal Coomassie blue (Invitrogen, Carlsbad, Calif.).

Protein in-Gel Digestion.

The proteins on the gel were sectioned into 20 pieces, minced and destained using 50 mM ammonium bicarbonate buffer (pH 8.0) and acetonitrile. The proteins were reduced with 25 μl of 10 mM DTT and alkylated using 25 μl of 55 mM iodoacetamide. The proteins were digested using 25 μl of trypsin (6 ng/μl).

Mass Spectrometric Analysis.

Mass spectrometric analysis was performed by a hybrid Orbitrap LC-MS/MS instrument (Thermo Fisher).

Database Searching.

MS/MS spectra were searched using Mascot against the SwissProt database. Peptide tolerance was 20 ppm and fragment ion tolerance was 0.60 Da. Carbamidomethylation at cysteines was set as a fixed modification and oxidation of methionines was set as a variable modification.

Parsing Using Scaffold.

Mascot output files were further curated using Scaffold 3.5.1 analysis, resulting in a 0.2% protein false discovery rate (FDR) and a 5.3% peptide FDR. Further, identification of statistically significantly expressed proteins and heat maps were calculated using the R Statistical software package. Amino acid sequences corresponding to tryptic peptide masses identified in candidate proteins were subjected to the SCAFFOLD analysis software (for confidential protein identification) to rule out alternative protein identifications.

Pathway Analysis.

Pathway Analysis was performed using Ingenuity Pathway Analysis application (Ingenuity Systems, CA).

ChIP-Seq:

TIC results were compared to CD133(−) control cells for detection of genes with increased binding of Nanog. In parallel, isotype control antibody was used as a control. Briefly, cells were rinsed twice with PBS and treated with 1% formaldehyde for 20 min at room temperature to form DNA-protein crosslinks and sonicated to generate 200-500 bp chromatin fragments in size and incubated with anti-NANOG antibodies at 4° C. overnight. Protein A/G agarose beads were added to immune complexes at 4° C. Immunoprecipitates were washed three times in wash buffer. ChIP DNA was purified by phenol-chloroform extraction and ethanol precipitation. NANOG ChIP for CD133-control cells and TICs was carried out as described in an instruction manual of Chromatin Immunoprecipitation Assay Kit (Cat#17-295: Millipore Inc., Temecula, Calif.). The DNA segments obtained by this method were sequenced and further subjected to bioinformatics analysis.

Four pairs of TICs and Nanog-/CD133−/CD49f+ control cells (˜1×10⁵ per mouse) were isolated from four independent mouse liver tumors. ChIP was performed with NANOG antibody using CD133(+) as well as CD133(−) cell lines following a standard protocol as suggested by the manufacturer (Millipore). To generate sequencing library constructs, ChIP DNA fragments (1-10 ng) were used for adapter ligation, gel purification and PCR, followed by ligation. ChIP-seq library constructions and high-throughput DNA sequencing was performed using Illumina HiSeq 2000 (Illumina, San Diego, Calif., USA) using a 50 bp SE reads at the USC Genomic Core.

Bone Marrow Transplantation:

Bone marrow transplantation (BMT) was performed as previously described (Dapito et al., 2012; Seki et al., 2007) with modification from traditional protocols as previsouly described (Kisseleva et al., 2006; Tsung et al., 2005). Briefly, after Kupffer cells were depleted (Van Rooijen and Sanders, 1994), mice were lethally irradiated with 750 cGy followed by tail vein intravenous injection of 10 million bone marrow cells collected from the femurs/tibias of donor mice. Donor-derived bone marrow cells reconstitutes only 30% of Kupffer cells six months after BMT (Kennedy and Abkowitz, 1997). After 12 weeks following BMT, the efficiency of successful BMT was confirmed by harvesting splenocytes and determining LPS responsiveness using IL-6 mRNA induction by quantitative real-time PCR, as a readout. Diethynitrosamine (DEN) or vehicle (PBS) were intraperitoneally injected into mice 3 months after BMT.

Treatment with Alcohol Western Diet Ordiethylnitrosamine/Phenobarbital:

High-cholesterol high-fat diet was used, containing very similar diet components (TD.03350: Harkan Teklad, Inc.) as previously described (Haluzik et al., 2004; Van Heek et al., 1997). Mice were fed alcohol Western diet (Dyets Inc. Cat#D710362) or dextrin control diet (Dyets Inc. Cat#D710027) for 12 months. This alcohol WD is modified from Lieber-DeCarli (L-D) alcohol diet and contains 3.5% ethanol, high-cholesterol and high-saturated fat (1% w/w chol, 21% Cal lard, 4% Cal corn oil, Dyets Inc.). For the chemical carcinogenesis mouse model, diethylnitrosamine (DEN) was intraperitoneally injected at four weeks of age and phenobarbital was fed in drinking water from eight weeks of age to euthanasia as previously established (Machida et al., 2010).

Isolation of Human TICs:

CD133+/CD49f+TICs were isolated from HCC tissues obtained from alcoholic patients with or without HCV infection, as previously described (Chen et al., 2013; Gripon et al., 2002). Fresh liver cancer tissues were collected from the USC transplant surgery unit in collaboration with Dr. Linda Sher. Following harvest, liver cancer specimens were immediately digested with collagenase and DNase to obtain cell suspensions, which were washed and adjusted to a concentration of 2×10⁷ cells/ml. These cells were incubated with antibodies against CD133, CD49f, and CD45 (Becton Dickinson) and sorted by FACS to isolate CD133+CD49f+CD45− vs. CD133−CD49f+CD45− populations as previously described (Parent et al., 2004).

Bioinformatics Analysis of Mouse ChIP-Seq Data:

Approximately, 20 million reads were aligned with the mm9 reference genome using Bowtie 2 (version 0.12.7) to generate around 18 million aligned reads with mapping quality ≧20, allowing only two mismatches per alignment (Li and Durbin, 2009). Only uniquely mapped reads were retained and redundant reads were filtered out. Further, each read was extended in the sequencing orientation to a total of 200 bases to infer the coverage at each genomic position. The genome was divided into non-overlapping windows of 200 bp, and aligned reads were considered to be within a window of the midpoint of its estimated fragment. Mid-points in each window were counted, and empirical distributions of windows counts were created as described previously (Kim et al., 2013). The genomic bins, which contained statistically significant ChIP-Seq enrichment, were identified by comparison to a Poisson background model, assuming that background reads are spread randomly throughout the genome. In addition, fold-enrichment was calculated in CD133+ cells over CD133− cells. The mapping output files were also converted to browser-extensible data (BED) files. For visualization, wiggle tracks and TDF file were generated by computing mean read density over 25 bp bins of mouse genome with aligned and filtered reads from ChIP-seq data. Wiggle tracks were visualized in the IGV (Integrated Genomic Viewer)(Kim et al., 2013) as well as Seqmonk (Seqmonk v0.26.9). To assign ChIP-seq enriched regions to genes, a complete set of Refseq genes was downloaded from the UCSC genome dataset and, genes with enriched regions within 5 kb of their TSSs were called bound.

Gene Ontology Analysis:

Genes which are differentially associated with NANOG in TICs or control cells were functionally analyzed in the context of gene ontology and molecular networks by using the Ingenuity pathway software (IPA; www<dot>ingenuity<dot>com). Differentially enriched genes were categorized into various functional groups (threshold P<0.05) and mapped to genetic networks and gene enrichments in specific pathways were calculated.

For Gene Ontology (GO) analysis, the known NANOG motif obtained from TRANSFAC was used to scan the NANOG ChIP-seq data set. In order to gain insight into the functions of genes, gene ontology (GO) analysis was performed. A list of GO terms was compiled that showed statistically significant over-representation for different classes of functions, such as proto-oncogenes, tumor suppression, transcription factors, cell cycle and translational regulation, house-keeping genes, developmentally regulated genes, immunity and anti-microbial defense genes. Quantitative data were analyzed using Partek and Ingenuity software.

Mitochondria Labeling and Measurement of ROS Levels:

To evaluate the status of mitochondria in TICs, the MitoTracker® Mitochondrion-selective probes for total mitochondrial mass (MitoTracker® Deep Red FM Invitrogen: M22426) and for oxidized state mitochondria (MitoSOX™ Red mitochondrial superoxide indicator) were added to the media, respectively, and cells subjected to FACS analyses. ROS labeling was performed as per the instructions for CellROX® Oxidative stress reagent Probes (CellROX® green reagent, Invitrogen C10444). In brief, the cells were incubated with staining solution (100 nM) in culture media at 37° C. for 30 minutes. After staining was complete, cells were washed with PBS and analyzed by fluorescent microscopy or flow cytometry.

Fluorescence Microscopy:

Cells were fixed in 3.7% formaldehyde for 10 min, blocked in 0.2% BSA for 5 min, and incubated with NANOG antibody (1:100; Abcam) and pAMPK antibody (1:100, Cell Signaling) in 0.1% Triton-X100 and 1×PBS, pH 7.4 overnight at 4° C., followed by staining with FITC-conjugated rabbit anti-IgG Ab (1:500; Jackson ImmunoResearch) for 1 h. A LSM 5 Pa laser scanning microscope (Zeiss) was used to visualize mitochondrial morphology.

Fatty Acid β-Oxidation Assay:

Rates of fatty acid β-oxidation were determined, in which the rate of carbon dioxide production from the oxidation of [¹⁴C]palmitate was measured in Metabolomic Core facility of University of Southern California. Cells were cultured in the presence of [¹⁴C]palmitate-BSA complex and the released [¹⁴C]carbon dioxide trapped for 1 h at 37° C. onto filter paper soaked in 100 mM sodium hydroxide. The rate of (3-oxidation was calculated as the amount of trapped [¹⁴C]carbon dioxide in relative units produced per mg protein per hour.

ATP Production Measurements:

Relative ATP/cell assays were performed in 96-well plates. After cells were treated with inhibitors for 4 hr, culture media was removed. Cell Titer-Glo (100 μl: Promega) and CyQUANT (Invitrogen) were immediately added to each well. Luminescence and fluorescence readings were consecutively measured after room-temperature incubation for 10 min.

Determination of Cis-Elements for TLR4-Induced Nanog Promoter Activation:

To characterize the region required for TLR4-induced Nanog transcriptional induction, truncated, promoter-luciferase constructs were used to test the functional role of predicted and known cis-elements, including E2F1 and NF-κB in its enhancer, and others in the promoter. Six constructs, carrying either a −5421/+50, −4828/+50, −2342/+50, −900/+50, −332/+50 or −153/+50 Nanog genomic fragment were generated or obtained from Dr. Paul Robson at the Genome Institute of Singapore and Dr. Takashi Tada in Kyoto University (Kuroda et al., 2005). To generate pGL3(−5421/+50) construct, −5421/−4828 PCR fragments was ligated into −4828/+50 construct. Each reporter was co-transfected with Renilla luciferase plasmid (SV40-Renilla) to normalize reporter activity to transfection efficiency of TLR4-transduced Huh7 cells. Two days after transfection, the cells were stimulated with LPS for 24 hr, and the cell lysate was analyzed by a dual luciferase assay.

NANOG Enhancer and Promoter Assay Following Site-Directed Mutagenesis:

To test the roles of specific sequence elements within these regions, six mutant-luciferase plasmids were constructed by in vitro mutagenesis using QuikChange™ Site-Directed Mutagenesis Kit (Stratagene). For example, to examine the function of E2F, NF-κB, p53, and IRF-3 elements on LPS-induced Nanog transcriptional activity, 3-bp mutations were generated within the corresponding core conserved regions by base substitution. To ascertain whether this region serves as a TLR4-responsive enhancer through the E2F1 and NF-κB interaction, reporter constructs were used which include a 404-bp enhancer fragment inserted upstream or downstream of a luciferase reporter driven by an Oct4 minimal promoter. These constructs were obtained from Dr. Ng Huck Hui of the Genome Institute of Singapore (Wu et al., 2006). NF-κB and/or E2F binding sites were mutated by introducing 3 bp substitutions (Nanog Enh NF-κB and/or E2F mut-Luc) and tested for enhancer activity in TLR4-Huh7 cells in the presence or absence of LPS stimulation. As a positive control, Nanog enhancer reporter or other control vector was co-transfected with E2F and c-MYC expression plasmid into Huh7 cells. The parental vector construct without the enhancer insert was used as a negative control. All luciferase activities were measured relative to the Renilla luciferase. Basal luciferase promoter activity was set arbitrarily to 100% for all comparisons.

Cox6a2 and Acadvl Promoter Luciferase Assay:

The promoter regions of Cox6a2 and Acadvl were inserted into a pGL3 Firefly luciferase reporter vector as different truncation forms. Cox6a2 promoter constructs with luciferase reporter were gifts of Dr. Moreadith (Wan and Moreadith, 1995). The luciferase assay was performed as per vendor instructions (Promega). Briefly, 1 μg of pGL3 luciferase plasmid was transfected with Fugene. 100 ng of Renilla plasmid was co-transfected as an internal control. Cells were harvested 24 hr after transfection, and cell-free lysates were assayed for luciferase activity measured with the dual-luciferase reporter assay kit (Promega) using a luminometer.

Lentiviral Expression System:

The cDNA for ACADVL was subcloned into the lentiviral vector and dsRed expression cassette. Two TLR4 or scrambled shRNAs in the lentiviral vector of pLKO were purchased from Sigma-Aldrich. The lentivirus overexpression vectors were purchased from Applied Biological Materials and lentivirus shRNA vectors were purchased from Sigma-Aldrich. Lentivirus was made by transfecting 2×10⁶ HEK293T cells with 10 μg of lentiviral vector, 6.5 μg pCMV-AR8.2 (packaging vector), and 3.5 μg pCMV-VSV-G (envelope vector) using Fugene (Roche). Forty eight hours later, medium was collected, filtered, and concentrated using the Lenti-X concentrator (Clontech). Concentrated virus was added to TICs, followed by mixing for 2 hr at 37° C. in the presence of 8 μg/μl polybrene in DMEM/F12 medium.

Reverse Transcription and Real-Time PCR (qPCR):

Total RNA was extracted from the cells by RNeasy Mini kit (Qiagen). 1 μg of RNA was treated with DNase I (Invitrogen) and used for reverse-transcription (Omniscript RT kit, Qiagen). Quantitative real-time PCR was performed with Taqman Fast Advanced master mix (Invitrogen) using ABI 7900 system (Applied BioSystems). Taqman primers and probes for Actin (assay ID: Mm00607939_s1), Nanog (assay ID: Mm02384862_g1), Stat3 (assay ID: Mm01219775_m1), Esrrb (assay ID: Mm00442411_m1), Esr2 (assay ID: Mm00599821_m1), Pcx (assay ID: Mm00500992_m1), Atp6v1 g2 (assay ID: Mm01159330_g1), Atp5d (assay ID: Mm00502864_m1), Atp5h (assay ID: Mm02392026_g1), Atp8b2 (assay ID: Mm01220121_m1), Acaa2 (assay ID: Mm00624282_m1), Cox15 (assay ID: Mm00523096_m1), Cox6a2 (assay ID: Mm00438295_g1), Ndufs2 (assay ID: Mm00467603_g1), Ndufv2 (assay ID: Mm01239727_m1), Uqcrfs1 (assay ID: Mm00481849_m1), Idh1 (assay ID: Mm00516030_m1), Idh2 (assay ID: Mm006124290_m1), Tet1 (assay ID: Mm01169087_m1), Tet2 (assay ID: Mm00524395_m1) and Tet3 (assay ID: Mm00805756_m1) were obtained from Applied Biosystems.

Immunoblotting:

Cells were lysed in lysis buffer (50 mM Tris-HCl, pH 7.4, 150 mM NaCl, 1 mM EDTA, and 1% Triton-X100) containing 1× protease inhibitor cocktail (Sigma). Protein (50 μg/sample) was resolved by 8-15% SDS-PAGE, transferred to nitrocellulose membranes, and incubated for 1 hr with 5% milk/TBS-T and overnight with primary Abs in 5% BSA. Antibodies used were: TLR4 (Santa Cruz), NANOG (Abcam), TAK1 (Cell Signaling), TBK1 (Cell signaling), AMPKs (Cell signaling), E2F1 (Cell signaling), pE2F1(Ser337) (Santa Cruz), pE2F1(Ser332) (Thermo Scientific), ACADVL (Santa Cruz). ECL Plus (GE Healthcare) was used for chemo-luminescent detection.

XF24 Extracellular Flux Analyzer for Measurement of Cellular OCR and ECAR:

To measure cellular bioenergetics using extracellular flux, a Seahorse XF96 Extracellular Flux Analyzer was used following the published protocol (Ahfeldt et al., 2012; Ferrick et al., 2008). Functional assays of FAO and glycolysis in live cells showed that scrambled shRNA-transduced TICs had less glycolytic energetics (an embryonic pattern) (Onay-Besikci, 2006) as the baseline while Nanog-silenced TICs had similar glycolysis-dependency, but significant activation of FAO. Cells were plated in gelatin-coated XF 24-well cell culture microplates at 2-7.5×10⁴ cells/well (Seahorse Bioscience) and incubated in pre-warmed unbuffered DMEM medium (DMEM containing 2 mM GlutaMAX, 1 mM sodium pyruvate, 1.85 g l⁻¹ NaCl and 25 mM glucose) for 1 h. The oxygen consumption was measured by the XF24 extracellular flux analyzer (Seahorse Biosciences) in unbuffered DMEM assay medium supplemented with 1 mM pyruvate and 25 mM glucose after 45 to 60 min equilibration.

The characteristic function of mature hepatocytes is metabolism/thermogenesis, driven by the catabolic breakdown of lipids. To distinguish these tumor cells at a functional level, the oxygen consumption rate (OCR) and extracellular acidification rate (ECAR) were analyzed as previously described (Ahfeldt et al.). It was observed that the basal OCR and ECAR rates were highest in the Nanog-silenced TICs. Compounds were added that modulated mitochondrial function sequentially and measured the effect on OCR and ECAR after the addition of each compound. Oligomycin was first administered to determine ATP turnover and the degree of proton leakage. At the baseline, the Nanog-silenced TICs showed slightly elevated levels of proton leakage when compared to unprogrammed cells. After the addition of the electron transport chain decoupler (FCCP), the maximal respiratory capacity was measured. Nanog-silenced TICs showed significantly higher levels of OCR and ECAR when compared to the unprogrammed cells, whereas TICs did not. Finally, antimycin was administered to inhibit the flux of electrons through complex III and prevent oxygen consumption by the cytochrome c oxidase in the mitochondria as previously described. For determination of individual ETC complex activities, mitochondrial biogenesis was profiled by adding perturbation drugs: 2 μM oligomycin, 0.5 μM FCCP and 5 μM antimycin A/rotenone, in succession. OCR for complexes II-IV was measured by first inhibiting complex I with rotenone; OCR for complexes III-IV was measured by first inhibiting complex II with FCCP; and OCR for complex IV was measured by first inhibiting complex III with antimycin. The Etomoxir (ETO, 100 μM)-sensitive component of oxygen consumption rate (OCR) represents FAO. Absolute values of OCR were expressed as pmol min⁻¹ per 10⁶ cells and mpH min⁻¹ per 10⁶ cells. OCR and ECAR were determined by plotting the oxygen tension and acidification of the medium in the chamber as a function of time and normalized to protein concentration (picomoles per minute per milligram), respectively. OCR and ECAR were normalized by cell numbers in all experiments.

The ECAR was measured over time at 10 min intervals. The first three measurements were conducted to establish a baseline rate, followed by two measurements after the addition of oligomycin, an ATPase inhibitor (I). By uncoupling the proton gradient with FCCP, the maximum OCR rates were determined over the next two time intervals (II). By addition of glycolysis inhibitor (2-DG) or CPT1 inhibitor (ETO), the OCR rates were determined over the next two time intervals (III). Finally, at two time points, measurements were conducted after inhibition of the mitochondrial respiratory chain with antimycin/rotenon (IV).

Stable-Isotope Carbon Labeling is Traced for Glutaminolysis Analysis:

To test the glutamine utilization, TICs were incubated in 1 mM of [U-¹³C₅, 2,5-¹⁵N₂]-glutamine (Cambridge Isotope Laboratory, Cat # CNLM-1275-H-PK) for 4 hr. When [U-¹³C₅, 2,5-¹⁵N₂]-glutamine is taken up by cells, it loses the ¹⁵N on the 5^{th} carbon and is converted to [U-¹³C₅, 2-¹⁵N]-glutamate, which loses the ¹⁵N on the 2^{nd} carbon and becomes [U-¹³C₅]-glutamate after rapid equilibration with TCA cycle intermediate α-ketoglutarate. When glutamine and glutamate were analyzed by gas chromatography mass spectrometry (GC-MS) using electronic impact ionization (EI), their TFA derivatives gave rise to C2-C4 (m/z 152) and C2-C5 (m/z 198) fragments. Thus, the [U-¹³C₅, 2-¹⁵N]-glutamate has a C2-C4 fragment of m/z 156 (M4; contains 3×¹³C and a ¹⁵N) and a C2-C5 fragment of m/z 204 (M5; contains 4×¹³C and a ¹⁵N), which represent the relative abundance of glutamine taken up by the TICs. On the other hand, [U-¹³C₅]-glutamate has a C2-C4 fragment of m/z 155 (M3; contains 3×¹³C) and a C2-C5 fragment of m/z 204 (M4; contains 4×¹³C). When the [U-¹³C₅]-glutamate enters TCA cycle metabolism, it will gradually lose the ¹³C carbon after each cycle and generate M2, M1, and M0 C2-C4 fragment and M3, M2, M1, and M0 C2-C5 fragment, which represent the TCA cycle activity. Vigabatrin (γ-aminobutyric acid transaminase: GABA-T) was added in cell culture media and incubated for 20 hr then 1 mM [U-¹³C₅, 2-¹⁵N]-glutamate was added. The sh-TLR4 or sh-Nanog silencing reduced glutamine uptake by the TICs as evident by decreased percentages of M3 and M4 glutamate (C2-C4) and M4 and M5 (C2-C5) fragments.

Stable-Isotope Carbon Labeling is Traced for Flux Analysis:

Cells were cultured in DMEM/F12 medium (17.5 mM unlabeled glucose) supplemented with 7.5 mM [U¹³C₆]-glucose (Cambridge Isotope Laboratories) for 48 hr and total ion chromatography of fatty acids was performed by stable isotope tracing using [U¹³C₆]-glucose for 48 hr. Three independent replicates of 2×10⁶ cells for each cell line were collected, and the cell pellets were suspended in 0.5 ml of water and lysed by sonication. Cell debris was separated by centrifugation and proteins precipitated by treating the clarified supernatant with 1 ml of cold acetone. The final supernatant was air-dried and the free glutamic acid was converted to its trifluoroacetamide butyl ester for GC-MS analysis (Lee, 1996). Rate of fatty acid synthesis is represented by Oleate C18:1/Palmitoleate C16:1 ratio, demonstrating that Nanog silencing reduces fatty acid chain elongation. In addition, CO₂ production of TICs is very low, indicating that TLR4/NANOG induction in TICs inhibits oxygen consumption through inhibition of FA oxidation and TCA cycle entrance.

In Vivo Rescue Experiments of OXPHOS Genes and Inhibition of FAO by Implantation of TICs into Immunocompromised Mice:

The effect of restoration of an OXPHOS gene and/or inhibition of FAO for effect on tumorigenicity of TICs in a xenograft model was examined. Cryopreserved human TICs obtained from liver tumors were tested for tumorigenicity in NOG mice. Prior to implantation, these cells were expanded through several passages and infected with the lentiviral vector expressing Cox6a2 cDNA and dsRed (as a fluorescence tracing marker for in vivo imaging) (MOI 10). Ten days post-lentivirus infection, TICs (1×10⁴) were subcutaneously injected into 6-8-week-old NOG mice. Tumor growth was monitored and palpable tumors were measured by caliper every 4 days for 44 days.

Statistical Considerations:

Log-rank tests and Cox regression was used to determine if differences between groups were significant (α=0.05). The growth of liver tumors was monitored by caliper. The normal chow fed mice served as the control to confirm that the alcohol Western diet had the intended effect. Data are presented as mean±S.D. A two-tailed t-test was used for most comparisons, with p<0.05 considered statistically significant. For the parameters measured in the experiment above, two-tailed non-paired Student's t-test was used for comparison between two groups, and p values less than 0.05 were considered significant. ANOVA and Fisher's test was used for comparison of more than two groups.

### Example 3

### NANOG Reprograms TIC Metabolism: Data and Analysis

Microarray and Proteomics Analysis of Three Different Liver Disease models:

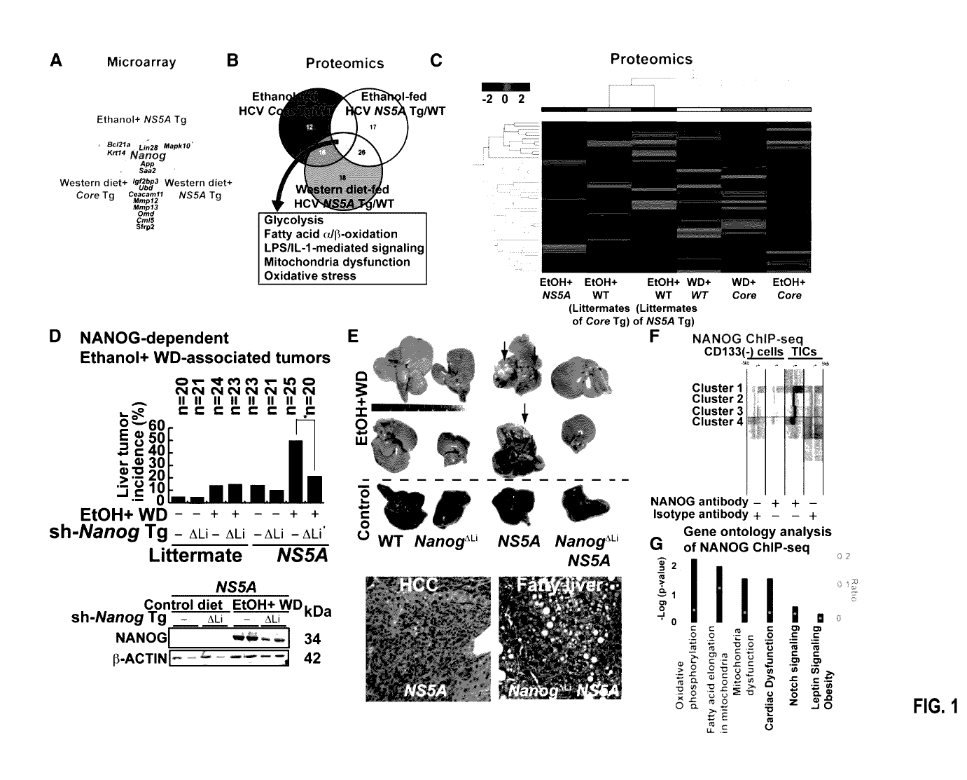

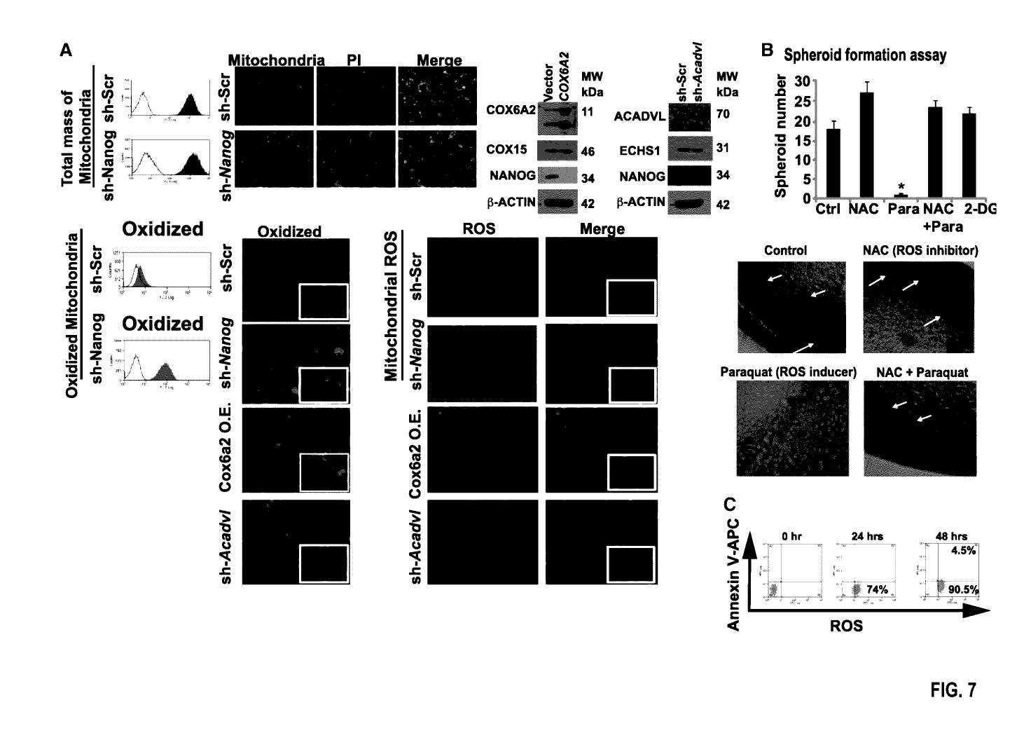

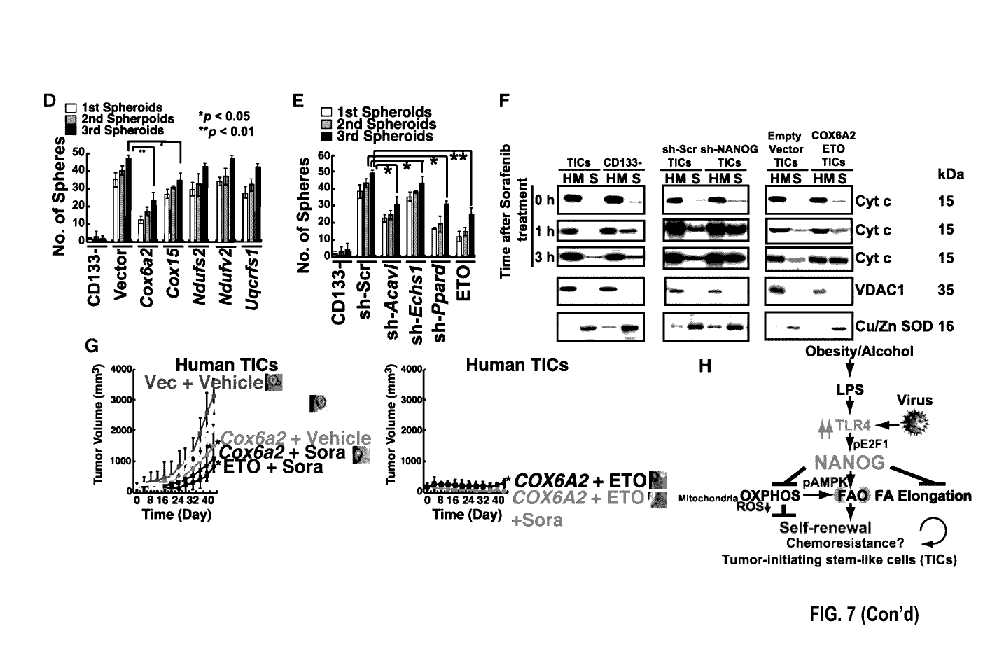

Liver specimens from the alcohol- or obesity-HCV-induced tumor models were profiled using microarray and identified Nanog as the most consistently up-regulated gene (FIG. 1A). Using proteomics approach, it was further showed that enzymes involved in glycolysis, fatty acid metabolism and mitochondrial respiration were similarly dysregulated in the three liver tumor models (FIGS. 1B, 1C, 7A, 7B and Table 1 as shown below).

TABLE 1
Differentially expressed 48 Signature proteins in three groups

| | | | Ethano + Ns5a vs | | Ethanol + Core vs | | WD + Core | |
|---|---|---|---|---|---|---|---|---|
| SwissProt | Accession | | Thanol + WT | | Ethanol + WT | | vs WD + WT | |
| (name) | number | Ensembl Gene | FC | Regulation | FC | Regulation | FC | Regulation |
| HMCS2 | P54869 | ENSMUSG00000027875 | 0.888888889 | DOWN | 0.857142857 | DOWN | 2.222222222 | UP |
| HSP7C | P63017 | ENSMUSG00000015656 | 0.833333333 | DOWN | 4.666666667 | UP | 0.6 | DOWN |
| DAK | Q8VC30 | ENSMUSG00000034371 | 0.666666667 | DOWN | 2.75 | UP | 0.533333333 | DOWN |
| METK1 | Q91X83 | ENSMUSG00000037798 | 0.666666667 | DOWN | 10 | UP | | |
| HS90B | P11499 | ENSMUSG00000023944 | 0.6 | DOWN | 2.666666667 | UP | 0.6 | DOWN |
| AATM | P05202 | ENSMUSG00000031672 | 0.833333333 | DOWN | 3 | UP | 1.4 | UP |
| TPIS | P17751 | ENSMUSG00000023456 | 0.777777778 | DOWN | 2.333333333 | UP | 1.333333333 | UP |
| HPPD | P49429 | ENSMUSG00000029445 | 0.75 | DOWN | 2 | UP | 2 | UP |
| ALDH2 | P47738 | ENSMUSG00000029455 | 0.733333333 | DOWN | 2 | UP | 1.5625 | UP |
| PRDX5 | P99029 | ENSMUSG00000024953 | 0.666666667 | DOWN | 3 | UP | 4 | UP |
| ACTB | P60710 | ENSMUSG00000029580 | 0.65 | DOWN | 2.6 | UP | 1.545454545 | UP |
| MIF | P34884 | ENSMUSG00000033307 | 0.571428571 | DOWN | 1.25 | UP | 1.25 | UP |
| ETFB | Q9DCW4 | ENSMUSG00000004610 | 0.5 | DOWN | 1.428571429 | UP | 1.5 | UP |
| UBIQ | P62991 | ENSMUSG00000008348 | 0.5 | DOWN | 3.5 | UP | 1.375 | UP |
| F16P1 | Q9QXD6 | ENSMUSG00000069805 | 10 | UP | 0.75 | DOWN | 0.636363636 | DOWN |
| FTHFD | Q8R0Y6 | ENSMUSG00000030088 | 2.294117647 | UP | 0.666666667 | DOWN | 0.846153846 | DOWN |
| BLVRB | Q923D2 | ENSMUSG00000040466 | 1.25 | UP | 0.666666667 | DOWN | 0.714285714 | DOWN |
| IREB1 | P28271 | ENSMUSG00000028405 | 4 | UP | 0.5 | DOWN | 2 | UP |
| PROF1 | P62962 | ENSMUSG00000018293 | 4 | UP | 0.8 | DOWN | 1.25 | UP |
| CAH3 | P16015 | ENSMUSG00000027559 | 1.863636364 | UP | 0.566666667 | DOWN | 1.4 | UP |
| FTCD | Q91XD4 | ENSMUSG00000001155 | 1.5 | UP | 0.5 | DOWN | 1.5 | UP |
| HBB1 | P02088 | ENSMUSG00000052305 | 1.341176471 | UP | 0.698412698 | DOWN | 1.824561404 | UP |
| CPSM | Q8C196 | ENSMUSG00000025991 | 1.333333333 | UP | 0.954887218 | DOWN | 1.27388535 | UP |
| GFRP | P99025 | ENSMUSG00000046814 | 3 | UP | 1.5 | UP | 0.5 | DOWN |
| ASSY | P16460 | ENSMUSG00000076441 | 2.083333333 | UP | 1.125 | UP | 0.785714286 | DOWN |
| ARLY | Q91YI0 | ENSMUSG00000025533 | 2 | UP | 4.4 | UP | 0.75 | DOWN |
| INMT | P40936 | ENSMUSG00000003477 | 2 | UP | 2.5 | UP | 0.75 | DOWN |
| PHS | P61458 | ENSMUSG00000020098 | 2 | UP | 2 | UP | 0.75 | DOWN |
| ENOA | P17182 | ENSMUSG00000059040 | 1.75 | UP | 2 | UP | 0.818181818 | DOWN |
| FABPL | P12710 | ENSMUSG00000054422 | 1.666666667 | UP | 1.259259259 | UP | 0.888888889 | DOWN |
| CBR1 | P48758 | ENSMUSG00000051483 | 1.5 | UP | 6 | UP | 0.666666667 | DOWN |
| DHB5 | P70694 | ENSMUSG00000021210 | 8 | UP | 3 | UP | 2 | UP |
| GPX1 | P11352 | ENSMUSG00000063856 | 5 | UP | 5 | UP | 2.333333333 | UP |
| PARK7 | Q99LX0 | ENSMUSG00000028964 | 5 | UP | 1.333333333 | UP | 2 | UP |
| PRDX6 | O08709 | ENSMUSG00000026701 | 4.5 | UP | 3.666666667 | UP | 1.25 | UP |
| CATA | P24270 | ENSMUSG00000027187 | 4 | UP | 3 | UP | 2 | UP |
| THIM | Q8BWT1 | ENSMUSG00000036880 | 3.5 | UP | 2.5 | UP | 1.222222222 | UP |
| MAAI | Q9WVL0 | ENSMUSG00000021033 | 3.2 | UP | 1.416666667 | UP | 1.8 | UP |
| KHK | P97328 | ENSMUSG00000029162 | 3 | UP | 2 | UP | 1.5 | UP |
| ABHEB | Q8VCR7 | ENSMUSG00000042073 | 2 | UP | 2.5 | UP | 1.666666667 | UP |
| GLRX1 | Q9QUH0 | ENSMUSG00000021591 | 2 | UP | 2 | UP | 2 | UP |
| K2C5 | Q922U2 | ENSMUSG00000061527 | 2 | UP | 1.5 | UP | 3 | UP |
| PEBP1 | P70296 | ENSMUSG00000032959 | 2 | UP | 2 | UP | 1.333333333 | UP |
| EF1A1 | P10126 | ENSMUSG00000037742 | 1.8 | UP | 1.25 | UP | 3 | UP |
| ARGI1 | Q61176 | ENSMUSG00000019987 | 1.777777778 | UP | 3 | UP | 1.833333333 | UP |
| GSTP1 | P19157 | ENSMUSG00000060803 | 1.357142857 | UP | 1.714285714 | UP | 1.333333333 | UP |
| ACBP | P31786 | ENSMUSG00000026385 | 1.333333333 | UP | 6 | UP | 1.333333333 | UP |
| G3P | P16858 | ENSMUSG00000057666 | 1.25 | UP | 2 | UP | 1.375 | UP |

NANOG Plays a Critical Role in Liver Oncogenesis:

In addition to the effects of diet and alcohol on HCC in wt mice, nearly 50% of the HCV transgenic mice fed ethanol-containing Western diet (WD: high in cholesterol and saturated fat) developed liver tumors. This incidence was reduced by 80% in the liver-specific Nanog knockdown (ΔLi) cohort (FIGS. 1D, 1E and Table 2 for histological scores), thus demonstrating the critical role of Nanog in tumor development via obesity/alcohol-HCV interactions.

TABLE 2
Average scores of liver histology in HCV Core and/or NS5A Tg
mice fed with the ethanol or Western diet (WD) for 12 months.

| | | Fatty | Spotty | Inflammation |
|---|---|---|---|---|
| | Diet | liver (0-4+) | necrosis (0-2+) | (0-2+) |
| sh-Nanog | LFD + Dextrin | 0.3 | 0 | 0.1 |
| Alb-Cre; sh-Nanog | LFD + Dextrin | 0.3 | 0 | 0.1 |
| sh-Nanog; NS5A Tg | LFD + Dextrin | 0.4 | 0.1 | 0.1 |
| Alb-Cre; sh-Nanog; NS5A Tg | LFD + Dextrin | 0.3 | 0 | 0.2 |
| sh-Nanog | EtOH + WD | 2.4 | 0.4 | 0.4 |
| Alb-Cre; sh-Nanog | EtOH + WD | 1.7 | 0.2 | 0.8 |
| sh-Nanog; NS5A Tg | EtOH + WD | 3.4 | 0.9 | 1.6 |
| Alb-Cre; sh-Nanog; NS5A Tg | EtOH + WD | 1.5 | 0.3 | 0.5 |
| Non-Tg | Dextrin | 0.3 | 0.1 | 0 |
| Core Tg | Dextrin | 0.4 | 0.1 | 0.2 |
| Core/NS5A Tg | Dextrin | 0.5 | 0.3 | 0.1 |
| Tlr4−/− | Dextrin | 0.2 | 0 | 0.4 |
| Tlr4−/− Core Tg | Dextrin | 0.5 | 0.1 | 0.1 |
| Tlr4−/− Core/NS5A Tg | Dextrin | 0.4 | 0.2 | 0.1 |
| Non-Tg | LFD | 0.2 | 0 | 0.1 |
| Core Tg | LFD | 0.4 | 0.1 | 0.2 |
| Core/NS5A Tg | LFD | 0.5 | 0.2 | 0.3 |
| Tlr4−/− Tg | LFD | 0.2 | 0 | 0.5 |
| Tlr4−/− Core Tg | LFD | 0.3 | 0.2 | 0.3 |
| Tlr4 −/− Core/NS5A Tg | LFD | 0.3 | 0.1 | 0.4 |
| Non-Tg | Ethanol | 1.0 | 0.1 | 0.2 |
| Core Tg | Ethanol | 1.3 | 0.2 | 0.9 |
| Core/Ns5a Tg | Ethanol | 1.9 | 0.4 | 1.1 |
| Tlr4−/− | Ethanol | 0.4 | 0.1 | 0.4 |
| Tlr4−/− Core Tg | Ethanol | 0.8 | 0.2 | 0.6 |
| Tlr4−/− Core/NS5A Tg | Ethanol | 1.1 | 0.2 | 0.8 |
| Non-Tg | WD | 2.2 | 0.5 | 0.7 |
| Core Tg | WD | 3.3 | 0.6 | 1.1 |
| Core/NS5A Tg | WD | 3.4 | 0.7 | 1.3 |
| Tlr4−/− Tg | WD | 1.0 | 0.1 | 0.6 |
| Tlr4−/− Core Tg | WD | 1.3 | 0.2 | 0.7 |
| Tlr4 −/− Core/NS5A Tg | WD | 1.6 | 0.3 | 0.8 |

Fatty liver, 2+: 25%~50% heaptocytes with fat; 3+: 50%~75% with fat; 4+: >75% with fat. Submassive necrosis/inflammation, 1+: lesions encompassing less than 1/3 acinus; 2+: lesions larger than whole acini.
LFD: Low fat diet
HCFD: High-cholesterol high-fat diet

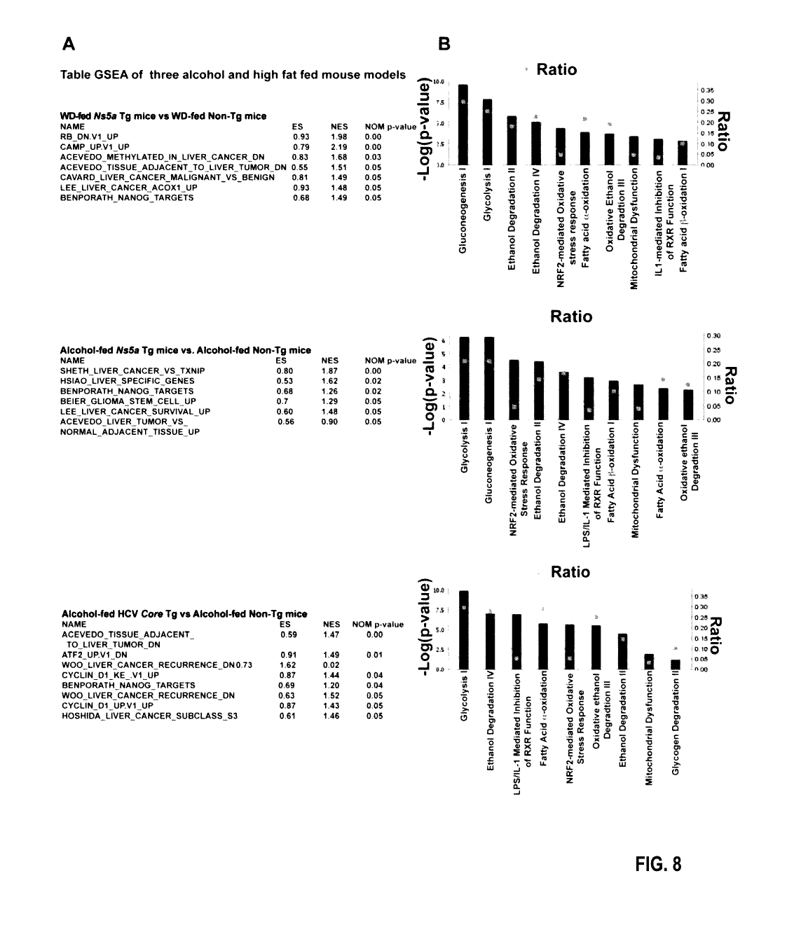

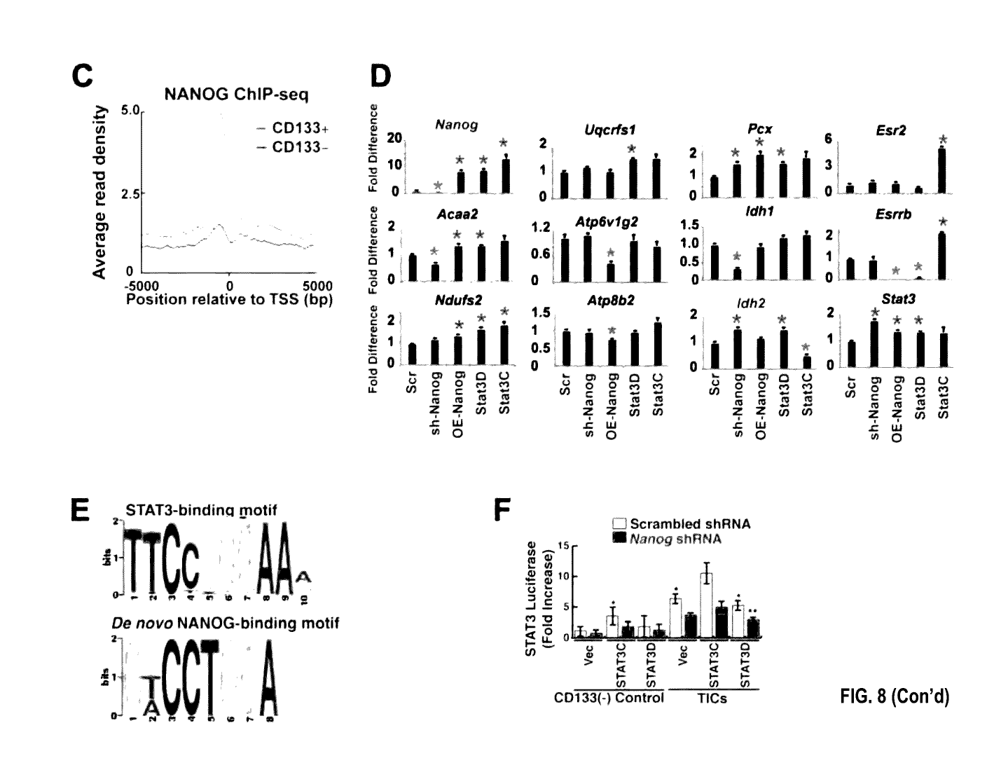

To investigate the underlying mechanism of TIC-mediated tumorigenicity, a genome-wide transcriptional profiling of NANOG-promoter interactions in TICs were conducted with a ChIP-seq approach using a NANOG-specific antibody. NANOG enrichment proximal to transcription start sites (TSS) in TICs were identified compared to CD133(−) cells (FIG. 1F, FIG. 8C). An Ingenuity Pathway analysis implicated the involvement of mitochondrial functions, including OXPHOS-related (Ndufs2, Ndufv2, Uqcrfs1, Cox15, Cox6a2, Atp6v1g2, Atp5d and Atp5h), fatty acid β-oxidation (FAO) genes (Acaa2, Acads, Acadvl and Echs1) and antioxidant genes involved in non-canonical glutamine metabolism (Got2 and Glutathione reductase: Gsr) (FIG. 1G and FIG. 8D). Furthermore, a bioinformatics sequence analysis of NANOG-enriched promoter fragments showed the presence of other consensus binding sites similar to the STAT3-consensus-binding motif (FIG. 8E). NANOG has been shown to physically binds STAT3 (Tones and Watt, 2008). To demonstrate that NANOG induction resulted in increased occupancy of the STAT3 promoter; Nanog silenced cells were used which are in reduced STAT3-mediated transcriptional activity (FIG. 8F), indicating that Nanog interaction with STAT3 is functionally relevant.

The Tumor Incidence in Several HCC Mouse Models is TLR4/NANOG-Dependent:

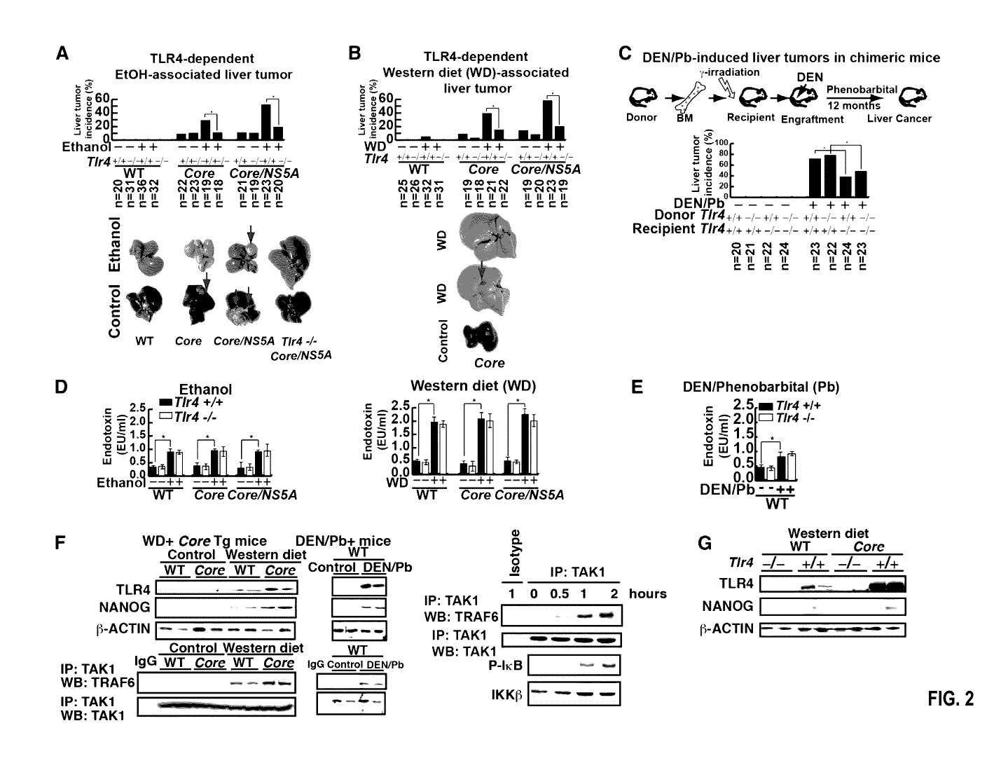

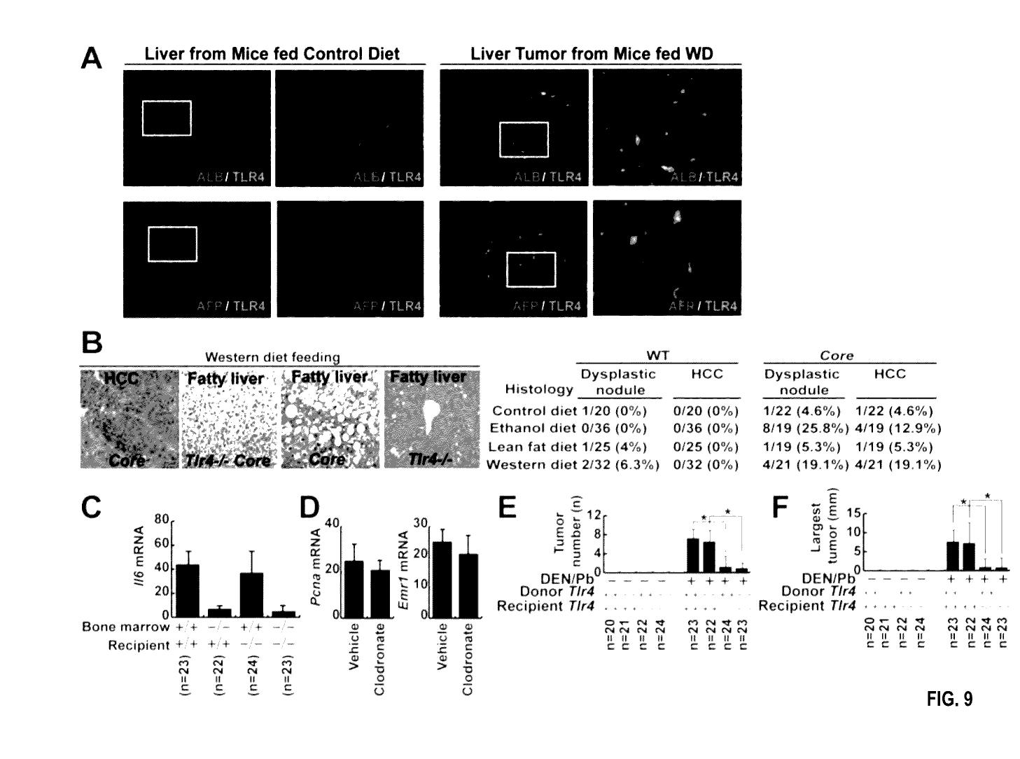

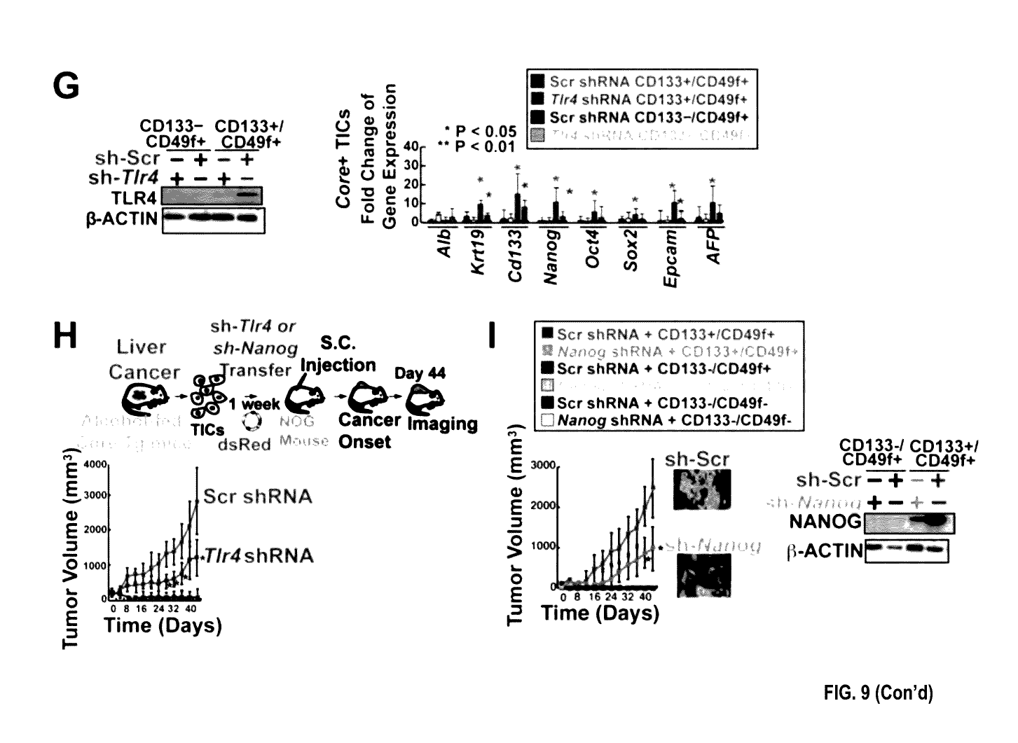

Long-term (12 months) feeding of alcohol diet or a Western diet induced liver tumors in overexpressing HCV non-structural protein NS5A (Majumder et al., 2002), HCV structural protein Core or Core/NS5A transgenic (Tg) mice. Liver tumor incidence was significantly reduced in mice with a Tlr4−/− background (FIGS. 2A and 2B). Tumors from NS5A Tg mice fed WD expressed α-fetoprotein (AFP) (FIG. 9A) and histological analysis confirmed that morphological feature of tumors includes HCC and dysplastic nodules (FIG. 9B and Table 2 for histological scores). Plasma levels of lipopolysaccharide (LPS), a ligand of TLR4, were equally elevated in both Tlr4+/+ and Tlr4−/− mice fed a WD (FIG. 2D).

To determine whether resident liver cells (e.g., hepatocytes) or bone marrow (BM)-derived cells (Kupffer/lymphoid cells) are the primary site of TLR4-dependent oncogenic effects, cross-BM transplantation experiments were performed between Tlr4−/− and Tlr4+/+ mice prior to Diethylnitrosamine/Phenobarbital (DEN-Pb) treatment (FIG. 2C). Chimeric mice were generated by transplantation of BM from either Tlr4+/+ or Tlr4−/− mice into irradiated Tlr4+/+ or Tlr4−/− recipients, as previously described (Dapito et al., 2012). To test whether Tlr4−/− BM engrafted irradiated mice recipients lacked LPS-mediated IL-6 induction, LPS-mediated IL-6 induction was analyzed in splenocytes isolated from engrafted recipient mice (FIGS. 9C and 9D). Transplantation of Tlr4−/− bone marrow cells into Tlr4+/+ mice had no influence on tumor incidence compared to transplantation of Tlr4+/+BM cells into Tlr4+/+ mice. In contrast, transplantation of Tlr4+/+BM cells into Tlr4−/− recipient mice resulted in significantly decreased tumor incidence, indicating that liver parenchymal cells, but not bone marrow-derived immune cells, were responsible for tumor development as previously described (Dapito et al., 2012) (FIG. 2C). Additionally the DEN-Pb-induced HCC tissues exhibited upregulated and activated TLR4 (FIG. 2F, right panel) with increased plasma endotoxin levels (FIG. 2E). These results demonstrate that elevated endotoxin and TLR4 levels in liver parenchymal cells promote HCC progression.

It was previously showed that ectopic TLR4 expression in hepatocytes/hepatoblasts mediated by HCV NS5A activates the stem cell marker Nanog to promote HCC development (Chen et al., 2013). It was found that TLR4-NANOG signaling is activated in other HCC models, such as HCV viral protein Core-WD and DEN-Pb-induced HCC models (FIG. 2F). The expression levels of TLR4 and its downstream signaling proteins TAK1-TRAF6 were analyzed, revealing that TAK1-TRAF6-association is increased in WD-fed mice and carcinogen-injected mice (FIG. 2F). Ubiquitination links TAK1/TBP1/2 complex with TRAF6-TRIF complex. Accordingly, Core-Tlr4−/− mice fed a WD did not express NANOG (FIG. 2G). The absence of Tlr4 in WD fed mice, prevented tumorigenesis, thus supporting a causal link between Tlr4 and NANOG.

Lastly, TICs from HCC mouse models were isolated and analyzed whether TLR4 and NANOG influenced their tumor initiating property in immunocompromised mice. Silencing Tlr4 in TICs inhibited stemness gene expression as determined by qRT-PCR (FIG. 9G). Silencing Tlr4 or Nanog inhibited tumor growth in immune-compromised mice (FIGS. 9H and 9I), indicating that TLR4-NANOG plays a key role for liver oncogenesis.

TLR4-TAK1/TBK1-Mediated E2F1 Phosphorylation Transactivates NANOG Through E2F1-Binding Sites:

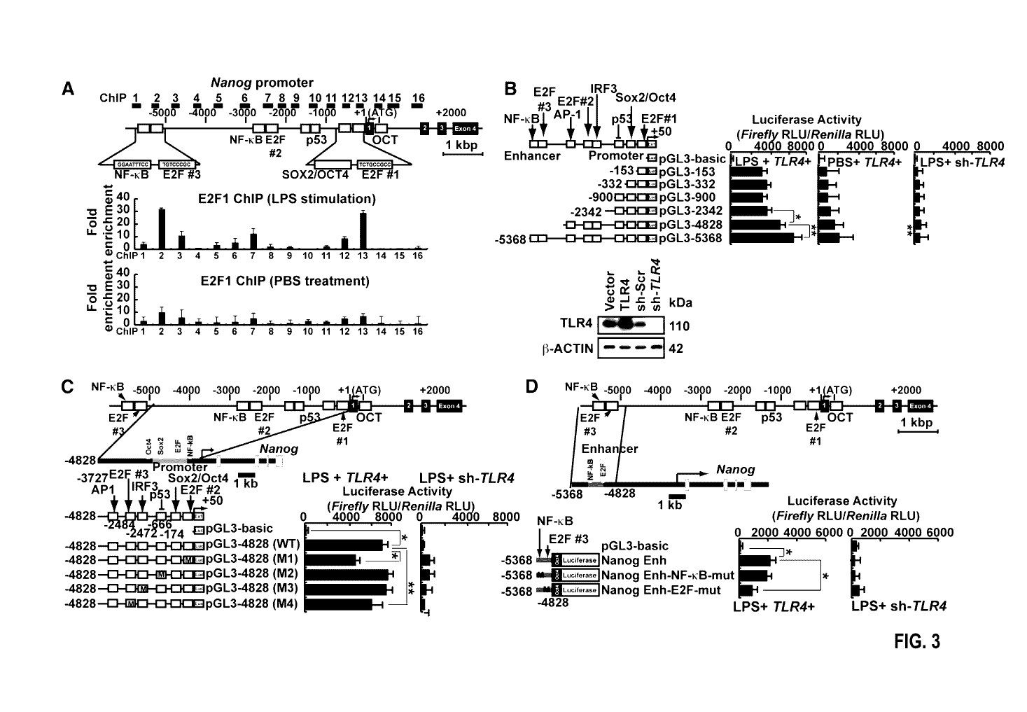

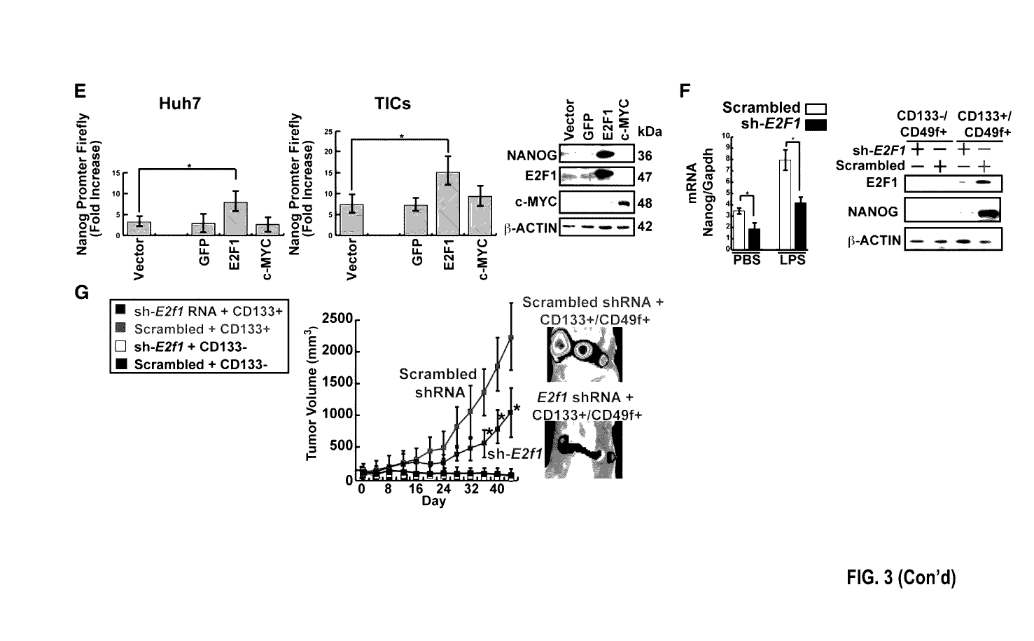

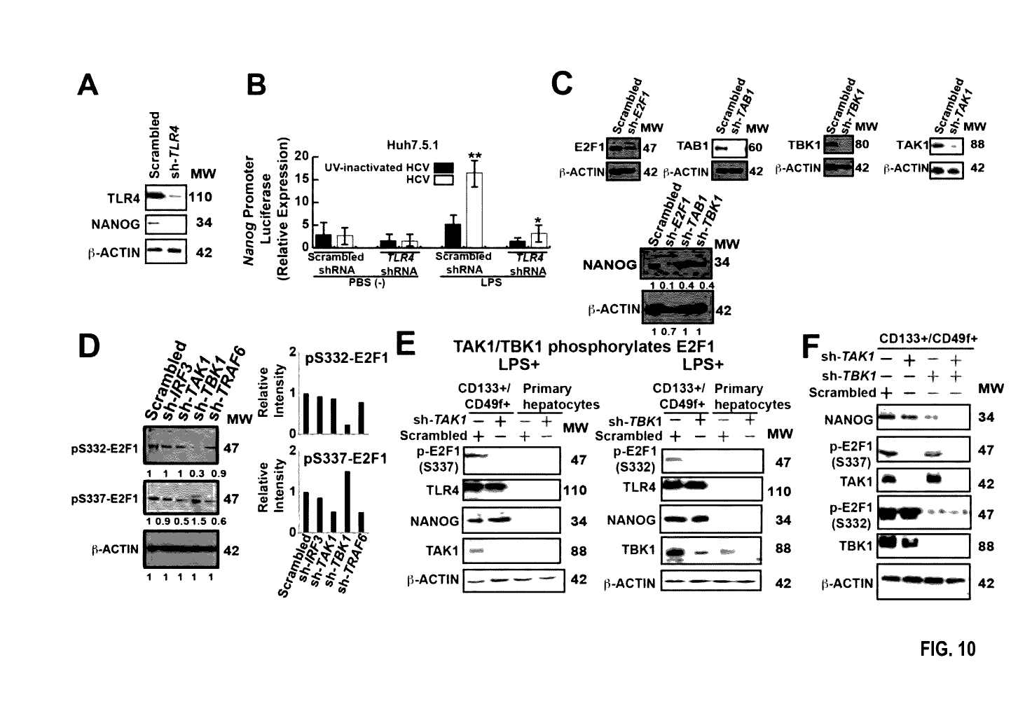

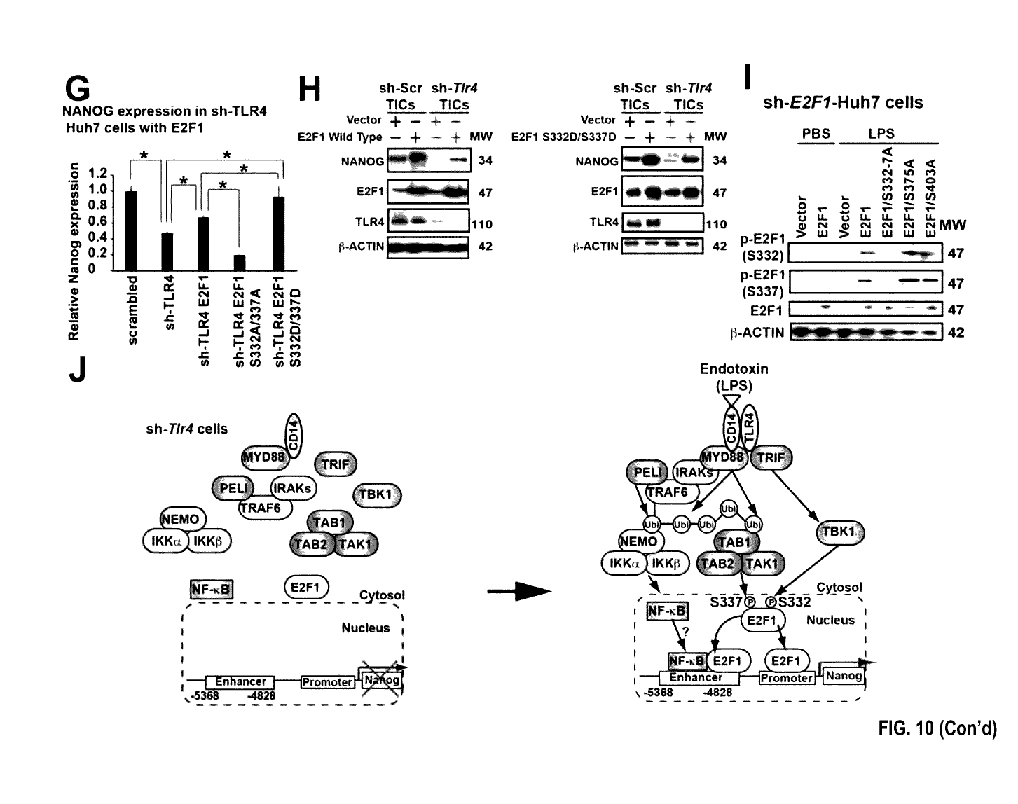

To understand the regulation of NANOG in TLR4 activation, endogenous NANOG promoter activity was monitored. HCV-infected Huh7 cells stimulated with LPS showed increased NANOG promoter activity (FIGS. 10A and 10B). A genome-wide mapping of mouse ES cell transcription factors showed binding of E2F1, ESRRB, and TCFCP2I1 to the Nanog enhancer (Wang et al., 2007; Wu et al., 2006). To determine if E2F1 binds to its cis-elements, ChIP-qPCR analysis was performed. It was observed that E2F1 was enriched in the Nanog distal enhancer and the promoter proximal region in response to LPS (FIG. 3A). Deletion of the distal enhancer region between nucleotides −5368 to −4828 and the promoter proximal region from −153 to +50 containing E2F1 binding sites reduced Nanog promoter activity following LPS stimulation in Huh7 cells (FIG. 3B). Furthermore, base substitution mutations of the promoter proximal region (−153 to +50; FIG. 3C) and the distal enhancer (nt −5368 to −4828; FIG. 3D) reduced NANOG promoter activity.

E2F1 overexpression in TICs and Huh7 cells significantly increased Nanog promoter activity and protein level (FIG. 3E), indicating that E2F1 transcriptionally activates NANOG in TICs. Furthermore, shRNA-mediated knockdown of E2F1 reduced TLR4-induced NANOG transcriptional activation (FIG. 3F) and subsequent tumor development in immunocompromised NOG mice (FIG. 3G).

To determine if TLR4 signaling activates E2F1 via phosphorylation, candidate adapter molecules/kinases in the TLR4 signaling cascade, namely TBK1, TAB1, IRF3, TRAF6 and TAK1 were analyzed. Using a lentiviral shRNA-mediated knockdown approach in TICs, it was demonstrated that TLR4-activated TAK1 and TBK1 resulting in E2F1 phosphorylation at Ser337 and Ser332, respectively (FIGS. 10C and 10D). Knockdown of either TAK1 or TBK1 in TICs did not significantly reduce TLR4-mediated NANOG induction (FIG. 10E). However, silencing of both TAK1 and TBK1 significantly reduced NANOG protein levels (FIG. 10F). Furthermore, transduction of the phospho-mimetic mutant of E2F1 (S332D/S337D) more efficiently transactivated NANOG in comparison to the wt E2F1 in sh-Tlr4-Huh7 cells (FIG. 10G). Overexpression of non-phosphorylatable mutant of E2F1 (S332A/S337A) inhibited induction of NANOG mRNA in Huh7 cells (FIG. 10G), indicating that phosphorylation of E2F1 is crucial for NANOG expression and full activation of Nanog-dependent promoters.

TICs were further analyzed following transduction of shRNAs targeting Tlr4 in combination with wild type E2F1 or E2F1 (S332A/S337A) mutants to test if constitutively active E2F1 transactivated NANOG (FIG. 10H). It was observed that constitutively active E2F1 in both Tlr4+/+TICs and in sh-Tlr4-TICs significantly induced NANOG expression while in shTlr4-TICs, E2F1 overexpression did not affect NANOG expression (FIG. 10H, left panel), consistent with a requirement of E2F1 post-translational modification downstream of TLR4 signaling to induce NANOG (FIG. 10I). Thus, these results demonstrated that TLR4-mediated activation of NANOG by LPS required phosphorylation of E2F1 at both S332 and S337 by TBK1 and TAK1, respectively (FIG. 10J).

NANOG Reduces Mitochondrial OXPHOS:

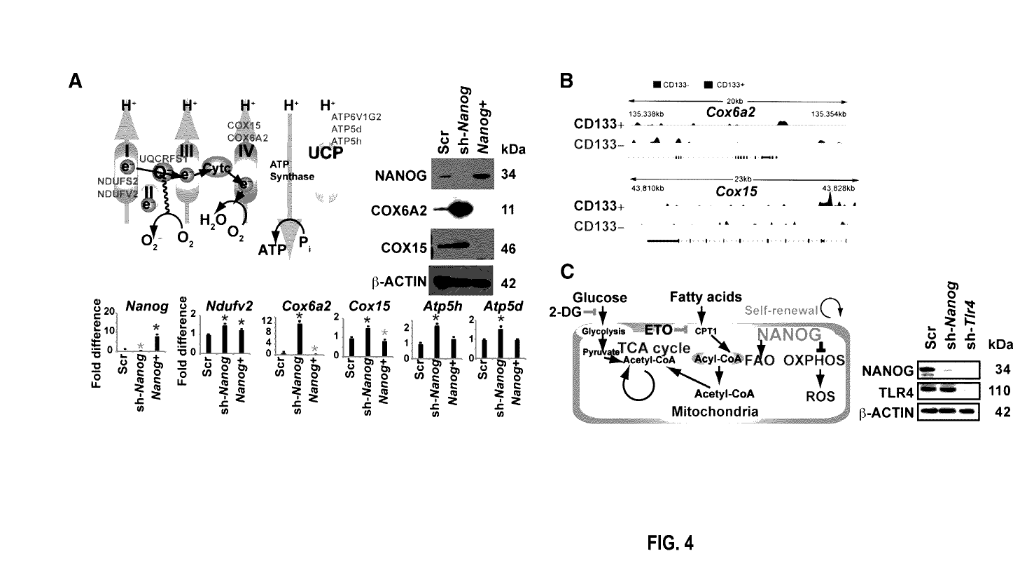

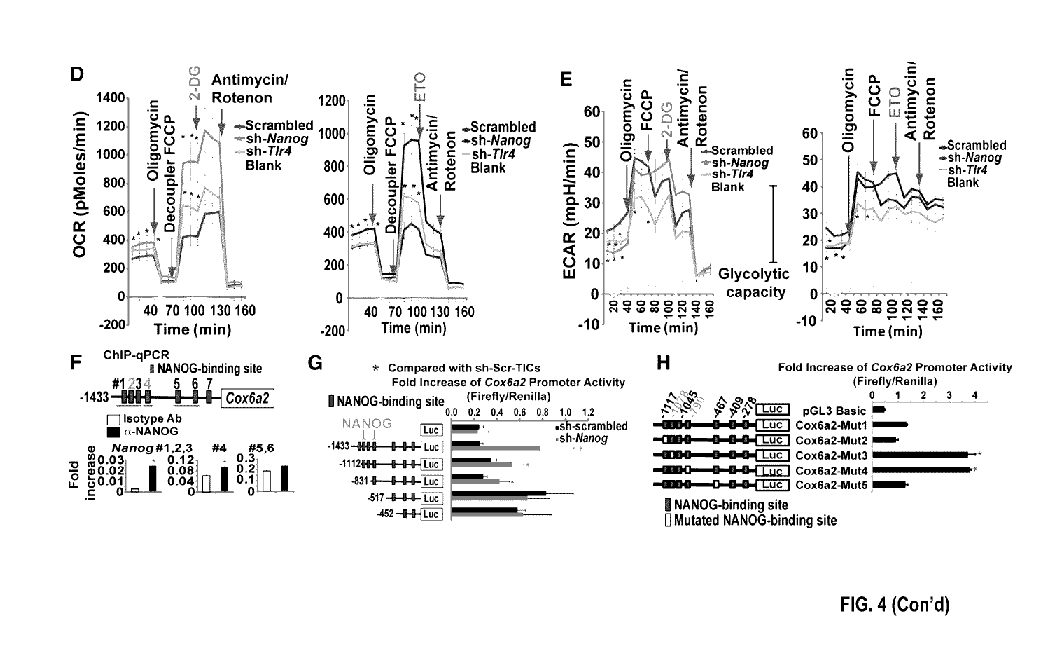

Although the NANOG regulon comprises a large number of genes, the importance of metabolic genes was examined, especially those participating in oxidative phosphorylation based on the gene ontology analysis of Nanog ChIP-seq results. Nanog overexpression decreased OXPHOS activity, whereas Nanog knockdown using shRNA significantly up-regulated the OXPHOS genes and corresponding respiratory activity in TICs (FIG. 4A).

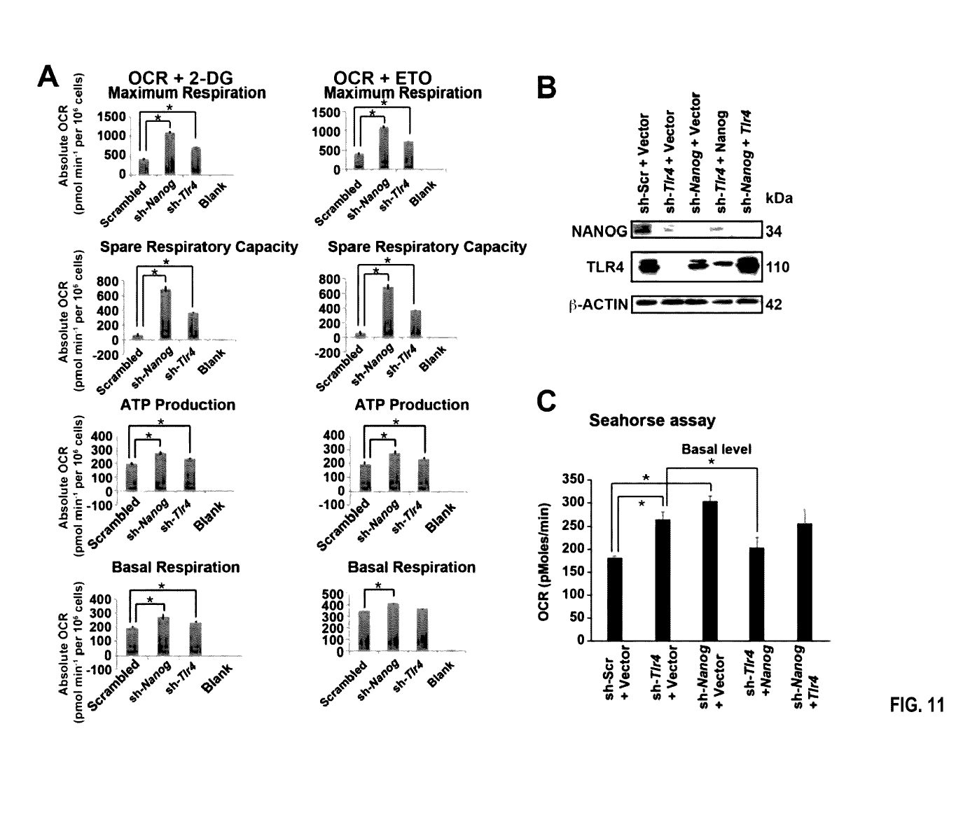

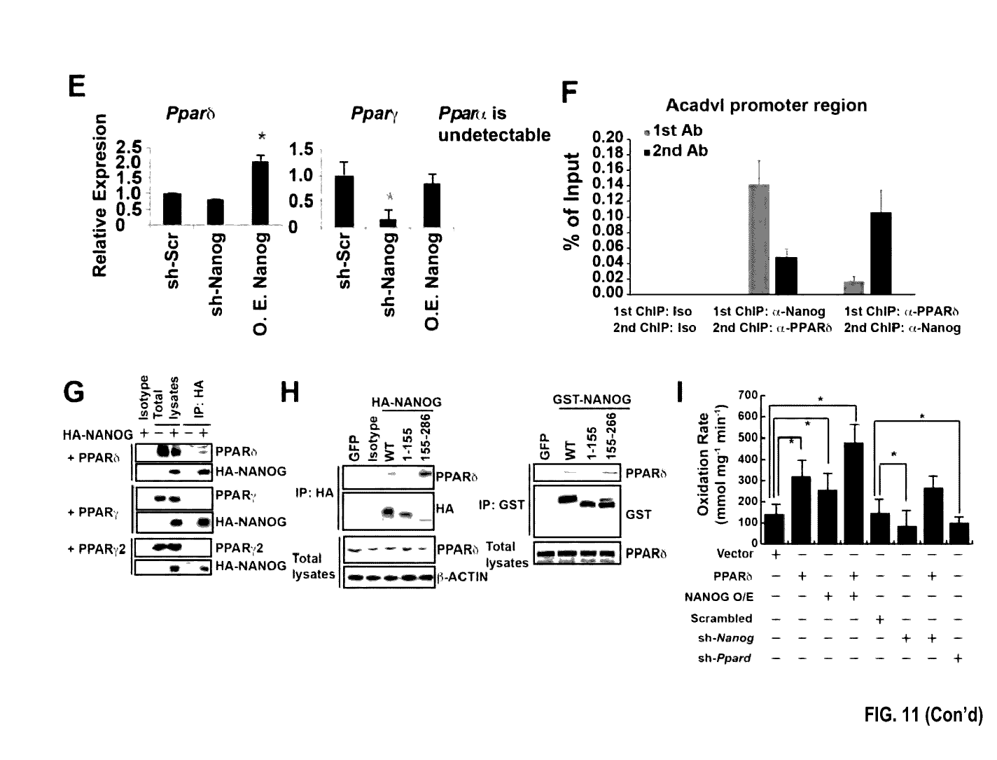

To test if NANOG regulates mitochondrial respiration, the oxygen consumption rate (OCR) in TICs was examined. The basal OCR rates increased in Nanog- or Tlr4-silenced TICs, compared to untransduced cells (FIG. 4D and FIGS. 11A-11C). Silencing Nanog reduced glycolytic activity as demonstrated by a decline of extracellular acidification rate (ECAR) (FIG. 4E). Addition of Etomoxir (ETO), an inhibitor of FA transporter (CPT1), antagonized the FCCP-induced OCR in Nanog silenced TICs (FIGS. 4C-4D), indicating NANOG inhibits mitochondrial respiration.

The expression of the cytochrome c oxidase subunit 6A (Cox6a2) gene was analyzed since it was the most downregulated OXPHOS gene in the ChIP-seq (FIG. 4B) and qRT-PCR (FIG. 4A) analyses. Additionally, Nanog enrichment was observed in the Cox6a2 promoter of TICs via ChIP-qPCR analysis (FIG. 4F). Knockdown of Nanog in TICs increased transcription from the Cox6a2 promoter (nt −1433 to +17), which was suppressed in TICs. A deletion in the Cox6a2 promoter from nt −1433 to −518 significantly increased the Cox6a2 promoter activity in TICs (FIG. 4G), indicating that this region had negative-regulatory activity. Further bioinformatics analysis confirmed putative Nanog binding sites in the region spanning nt −1433 to −518. Mutations in the Nanog binding sites (−1078 and −790) restored Cox6a2 promoter activity in TICs (FIG. 4H). These results show that decreased mitochondrial activity in TICs resulted from Nanog-mediated repression of Cox6a2.

NANOG Promotes Mitochondrial FAO:

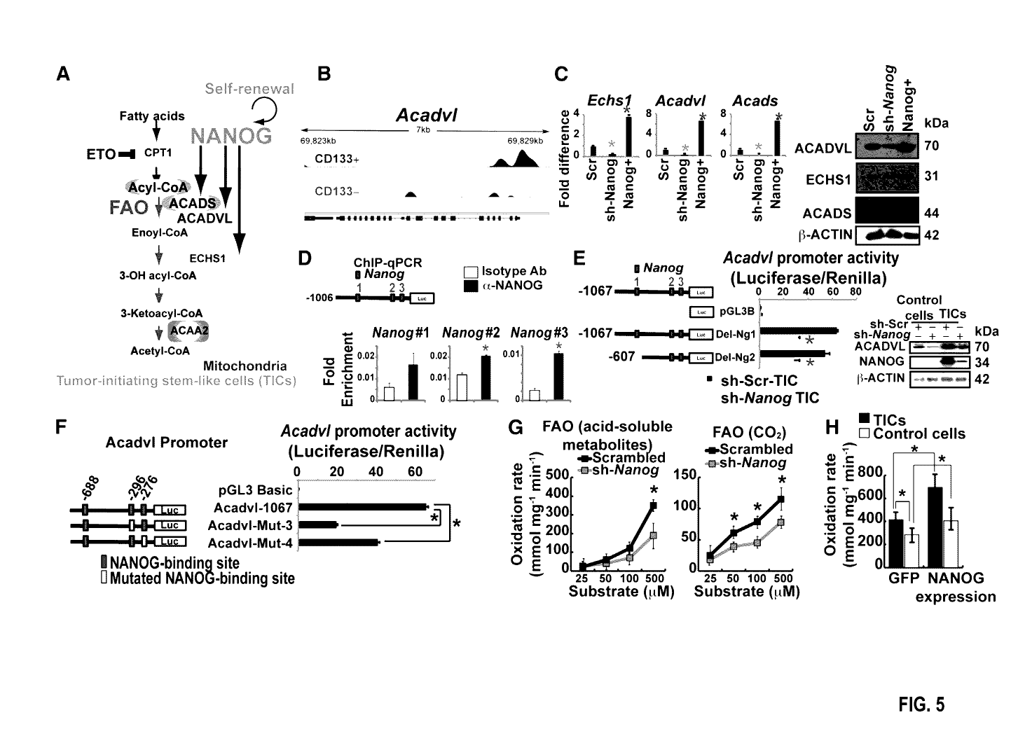

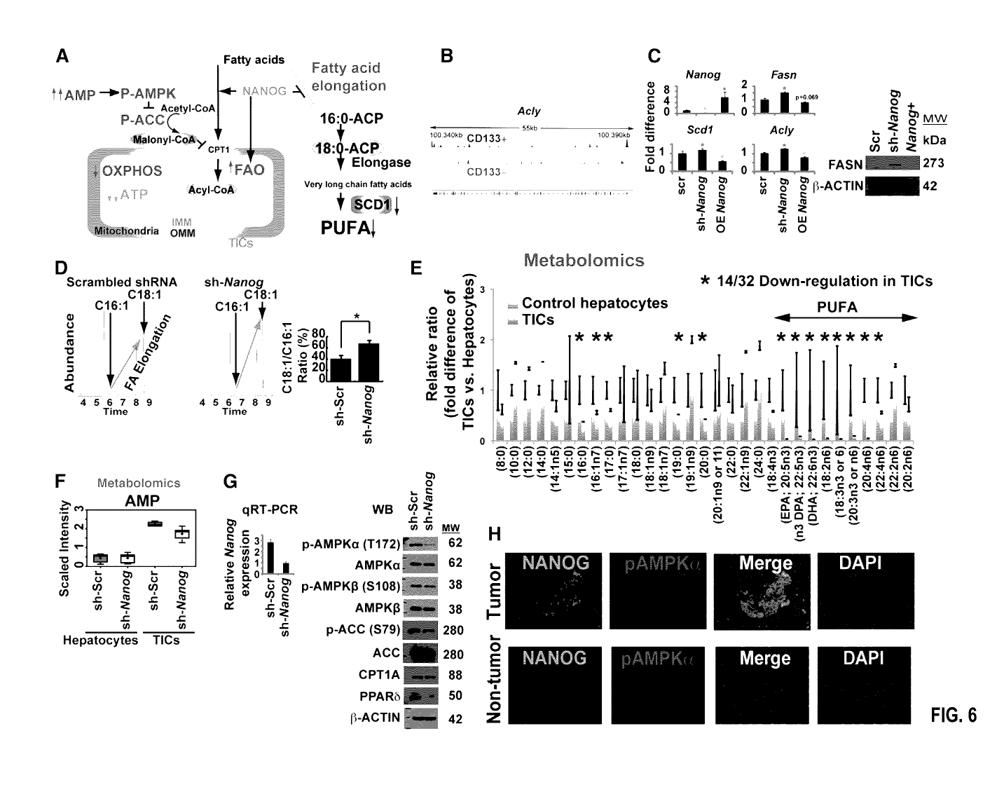

Since silencing NANOG increased OXPHOS levels in TICs (FIG. 4D, left panel), it was reasoned that NANOG should activate additional catabolic pathways, such as FAO, in order to meet the cellular energy demands (FIG. 5A). Based on the gene ontology analysis of the Nanog-ChIP-seq data, the lipid metabolism pathways appeared to be a critical property of TICs (FIG. 1G; FIG. 5B), suggesting that fatty acids were an alternative energy source (FIG. 5A). Targeted expression analysis by qRT-PCR and immunoblot analyses showed that genes associated with the FAO pathway (viz., Acadvl, Echs1, Acads) were significantly expressed in TICs; Nanog knockdown in TICs down-regulated both transcripts and protein levels of FAO associated genes (FIG. 5C).

The analysis of Acadvl was focused in TICs since a significant enrichment of Nanog was observed in the Acadvl promoter by ChIP-seq (FIG. 5B), which was further validated by ChIP-qPCR analyses (FIG. 5D). In reporter assays, full-length Acadvl promoter (nt −1067 to +1), and a truncated promoter (nt −607 to +1) showed significant decreases in their activities when Nanog was silenced in TICs (FIG. 5E). Mutations in two of the three Nanog binding sites further reduced Acadvl promoter activity (FIG. 5F) indicating that Nanog binding sites proximal to the transcription start site were essential for Acadvl transactivation.

To determine if NANOG induced FAO in TICs, FAO flux analysis was used with ¹⁴C-radiolabeled-palmitic acid for production of acid-soluble ¹⁴C metabolites and ¹⁴CO₂. NANOG⁺-TICs have significantly higher levels of FAO activity (oxidation rate) under physiological conditions (FIG. 5H). Silencing of Nanog in TICs resulted in significantly reduced FAO activity, indicating TICs dependence on NANOG for FAO activation (FIG. 5G).

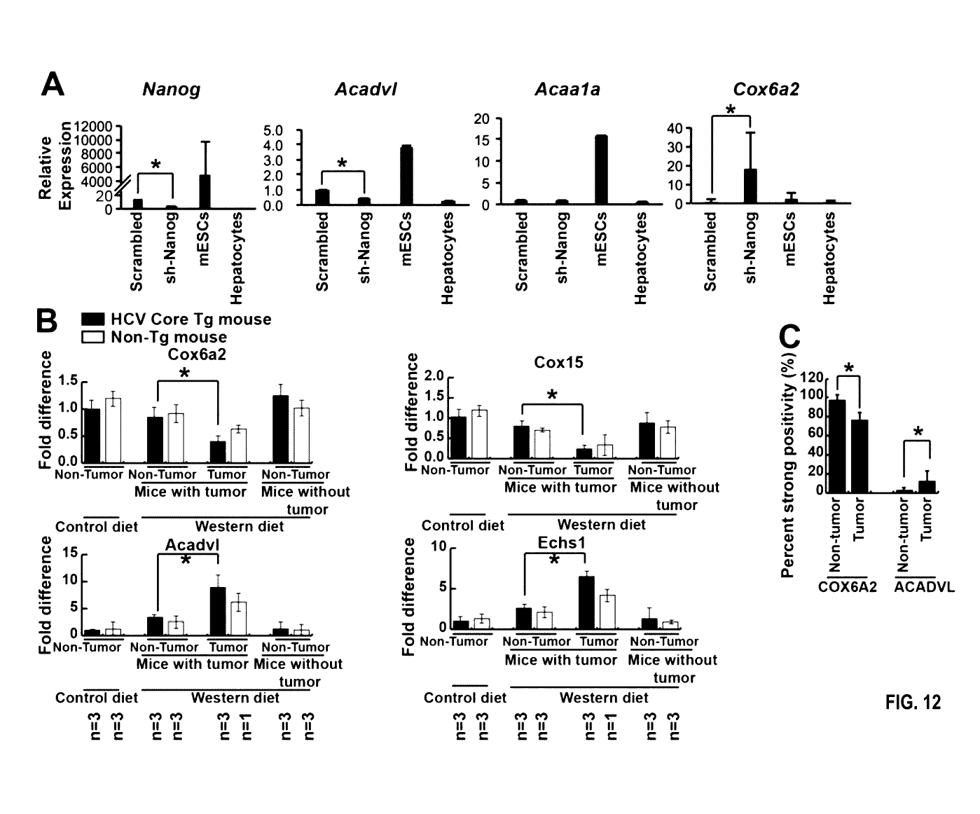

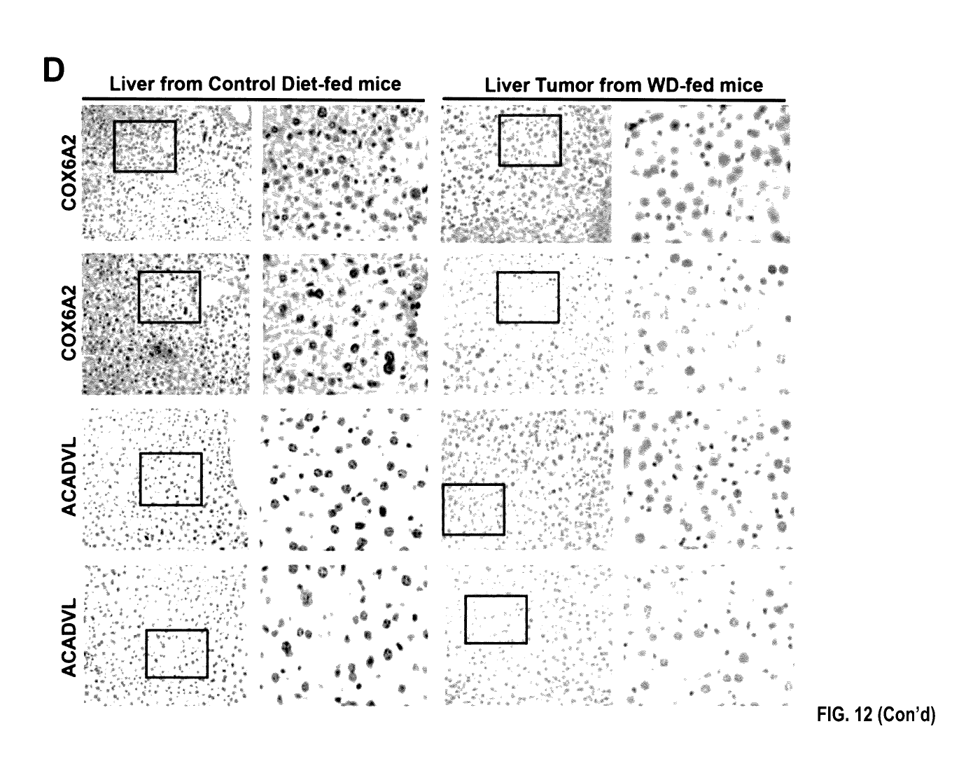

To determine whether NANOG contributed to peroxisome FAO activity, the contribution of Nanog-target Acaa1a, a peroxisome FAO-related gene, was assayed by shRNA-transduced TICs. It was observed that Nanog silencing did not alter Acaa1a expression (FIG. 12A), thus substantiating the finding that, NANOG regulated the mitochondrial FAO genes of TICs for satisfying energy requirements.

It was next determined if the observed effect of Nanog on Cox6a2, and activating effect on Acadvl were related to tumor incidence. Cox6a2, Cox15, Acadvl and Echs1 mRNAs were quantified by qRT-PCR in tumor-bearing mice and compared to non-tumor-bearing mice. This in vivo analysis further corroborated the ex vivo findings that mitochondrial FAO genes (Acadvl and Echs1) were turned on in the cancerous regions whilst not in non-cancerous liver tissues. In particular, tumors in endogenous cancer models exhibited lower levels of Cox6a2 and Cox15 (FIGS. 12B-12D). NANOG suppression in whole liver reduced hepatocarcinogenesis. Acadvl is overexpressed in a small fraction of tumors (8%) (FIGS. 12B-12D), demonstrating that not all tumor cells have elevated levels of ACADVL. These results strongly support the model that in TICs, NANOG regulates mitochondrial FAO gene expression to sustain energy production in TICs for tumor survival and growth.

PPARδ Physically Interacts with NANOG:

TICs downregulated key transcription factors involved in cell differentiation. Based on our NANOG ChIP-seq analysis in TICs, the peroxisome proliferator-activated receptors (PPARs; nuclear receptor proteins which function as transcription factors in cell differentiation) were strongly associated with NANOG. To determine whether the expression of NANOG specifically regulated PPAR (α, δ, γ, γ2) and repressed their pro-differentiation activities, Nanog loss or gain of function analysis was performed.

Since activation of PPARδ is known to increase FAO in cells (Gutgesell et al., 2009) the induction of PPARδ was next studied upon overexpression of Nanog (FIG. 12E). Sequential-ChIP-qPCR analysis on the Acadvl locus in TICs demonstrated that both NANOG and PPARδ were co-enriched at the Acadvl locus (FIG. 12F) thereby suggesting a possible cooperative interaction to increase FAO in TICs. Co-immunoprecipitation of HA-NANOG (FIG. 12G) and PPARs demonstrated that NANOG bound specifically to PPARδ, but not to PPARγ, PPARγ2, or PPARα. Further truncation analysis of the Acadvl promoter demonstrated that the C-terminal end of PPARδ (aa 155-266), but not the N-terminal region (aa 1-155), interacted with NANOG (FIG. 12H). This indicated that the C-terminus of PPARδ (155-266) mediated interaction with NANOG for regulation of PPARδ-dependent transcription. Overexpression of either PPARδ or NANOG in TICs increased the rate of FAO (FIG. 12I). Conversely, Nanog silencing reduced the rate of FAO (FIG. 12I), which was consistent with the observation that NANOG interacted with PPARδ in the Acadvl locus to increase FAO.

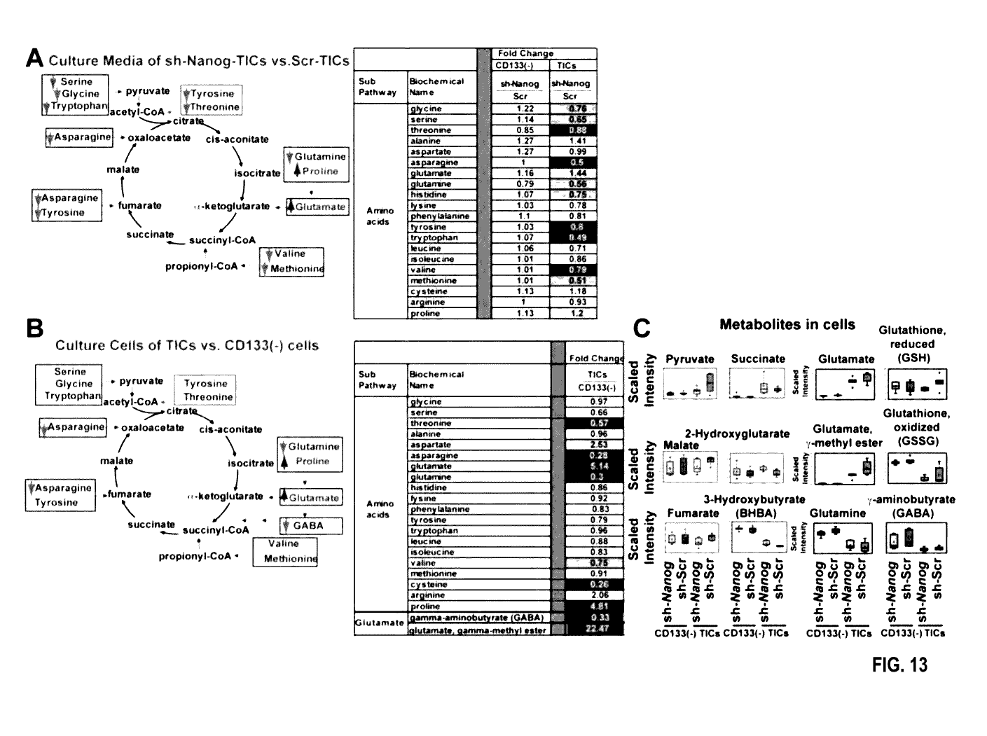

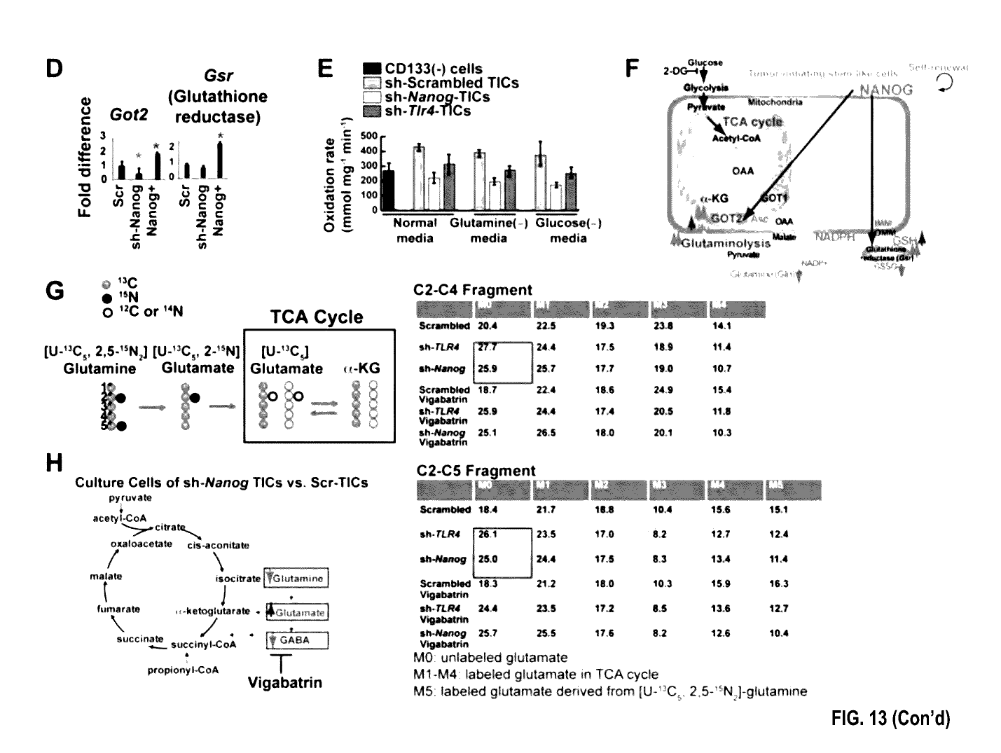

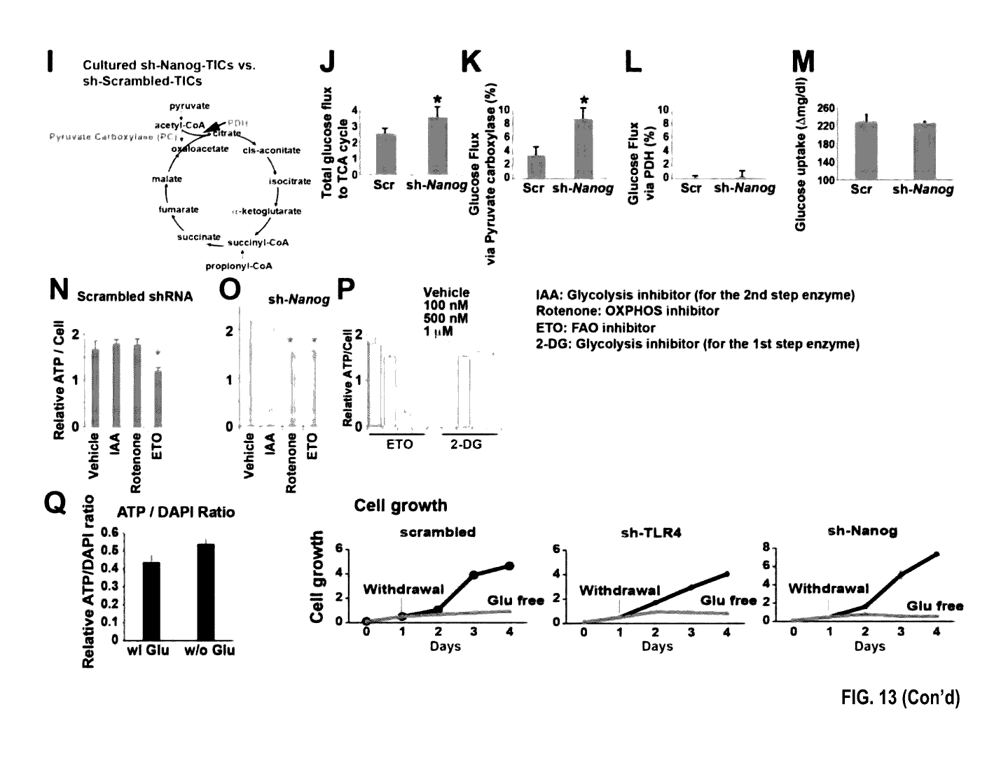

Metabolomics analysis was next performed to assesstricarboxylic acid cycle (TCA cycle) activity in TICs. NANOG-deficient TICs exhibited elevated external levels of glutamate, proline and α-ketoglutarate in growth media in comparison to those in scrambled shRNA-transduced TICs (FIG. 13A), whilst many other amino acids (e.g., threonine, asparagine and tryptophan) were reduced in media from NANOG-deficient TICs (FIG. 13A). This indicated that sh-Nanog TICs consumed more amino acids, reflecting a change in respiration activity. Several amino acids (glutamate, glutamate γ-methylester and proline) increased within TICs in comparison to those in normal hepatocytes while other amino acids (asparagine, glutamine and cysteine) were depleted (FIGS. 13B and 13C). The oxidized form of antioxidant glutathione (GSH) was reduced in TICs in comparison to that of primary hepatocytes (FIG. 13C). Indeed, NANOG ChIP-seq analysis identified antioxidant genes involved in non-canonical glutamine metabolism (Got2 and Glutathione reductase, Gsr) (FIG. 13D). These observations suggested increased energy production occurred through the TCA cycle or alternatively decreased utilization of citrate for fatty acid synthesis was occurring in NANOG-deficient TICs (FIGS. 13E and 13F).

To assess functionality of the mitochondrial TCA cycle in TICs, the possibility that glutamine utilization (an anaplerosis fuel in cancer cells) was tested in the TCA cycle occurred in TICs. The sh-Tlr4 or sh-Nanog silenced TICs showed reduced glutamine uptake as evident by decreased percentage of M3 and M4 glutamate (C2-C4) and M4 and M5 (C2-C5) fragments (FIGS. 13G and 13H). However, sh-TLR4 or sh-Nanog silencing of these cells enhanced TCA cycle activity, as demonstrated by the increased generation of M0 and M1 glutamate in both fragments. These results showed that sh-Tlr4 or sh-Nanog silencing promoted glutamine oxidation in mitochondria. It was also observed Nanog silencing induced increased glucose flux through the TCA cycle via pyruvate carboxylase pathway, but not PDH (FIGS. 13I-13M). Finally, glutamine withdrawal reduced cell growth rate in all TICs in the presence or absence of TLR4 or NANOG (FIG. 13Q). These results showed TICs have striking intrinsic differences in cellular metabolism, compared to normal cell types.

NANOG Orchestrates Mitochondrial Metabolic Reprogramming:

It has been observed that NANOG was important for OXPHOS inhibition and FAO activation. The effect of NANOG was studied on inhibition of fatty acid elongation enzymes by NANOG (FIG. 6A). ChIP-seq showed that NANOG was enriched in the Acly promoter (FIG. 6B). Moreover, fatty acid synthesis genes (Scdl, Fasn and Acly) were derepressed in sh-Nanog-TICs compared to sh-scrambled-TICs (FIG. 6C). Based on the fatty acid elongation profile determined by GC/MS, the rate of fatty acid elongation [ratio of oleate/palmitoleate (C18:1/C16:1 fatty acids)] increased in Nanog-silenced TICs (FIG. 6D), indicating that NANOG inhibited fatty acid elongation.

Further analysis of FAO in TICs was performed by examining fatty acid substrate utilization by metabolomic analysis. Mouse TICs were examined for their ability to metabolize free fatty acids of varying carbon length and C—C bond unsaturation. As summarized in FIG. 6E, TICs showed lower levels of long-chain FA (C16 to C22) utilization as compared to scrambled shRNA-transduced normal hepatocytes. These data indicated TICs differed from normal hepatocytes in their ability to metabolize long-chain fatty acids; in fact TICs may be more efficient at metabolizing longer chain fatty acids (>22). From these studies, the causal roles of NANOG in mitochondrial metabolic reprogramming was elucidated through activation of FAO, which was found to be required for self-renewal of TICs.

AMP/ADP is converted into ATP during OXPHOS. Metabolite analysis of TICs revealed that the AMP level was higher in scrambled shRNA-transduced TICs when compared to Nanog-silenced TICs (FIG. 6F), while the ATP level was lower in TICs, but higher in sh-Nanog-TICs (Fig. S6N). As Nanog-mediated inhibition of OXPHOS is expected to promote AMP/ADP accumulation and AMPK activation (Hawley et al., 1996), phosphorylation of AMPK was examined. The level of phospho-AMPKa was reduced in TICs following Nanog silencing (FIG. 6G), suggesting that NANOG inhibition of OXPHOS promoted the accumulation of AMP, leading to activation of AMPKa via phosphorylation (Hawley et al., 1996). IF microscopy showed phosphorylated AMPKa levels were increased in human TICs (FIG. 6H), consistent with metabolic AMP accumulation.

NANOG restoration in sh-Tlr4-TICs resulted in comparable levels of OCR compared to that of scrambled control TICs (FIG. 13O). ETO treatment reduced ATP production while glycolysis inhibitor (2-DG) did not significantly lower ATP production in TICs (FIG. 13P). Taken together, NANOG apparently plays an important role in the metabolic reprogramming of TICs through down-regulation of the OXPHOS pathway and up-regulation of the FAO pathway.

NANOG Prevents Mitochondrial ROS Production and Maintains Self-Renewal Ability:

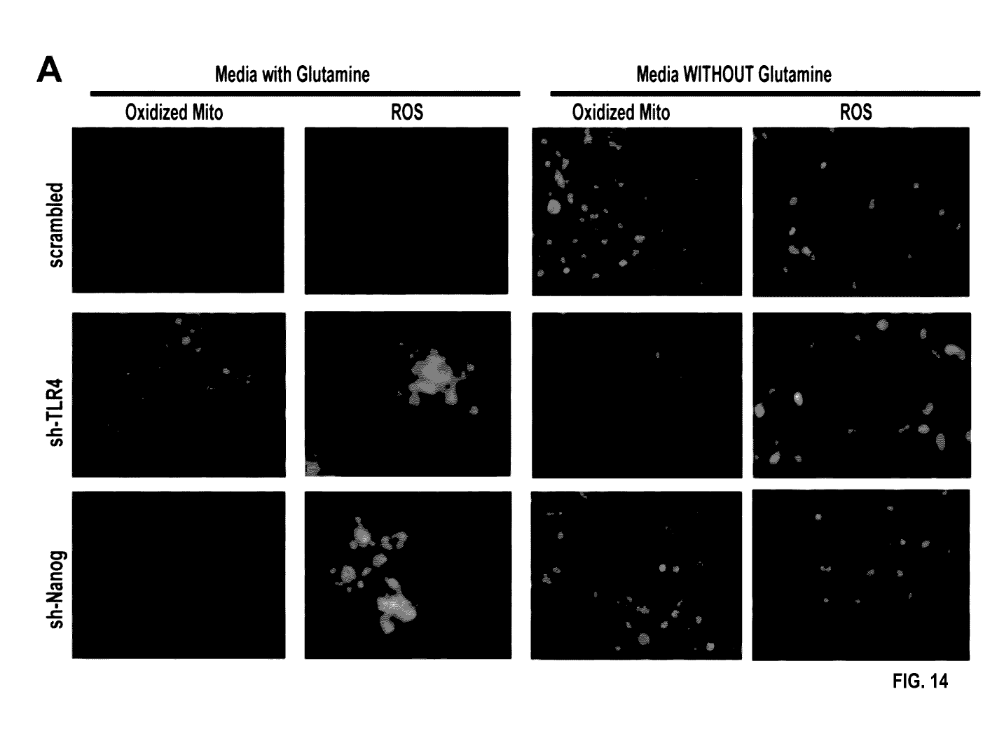

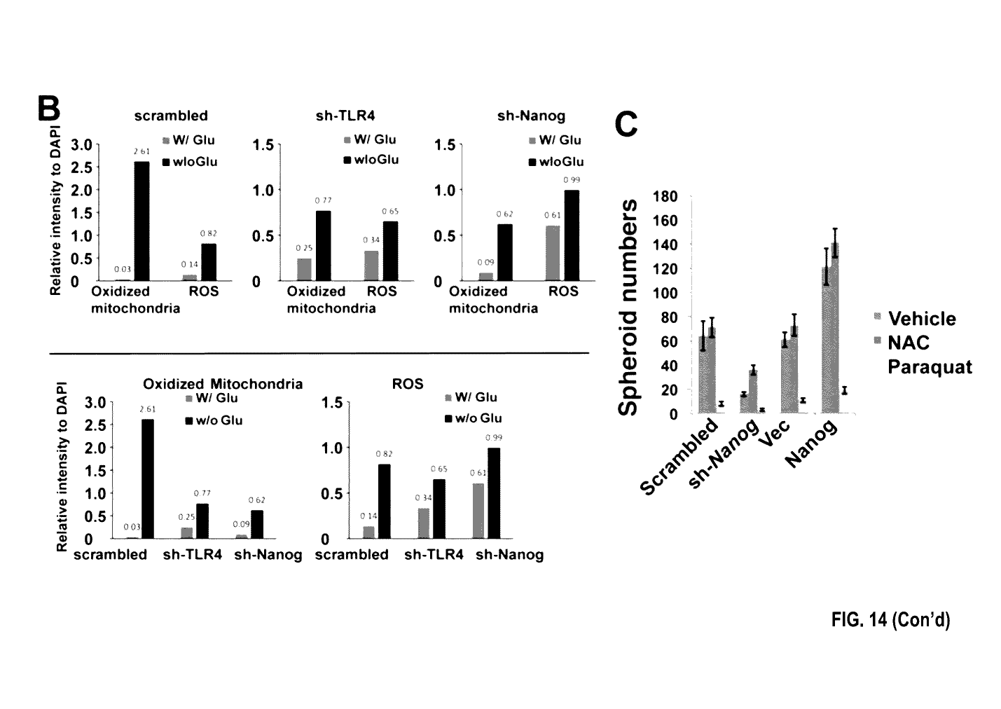

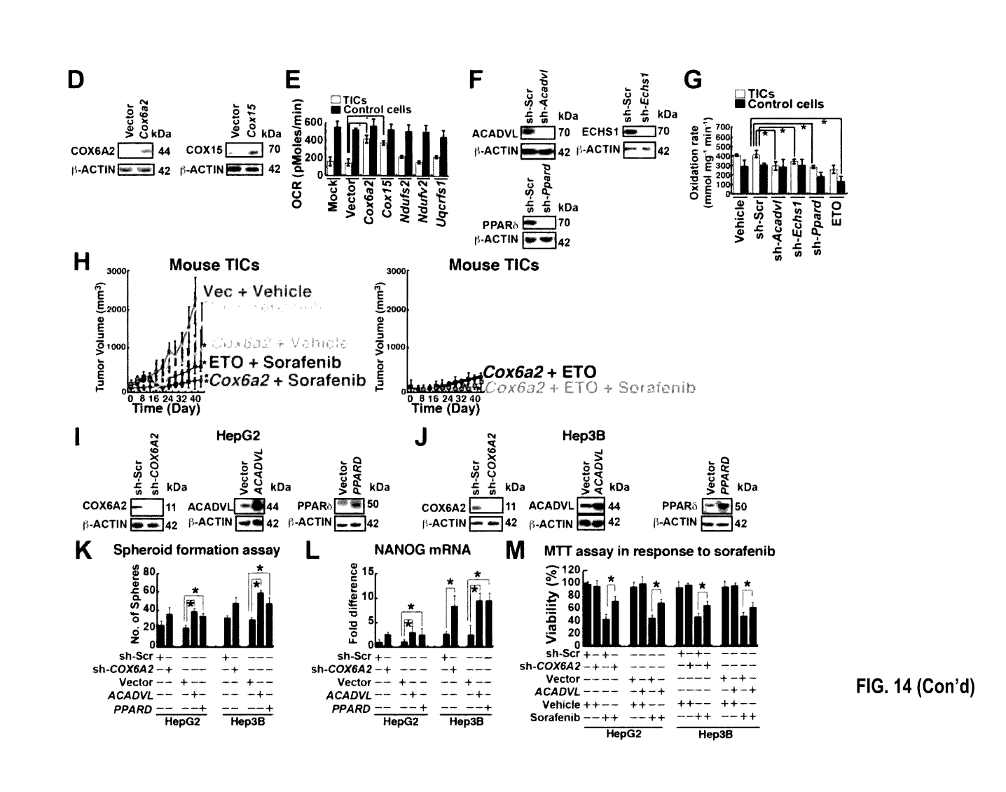

The respiratory status of mitochondria was next evaluated with respect to reactive oxygen species (ROS), a major by-product of the mitochondrial respiratory chain, in TICs with or without Nanog silencing. It was observed that more oxidatively active mitochondria were present in sh-Nanog TICs (FIG. 7A). Consequently ROS production was higher in Nanog-silenced TICs over scrambled shRNA-treated TICs (FIG. 7A, right), indicating that NANOG suppressed basal mitochondrial ROS production. Acadvl-silencing of TICs exhibited increased ROS production compared to control cells, indicating that downregulation of Acadvl promoted ROS accumulation (FIG. 7A, bottom). Glutamine withdrawal promoted ROS production in TICs (FIGS. 14A and 14B), indicating that glutaminolysis-mediated antioxidant products reduced ROS levels and supported cell proliferation. Treatment of TICs with Paraquat—an inducer of ROS, increased ROS production and significantly reduced tumor spheroid formation of TICs (FIGS. 7B and 7C). Similarly Nanog silencing reduced ROS-dependent spheroid formation (FIG. 14C). These results indicated that increased ROS production suppressed self-renewal ability.

To determine if NANOG-regulated OXPHOS gene(s) regulates mitochondrial metabolism, NANOG-regulated OXPHOS gene(s) were overexpressed in TICs. TICs transduced with Cox6a2 or Cox15 expression vectors showed two- and three-fold greater O₂ consumption, respectively (FIG. 14E). In order to test if restoration of OXPHOS gene(s) inhibited self-renewal ability, spheroid formation assays were performed on serially passaged TICs following OXPHOS gene transduction (FIG. 14D). Indeed, overexpression of either COX6A2 or COX15 inhibited spheroid formation ability (FIG. 7D).

Along similar lines human TICs were transduced with shRNAs which targeted various FAO genes and serially-passaged for appearance of spheroid cell masses. It was found that the spheroid numbers were reduced in the FAO gene-silenced group, indicating that the self-renewal ability of TICs was inhibited (FIG. 7E). Transduction of shRNAs-targeting Echs1 and Acadvl in TICs (FIG. 14F) inhibited the FAO activity of TICs (FIG. 14G). These results demonstrated the importance of NANOG in maintenance of TIC self-renewal ability by reducing mitochondrial respiration and ROS production.

NANOG Orchestrates TIC Oncogenicity and Therapeutic Resistance Mechanisms Via Mitochondrial Metabolic Reprogramming:

To address the effects of alteration of OXPHOS/FAO gene expression on the efficacy of the chemotherapeutic agent, sorafenib (Llovet and Bruix, 2008), the roles of the NANOG-repressed OXPHOS gene (Cox6a2) or the FAO inhibitor, ETO, were tested on sorafenib chemoresistance. OXPHOS genes was overexpressed or ETO was employed as an inhibitor of FAO in TICs and assessed their effects on cellular sorafenib sensitivity in an orthotopic tumor transplantation model in alcohol-fed mice. These results indicated causal roles of NANOG repression on OXPHOS and elevated expression of FAO genes in chemoresistance in a human and mouse TICs-xenograft mouse model (FIGS. 7G and 14H).

To test if restoration of OXPHOS and/or inhibition of FAO promote sorafenib-mediated apoptosis through the mitochondrial-pathway, cytochrome c release was examined in the mitochondria-enriched, heavy membrane fraction (HM) of total cell extract. It was observed that, following sorafenib treatment, cytochrome c was translocated from mitochondria into the soluble fraction (cytoplasm) of hepatocytes within 1-3 hours post treatment while cytochrome c in TICs remained mostly in the heavy membrane (HM) fraction (mitochondria) (FIG. 7F). The silencing of Nanog or overexpression of Cox6a2 and/or addition of FAO inhibitor (ETO) enhanced cytochrome c release from mitochondria in response to sorafenib treatment (FIG. 7F). Purity of the mitochondria-enriched fractions (HM) and cytosolic fractions (S), fraction-specific proteins was confirmed by marker enzymes. Voltage-dependent anion channel 1 (VDAC1), a major outer mitochondrial protein was detected in HM fractions, whereas copper zinc superoxide dismutase (Cu/Zn SOD), a cytosolic protein, was found in cytosolic fractions (FIG. 7F), indicating that the cell fractionation process was effective and supported this model. These results demonstrated causal roles of NANOG-mediated repression of OXPHOS and induction of FAO genes in chemoresistance.

Non-TIC cancer cells (HepG2 and Hep3B) were transduced with sh-COX6A2 or ACADVL expression vector and protein levels were validated by immunoblots (FIGS. 14I and 14J). Reduction of OXPHOS genes (i.e., Cox6a2) or overexpression of a FAO gene (i.e., Acadvl) in human, non-TIC HCC cell lines (HepG2 and Hep3B) promoted self-renewal ability (Fig. S7K) and NANOG expression (FIG. 14L) by spheroid formation assays and qRT-PCR analysis of NANOG. Thus, this relationship of OXPHOS gene expression to cell proliferation may be a more general property rather than restricted to TICs. Silencing and overexpression of Acadvl in non-TIC cancer cells conferred sorafenib resistance (FIG. 14M), indicating that alterations in the NANOG-OXPHOS pathway promotes a more stem-like property of cancer cells; thus rendering HCC more malignant and therapy-resistant.

NANOG Suppresses OXPHOS and Activates FAO, Thus Inhibiting OCR and ROS Production, Conferring a Tumor Chemoresistant State:

Complementary NANOG ChIP-seq and metabolomics studies of TICs demonstrated that NANOG induced by TLR4 suppressed mitochondrial OXPHOS and activated FAO, thus inhibiting OCR and ROS production. This conferred a tumor chemoresistant state which could be abrogated by NANOG-targeted gene silencing (FIG. 7H). The findings demonstrated a NANOG-dependent downstream effect on mitochondrial function in TICs that contributed to the mechanism of chemotherapy resistance. These metabolic reprogramming promoted self-renewal/oncogenesis, and explained how NANOG activation inhibited therapy-mediated apoptosis by quenching ROS production. Restoration of OXPHOS and activation of decreased FAO reduces tumorigenic capacity of TICs and increases susceptibility to chemotherapy.

As TICs rely on active FAO for their maintenance and function, FAO inhibitor suppresses self-renewal of leukemia-initiating cells (LICs) (Samudio et al., 2010). The effects of FAO gene silencing was experimentally reversed and restored the original TIC phenotype by overexpression of FAO genes (FIGS. 14K and 14L). Thus the fate of stem cells is metabolically switched by FAO (Ito et al., 2012). Potential mechanisms by which elevation of FAO maintains self-renewal ability include: (i) shunting of long-chain FA away from lipid and cell membrane synthesis; (ii) downregulation of ROS through production of NADPH to avoid loss of TICs; and (iii) reduction of metabolic resistance to chemotherapy. By these criteria, NANOG function could be construed to serve as a gatekeeper for FAO activity.

Role of FAO on TIC Self-Renewal, Growth and Chemoresistance:

The concept of targeting FAO for intervention is of high therapeutic relevance (Valent et al., 2012) since FAO-dependent NADPH production promotes survival of leukemia cells (Caro et al., 2012; Samudio et al., 2010). Although the BH3-iregulatory activity of proteins involved in FAO (via fatty acid transporter CPT1) (Giordano et al., 2005; Paumen et al., 1997) promotes the survival of leukemia cells (Samudio et al., 2010), inhibition of FAO facilitates BAK and BAX oligomerization, leading to cell death (Samudio et al., 2010). FAO inhibition leads to a cytotoxic increase of lipids, thus preventing this cytotoxicity might be benefit to cell survival (Samudio et al., 2010; Vickers, 2009). TICs rely on active FAO for their maintenance and function, thus inhibition of FAO could similarly affect leukemia-initiating cells (LICs) (Samudio et al., 2010). The genetic ablation of another FAO-regulatory protein, liver kinase B1 (LKB 1) similarly results in the depletion of the stem cell pool (Gan et al., 2010; Gurumurthy et al., 2010; Nakada et al., 2010). The effects of FAO gene silencing was reversed and the original TIC phenotype was restored by overexpression experiments of FAO genes followed using xenograft injection models.

The stem cell fate is metabolically switched by FAO (Ito et al., 2012). Self-renewal ability is promoted by elevation of FAO while de novo FA biosynthesis is inhibited in TICs. Potential mechanisms by which elevation of FAO maintains self-renewal ability include: (i) shunting of long-chain FA away from lipid and cell membrane synthesis; (ii) downregulation of ROS through production of NADPH to avoid loss of TICs; and (iii) reduction of metabolic resistance to chemotherapy. By these criteria, NANOG function could be a construed to be a gatekeeper for FAO. Notably, to date, no role has been ascribed to NANOG with respect to FAO inhibition in TICs.

As shown, Acadvl was repressed by NANOG. Acadvl encodes the inner mitochondrial membrane enzyme that catalyzes the first step of long-chain FAO and is capable of accommodating substrate acyl chain lengths as long as 24 carbons (Tucci et al., 2013). Acadvl−/− mice have reduced FAO activity and exhibit mitochondrial dysfunction, leading to hepatic steatosis, diacylglycerol accumulation and hepatic insulin resistance (Aoyama et al., 1995; Kurtz et al., 1998; Zhang et al., 2007; Zhang et al., 2003). Another FAO gene repressed by NANOG is Echs1 that encodes enoyl-CoA hydratase, an enzyme that hydrates the double bond between the second and third carbons on acyl-CoA to produce acetyl CoA and energy. Acadvl-silencing of TICs would be expected to exhibit increased ROS compared to control cells, indicating that downregulation of Acadvl promotes ROS accumulation.

Impact of FAO and OXPHOS on TIC Drug-Resistance:

Sorafenib is used as a monotherapy agent for the treatment of HCC; however, clinical experience reveals an eventual chemoresistance to sorafenib in HCC patients (Shen et al.; Villanueva et al., 2008). This chemoresistance may result from expansion of TICs. Indeed, antagonism of NANOG in TICs, enhances the efficacy of sorafenib in tumor-bearing mice and achieves ˜90% suppression of tumor growth (Huynh et al., 2009).

NANOG induced metabolic changes result in diminished mitochondrial oxygen consumption and ROS production, in turn protecting TICs from cell death caused by chemotherapeutic drugs such as rapamycin and sorafenib. In support of such a process, Nanog silencing restored OXPHOS, mitochondrial ROS generation, mitochondrial cytochrome c release, and apoptosis resulting from such chemotherapeutic treatment(s). As shown, NANOG downregulated OXPHOS genes (i.e., Cox6a2, Cox15) and up-regulated FAO gene expression (i.e., Acadvl, Echs1); therefore, reversal of NANOG-dependent effects on OXPHOS and FAO gene may offer a noteworthy strategy of countering therapeutic drug resistance associated with NANOG activation. The data showed that NANOG reprogramming of mitochondrial metabolism was indeed responsible for human TIC oncogenicity and chemo-resistance.

Microarray and Proteomics Analysis of Three Different Liver Disease Models:

The microarray data identified the downstream genes of Toll-like receptor 4 (TLR4) signaling, including four matrix metalloproteinases (MMP) that are also activated by inflammatory cytokines. In general, the presence of NS5A protein in mouse livers increased the expression of several stress response proteins (e.g., Hsp), stem cell factors (e.g., Nanog), and matrix metalloproteinases (MMP12 and MMP13). As MMP12, MMP13, and Nanog are known downstream targets of TLR4, these genes are likely to be induced by other effectors of TLR4 signaling in these mice. An example of such an occurrence was observed for a key FAO pathway enzyme: Acetyl-CoA acyltransferase (Acaa2), which was up-regulated in liver specimens from all three disease models (alcohol-fed Ns5a Tg, 12-month-Alcohol-fed-Core Tg mice, and 12-month-Western diet-fed Core Tg) but not in wild type control animals subjected to the same experimental diets.

E2F1 Transactivates NANOG:

Phosphorylation of E2F1 promotes its DNA binding activity. In addition to the Oct4-Sox2 heterodimer (Kuroda et al., 2005; Liang et al., 2008; Lin et al., 2005; Rodda et al., 2005; Storm et al., 2007), Oct-4 itself is an activator of the NANOG promoter (Wu da and Yao, 2005). TLR4 activation increases binding of E2F1 and p65/p50 to promoters of inflammatory cytokine genes such as Tnfα and Il-1β in a cooperative manner (Lim et al., 2007). The hypophosphorylated Rb is established as the most crucial regulator of E2F1 activity and the E2F1-Rb complex acts as an active transcriptional repressor. A sequential phosphorylation of E2F1 by cyclin-dependent kinases (cdks) promotes transcription by release of free E2F1 (Harbour and Dean, 2000; Lundberg and Weinberg, 1998). E2F1, one of several transcriptional activators of Nanog (Spender and Inman, 2009), is also induced by LPS in TICs but not in CD133(−) control cells, indicating cell lineage specific Nanog expression via increased E2F1 levels in TICs.

An analogy can be drawn to NANOG promoter from what is known about other gene promoter since E2F1 is heterodimerizing with DP monomer. Currently, six different E2F family members (E2F1 through E2F6) and three DP proteins (DP1 through DP3) have been identified in mammals. The heterodimerization of E2F and DP subunits are essential for both DNA binding and E2F-site-dependent transactivation because E2F and DP homodimers have minimal affinity for DNA (Johnson and Schneider-Broussard, 1998). As E2F is a heterodimeric factor composed of an E2F and a DP family member (Campanero et al., 1999), both DP1 and E2F1 may transactivate NANOG. E2F1/NF-□B sites in the enhancer and Oct4/Sox2 in the promoter have interactive relationships using IκBα super repressor. The enhancer region is positively regulated by STAT3, Nanog-Sall4 complex (Jiang et al., 2008; Suzuki et al., 2006; Wu et al., 2006).

Role of ROS and PPARs on TIC Self-Renewal Ability:

ROS inhibits stemness genes and self-renewal ability via activation of the p38 MAPK pathway (Ito et al., 2006), leading to BMI protein degradation and FOXO3 activation (Sato et al., 2014). This pathway is subject to regulation by alcohol and HCV (Tikhanovich et al., 2014). Overexpression of BMI promotes chemoresistance (Siddique and Saleem, 2012) through changes in the cell cycle, immortalization and intracellular GSH (antioxidant molecule in mitochondria) levels in stem cells and TICs (Wang et al., 2011). As another example, disruption of ATM promotes ROS production and leads to stem cell depletion (Ito et al., 2004). Therefore, general ROS production depletes the stem cell compartment and intrinsic self-renewal ability of these cells.

PPAR transcription factors (in particular, PPAR-δ and PPAR-α) have effects on all aspects of lipid metabolism (nutrient sensing and metabolic reprogramming), however in this system it is also important for release from stemness (differentiation). The novelty of this system is that lipid metabolism is involved in the transition from a normal cell to a transformed cell type to a TICs. These pathways impact the activation of the mitochondrial and peroxisomal FAO transcriptional program. In particular, changes occur in FA, sending uptake and intracellular binding, ketogenesis, triglyceride turnover, gluconeogenesis, and bile synthesis/secretion (Kersten et al., 1999). For example, PPARγ agonists inhibit TIC proliferation by inhibition of NANOG and SOX2 (Pestereva et al., 2012). Overexpression of PPAR-α and PPAR-δ promotes differentiation through FA uptake, utilization, and catabolism; whereas inhibition of PPARα signaling increases expression of pluripotency markers by deletion of Ppar-δ as well as inhibition of FAO.

Cancer Diagnostic and Treatment Tools:

Also disclosed herein are methods for treating HCC patients based on selected RNA profiling by using personalized precision medicine. The method will allow an individual's complete genetic profiling in just a few hours (FIG. 15). In the present method of diagnosing HCC, following a surgery, a pathologist prepares a series of pathological samples from surgically removed HCC tissues. With its rapidness, the method has contributed significantly to the treatment of HCC cancer (FIG. 15). Since the experiment does not have to be sent to next-generation sequence lab, the diagnosis is quick by regular routine qRT-PCR.

According to one embodiment, a method of identifying subjects with metastatic hepatocellular carcinoma (HCC) for tumor-initiating stem-like cell (TIC) targeted therapy comprises obtaining whole blood from a subject; retrieving circulating tumor cells (CTCs) and/or TICs from the whole blood; performing quantitative reverse transcriptase-PCR (qRT) PCR on retrieved CTCs and/or TICs; and identifying genes selected from the group consisting of NANOG, TWIST1, LIN28, MSI2, ACADVL, BIRC5, miR-22, LepR, YAP1 and IGF2BP3 that are upregulated and/or genes selected from the group consisting of COX6A2, COX15, TET1, TET2 and PTEN that are downregulated.

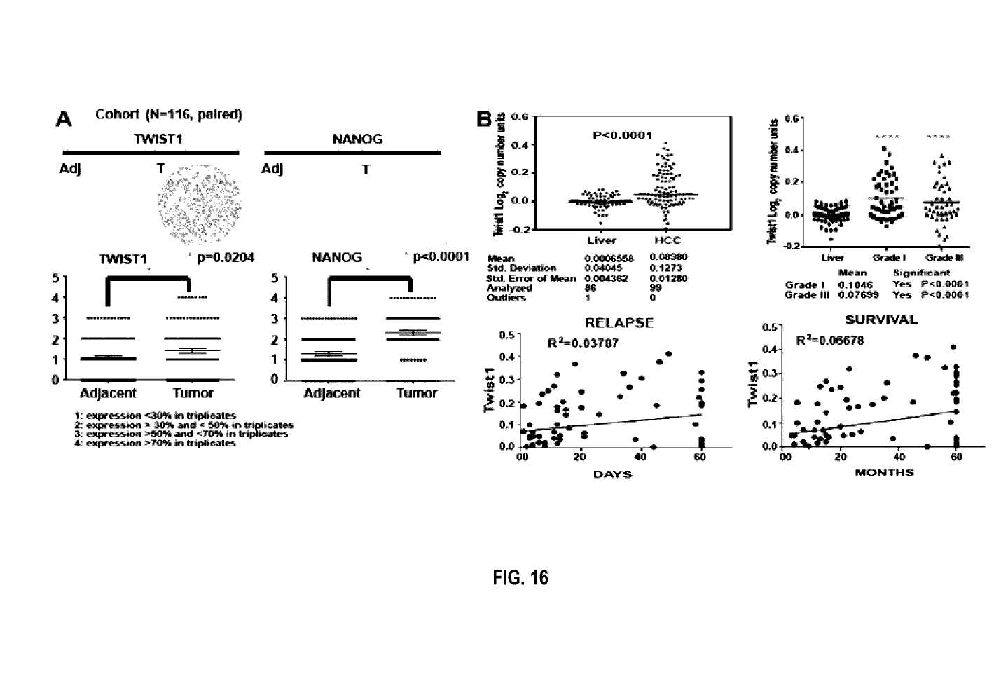

Cancer diagnostic tools (evidence-based medicine) were developed using panel of 15 gene sets for novel repurposed FDA-approved drug combination for metastatic unresectable liver cancer patients. Majority of patients suffers from recurrence and metastatic cancer. Diagnosis was done in CTCs in blood stream and surgically resected liver cancer tissues. Readout is tumor size shrinkage and survival rate and recurrence rate using CT-scan and ultrasound techniques. New chemotherapy against chemotherapy-resistant TICs is established and application for clinical trials to FDA within 1 year. These studies should ultimately lead to a well-tolerated and potentially curative treatment for relapsing and refractory aggressive HCCs. A new TIC-targeted chemotherapy was established using next-generation-sequence technology in unresectable metastatic HCC patients. The hypothesis was that NANOG is the nexus for the formation of chemoresistant TICs in HCC. Therefore, targeting of NANOG expression and function should lead to elimination of the TIC subpopulation. The role of NANOG-mediated oncogenesis was established in chemoresistant TICs isolated from HCC patients and designed novel therapeutic modalities for HCC. Paired IHC analyses of 142 patient samples (116 as a tissue microarray analysis) were performed to validate the significance of TWIST1 and NANOG in human tissue sections (FIG. 16A). To gain insights on the correlation of Twist1 with grade, survival, and relapse in HCC patients, in silico analysis was performed using the gene expression profile data. Analyses of two independent libraries from the repository demonstrated the significant impact of TWIST1 on HCC (FIG. 16B).

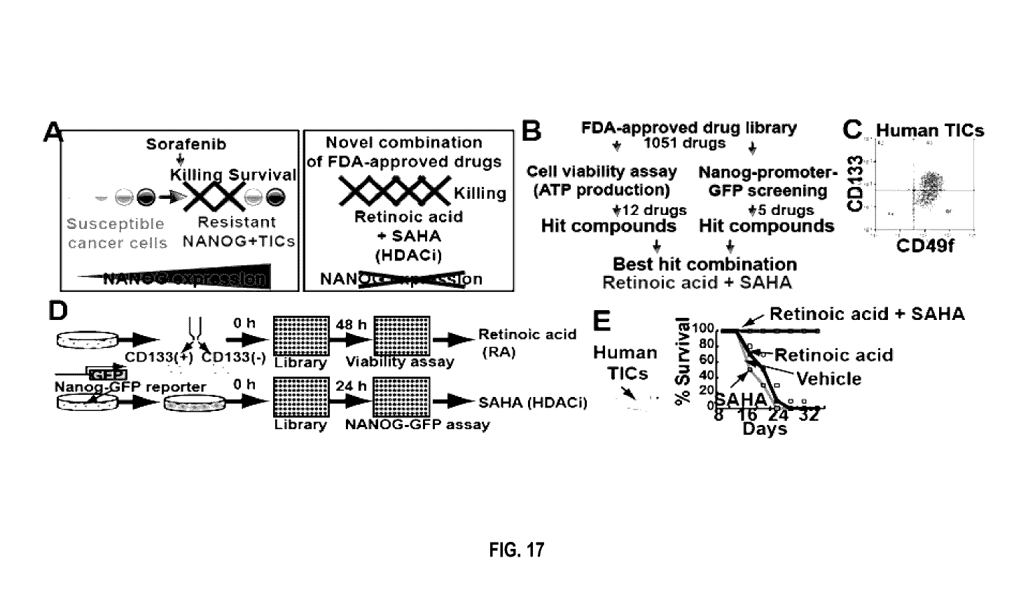

NANOG-dependent mechanisms underlying TIC chemoresistance were identified and characterized based on this drug screen via comparison with non-tumor cells (FIG. 17A). For identification of specific drugs that directly target the TIC population, drug screening was first performed on TICs, and found that retinoic acid (ATRA) specifically inhibited viability (ATP production) (FIGS. 17B-17D). Additionally, transduction of human TICs with a lentivirus Nanog-GFP reporter was used to perform high-throughput screening of drugs (FIG. 17D).

Responses to Selective TIC Inhibitors Using Subcutaneous Xenograft Transplantation of the TICs in Immunodeficient Mice:

To establish prognostic biomarkers for the best combination chemotherapy, five different patient-derived TICs were tested for responses as xenografts in NSG mice for the best combination chemotherapy based on mutation/transcriptome subtypes. Anti-tumorigenic ability by ATRA+SAHA targeting TICs encapsulated with SAHA was validated (Table 3). ATRA (8, 10 or 12 mg/kg) and SAHA (100 or 150 mg/m²) were administered (i.p.) every 5 days/week upon reaching 100 mm³ of tumor volume after tumor cell injection (Woodrum et al.). As placebo treatment, solvent (DMSO) in an equal total volume (0.2 ml/mouse) was injected. Tumor growth kinetics was studied. Combination of ATRA with SAHA further inhibited the self-renewal abilities of TICs in vivo (FIG. 17E). These results suggested that the drug combination reduced NANOG expression and induced apoptosis of TICs.

TABLE 3
Overall Design of in vivo Experiments - with HCC mouse models

| | | SAHA 8 mg/kg | SAHA 10 mg/kg | SAHA 12 mg/kg |
|---|---|---|---|---|
| Chemotherapy | Placebo | (human: 300 mg) | (human: 400 mg) | (human: 500 mg) |
| Placebo | 0% (0/20) | 10% (2/20) | 5% (1/20) | 10% (2/20) |
| ATRA 100 mg/m²/ | 22% (7/32) | 53% (17/32) | 91% (29/32) | 94% (30/32) |
| day | | | | |
| ATRA 150 mg/m²/ | 19% (6/32) | 88% (28/32) | 100% (32/32) | 100% (32/32) |
| day | | | | |

Prognostic Role of Biomarkers Identified by Single-Cell PCR:

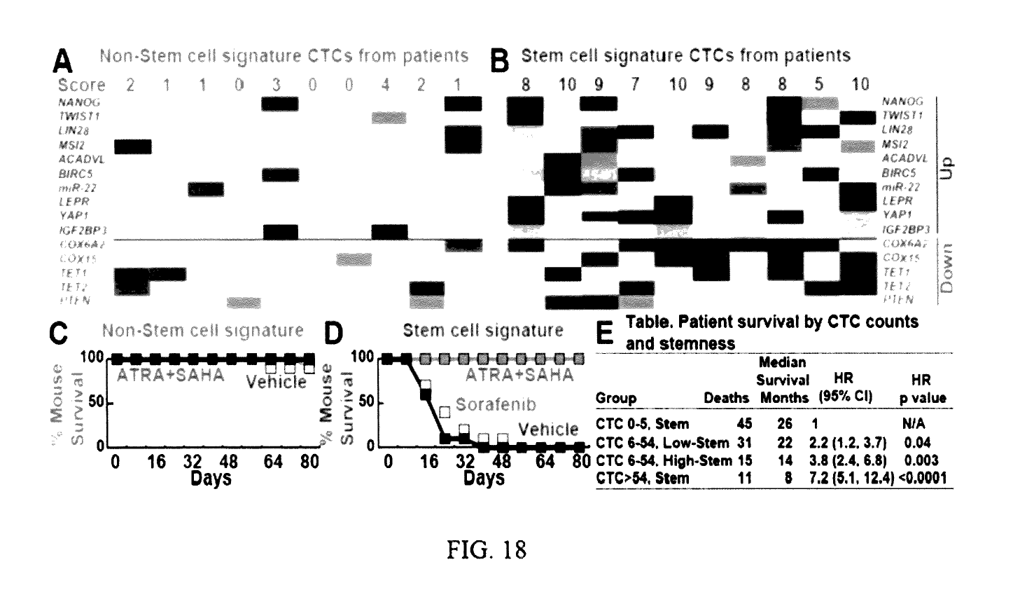

To diagnose TIC-targeted therapy is required or not, CTCs were isolated from patient PBMCs by biophysical characteristics and counted. To diagnose and identify markers for stratification of HCC patients, DEPArray automated cell isolation platforms capture CTCs within a new, patented microfluidic chip to recover tumor cells from whole blood. DEPArray-2nd generation (Silicon Biosystems) located in the USC Norris Comprehensive Cancer Center (HNRT 6516) under collaboration with Dr. Amir Goldkorn at USC. Target cells obtained by DEPArray can be directly sequenced and provides walk-away automation and processes. To gain insight into the potential functional implications, the gene-expression pattern of genes associated was compared with stemness (that is, NANOG and LIN28) using RNA sequencing in normal and cancer tissues. Whether quantitative expression levels of genes associated with stemness was evaluated could be used as a substitute measure for the malignancy of the corresponding tumors and serve to stratify HCC patients and predict clinical outcome in response to the novel combination chemotherapy. The same Fluidigm C1 Single-Cell Auto Prep System was used but to primary HCC from ten patients by dissociating and flow sorting cells into populations for C1 chips (FIG. 18A) (Patel et al., 2014; Shalek et al., 2014). The RNA-seq analysis identified sensitive and exclusive markers of TICs. Using the database, prominently up- or downregulated signature of CTC or TICs were identified as following:
Upregulated genes: NANOG, TWIST1, LIN28, MSI2, ACADVL, BIRC5, miR-22, LepR, YAP1 and IGF2BP3
Downregulated genes: COX6A2, COX15, TET1, TET2 and PTEN

If patient underwent liver transplantation and HCCs were surgically resected, these HCC and non-HCC tissues were sequenced by RNA-seq. If the stemness signature is prominent in signature gene panels (Score: more than 4), TIC-targeted therapy was initiated. If these signature genes were not prominently regulated (Score: less than 5), conventional chemotherapy target actionable mutations were searched by Exome-seq. Targetable and actionable mutations were pharmacologically targeted (data not shown).

A computer-assisted method was used to determine the threshold level between positive and negative expression and compared the clinical outcome of HCC patients in the three groups containing 10 patients with stemness signature and another 10 patients with non-stemness signature (FIGS. 18A and 18B). The survival outcomes of human HCC patients was analyzed after stratification into distinct gene-expression subsets, based on the expression of 15 RNA sets (FIGS. 18C and 18D). A multivariate analysis indicated that the 15-gene grouping system had the prognostic value and even better if number of CTC is considered together (FIG. 18E). These non-invasive RNA-seq data were extremely valuable, to diagnose which patients should be registered for TIC-targeted therapy.

In order to find compounds with minimum cytotoxicity and maximum anti-NANOG activity, the screen was performed using two methods. In order to investigate the underlying mechanism of TIC-chemoresistance, a genome-wide analysis was conducted of Nanog-promoter interactions employing the ChIP-seq method with Nanog-specific antibody in TICs. ChIP-seq analysis identified a significant level of Nanog enrichment proximal to transcription start sites (TSS) on a genome-wide basis with a distinctive pattern for the regulon with NANOG enrichment. An Ingenuity Pathway analysis on the set of NANOG-enriched gene promoters identified from the ChIP-seq data, implicated the involvement of major mitochondrial functions, including oxidative phosphorylation (OXPHOS)-related genes (Cox15, Cox6a2), fatty acid β-oxidation (FAO) genes (Acadvl). Twist1, BIRC5 and MSI2 have been identified as the convergent target for cancer metastasis. An important discovery is that novel diagnostic markers were identified due to CTC/TIC-mediated metastasis and poor prognosis.

Several mechanisms of actions of NANOG were elucidated in the maintenance and chemotherapy resistance of TICs involving not only the direct activation of self-renewal via stemness genes, but also the subsequent metabolic reprogramming in these cells leading to amplification of TIC oncogenic activity and their overall survival. The data showed that NANOG reprogramming of mitochondrial metabolism was indeed responsible for human TIC oncogenicity and chemoresistance. The metabolic bases of altered cell functions and cell fate in TICs define potentially new approaches for chemo-sensitization and elimination of TICs for more efficacious HCC therapies. These studies have led to a paradigm shift in the understanding the underlying basis of alcohol/HCV-associated cancer, thus facilitating future development of new personalized treatment strategies targeted towards NANOG+TICs arising from obesity, alcohol, or HCV-related HCC. In addition, cancer diagnostic tools (evidence-based medicine) were developed using panel of 15 gene sets for novel repurposed FDA-approved drug combination for metastatic unresectable liver cancer patients.

### Example 4

### NANOG and STAT3 Pathway: Summary

Long-term consumption of a HCFD elevates levels of gut-derived bacterial endotoxin in the plasma. Increased expression of TLR4 (a receptor for endotoxin) was previously demonstrated in hepatocytes of NS5A-Tg mice. Based on these findings, it was postulated that synergism between HCV and obesity in liver disease progression involved TLR4-dependent signaling. It was also reasoned that the TLR4-NANOG pathway might play a major role in mediating the synergism between obesity and HCV in the pathogenesis of HCC via generation of CD133+/Nanog+TICs. RNA microarray analysis on TICs derived from HCFD fed mice showed a significant increase in Twist1. It was previously demonstrated that Leptin and its receptor (OB-R) augmented pSTAT3 in TICs, these results led us to hypothesize that adipose tissue-derived leptin-pSTAT3 and TLR4-NANOG signals are needed for activation of Twist1 in TICs. Here, evidence is provide that TLR4 drives oncogenesis in part through the transcriptional induction of Twist1, a master regulator of epithelial mesenchymal transition (EMT), to generate cells with stem-like properties and a predisposition to the EMT. This signaling module therefore represents a new candidate target in the treatment of obesity- and HCV-associated HCC.

HCV-NS5A from a transgene (NS5A Tg) was expressed in Tlr4−/− (C57B16/10ScN), and wild type control mice. Mice were fed a HCFD for 12 months. TICs were identified and isolated based on being CD133+, CD49f+, and CD45−. 142 paraffin-embedded sections of different stage HCCs and adjacent non-tumor areas were obtained from the same patients, and performed gene expression, immunofluorescence, and immunohistochemical analyses.

A higher proportion of NS5A Tg mice developed liver tumors (39%) than mice that did not express HCV NS5A following the HCFD (6%); only 9% of Tlr4−/−NS5A Tg mice fed HCFD developed liver tumors. Livers from NS5A Tg mice fed the HCFD had increased levels of TLR4, NANOG, pSTAT3, and TWIST1 proteins, and increases in Tlr4, Nanog, Stat3, and Twist1 mRNAs. In TICs from NS5A Tg mice. NANOG and pSTAT3 directly interacts to activate expression of Twist1. Levels of TLR4, NANOG, pSTAT3, and TWIST were increased in HCC compared with non-tumor tissues from patients.

HCFD and HCV-NS5A together stimulated TLR4-NANOG and the OB-R-pSTAT3 signaling pathways resulting in liver tumorigenesis through an exaggerated mesenchymal phenotype with prominent Twist1-expressing TICs.

### Example 5

### NANOG and STAT3 Pathway: Experimental Procedure

Isolation of Mouse TICs Using FACS:

Tumor-initiating stem-like cells (TICs) were isolated from liver tumors in HCV-NS5A transgenic mice fed ad lib with an ethanol-containing liquid diet high in cholesterol and saturated fat (HCFD) (as previously described). Briefly tumors were surgically resected and mechanically dissociated by scissors. The tissue homogenate was digested with collagenase IV (BD Biosciences) and dispase (Sigma) mixture by incubation at 37° C. for 2 hours. The resulting single cell suspensions were incubated with anti-CD133, anti-CD49f and anti-CD45 antibodies and separated using FACS sorting, according to the manufacturer's protocol as previously described. Isolated TICs were maintained in Dulbecco's modified Eagle's medium nutrient mixture F-12 (DMEM/F12) containing 10% fetal bovine serum (FBS), 1% nucleosides, 1 μM dexamethasone, epidermal growth factor (EGF), 1 μg/ml penicillin, 1 μg/ml streptomycin and 1% nonessential amino acids (NEAA). CD133+TICs and CD133− control cells were cryopreserved in 60% FBS, 20% DMEM/F12, and 20% DMSO.

Plasmids, Production and Propagation of Lentivirus and Retrovirus Vectors:

The NS5A expression plasmid was constructed by inserting HCV-NS5A cDNA downstream of the CMV promoter into pcDNA3.1 (Invitrogen). All retroviruses were based on lentivirus (pPAX2: Addgene) or MMTV vectors (pVPack-GP: Stratagene). Lentivirus vectors were prepared by standard procedures using HEK293T cells. The packaging vector pPAX2 (Addgene), amphotropic envelope gene (VSV glycoprotein), packaging vector expressing GAG-POL: pMDV (Addgene), and shRNA expression cassette were co-transfected into HEK293T cells using BioT transfection reagent (Bioland Scientific LLC). Retroviruses expressing Stat3C and Stat3D were obtained from Prof. Daniel C. Link (Washington University of School of Medicine). Retroviruses expressing Stat3C and Stat3D were produced using Phoenix cells/HEK293T. 48 hours post transfection, the virus containing, cell supernatants were harvested, purified, mixed with polybrene (4 μg/ml), and used to infect cells (Huh7/TICs). The lentivirus titers were determined using LentiX-gostick (Clontech). Human GIPZ lentiviral shRNAmir target gene set was used for human toll-like receptor 4 (TLR4) (RHS4531-NR_024169, RHS4430-98525129, RHS4430-98843572, and RHS4430-99137800) (Open Biosystems). To increase silencing effects and to reduce off-target effects, a combination of shRNA lentiviruses were used to knock down target genes. MOI was calculated on a case by case basis depending on empirical transduction efficiency. The TWIST1-pGL3 reporter constructs were obtained from Prof. Nakamura (Tokyo Medical and Dental University).⁷

Tumor Collection and Analysis:

Tumor-bearing animals were sacrificed at day 30 or 35 (depending on the cell number injected) or whenever the tumor size exceeded the limit, and tumors were collected and measured for volume and weight. The tumor tissues were divided for (1) fixation with neutrally buffered 10% formalin for H&E staining and histological evaluation of the tumor; (2) fixation with 4% paraformaldehyde followed by sucrose treatment for subsequent immunostaining; and (3) snap-freezing in liquid N₂ for mRNA and protein analysis.

Endotoxin Measurement:

For endotoxin measurements, blood was collected from the inferior vena cava with pyrogen-free heparin as previously described. Extreme precautions were taken to avoid or eliminate pyrogen and endotoxin contamination of all surgical instruments and laboratory supplies. Blood samples were transferred into appropriate glass tubes made pyrogen-free by heat-treatment at 180° C. for 24 hours. Pyrogen-free water was supplied by the manufacturer (Kinetic-QCL, Santa Clara, Calif.; Biowhittaker). Just prior to assay, plasma samples were diluted and heated to 75° C. for 10 minutes to denature endotoxin-binding proteins that can mask endotoxin detection. Levels of endotoxin were measured using the Limulus amebocyte lysate pyrogen test and a kinetic program (Kinetic test, Kinetic-QCL, Santa Clara, Calif.; Biowhittaker). The threshold of detection is 0.1 pg/ml.

Histology & Immunohistochemistry:

Tissue samples were either fixed in 10% neutral buffered formalin and cryopreserved (Cryomatrix™) or with 4% paraformaldehyde (PFA) and embedded in paraffin, followed by thin-sectioning and mounted on glass slides. Samples were stained with either hematoxylin & eosin (H&E) or processed for immunostaining as appropriate. For the latter, primary antibodies against NANOG (Rabbit ab80892, Abcam), pSTAT3 (Rabbit #9134, Cell Signaling technology), TLR4 (Mouse monoclonal antibody, SC293072, Santa Cruz), TLR4 (goat sc-8694, Santa Cruz Biotechnology), or TWIST1 (Rabbit polyclonal antibody, sc-15393, Santa Cruz Biotechnology) were used along with their respective secondary antibodies. Slides were mounted for microscopy using xylene based mounting media, which includes hematoxylin for nuclei counterstaining (Vector Laboratories), as per the manufacturer's recommendations. The stained samples were then subjected to morphometric analysis. To determine the specificity of IHC, serial sections were similarly processed except primary antibodies were omitted. The area of interest was quantified using Metamorph software. The data shown represent the means±SD.

Quantitative Real-Time PCR (qRT-PCR):

Total RNA was extracted by using TRIzol Reagent (Invitrogen) and purified using the RNeasy mini kit (QIAGEN) according to the manufacturer's protocol. RNA concentration and purity were determined by A₂₆₀ and A₂₆₀/A₂₈₀ ratios, respectively. The RNA samples were treated with DNase I (Invitrogen) to remove residual traces of DNA. cDNA was obtained from 1 μg of total RNA, using SuperScript III reverse transcriptase (Invitrogen) and random primers in a final volume of 10 μl. cDNAs were amplified by PCR using the primer pairs listed in Table 4. Quantitative real-time PCR was performed on an ABI 7300 HT Real-Time PCR machine using 2×SYBR Green Master Mix (Applied Biosystems). Conditions for all reactions: 1 cycle at 50° C. for 2 min, followed by 1 cycle at 95° C. for 10 min, followed by 40 cycles at 95° C. for 15 s and 60° C. for 1 min. Specificity of the PCR products were tested by thermal dissociation curves. Gene expression was determined as relative ratio to β-Actin or GAPDH control via the ΔCt method. The data shown represents the means±standard deviation (SD).

TABLE 4
qRT-PCR primer sets

| Gene | Forward primer (5'-3') | Reverse primer (5'-3') |
|---|---|---|
| Twist-1 | AGA TGT CAT TGT TTC CAG AGA | TTA GTT ATC CAG CTC CAG AGT |
| | (SEQ ID NO.: 1) | (SEQ ID NO.: 2) |
| Nanog | AGG GTC TGC TA TGA GAT GCT | CAA CCA CTG GTT TTT CTG CCA |
| | (SEQ ID NO.: 3) | (SEQ ID NO.: 4) |
| Stat3 | GCC ACG TTG GTG TTT CAT AAT C | TTC GAA GGT TGT GCT GAT AGA G |
| | (SEQ ID NO.: 5) | (SEQ ID NO.: 6) |
| Tlr4 | ATG GCA TGG CTT ACA CCA CC | GAG GCC AAT TTT GTC TCC ACA |
| | (SEQ ID NO.: 7) | (SEQ ID NO.: 8) |
| E-cad | CTG CTG CTC CTA CTG TTT CTA C | TCT TCT TCT CCA CCT CCT TCT |
| | (SEQ ID NO.: 9) | (SEQ ID NO.: 10) |
| N-cad | CAG GGT GGA CGT CAT TGT AG | AGG GTC TCC ACC ACT GAT TC |
| | (SEQ ID NO.: 11) | (SEQ ID NO.: 12) |
| Gapdh | TGG ATT TGG ACG CAT TGG TC | TTT GCA CTG GTA CGT GTT GAT |
| | (SEQ ID NO.: 13) | (SEQ ID NO.: 14) |

Gene Array Analysis of Liver Tumors:

For identifying anti-apoptotic or proto-oncogenic proteins, serial cytosections of the mice liver tissues were prepared, stained them with H&E, and collected hepatocytes under homeostatic conditions, dysplastic, or transformed morphology by using laser-capture microscopy as described. In order to identify changes associated with HCFD, comparative analysis were performed on the cells isolated from livers of mice fed HCFD. A gene microarray analysis requires a minimum of 100-200 cells and proteomic analysis requires approximately 50,000-100,000 cells for each cell phenotype. The cells were lysed for RNA or protein extraction for gene chip analysis and 1D gel MS/MS analysis. The cells collected from each group of three animals were isolated for RNA or protein individually and later combined to create a representative sample pool and provide sufficient amounts of material for analysis. For gene profiling, the Affymetrix mouse gene chip (GeneChip Mouse Genome 430A 2.0) was used, and analyses were performed in the Genome Core Facility at Los Angeles Children's Hospital. Five individually extracted, mouse liver RNA specimens were pooled for each experimental group for microarray analysis. Data analysis was performed by using Partek Pro 5.1 (Partek Inc.). The normalization of the array data and statistical analysis were performed as described previously.

Proliferation Assay:

TICs were initially seeded at 5×10⁴ cells per well in a 6-well plate. Cell number and viability were measured at day 0, 2, 3, and 4 by the Countess™ automated cell counter (Invitrogen) with trypan blue exclusion. All experiments were carried out using three biological replicates and were repeated three times. The data shown represent the means±SD.

Wound Healing (Migration) Assay:

Cells were seeded in a 6-well plate and cultured until fully confluent. The confluent cell monolayer was slightly and quickly wounded with a linear scratch made with a sterile 200/100 μl pipette tip. The debris were removed, and the edges of the scratch were levelled with PBS washing. The open gap was inspected and photographed microscopically (10× object, Nikon) at 1 and 24 hours. All experiments were carried out using three biological replicates and were repeated three times. The data shown represents the mean±SD.

Soft-Agar Colony Formation Assay:

Cells (2.5×10³) were seeded in 0.35% agarose in TIC growth medium on a layer of 0.5% agar in the TIC growth medium. Cells were incubated for 10-14 days at 37° C. in a humidified atmosphere containing 5% CO₂ in air. The TIC growth medium (0.5 ml) was changed two or three times a week, as needed. At the end of the incubation period, colonies were stained with crystal violet (CV) followed by scanning for colony counts. The CV stain was also read at OD540. All experiments were carried out using three biological replicates and were repeated three times. The data shown represent the means±SD.

Site-Directed Mutagenesis:

Site-directed mutagenesis was performed as per a PCR-based mutagenesis kit (Quikchange site-directed mutagenesis kit, Stratagene, USA) with Advantage polymerase (Clontech). Consensus NANOG and STAT3 binding sites AATGG (SEQ ID NO.: 15) and TTCCTATAA (SEQ ID NO.: 16) have been previously observed in vitro. The TWIST1 plasmid −209/+1, containing putative NANOG binding sites (5′-TAAT(G/T)(G/T)-3′ (SEQ ID NO.: 17) or 5′-[CG][GA][CG]C[GC]ATTAN[GC]-3′) (SEQ ID NO.: 18) and STAT3 binding sites (5-TTC(C/T)N(A/G)GAA-3) (SEQ ID NO.: 19), were mutated utilizing a forward mutagenic primer and a reverse primer as previously described. The mutated sequences were confirmed by DNA sequencing. Primers used in this analysis are listed in Table 5. The data shown represent the means±SD.

TABLE 5
In vitro mutagenesis primer sets

| Gene | Forward primer (5′-3′) | Reverse primer (5′-3′) |
|---|---|---|
| Nanog-mut1 | GTT TGG GAG GAC GAA GGA GAC | CCT TCC TCG GGG TCT CCT TCG |
| Proximal | CCC GAG GAA GG | TCC TCC CAA AC |
| | (SEQ ID NO.: 20) | (SEQ ID NO.: 21) |
| Nanog-mut 2 | AGG TCG TTT TTG CCT GGT TTG GGA | CGT CCT CCC AAA CCA GGC AAA |
| Distal | GGA CG | AAC GAC CT |
| | (SEQ ID NO.: 22) | (SEQ ID NO.: 23) |
| Stat3-mut1 | TTT CCT ATA AAA CAT GAT TAC GTC | CGT GAG GAG GAG GGA CGT AAT |
| Proximal | CCT CCT CCT CAC G | CAT GTT TTA TAG GAA A |
| | (SEQ ID NO.: 25) | (SEQ ID NO.: 26) |
| Stat3-mut 2 | CTG GAA AGC GGA AAC TAT GAT TAC | GGG ACT TTT CGA AGT TCG TAA |
| Distal | GAA CTT CGA AAA GTC CC | TCA TAG TTT CCG CTT TCC AG |
| | (SEQ ID NO.: 27) | (SEQ ID NO.: 28) |

Confocal Immunofluorescent Microscopy:

Immunofluoroscence staining of cryosections or paraffin sections was performed using primary antibodies against NANOG (Rabbit ab80892, Abeam), P-Stat3 (Rabbit #9134, Cell Signaling technology), TLR4 (Mouse monoclonal antibody, SC293072, Santa Cruz), TLR4 (goat sc-8694, Santa Cruz Biotechnology), or TWIST1 (Rabbit polyclonal antibody, sc-15393, Santa Cruz Biotechnology). Specimens were mounted on glass slides according to the manufacturer's recommendations using mounting media which included DAPI for nuclei counterstaining (Vector Laboratories). To determine the specificity of IF, serial sections were similarly processed except primary antibodies were omitted. Images were captured on a Zeiss LSM510 confocal microscope using sequential acquisition imaging. The degree of staining was categorized by the extent and the intensity of staining. Image analysis of nuclear translocation was performed using Metamorph or ImageJ v3.91 software (http://rsb.info.nih.gov/ij). A minimum of 10 high power fields were selected for image analysis. To avoid experimental bias for the staining colocalization of TLR4/NANOG/pSTAT3 with TWIST1, nuclear (DAPI) staining was used to identify fields with near-confluent cells for the purpose of maximizing the cell numbers used for analysis. The selected fields were then evaluated for the expression of TLR4, pSTAT3, NANOG, and TWIST1. Quantitative fluorescence data were exported from ImageJ generated histograms in Microsoft Excel software for further analysis and presentation. The data shown represent the means±SD.

Tissue Microarray Analysis (TMA):

The HCC TMA was constructed as previously described.²¹ Briefly, archived liver cases were reviewed, and areas containing HCC and benign hepatic parenchyma were marked for sampling. Three cores per HCC and matched benign from the same patient, measuring 0.6 mm in diameter, were obtained from selected regions in each donor paraffin block and transferred to a recipient paraffin block.

Spheroid Assay:

TICs (50 cells) were seeded onto Ultra low attachment 96-well plates (Corning Inc.), followed by incubation at 37° C. in a humidified atmosphere containing 5% CO₂ for 14 days. 100 μl/well of TIC growth medium was replaced twice a week. The number of colonies was counted under bright-field microscopy, and the proliferation was measured using counting numbers of spheroides and Luminescent Cell Viability Assay (Promega) followed by manufacturer's instructions. All experiments were carried out using 24 biological replicates and were repeated three times. The data shown represent the means±SD.

Immunoblotting:

Total cell lysates were prepared by lysing the cells in cold NP-40 buffer (150 mM NaCl, 1.0% NP-40, 10% Glycerol, and 50 mM Tris, pH 8.0) containing complete protease inhibitor mixture (Roche) for 1 h on ice, followed by centrifugation at 14,000 RPM for 15 min and collection of the clarified supernatant. Protein concentrations were determined using the DC protein assay Kit (Bio-Rad), and the supernatant was mixed with 6×Laemmli sample buffer. Proteins were separated on 10% SDS-PAGE and transferred to nitrocellulose membranes (Thermo). The membranes were blocked with 5% non-fat milk+0.1% tween-20 for 1 h, followed by incubation with the primary antibodies: TWIST1 (Santa Cruz Biotechnology), E-CADHERIN (BD Biosciences), N-CADHERIN (Santa Cruz Biotechnology), TLR4 (Santa Cruz Biotechnology), NANOG (Abcam), pSTAT3 (Cell signaling Technology) and P-ACTIN (sigma) (all at 1:1,000 dilution) at 4° C. overnight. Horseradish peroxidase-conjugated IgG (Santa Cruz Biotechnology; 1:2,000) was used to treat the membranes for 1 h at room temperature, and visualized with SuperSignal® West Pico Chemiluminescent substrate (Thermo). The immunoreactive bands were detected with Premium Clear Blue X-Ray films (Bioland Scientific LLC). Quantification of the bands was performed using ImageJ software. The data shown represent the means±SD. Antibodies used for these studies are listed in Table 6 as follows.

TABLE 6
Antibody list

| Antibody | Manufacturer |
|---|---|
| TWIST1 | SC-15393 (Santa Cruz Bio Technology) |
| NANOG | ab80892 (Abcam) |
| STAT3 | #9139S (Cell Signalling) |
| P-STAT3 | #9134S (Cell Signalling) |
| TAK1 | SC-7162 (Santa Cruz Bio Technology) |
| TRAF6 | SC-7221 (Santa Cruz Bio Technology) |
| IKK-B | SC-8014 (Santa Cruz Bio Technology) |
| P-IKK-B | #2694 (Cell Signalling) |
| B-ACTIN | A5441 (SIGMA) |

Promoter Luciferase Reporter Assays:

TICs obtained from NS5A transgenic mice (<10 passages in culture) were cultured in six-well plates and cotransfected using BioT (Bio land Scientific) with 1 μg Twist1 promoter-fused to Firefly luciferase reporter and 50 ng (SV40) Renilla luciferase expression vector to control for transfection efficiency. Forty-eight hours after transfection, cells were lysed in 1× passive lysis buffer, and luciferase activity was measured using the Dual-Glo Luciferase System (Promega) using a Lumat LB9501 luminometer (Berthold). At least three independent biological replicates were used for this experiment and were performed for at least total of three determination. Plasmids used in this assay are listed in Table 5. The data shown represent the means±SD.

Subcutaneous Xenograft Transplantation of the TICs into Immunodeficient Mice:

NOG mice were purchased from Taconic and housed under pathogen-free conditions in accordance with approved Institutional Animal Care and Use Committee protocols. TICs (10⁵) in 100 μl solution were mixed with 100 μl Matrigel (BD Biosciences) and were injected into the dorsal flanks of female NOG mice 8-9 weeks of age. Mice were anesthetized with ketamine (80 mg/kg) and xylazine (10 mg/kg) cocktail through I.P. during the procedure. The tumor volume was measured with a caliper and calculated according to the formula V=[a×(b²)]/2, where “V’ represents tumor volume, “a” represents the largest, and “b” the smallest superficial diameter. The data shown represents the mean±SD.

Live Animal Imaging:

The tumor bearing mice was monitored using noninvasive imaging by whole-body GFP imaging utilizing the bioluminescence imaging system (IVIS 200 Imaging Series, Xenogen) at day 21 and 35.

Chromatin Immunoprecipitation (ChIP) and Re-ChIP Analysis:

CD133+ liver TICs grown in 10-cm cell culture dishes following LPS and leptin treatment were fixed for 10 min at room temperature by addition of 1% paraformaldehyde to the growth medium. Cells were washed twice in cold PBS supplemented with complete protease inhibitor mixture and gently scraped from the plate. Cell lysis and chromatin immunoprecipitation (ChIP) were performed using the ChIP Assay Kit (Millipore). For chromatin fragmentation, cells were sonicated using a Branson Sonifier 450 on power setting 4 in 30-s bursts with 1 min cooling on ice for a total sonication time of 4 min. For immunoprecipitations, 8βg of each antibody was used. Anti-Nanog (Abcam) and Anti Stat3 (Cell signaling technology) monoclonal antibody were used for immunoprecipitation. Preimmune IgG was used as the antibody specificity control. Immunoprecipitated dNa was quantified for Twist1 promoters using q-PCR primers which are listed in Table 7. The Re-ChIP or Sequential ChIP analysis was performed according to the manufacture's protocol (Active Motif Re-ChIP IT®), whereas all the initial sample preparation where the same as explained above. The data shown represent the means±SD.

TABLE 7
ChIP-qPCR primer sets

| Gene | Forward primer (5′-3′) | Reverse primer (5′-3′) |
|---|---|---|
| Nanog- Proximal | ATG GTT TGG GAG GAC GAG TTA | AAA GTT TCC GCT TTC CAG TCC |
| Binding Site | (SEQ ID NO.: 29) | (SEQ ID NO.: 30) |
| Stat3-Distal | GGA CTG GAA AGC GGA AAC T | GCA GAC TTG GAG GCT CTT ATA C |
| Binding Site | (SEQ ID NO.: 31) | (SEQ ID NO.: 32) |
| Stat3-Proximal | GCC AGG TCG TTT TTG AAT GG | CGT GCA GGC GGA AAG TTT GG |
| Binding Site | (SEQ ID NO.: 33) | (SEQ ID NO.: 34) |
| Specificity | CCC AGC AAT CCC AAA TCG G | CAG CAA TGG CAA CAG CTT CTA |
| control -1 | (SEQ ID NO.: 35) | (SEQ ID NO.: 36) |
| Specificity | CTC ACG TCA GGC CAA TGA | GAG AGC TGC AGA CTT GGA G |
| control - 2 | (SEQ ID NO.: 37) | (SEQ ID NO.: 38) |

Statistical Analysis:

Statistical significance was estimated by un-paired, two-tailed Student's t test. P values are indicated in the figures. Bars represent the mean and error bars the SD. For most of the figures, statistical significance is represented by asterisks above each column: *P<0.05, **P<0.005, ***P<0.001 and ****P<0.0001. Some figures have been represented with pound sign or ampersand, details of which are given in the respective figure legends. For FIG. 7 B, statistical significance was calculated using two-way ANOVA method. In this specific analysis the time point used was where all mice were still alive, before any required euthanasia.

Mouse Studies:

All experiments on mice were approved by the USC Institutional Animal Care and Use Committee. Transgenic mice expressing the HCV-NS5A gene under control of the ApoE promoter were obtained from Prof. Ratna Ray (Saint Louis University, St. Louis, Mo.). TLR4-deficient mice (C57Bl6/10ScN), control mice (C57Bl6/10ScSn) and C57Bl/6 mice were purchased from Jackson Laboratories. To generate WT, NS5A, Tlr4−/−, and Tlr4−/−NS5A mice on a more congenic genetic background, NS5A Tg (FVB strain) and Tlr4−/− mice were crossbred on a C57BL/6 background (Jackson Laboratories) more than 8 generations at USC. Littermates on mixed C57BL/6-NS5A transgenic and Tlr4−/− mice (Jackson Labs) were intercrossed at least eight generations to produce WT, NS5A, Tlr4−/−, and Tlr4−/− NS5A mice on a more congenic genetic background. Both genders of mice were used for experiments. High-cholesterol high-fat diet was modified from TD.03350 (Harkan Teklad; Inc.) as previously described, where indicated mice were fed ad lib with an ethanol-containing Lieber-DeCarli diet containing 3.5% ethanol or isocaloric dextrin (Bioserv, Frenchtown, N.J.) high in cholesterol and saturated fat (HCFD) beginning at eight weeks of age for a period of 12 months. Other mice were fed modified high fat AIN-93G purified ethanol liquid diet with anhydrous milkfat, lard, corn oil and 1% cholesterol (DYET#710362: DYETS, Inc.) or Lieber-DeCarli Regular Control Diet (DYET#710027).

Human Subjects:

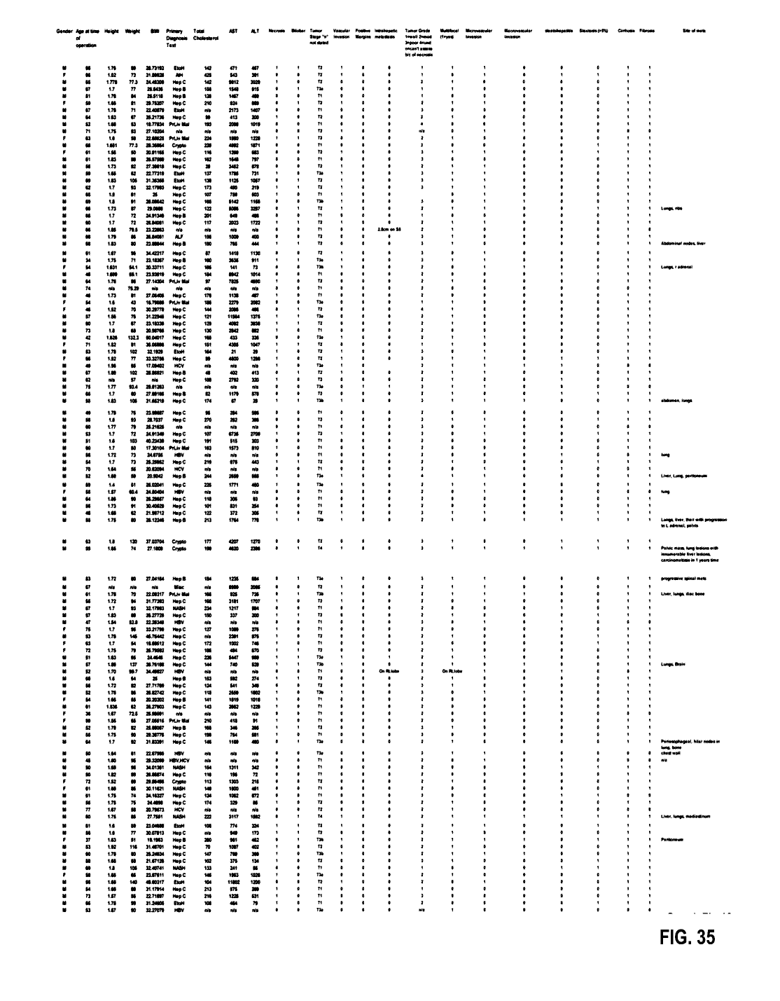

Paraffin embedded tissue sections were obtained in accordance with the approved Institutional Review Board (IRB). There were three institutions [University of Southern California, University of California at Los Angeles (UCLA) and University of Minnesota] that gave Institutional Review Board (IRB) approval for the supplied specimens. Specimens were obtained from the Liver Tissue Cell Distribution System (LTCDS) at the University of Minnesota according to the following criteria: surgically excised HCC tissues from 8 patients+/−HCV infection, +/− history of alcoholism, +/− obesity/diabetes/BMI>30. Eighteen specimens were also obtained from the Hepatobiliary and Liver Transplantation Service at the USC Keck School of Medicine. One hundred sixteen cases of HCC were identified from 2002-2011 by searching the UCLA Department of Pathology database using the following search terms: liver, hepatocellular carcinoma, resection, and transplant. All patient identifiers were removed to protect confidentiality. Samples were obtained from both genders between the ages of 42 and 80. Histologically, all samples displayed varying degrees of microvesicular and macrovesicular steatosis and inflammation in addition to different stages of HCC. These paired-116 specimens were the livers that had been dissected with the tumor and adjacent non-cancerous areas from the same patients. Clinicopathological information is described in FIG. 35 summarized in Table 8.

TABLE 8
Clinicopathological Features Of Patients
With Hepatocellular Carcinoma

| Feature | Value | |
|---|---|---|
| Age (years) | 59 ± 8.51* | (34-77) |
| Gender | | |
| Male | 93 | |
| Female | 20 | |
| BMI | 27 ± 0.05* | (19-50) |
| Total Cholesterol | 157 ± 0.48* | (48-425) |
| AST | 2124 ± 21.74* | (21-11802) |
| ALT | 843 ± 7.25* | (55-3838) |
| Primary | | |
| Diagnosis | | |

| | |
|---|---|
| PrLive Mal | 7 |
| NASH | 5 |
| Crypto | 4 |
| HBV | 21 |
| HCV | 60 |
| Tumor stage | |
| T1 | 40 |
| T2 | 46 |
| T3a | 16 |
| T3b | 9 |
| T4 | 2 |
| Tumor Grade | |
| 1 | 19 |
| 2 | 64 |
| 3 | 35 |
| 4 | 3 |
| Metastasis | 16 |

*Value = Mean ± SEM

### Example 6

### NANOG and STAT3 Pathway: Data and Analysis

HCFD Promotes Liver Oncogenesis in NS5A Tg Mice in a TLR4-Dependent Manner:

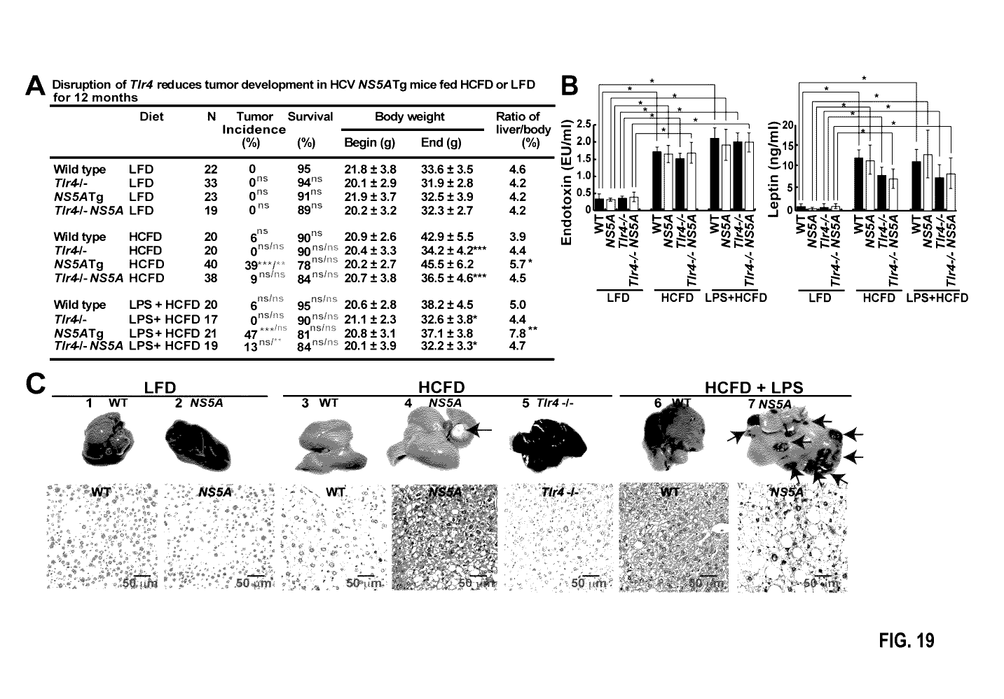

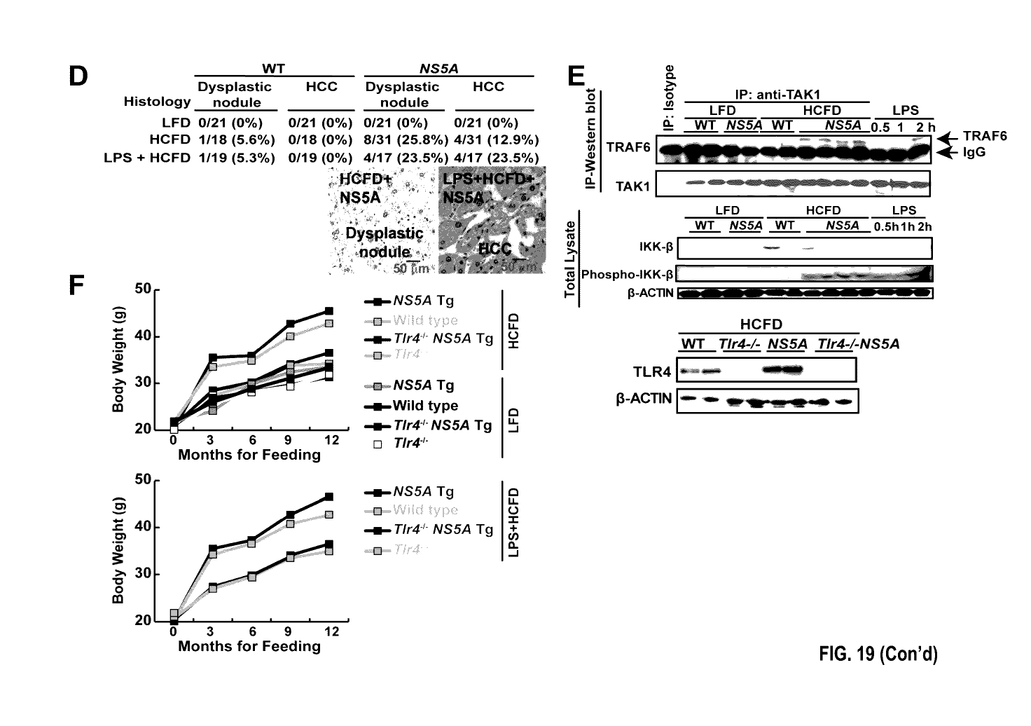

An in vivo loss of function strategy was employed to test the role of TLR4 in this interplay between NS5A and obesity. Hepatocyte-specific NS5A Tg, and wild-type (WT) mice with or without TLR4 deficiency (Tlr4^{-/-}) were maintained on low-fat diet (LFD) or an HCFD with or without supplemental LPS for 12 months (FIG. 19A). HCFD consumption resulted in an obese population (WT and NS5A Tg mice); however, this outcome was remarkably prevented by TLR4 deficiency in either genotype (FIGS. 19A and 19F). In HCFD mice, a liver tumor incidence of 39% was observed in NS5A Tg mice compared to 6% in WT mice. By contrast, a significant decrease of tumor incidence to 9% in was observed Tlr4^{-/-} NS5A Tg mice (FIGS. 19A and 19C). Conversely, LPS supplementation in the HCFD (100 mg/kg) further increased the incidence to 47% in NS5A Tg mice (FIG. 19A). This observation indicated a significant contribution of the LPS-TLR4 pathway in hepatocarcinogenesis. Additionally, the presence of NS5A in HCFD-fed mice significantly increased the liver to body ratio which coincided with severe liver hepatomegaly and inflammation (FIGS. 19A, 19C and Table 9 below).

TABLE 9
Liver histological grading of hcv ns5a tg mice fed the low-fat diet, HCFD or
HCFD + LPS for 12 months.

| | | Fatty liver | Spotty necrosis | Dysplasia | Inflammation |
|---|---|---|---|---|---|
| | Diet | (0-4+) | (0-2+) | (0-4+) | (0-2+) |
| WT | Low fat diet | 0.1 ± 0.1 | 0.1 ± 0.2 | 0.2 ± 0.1 | 0.2 ± 0.3 |
| NS5A Tg | Low fat diet | 0.1 ± 0.2 | 0.1 ± 0.1 | 0.1 ± 0.1^{NS} | 0.2 ± 0.2^{NS} |
| Tlr4 −/− | Low fat diet | 0 | 0.1 ± 0.3 | 0.1 ± 0.4^{NS} | 0.1 ± 0.2^{NS} |
| Tlr4 −/− NS5A Tg | Low fat diet | 0.1 ± 0.1 | 0.2 ± 0.1 | 0.2 ± 0.3^{NS} | 0.2 ± 0.3^{NS} |
| WT | HCFD | 2.3 ± 0.8 | 0.6 ± 0.2 | 0.2 ± 0.1^{NS} | 0.6 ± 0.4^{NS} |
| NS5A Tg | HCFD | 3.5 ± 0.6 | 1.1 ± 0.3* | 1.4 ± 0.5*/* | 1.2 ± 0.4*/* |
| Tlr4 −/− | HCFD | 1.0 ± 0.7 | 0.4 ± 0.3 | 0.1 ± 0.2^{NS/NS} | 0.4 ± 0.4^{NS/NS} |
| Tlr4 −/− NS5A Tg | HCFD | 1.2 ± 0.8 | 0.5 ± 0.5 | 0.3 ± 0.5^{NS/NS} | 0.5 ± 0.2^{NS/NS} |
| WT | HCFD + LPS | 2.7 ± 0.7 | 0.7 ± 0.3 | 0.3 ± 0.3^{NS/NS} | 0.7 ± 0.3^{NS/NS} |
| NS5A Tg | HCFD + LPS | 3.7 ± 1.1 | 1.3 ± 0.5* | 1.8 ± 0.6**/** | 1.7 ± 0.6**/** |
| Tlr4 −/− | HCFD + LPS | 1.2 ± 0.5 | 0.3 ± 0.3 | 0.2 ± 0.6^{NS/NS} | 0.2 ± 0.4^{NS/NS} |
| Tlr4 −/− NS5A Tg | HCFD + LPS | 1.5 ± 0.7 | 0.3 ± 0.6* | 0.5 ± 0.4^{NS/NS} | 0.4 ± 0.5^{NS/NS} |

*P < 0.05, compared to respective HCFD diet-fed WT mice;
**P < 0.05, compared to respective HCFD diet-fed NS5A Tg mice;
#P < 0.05, compared to HCFD plus LPS-fed WT mice. Fatty liver, 2+: 25%~50% heaptocytes with fat; 3+: 50%~75% with fat;
4+: >75% with fat. Inflammation, 1+: lesions encompassing less than ⅓ acinus; 2+: lesions larger than whole acini. (WT-HCFD; *, P < 0.05 **, P < 0.01 ***, P < 0.005, geen scripts and symbols - statistical analysis in comparison to low fat diet (LFD), purple scripts and symbols - statistical analysis in comparison to HCFD)

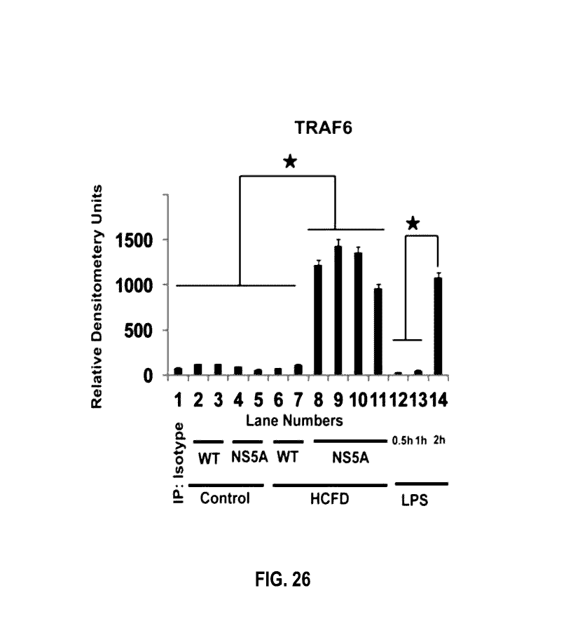

As predicted, HCFD, and HCFD+LPS feeding markedly raised plasma endotoxin and leptin levels in all tested cohorts (FIG. 19B). Several liver malignancies were observed in NS5A Tg mice, but not in the control animals. Additional observed pathologies included NASH-like bloating (FIG. 19C), dysplastic nodules (non-malignant) and HCCs (FIG. 19D). Activation of TLR4 signaling was assessed by co-IP of TGF-α-activated kinase 1 (TAK1)—tumor necrosis factor receptor-associated factor 6 (TRAF6) and immunoblotting for p-IKK-β. Concomitant TLR4 activation through TRAF6-TAK1-p-IKK-β was evident in HCFD-fed NS5A Tg (FIG. 19E, and FIG. 26), but not in LFD-fed cohorts. As a positive control for TLR4 activation parameters, a single i. p. dose of LPS (2 mg/kg) was given to chow-fed WT mice prior to sample collection (last three lanes of FIG. 19E top). Collectively, these results demonstrated that HCV-NS5A and HCFD acted synergistically to induce liver tumors in a manner dependent on TLR4.

Twist1 Identified as One of the Most Conspicuously Upregulated Genes in TLR4-Dependent NS5A- and HCFD-Driven Hepatocarcinogenesis:

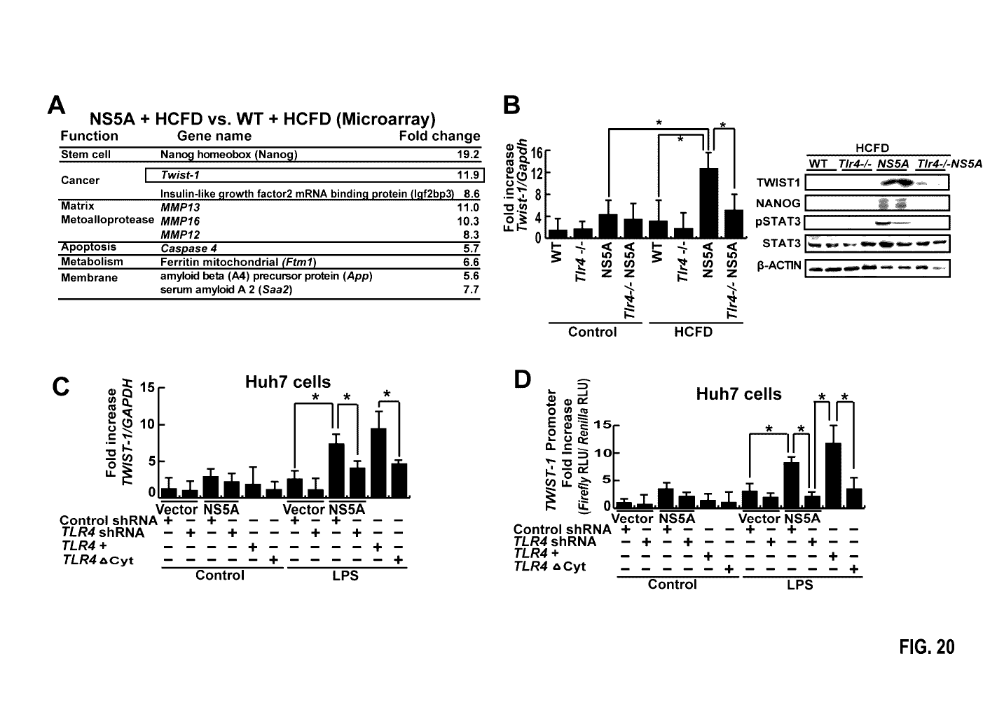

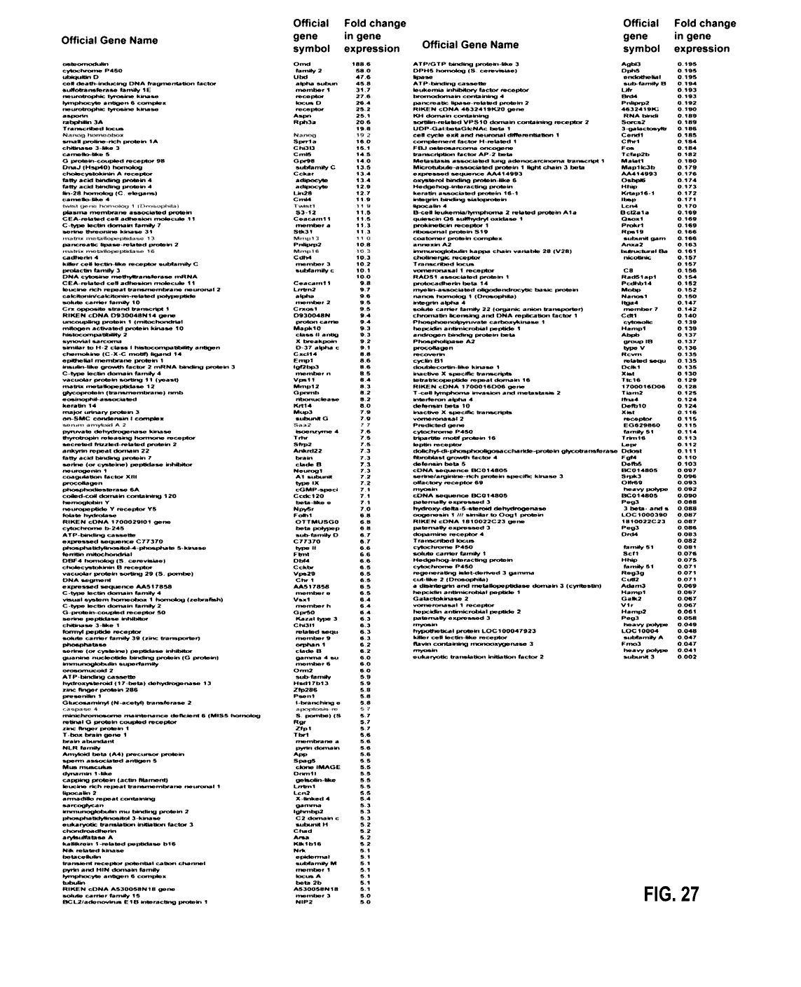

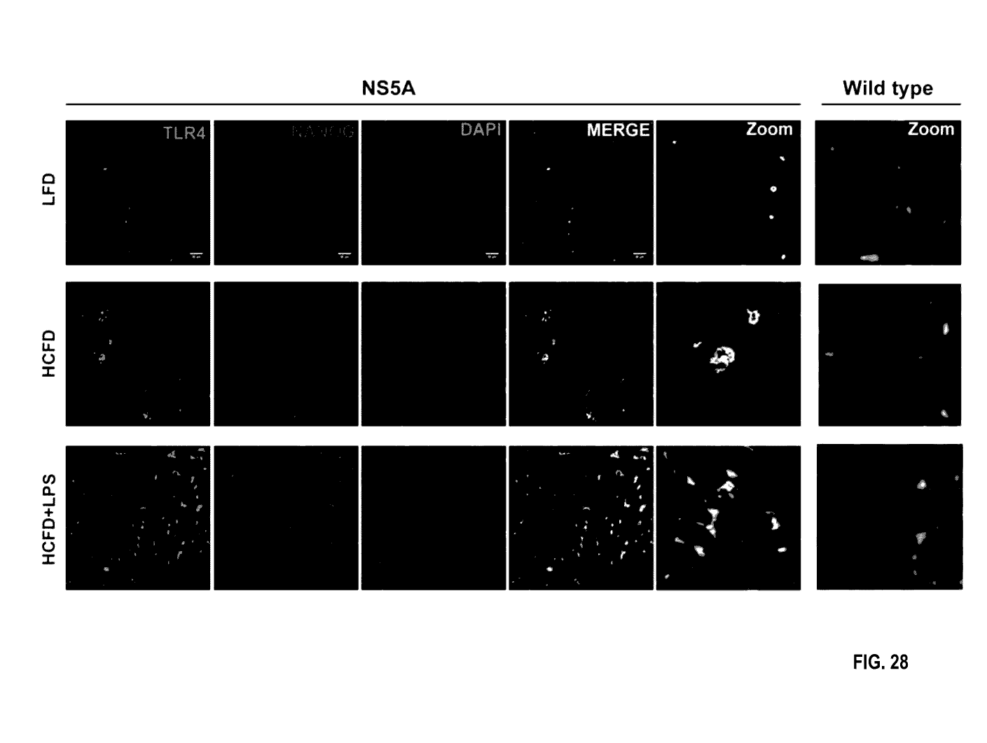

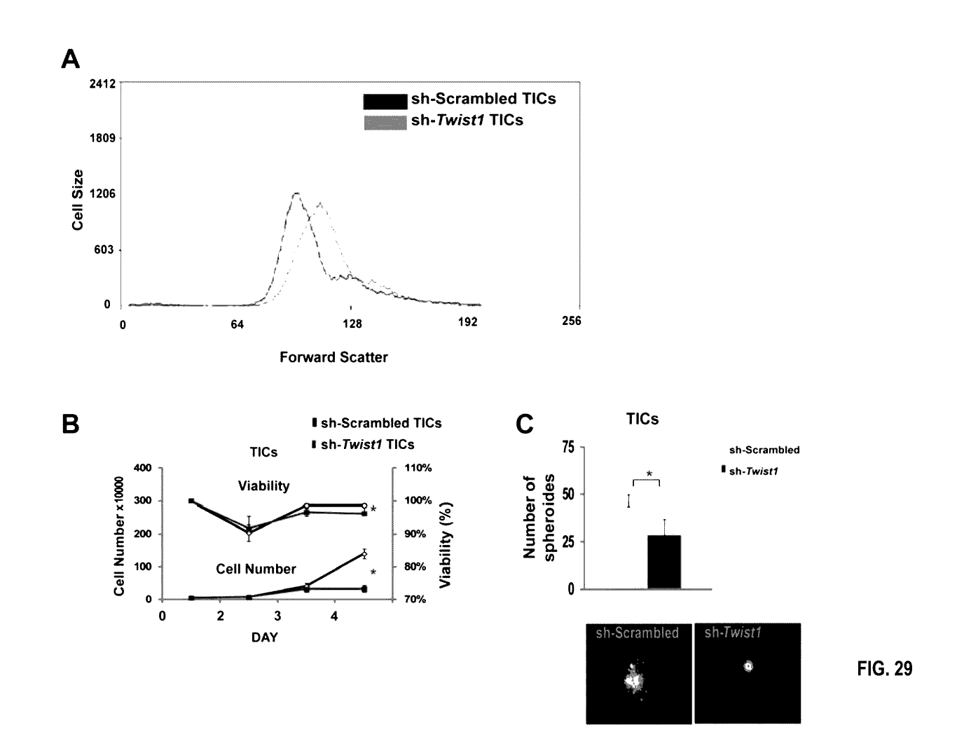

To understand the molecular basis of enhanced liver oncogenesis in HCFD-NS5A mice, RNA microarray analysis was performed. This identified 131 differentially upregulated and 43 down-regulated transcripts in HCFD-fed NS5A Tg mice (FIG. 20A and FIG. 27). Some of the more highly upregulated transcripts of different functional categories are listed in FIG. 20A. These include the stemness marker Nanog, oncogene Igf2bp3, and EMT and tumor metastasis regulator Twist1. Nanog and lgf2bp3 have been found to be critical in self-renewal and tumorigenic activity of TICs isolated from liver tumors of alcohol-fed NS5A mice. To confirm that TLR4 activation in the liver is from TICs, immunofluorescence staining was performed on control, HCFD, and HCFD+LPS livers (FIG. 28). This analysis confirmed that the source of TLR4 in the HCFD and HCFD+LFD livers is from TICs (TLR4 co-staining with NANOG) and not from the resident macrophages (Kupffer cells). For this study, the molecular mechanisms through which Twist1 promoted EMT and tumor metastasis in HCFD-fed NS5A derived TICs was further examined. To substantiate the microarray data. quantitative real time PCR (qRT-PCR) analysis was performed to measure Twist1 gene expression. As expected, Twist1 mRNA was significantly induced in HCFD-fed NS5A Tg mice compared to HCFD-fed WT mice or LFD-fed NS5A Tg mice (FIG. 20B). These analyses also revealed that Twist1 transcription was reduced in the HCFD-fed Th4^{-/-} NS5A Tg cohort (FIG. 20B), suggesting that the presence of TLR4 was permissive or required for Twist1 induction.

TLR4 Signaling Transactivates Twist1:

To further establish whether TLR4 regulates TWIST1, human HCC cell line Huh7 cells were transfected with an NS5A gene expression vector. Lentivirus expressing TLR4 or scrambled shRNA was then transduced in these NS5A/vector expressing cells and these cells were further stimulated with or without LPS. As shown in FIG. 20C, LPS treatment upregulated TWIST1 mRNA levels in NS5A-transfected Huh7 cells transduced with scrambled-shRNA, but not in any other groups with shRNA knockdown of TLR4. TWIST1 induction was significantly abrogated by TLR4 blockade. When a dominant-negative variant of TLR4 lacking the cytoplasmic domain (mutant TLR4; TLR4ACyt) was transduced into these cells, a similar and more conspicuous reduction of TWIST1 expression was observed. It was then tested whether TLR4 signaling can transcriptionally activate TWIST1. Huh7 cells were transfected with TWIST1 promoter (nt −700/−1) luciferase plasmid constructs and assayed for activity upon LPS treatment. A potent TWIST1 promoter activity was observed that was responsive to the LPS-TLR4 signaling axis (FIG. 20D), indicating that TLR4 does indeed transactivate TWIST1.

Twist1 Blockade Reduces TIC Self-Renewal, Migration and Tumorigenesis:

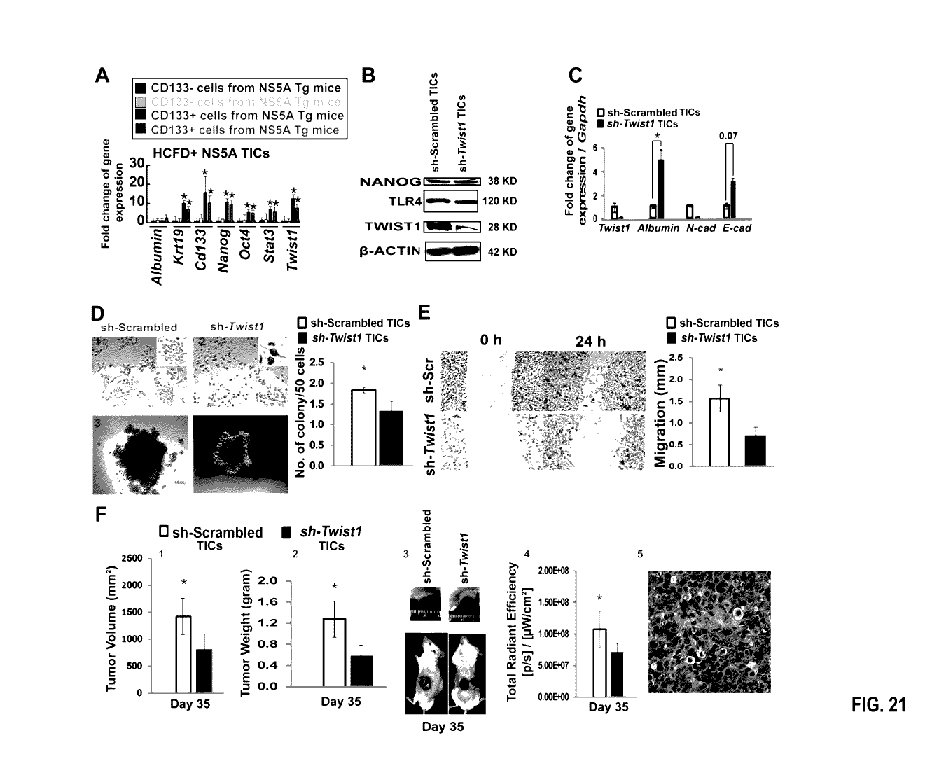

To demonstrate that TLR4 is responsible for Twist1 induction in TICs, CD133+/CD49f+/CD45-cells were isolated for examination of gene expression to show that these cells indeed express higher levels of stemness genes and Twist1 (FIG. 21A). The functionality of Twist1 in TICs was analyzed by silencing expression using lentivirus expressing Twist1 shRNA. Twist1 silencing did not affect TLR4 or NANOG (downstream of LPS-TLR4 axis) protein expression (FIG. 21B), but upregulated epithelial cell markers Albumin and E-cadherin expression while down regulating expression of a mesenchymal cell marker, N-cadherin (FIG. 21C); thus indicating that Twist1 silencing changes the mesenchymal phenotype to the epithelial phenotype. These data indicated that Twist1 acts downstream of the TLR4 signaling cascade and contributes significantly to the maintenance of mesenchymal phenotype based on its effect on Albumin, E-cadherin and N-cadherin. To further investigate this phenomenon, the phenotypic changes were assessed in TICs after Twist1 blockade. TIC morphology was altered from a spindle (mesenchymal) shape to a tadpole-like (epithelial) shape (FIG. 21D, inset); there also was increased cell size (FIG. 29A). Moreover, Twist1 blockade significantly reduced cell proliferation (FIG. 29B), self-renewal ability as assayed by colony formation in soft agar (FIG. 21D), spheroid formation (FIG. 29C) and cell migration by scratch assay (FIG. 21E). Implanted cells were then tested for tumorigenic potential in NOG mice. Subcutaneously transplanted Twist1 or scrambled shRNA TICs were monitored for tumor size over a period of 35 days. Gross and optical-image analysis of live tumor-bearing mice showed reduced tumor size in Twist1 knockdown groups (FIG. 21F, panels 3 and 4). As expected, tumor volume and weight were significantly reduced (FIG. 21F, panels 1 and 2). Histological examination of xenografted TICs showed that the resulting tumor exhibited an HCC morphology (FIG. 21F panel 5). These results revealed that Twist1, regulated through the LPS-TLR4 axis, plays a significant role in maintaining the mesenchymal and tumorigenic properties of TICs.

NANOG and pSTAT3 Regulate Twist1:

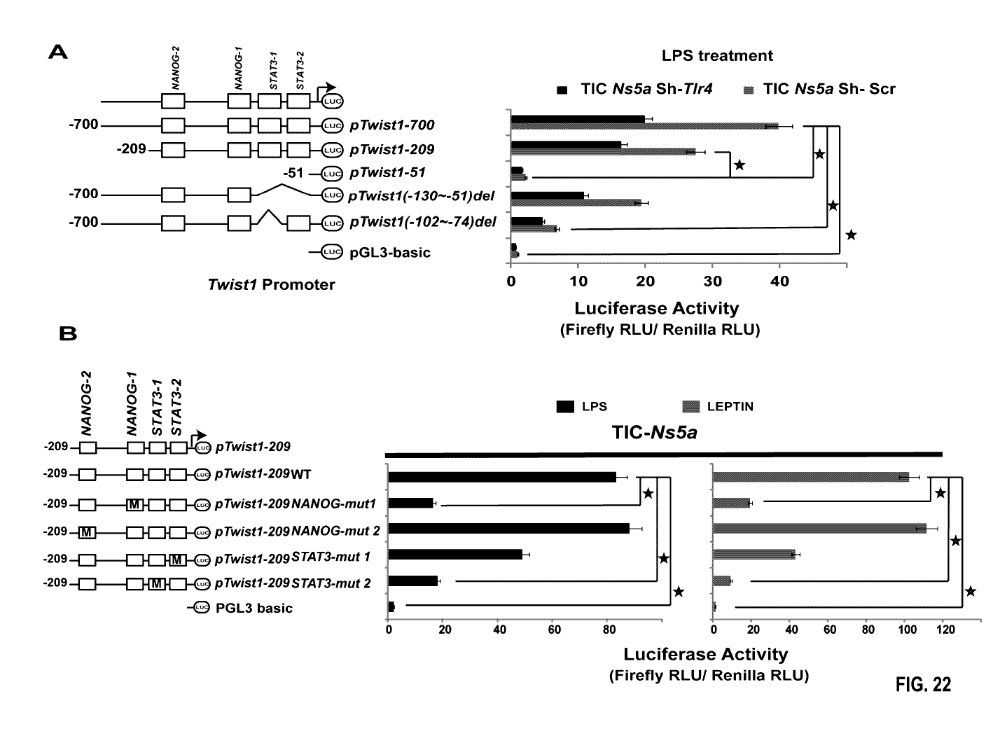

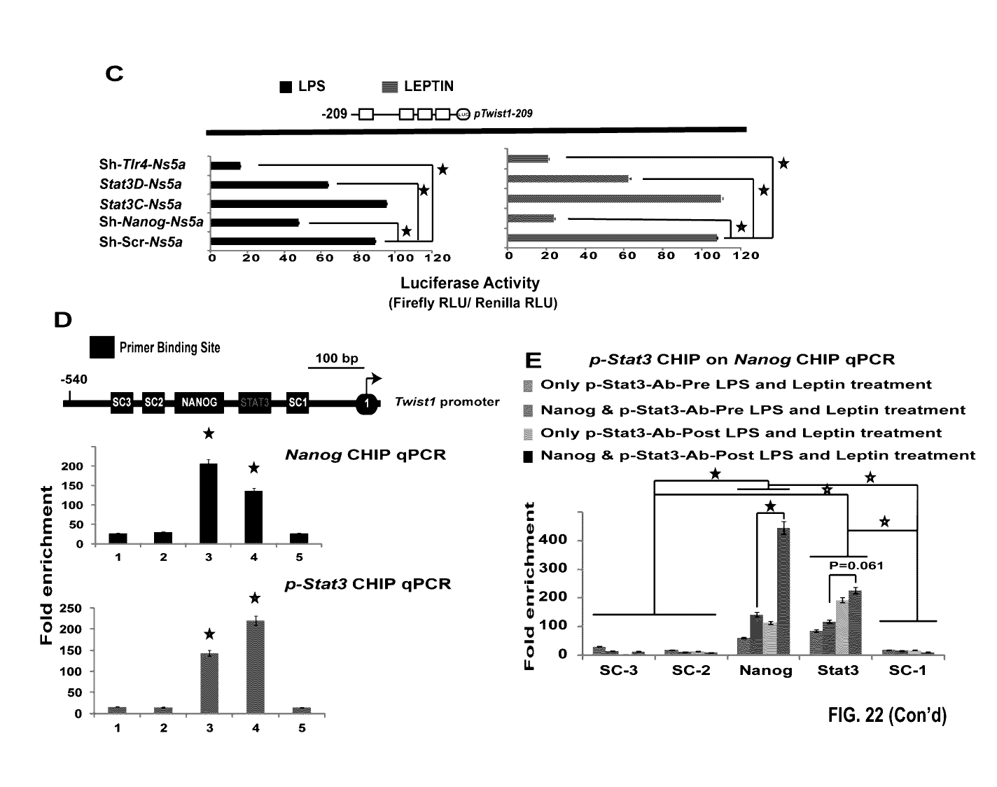

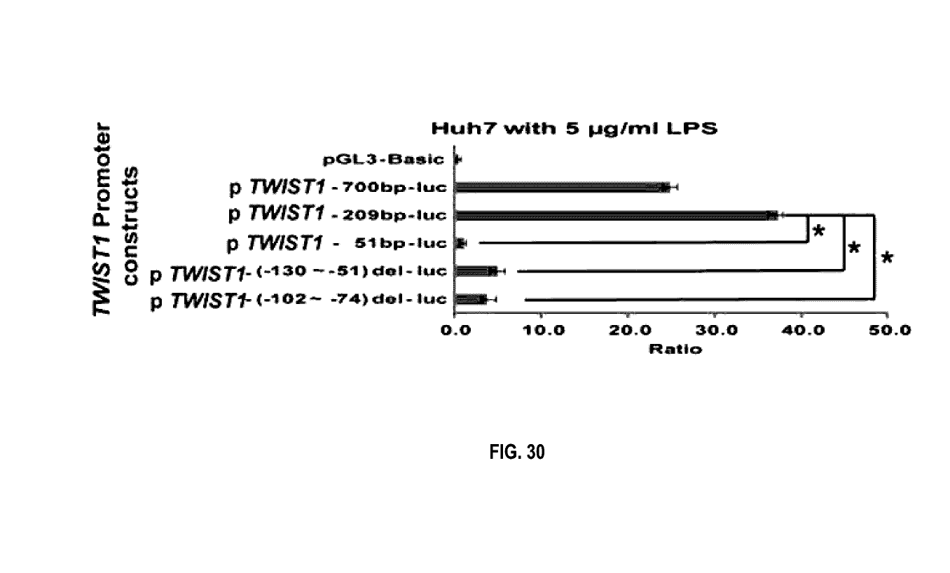

The molecular mechanisms responsible for TLR4-dependent activation of Twist1 was next investigated. Twist1 promoter-reporter assays were carried out, using promoter constructs²⁶ containing either WT (nt −700 to −1) or mutated regions upstream of the transcription initiation/start site (TSS). The activation of these reporter constructs was analyzed in cells transduced with either scrambled or Tlr4 shRNA. From this analysis, it was established that the region between −209 to −51 is essential for the basal and Tlr4-dependent induction of Twist1 in TICs (FIG. 22A; Huh7 cells in FIG. 30). In particular, a deletion between nts −102 and −74 markedly reduced Twist1 promoter activity, indicating that this region contained essential cis-elements. Long-term treatment of mice with HCFD activated Tlr4-Nanog signaling and increased leptin and endotoxin levels in the plasma. Furthermore, it was previously demonstrated that leptin and its receptor (OB-R) augmented pSTAT3 in TICs. In addition, NANOG is known to cooperate with STAT3 for maintenance of pluripotency in mouse embryonic stem cells. Thus, it was reasoned that, for activation of Twist1 in TICs, the adipose tissue-derived leptin-pSTAT3 signal and the TLR4-NANOG signal are needed. In silico analysis using Transcription Element Search System (TESS) and Transfac® identified consensus NANOG and STAT3 binding sites on the Twist1 promoter region. To evaluate the functions of these transcription factors, (FIG. 22B) the respective NANOG and STAT3 binding sites were mutated in the corresponding luciferase reporter construct and discovered that the STAT3-1 (STAT3 site distal to TSS) and NANOG-1 (NANOG site proximal to TSS) sites were critical for Twist1 promoter activity. As shown in FIG. 22B, mutations on these specific binding sites markedly attenuated reporter responsiveness to both LPS and leptin induction. In addition, when key upstream cellular signals (Tlr4, Nanog and Stat3) were blocked, Twist1 promoter activity was significantly abrogated (FIG. 22C). This result was further substantiated after chromatin immunoprecipitation-quantitative PCR (ChIP-qPCR) analysis with antibodies specific for NANOG and pSTAT3 (FIG. 22D). Single antibody IP of either NANOG or pSTAT3 enriched the NANOG-1 and STAT3-1 binding sites in qPCR, signifying that these two transcription factors might cooperatively transactivate Twist1 in response to LPS and leptin. As further validation of this model, sequential-ChIP analysis was performed. As shown in FIG. 4E, NANOG and pSTAT3 mutually bound each other in the process of transactivating Twist1.

Mouse and Human HCC have Accentuated Expression of TLR4, p-STAT3, and TWIST1:

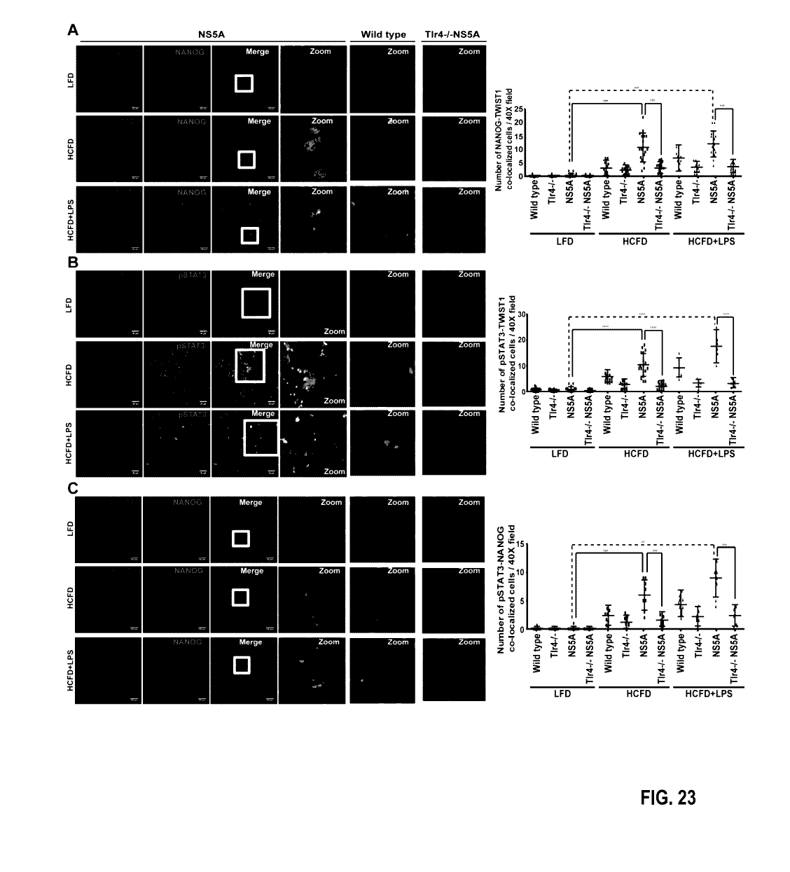

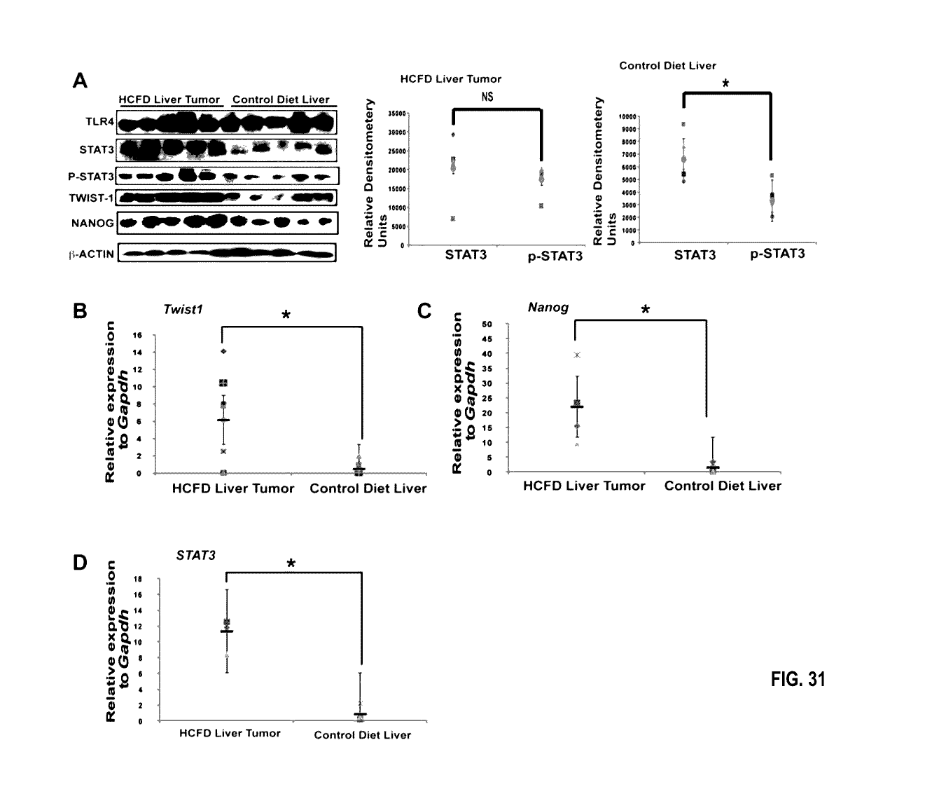

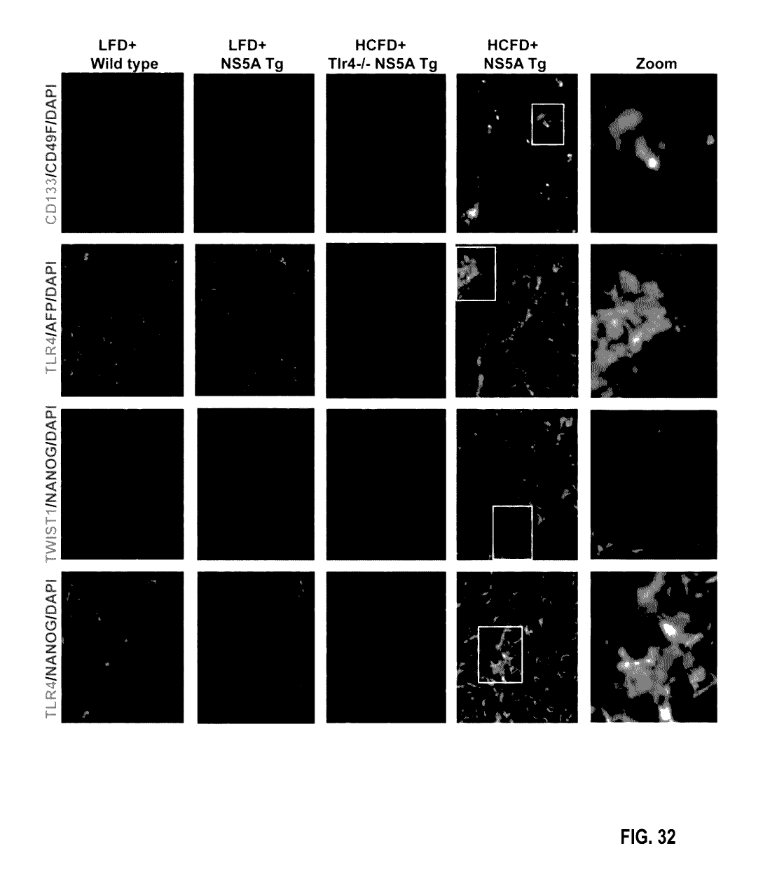

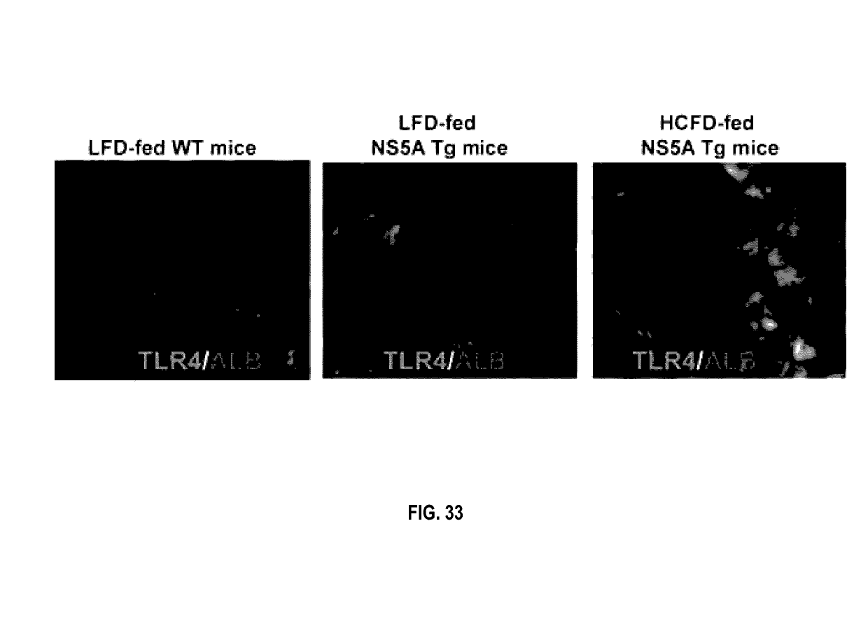

The involvement of both LPS-TLR4-NANOG and Lepin-OB-R-pSTAT3 signaling pathways for Twist1 induction was examined by immunoblotting analysis of lysates from liver tumors isolated from HCFD-fed NS5A Tg mice and normal livers of chow-fed mice. As expected, TLR4, STAT3, pSTAT3 and TWIST1 were all upregulated (FIG. 31A). The mRNA levels of TLR4, STAT3 and TWIST1 were also elevated in qRT-PCR analysis (FIGS. 31B-31D). Furthermore, immunostaining demonstrated co-localization of TWIST1 with pSTAT3, and NANOG as well as co-localization of pSTAT3 with NANOG in tumor-bearing HCFD and HCFD+LPS NS5A Tg liver specimens (FIG. 23 and FIG. 32), but less co-localization or fewer numbers of CD133+/CD49F+ or AFP+ cells in LFD-fed NS5A Tg or HCFD-fed Tlr4−/− NS5A Tg mice (FIG. 32). The major source of TLR4 in the liver of wild type mice is from non-parenchymal cell, including the Kupffer cells and stellate cells. The TICs derived from mice models have significant induction of TLR4. As shown in FIG. 32 the low fat diet (LFD) cohort IF staining shows TLR4 positive cells, which are presumably Kupffer cells or stellate cells. But in HCFD and HCFD+LPS the TLR4 positive cells have NANOG co-expression, indicating that TLR4 origin is not only from Kupffer cells or stellate cells but rather from the TICs or hepatocytes. This is further corroborated in FIG. 32 where co-staining of TWIST1-NANOG and CD133-CD49F is present in HCFD but not in LFD. Non-parenchymal areas of both mice fed HCFD and LFD have TLR4 staining while co-staining of TLR4-NANOG or TLR4-AFP are present mainly in HCFD group but not in LFD group and in groups of Tlr4−/−NS5A Tg mice. In liver of NS5A Tg mice, both parenchymal (ALB: Albumin+) and non-parenchymal staining of TLR4 are positive (FIG. 33) while non-parenchymal area of wild type mice fed LFD mainly have positive staining of TLR4 (FIG. 32), indicating that hepatocytes and TICs of NS5A Tg mice have elevated levels of TLR4, which are associated with strong staining patterns of AFP and TWIST1.

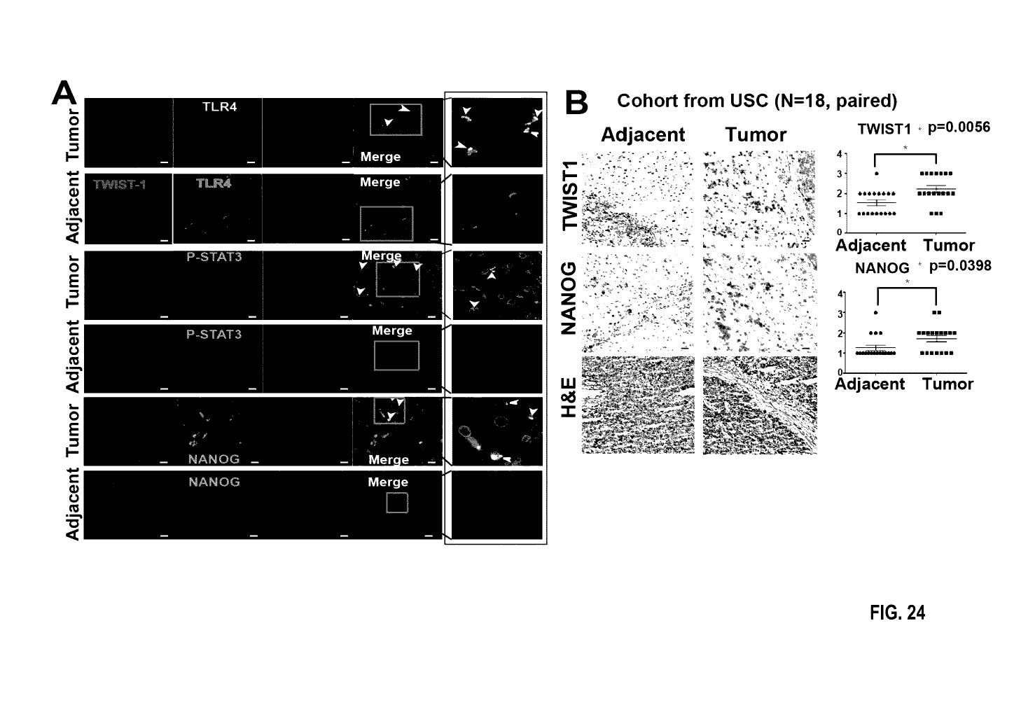

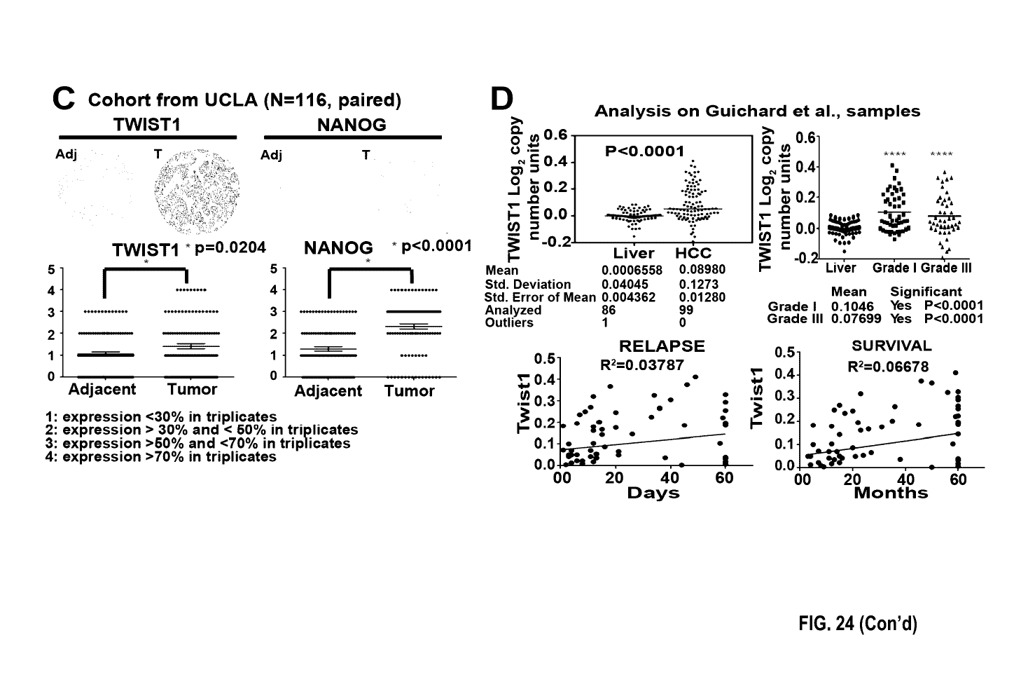

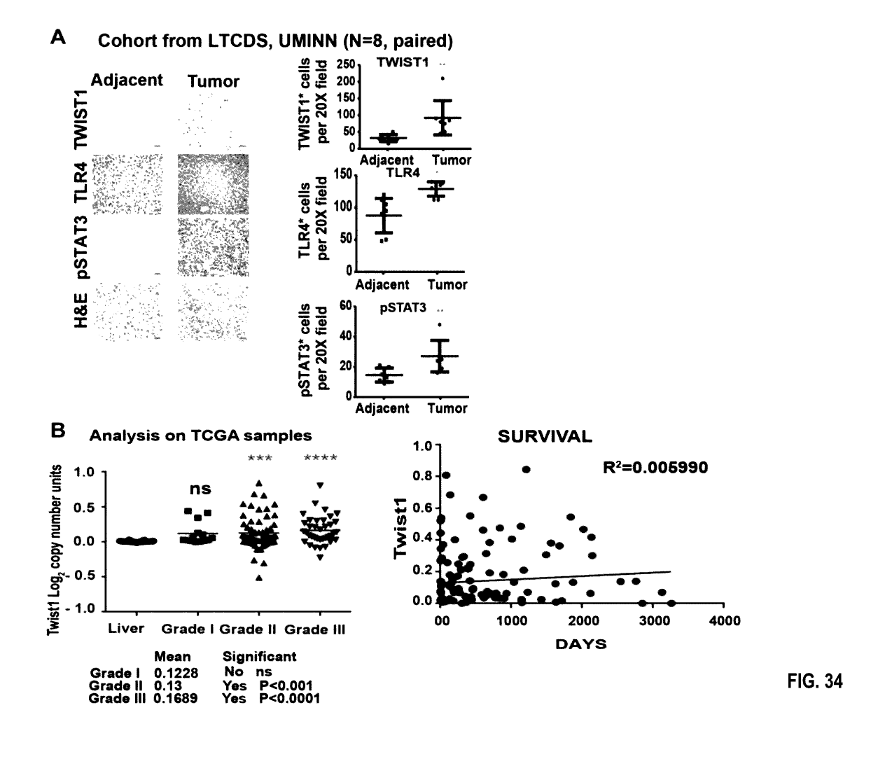

The clinical relevance of the findings was next assessed by analyzing the expression of these proteins in patient-derived HCC samples. Immunofluorescence staining detected co-localization of TWIST1 with TLR4, pSTAT3 and NANOG (FIG. 24A and FIG. 35). Moreover, paired IHC analyses of 142 patient samples (116 as a tissue microarray analysis, FIG. 34 and Table 8) were performed to validate the significance of TWIST1 and NANOG in human tissue sections from three different cohorts (FIGS. 24B-24C and FIG. 34A). To corroborate the findings and to gain insights on the correlation of Twist1 with grade, survival, and relapse in HCC patients, in silico analysis was performed using the Oncomine Gene browser. Two independent libraries from the repository were analyzed: TCGA Liver (The Cancer Genome Atlas; probing 97 HCC and 59 paired normal liver tissue) and Guichard Liver (probing 99 HCC and 86 normal liver). Both demonstrated the significant impact of TWIST1 on HCC (FIG. 24D and FIG. 34B).

TWIST1 Overexpression Promotes Tumor Formation:

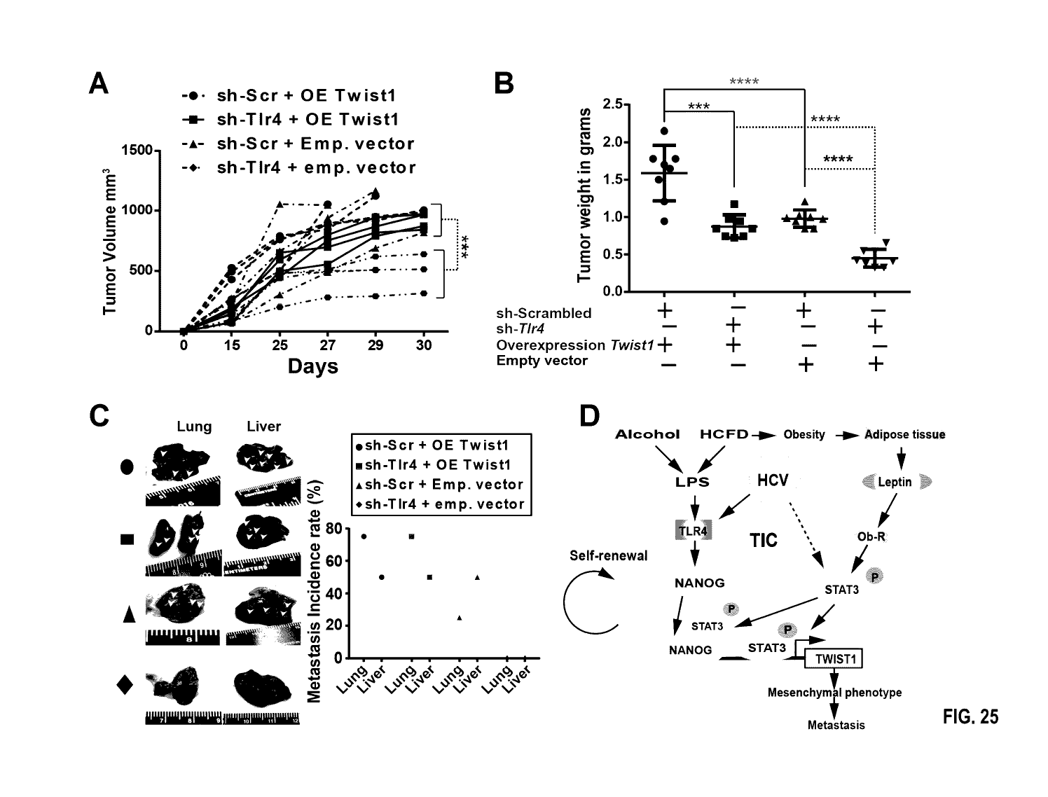

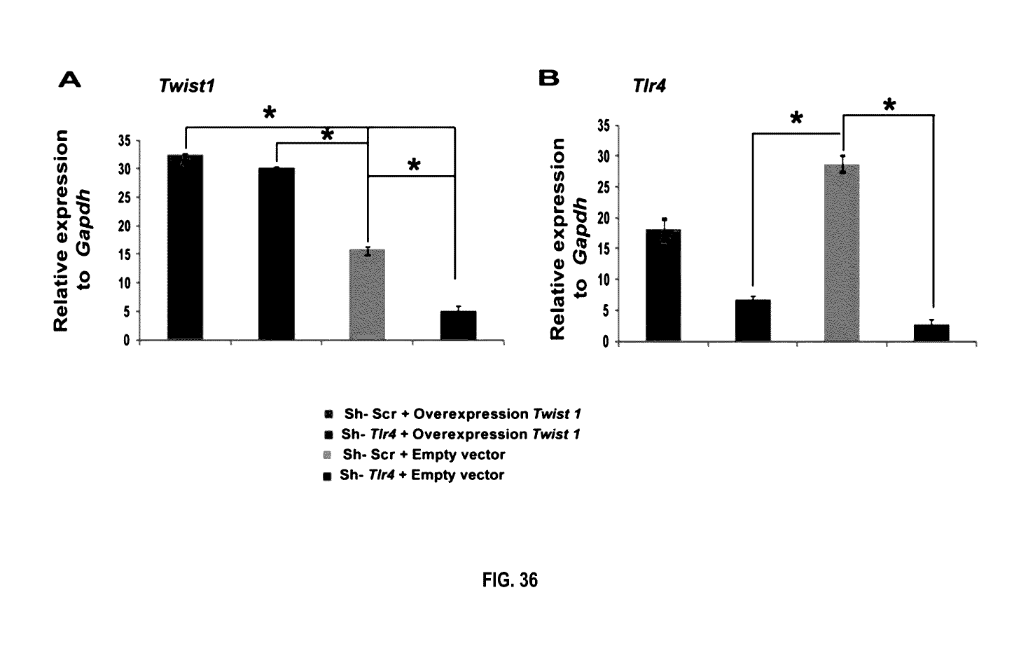

The results indicated that Twist1 silencing reduces TIC-derived tumorigenesis (FIG. 21F) and that Twist1 is downstream of TLR4 (FIG. 22). Whether overexpression of Twist1 beyond basal level in TICs can enhance its role in malignant tumor development and metastasis was then investigated. Additionally, It was asked how Tlr4 silencing can influence this outcome. To test this hypothesis, TICs expressing scrambled or Tlr4 shRNA (FIG. 36), TICs containing empty vector or TICs constitutively expressing Twist1 were transplanted into NOG recipient mice (FIG. 36A). Overexpression of Twist1 indeed promoted tumor growth and significantly increased final tumor volume and weight (FIGS. 25A-25B). Concomitant Tlr4 silencing (FIG. 36B) reduced the overall tumor volume and weight, indicating that TLR4 acts upstream of Twist1. Constitutive overexpression of Twist1 resulted in increased metastasis to the lung and the liver, suggesting that it has an important role in metastatic progression (FIG. 25C).

TICs comprise a small percentage of cells with stem-like properties resident in tumors and have been documented in a wide variety of cancerous tissues. EMT remodels cells and thus plays a key role in the acquisition of malignant traits. In this report, it was demonstrated that TLR4 is required for liver oncogenesis and the expansion of liver TICs in HCFD-fed HCV-NS5A Tg mice. Analysis of gene expression in TICs revealed that Twist1, a master regulator of EMT was increased 11-fold, which was not observed in TICs derived from alcohol diet fed NS5A Tg mice. The findings described an unexpected convergence of the NANOG and STAT3 signaling pathways. An important functional link has been identified between the NANOG pathway, by activation of upstream LPS-TLR4 signaling and the STAT3 pathway, driven by leptin-OB-R signaling. These two pathways cooperate to activate Twist1 and augment TIC motility (FIG. 25D).

These data implicate that life-style diseases, including obesity and alcoholism, promote genesis, mesenchymal phenotype and metastatic characteristics of TICs through synergistic interactions between LPS-TLR4-NANOG pathway and Leptin-Ob-R-STAT3 (FIG. 25D). Therefore, investigation of the effects of combination of inhibitors to prevent this synergistic interaction, including TLR4 antagonist or inhibitors targeting STAT3, NANOG and/or TWIST1, is warranted for further investigation in pre-clinical mouse models.

A synergistic interaction was demonstrated between alcohol consumption and HCFD, resulting in the highest observed tumor incidence in NS5A Tg mice (FIG. 20A). Additionally, a classical TLR4 activation was observed through canonical TAK-1, TRAF6 and pIKK-β signaling in both the HCFD- as well as HCFD-fed NS5A Tg mouse models (FIG. 19). It was observed from RNA microarray analysis that Twist1 was increased 11.9-fold in NS5A Tg mice fed HCFD (FIG. 20A). Long-term treatment of mice with HCFD activated Tlr4-Nanog signaling (FIG. 20D) and increased leptin and endotoxin levels in the plasma (FIG. 19B). A previous RNA microarray analysis of tissues from alcohol fed NS5A Tg mice did not exhibit Twist1 induction. These results led us to hypothesize that the adipose tissue-derived leptin-pSTAT3 axis and the TLR4-NANOG axis are needed for activation of Twist1 in TICs. Consequently the Twist1 promoter was analyzed for the functional importance of NANOG and pSTAT3 binding sites (FIG. 22). The experiments showed that relative to the TSS, both NANOG proximal and STAT3 distal sites were required for maximum response to leptin and LPS stimulation, respectively. It is postulated that this finding might be due to formation of a transcription complex comprised of these town binding proteins on the Twist1 promoter allowing contiguous stacking of these two transacting proteins.

In support of such a functional model, Watt et al., showed that Nanog interacts with Stat3 to regulate its own gene expression. Building upon their research, it was further established through sequential-ChIP-qPCR analysis (FIG. 22E) that these two transcription factors indeed interacted with one other to transactivate Twist1. The in vitro data were corroborated in mice and human tissue sections, where it was demonstrated by IHC and IF that TWIST1 co-localized with TLR4, P-STAT3 and NANOG. Nevertheless, future experiments are warranted to understand how these transcription factors activate Twist1. Potential mechanisms could be histone modifications in the Twist1 promoter or enhancer regions. Master regulators involved in EMT during wound healing process have a robust expression of poised enhancer marks. This is to methodically shift the cells to the native state post remodeling. An understanding of such epigenetics marks in HCC associated TICs and specifically targeting the epigenetics marks is needed in both mouse and patient derived models.

Moreover, it was observed that over-expression of Twist1 in the absence of Tlr4 can independently drive tumor formation and metastasis (FIG. 25) which underscores the importance of various TLR4 dependent oncogenic pathways. It is speculate that this phenomenon might be due to basal level expression of Tlr4 after shRNA treatment.

In conclusion, stemness markers NANOG and STAT3 are activated downstream of the LPS-TLR4 and leptin-OB-R pathways, respectively. NANOG and STAT3 cooperate to drive increased Twist1 levels, promoting the mesenchymal phenotype and metastasis in TICs (FIG. 25D) and contributing to HCC development.

### Example 7

### High Throughput Drug Screening: Summary

The TIC population possesses several key properties of normal stem cells including self-renewal, unlimited proliferative potential, and the ability to give rise to daughter cells. However, unlike highly organized normal stem cells, TICs show aberrant regulation of self-renewal and differentiation programs and produce daughter tumor cells that are in various stages of differentiation. TICs have been isolated from different types of solid tumors using various cell surface markers. Among these cell surface markers, CD133 was first used for TIC isolation in a Huh7 human hepatocellular carcinoma (HCC) cell line by Suetsugu et al. (2006). CD133 (+) TICs have the ability to proliferate faster (in vitro and in vivo) and have a preferential potential to form spheroids in primary and in subsequent passages. In vivo, CD133 (+) mouse xenograft models exhibit a greater tendency to develop tumors, even on serial transplantations. In vivo, CD133 (+) xenograft mice exhibit a greater ability for tumor growth and extended serial passage in recipient mice. The xenograft mice also show chemotherapy resistance through the AKT/PKB and Bcl-2 pathways (Ma et al., 2008).

CD133 (+) TICs highly express stemness-associated genes such as Nanog, Sox2, Oct3/4, Beni-1, Notch, β-catenin, Sino, Nestin, ABCG2, and ABCB1 (Ma et al., 2010). Nanog is the homeobox family of DNA-binding transcription factors and promotes oncogenesis (Jeter et al., 2009, Sun et al., 2014), and plays an important role in the TIC population. The tumorigenic effects of NANOG are associated with cellular and molecular changes such as increased expression of CD133, ALDH1, CXCR4, and IGFBP5 (Jeter et al., 2011). NANOG is not only expressed in germ cell tumors (Hoei-Hansen, 2008) but also in other types of carcinomas including breast (Ezeh et al., 2005), cervix (Ye et al., 2008), oral cavity (Chiou et al., 2008), kidney (Bussolati et al., 2008), ovary (Zhang et al., 2008), liver (Xu et al., 2010), and prostate cancer (Shen et al., 2011). More importantly, overexpression of NANOG promotes tumor cell resistance to both apoptosis and therapeutic agents via the AKT pathway (Noh et al., 2012). Recently, it was showed that downregulation of Nanog expression significantly attenuates tumor growth (Jeter et al., 2009, Chen et al., 2013).

MicroRNAs (miRNAs) are small noncoding RNAs (17-22 nucleotides) that are involved in RNA silencing via translation inhibition or mRNA degradation. Increasing evidence has revealed that miRNAs play a critical role in tumorigenicity. For example, in HCC, miR-130b is upregulated in CD133 (+) TICs, leading to the downregulation of tumor protein 53-inducible protein 1 (TP53INP1) and enhanced self-renewal (Ma et al., 2010). Similarly, miR-155 targets TP53INP1 to regulate the self-renewal ability of liver TICs (Chiou et al., 2015). Overexpression of miR-150 in CD133 (+) TICs led to inhibition of self-renewal and tumor growth via interaction with the 3′UTR of c-Myb (Zhang et al., 2012). miR-22 promotes Hepatitis-B virus related HCC development through down-regulation of estrogen receptor alpha (ERα) transcription (Jiang et al., 2011). More interestingly, these miRNAs often concur with epigenetic regulators to alter target gene expression. For instance, miR-22 promotes genes associated with epithelial to mesenchymal transition (EMT) by directly downregulating members of the ten-eleven translocation (TET) family (Song et al., 2013). The miR-29 gene family is downregulated in lung cancer, which directly regulates the de novo DNA methyltransferases (DNMTs) DNMT3A and DNMT3B, and leads to aberrant DNA methylation (Fabbri et al., 2007). Recently, miR-34b was shown to regulate DNMTs and histone deacetylases (HDACs) in prostate cancer (Majid et al., 2013).

Small molecule screening for identifying agents targeting TICs is performed worldwide to select potential drug candidates. However, drug development is lengthy and only a small fraction of hits are successful and become available as clinical drug treatments (Roses, 2008). In this study, an FDA-approved drug library was employed for screening purposes for identification of drug candidates that selectively target TICs. Successful repurposing of FDA-approved drugs would greatly shorten the development cycle required for clinical application compared to de novo drug development. If identified, these drug(s) could work synergistically or in combination with current HCC treatment regimens. To find compounds with minimum cytotoxicity and maximum anti-NANOG activity, the screen was performed using three approaches. One is a viability-based assay and the other is by using a NANOG promoter based activity assay as a method for specifically identifying chemotherapeutic agent(s) that repress NANOG. These approaches will identify and characterize other NANOG-dependent mechanisms underlying TIC chemoresistance based on this drug screen via comparison with non-tumor cells. By these approaches, it was found the combination of all-trans retinoic acid (ATRA) and the HDAC inhibitor suberoylanilide hydroxamic acid (SAHA) could specifically target TICs. By conducting RNA sequencing, it was found that this combination could successfully eliminate the TIC population as a result of miR-22 regulation of DNA methylation. By conducting RNA sequencing, it was discovered that this combination can successfully eliminate the TIC population via miR-22 regulations of phosphatase and tensin homolog (PTEN) regulated apoptosis pathway and DNA methylation of Nanog promoter.

A critical barrier to improved cancer therapy is the recurrence of drug-resistant tumors expanded from tumor-initiating stem-like cells (TICs). Discovery of a drug that specifically targets the TIC population is critical for effective treatment. Drug screening was first performed on TICs for the identification of cell-type specific drugs and found that all-trans retinoic acid (ATRA) specifically inhibited cell viability. Additionally, transduction of human TICs with a lentivirus Nanog-GFP reporter was used to perform high-throughput screening for Nanog-inhibitory drugs. HDAC inhibitor (SAHA), among several candidates, suppressed Nanog expression. Moreover, combination of RA with SAHA synergistically reduced Nanog expression and inhibited the self-renewal abilities of TICs resulting in apoptosis in vitro and in vivo. Genome-wide transcriptome analysis by using of RNA-seq showed that combined treatment reduced microRNA-22, which induced phosphatase and tensin homolog (PTEN) and ten-eleven translocation (TET). PTEN-mediated FOXO activation promotes BIM-mediated apoptosis. TET induction demethylates p53-binding sites within the Nanog promoter proximal region. Taken together, ATRA and SAHA may serve as a novel strategy for HCC treatment.

Highlights from the high throughput drug screening analysis includes the following:
Three high-throughput screenings identified the best combination of repurposed FDA-approved drugs (ATRA and SAHA).
ATRA and SAHA inhibited self-renewal of TICs and suppressed tumor growth.
The drug combination reduced micro RNA-22, resulting in activation of PTEN and TET family genes.
The drug combination epigenetically altered DNA methylation of Nanog promoter leading to inactivation of Nanog in TICs.

### Example 8

### High Throughput Drug Screening: Experimental Procedures

Mouse TICs Isolation:

The mouse TICs were isolated as previously described (Chen et al., 2013). In brief, the TICs were isolated from liver tumors of HCV transgenic mice that were fed with alcohol for 12 months. The minced tumor was digested by collagenase (Roche) with Dnase I (Roche), followed by CD133/CD49f/CD45 staining for FACS or CD133 staining for MACS.

Cell Culture:

Huh7, HepG2, and Hep3B human HCC cell lines were cultured in DMEM (high-glucose) medium supplemented with 10% fetal bovine serum (FBS), non-essential amino acids (NEAA, Invitrogen), and Glutamine/Penicillin/Streptomycin (Invitrogen). The mouse TICs grown in DMEM/F12 medium (Sigma-Aldrich) supplemented with 10% FBS, non-essential amino acids (NEAA, Invitrogen), Glutamine/Penicillin/Streptomycin (Invitrogen), nucleosides (Sigma), 20 ng/ml mEGF (Invitrogen), and 100 nM dexamethasone (Sigma-Aldrich). Both cell lines were grown at 37° C. and 5% CO₂.

Chemical Screening and Analysis:

The FDA approved drug library (ENZO Life BML-2841-0100) containing 640 FDA approved drugs was selected to maximize chemical and pharmacological diversity. The library included 44 different drug categories including analgesics, COX2 inhibitors, and cholinergics.

Cell Viability Assay:

CD133(+) and CD133(−) Huh7 cells were freshly sorted using the MACS CD133 micro bead kit (Miltenyi Biotec, 130-050-801) and were seeded in 100 μl medium containing 5000 cells per well in a 96-well plate. Once the cells attached, the FDA approved drug library was added to each well to a final concentration of 20 μg/ml in duplicate. After incubation for 48 hours, the cell viability was determined by a luminescence assay. The selected drug candidates had to show a significant cell growth inhibition effect on the CD133 (+) population (percentage of cell viability less than 30%), but with no or only a minor effect on the CD133 (−) population (percentage of cell viability greater than 70%) compared to the vehicle control group (1% DMSO). After 16 hours, 1 μl of a 2 μg/ml drug solution was individually added to the 96 wells, resulting in a 20 μg/ml final concentration for most compounds. After 48 hours, CellTiter-Glo® Reagent (G8233, Promega) was added and the luminescence signal was measured with an automated plate reader. The raw data for each well was background-corrected by DMSO control wells on the same plate. The selected hit compounds exhibited a marked effect on CD133 (+) cells (cell viability<30%) and low/no effect on CD133 (−) cells (cell viability>70%).

Nanog-GFP Screening:

The Nanog-GFP liver cancer stem cell line was transduced with pGreenZeo-Nanog transcriptional reporter lentivirus vector (System Biosciences SR10031VA-1). The transduced cells were positively selected with zeomycin (10 μg/ml) and further sorted for the GFP-high population (˜20% of total population) for drug screening. Nanog-GFP liver cancer stem cells were then seeded in 100 μl medium containing 5000 cells per well in a 96-well plate. After 16 hours, 0.5 μl of a 2 μg/ml compound was added to each well, resulting in a 10 μg/ml final concentration for most compounds. After 12 hours, the cells were fixed with 1% Paraformadehyde and stained with DAPI; compounds were screened in duplicate. The GFP and DAPI images were acquired using a BD Pathway Bioimaging Systems instrument. Z-score was calculated from the data using the formula z=(X−u)/s.d., where u is the mean, s.d. is the standard deviation of the whole population and X is the sample value calculated based on the ratio of GFP intensity to DAPI intensity. The z-score of selected hits must be less than −1.0. The average of z-score of vehicle control is 2.0±1.04.

Combination Dose Determination:

Freshly sorted CD133 (+) Huh7 or mouse TICs were plated in 100 μl of medium containing 5000 cells per well in a 96-well plate. After 16 hours, 0.5 μl of a 2 μg/ml, 1 μg/ml, 0.2 μg/ml, 0.1 μg/ml, 0.02 μg/ml, or 0.01 μg/ml compound was added to each well in triplicate, resulting in a 10 μg/ml, 5 μg/ml, 1 μg/ml, 0.5 μg/ml, 0.1 μg/ml, and 0.05 μg/ml final concentration for most compounds, respectively. After 48 hours, the cells were either measured for cell viability by Cell-Glo® Reagent (Promega), or fixed with 1% PFA (paraformaldehyde) and stained with DAPI for high-throughput screening.

Annexin V Staining:

Annexin V staining was performed according to the manufacturer's instructions (A35110, Invitrogen). In brief, after drug treatment, the cells were washed twice with ice-cold phosphate-buffered saline (PBS) and detached by trypsin/EDTA. The cells were then incubated with 5 μl of Annexin V-APC in a 100 μl of cell suspension at room temperature for 15 minutes. After incubation, the cells were mixed with propidium iodide solution and analyzed by flow cytometry.

Caspase Activity Analysis:

The caspase activity assay was performed according to the manufacturer's instructions (G8090, G8200, and G8210, Promega). In brief, 10,000 cells were plated into each well of a 96-well plate. After the cells attached, the drugs (5 μg/ml of ATRA and 0.5 μg/ml of SAHA) were added to each well and incubated for 6, 12, 16, and 24 hours. After incubation, the Caspase-Glo® substrate reagent was added to each well followed by incubation for 30 minutes. After incubation, the luminescence signal was measured with a luminometer.

TUNEL Staining Assay:

The TUNEL staining was performed according to the manufacturer's instructions (4810-30-K, TREVIGEN). In brief, paraffin-embedded tumor sections from each group were de-paraffinized, re-hydrated, and washed twice in PBS. Samples were covered with Proteinase K solution for 30 minutes at room temperature and then washed two times in deionized water. Slides were immersed in quenching solution for 5 minutes at room temperature and then washed in PBS. Slides were incubated in TdT labeling buffer for 5 minutes, immersed with labeling reaction mix, and incubated at 37° C. for 1 hour in a humidity chamber. Samples were immersed in TdT stop buffer for 5 minutes and then washed twice in deionized water for 5 minutes each at room temperature. Samples were covered with Strep-HRP solution and incubated for 10 minutes at 37° C. and washed twice in PBS. Samples were incubated in DAB solution for 5 minutes and then washed in deionized water several times. The samples were counterstained with Methyl Green and mounted on slides for observation.

Tumor Spheroid Formation Assay:

Freshly sorted CD133(+)/(−) Huh7 cells were plated in a low binding culture plate (NUNC 145397) containing 100 cells per well in 100 μl of culture medium with ATRA (5 μg/ml), SAHA (0.5 μg/ml), or a combination of both. After 2 weeks, colony numbers were counted.

Anchorage-Independent Growth Assay:

Freshly sorted CD133 (+)/(−) Huh7 cells were mixed with 0.35% agarose containing 1000 cells per well in culture medium with retinoic acid (5 μg/ml), SAHA (0.5 μg/ml), or a combination of both. After 2 weeks, the colony numbers were counted.

Retinoic Acid Nanoparticles Conjugated with CD133:

CD133 was conjugated to the terminal amine functionality on a polyethylene glycol block of polylactide-polyethylene glycol (PLA-PEG) as previously described (Swaminathan et al., 2013). PLGA (30 mg) was dissolved in 1 ml chloroform containing ATRA (6 mg). An oil-in-water emulsion was formed by emulsifying the polymer drug solution in 6 ml of 2.5% w/v aqueous PVA solution by sonication (Sonicator®XL, Misonix, N.Y.) for 5 minutes in an ice bath. The PLA-PEG-CD133 conjugate was dissolved in chloroform (8 mg/100 μl) and added to the oil-in-water emulsion with stirring. The emulsion was stirred for 18 hours at ambient conditions followed by 2 hours under vacuum to remove residual chloroform. Nanoparticles were recovered by ultracentrifugation (35,000 rpm for 35 minutes at 4° C.; OptimaTMLE-80K, Beckman, Palo Alta, Calif.) and washed three times with deionized water to remove excess PVA and unencapsulated drugs. The nanoparticle suspension was then lyophilized (Labconco, FreeZone 4.5, Kansas City, Mo.). Before injection, the lyophilized nanoparticles were re-dissolved in PBS and filtered with a 0.22 micron filter.

In Vivo Tumorigenicity Experiments:

A half million freshly sorted CD133 (+) TICs were suspended in 100 μl of Matrigel™ (BD) and injected subcutaneously into NOD/Shi-scid/IL-2Rγ^{null}(NOG) mice, six mice per group. After the tumor volume reached 100 mm³, animals received one intravenous dose of CD133-conjugated RA nanoparticle (5 μg/ml) and SAHA (0.5 μg/ml) daily. The animals were monitored regularly for tumor growth and survival every day. All animals work was performed according to national and international guidelines. Animal studies were based on a protocol approved by the Institutional Animal Care and Use Committee at University of Southern California.

RNA Isolation and Real-Time PCR:

Total RNA was isolated using an RNeasy Mini Kit according to the manufacturer's protocol (Qiagen). Isolated total RNA (1 μg) was treated with DNase I (Invitrogen), and complementary DNA (cDNA) was synthesized using the Omniscript® Reverse Transcription kit (Qiagen). Synthesized cDNA was then subjected to quantitative real-time PCR using the TaqMan® Fast Advanced Master Mix (Invitrogen). The amplification protocol consisted of incubation at 50° C. for 2 minutes, activation at 95° C. for 20 seconds, denaturing at 95° C. for 2 seconds, and annealing and extension at 60° C. for 20 seconds for 40 cycles using an ABI 7900HT Sequence Detection System and SDS 2.0 software (Applied Biosystems). The TaqMan® primers used for quantitative real-time PCR included Nanog (Hs04399610_g1 for human, Mm02384862_g1 for mouse), miR22hg (Mm01246600_m1), and beta-Actin (Hs01060665_g1 for human, Mm00607939_s1 for mouse) as an internal control.

RNA Sequencing:

RNA sequencing samples were collected at 16 hours of treatment with ATRA (5 μg/ml), SAHA (0.5 μg/ml) or combination treatment. Total RNA for RNA sequencing was extracted using RNeasy Plus Mini Kit (Qiagen), which includes a DNA depletion column. DNase I treatment and rRNA depletion with Tibozero technology were performed before RNA sequencing. Sample quantity and quality was verified by spectrophotometry (NanoDrop 1000), fluorimetry (Qubit), and the Aglient Bioanalyzer 2100 profiler. RNA Integrity Number (RIN) values of >7.0 and OD260/280=2.0-2.2 were used for RNA-seq library preparation. Extracted RNA (1 μg) was used for RNA sequencing (Illumina HiSeq2500 system). Sequenced reads were cleaned according to a rigorous pre-processing workflow (Trimmomatic-0.32) before mapping them to the mouse genome (mm10) using SHRiMP2.2.3 (http://compbio.cs.toronto.edu/shrimp/). Cufflinks2.0.2 (cuffdiff2-Running Cuffdiff) was then used to perform differential expression analysis with a FDR cutoff of 0.05 (95% confidence interval). A Perl script was used after differential expression analysis to improve the readability of the results files. Quality control information was generated via Fastqc: www.bioinformatics.babraham.ac.uk/projects/fastqc/. The log 2 (fold change) seen in these files was such that fold change=Sample2_fpkmValue/Sample1_fpkmValue. All work was performed by the University of Rochester Genomics Research Center (URGRC). All gene expression profiles were analyzed by Partek Flow, Ingenuity Pathway Analysis and Gene Set Enrichment Analysis.

Bisulfite Sequencing:

Bisulfite sequencing was performed according to the manufacturer's instructions (D5005, Zymo Research). In brief, 2 μg of genomic DNA from each group was treated with CT conversion reagent in the following thermal cycle: 98° C. for 10 minutes, 64° C. for 2.5 hours, and 4° C. for storage for up to 20 hours. Converted DNA was treated with M-Desulphonation Buffer for 20 minutes at room temperature. After desulfonation, DNA was washed and eluted. Bisulfite-treated DNA (150 ng) was used for PCR.

Bisulfite PCR primers includes the following:

Oct4 binding siteForward:
(SEQ ID NO.: 38)
5′-TTTAATGTGAAGAGTAAGTAAGAAA-3′
Reverse:
(SEQ ID NO.: 39)
5′-ATAAAATAACCCAAACTAAAAAAAA-3′
p53 binding site Forward:
(SEQ ID NO.: 40)
5′-GTTTTTTGTAGAATAAAATTTAGGAAGA-3′
Reverse:
(SEQ ID NO.: 41)
5′-CAAACTTATCTACCACCATACCCAA-3′

Chromatin Immunoprecipitation Assays (ChIP-qPCR):

The cells were fixed in 1% formaldehyde for 10 minutes at room temperature and the reaction was quenched by 0.125M glycine. The cells were washed twice with ice-cold PBS, resuspended in lysis buffer [1% SDS, 10 mM EDTA, 50 mM Tris-HCl pH 8.0, 1 mM phenylmethylsulphonyl fluoride (PMSF), 1 ml per 10⁶ cells] and incubated on ice for 10 minutes. The cell suspension was sonicated 5 times for 1 minute each. The sonicated samples were centrifuged at 14,000 rpm at 4° C. for 15 minutes and the supernatant (input) was stored at −80° C. The supernatants (50 μl) were immunoprecipitated with 5 μg of relevant antibodies in RIPA buffer (1% Triton X-100, 0.1% deoxycholate, 140 mM NaCl, 1 mM PMSF) overnight at 4° C. under rotation. Protein G beads were incubated with 100 μg/ml sonicated salmon sperm DNA and 1 μg/ml bovine serum albumin in RIPA buffer under the same conditions. Blocked beads and immunoprecipitated samples were combined the next day and were incubated under rotation for 3 hours at 4° C. The immunoprecipitates were washed 7 times with RIPA wash buffer (1% Triton X-100, 0.1% DOC, 0.1% SDS, 500 mM NaCl, 1 mM PMSF). Input samples (10 μl) and beads were resuspended in 100 μl of 100 mM Tris-SDS and proteinase K to a final concentration of 200 μg/ml and incubated for 4 hours at 55° C. and then overnight at 65° C. The next day, samples were phenol-chloroform extracted and ethanol immunoprecipitated with NaOAc and 20 mg of glycogen as a carrier. DNAs from input and immunoprecipitated pellets were resuspended in 50 μl and 250 μl of TE buffer, respectively. The DNA content was analyzed using qPCR (5 μl per 20 μl reaction)

ChIP-qPCR primers includes the following:

Oct4 binding siteForward (-285):
(SEQ ID NO.: 43)
5′-AGTGAAATGAGGTAAAGCCTCT-3′
Reverse (-80):
(SEQ ID NO.: 44)
5′-TATTCTCCCAGGCACCCA-3′
p53 binding site Forward (-741):
(SEQ ID NO.: 45)
5′-TACAGTGAGAACTTGTCTCAAA-3′
Reverse (-541):
(SEQ ID NO.: 46)
5′-GAGCCTGTGTCCTGCTTA-3′

Western Blot Analysis:

Cells were lysed in Nonidet-P-40 lysis buffer (150 mM sodium chloride, 1% NP-40, and 50 mM Tris, pH 8.0) with proteinase inhibitor cocktail (Roche) and incubated on ice for 30 minutes. After incubation, the cell lysate was centrifuged at 14,000 rpm at 4° C. for 15 minutes. Protein concentration was determined using the Bio-Rad protein assay kit and 20 μg of total protein was used for the assay. The primary antibodies included: p53, (cleavage) Caspase 3, SIRT1, PTEN, AKT, pAKT(T308) (Cell signaling Technology), OCT4, DNMT1, DNMT3A, DNMT3B (Abcam), TET2 (Abiocode), p15, p19, p21, p27 (One world lab), CDK2, CDK4, CyclinD1, Cylcine E (Santa Cruz) and Beta-Actin (Sigma) as an internal control.

Statistical Analysis:

All data were expressed as standard error of the mean (SEM) for n≧3. Comparisons between groups were analyzed by ANOVA. p values less than 0.05 were considered statistically significant.

### Example 9

### High Throughput Drug Screening: Data and Analysis

Identification of FDA-Approved Drug(s) that can Specifically Target Tumor-Initiating Stem-Like Cells:

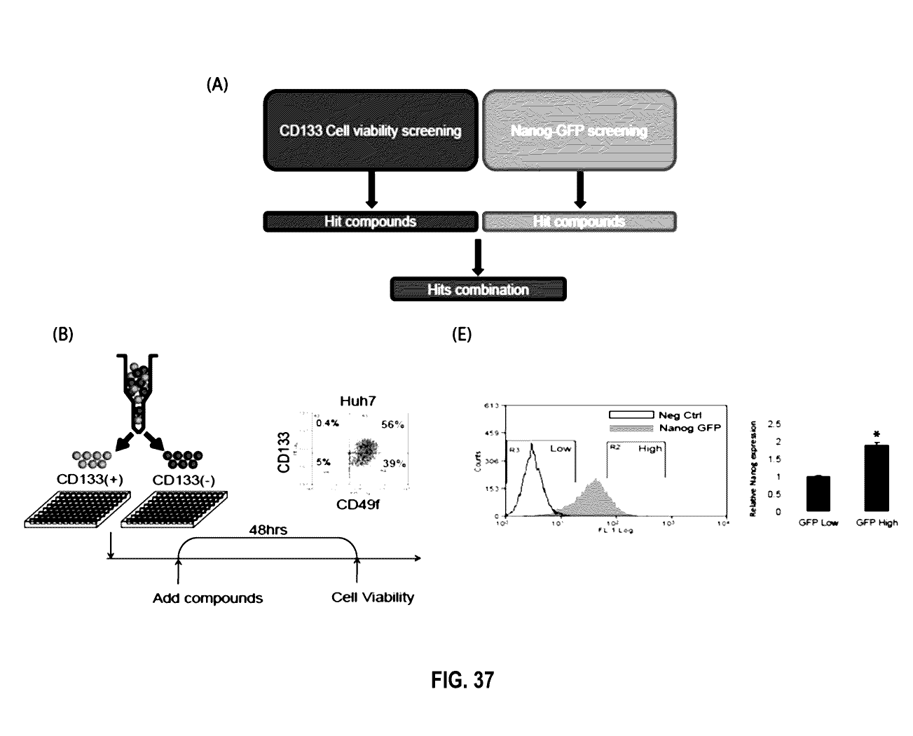

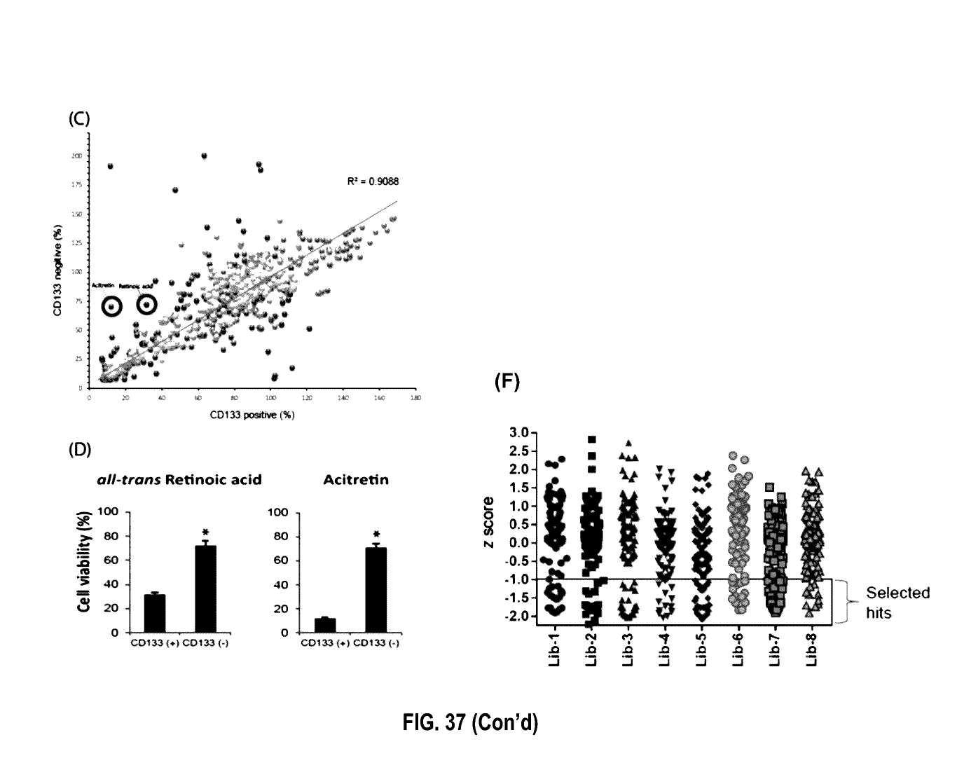

To identify the drug(s) targeting the TIC population, three different drug screenings were performed: (1) a CD133 cell viability screening; (2) a NANOG-GFP reporter cell screening; and (3) a combination screening (FIG. 37A). First, to select candidates that specifically inhibited the growth of CD133 (+) TICs, Huh7 cells were utilized, which is a human HCC cell line of which approximately 50-60% of the cell population constitutively expresses CD133 (FIG. 37B).

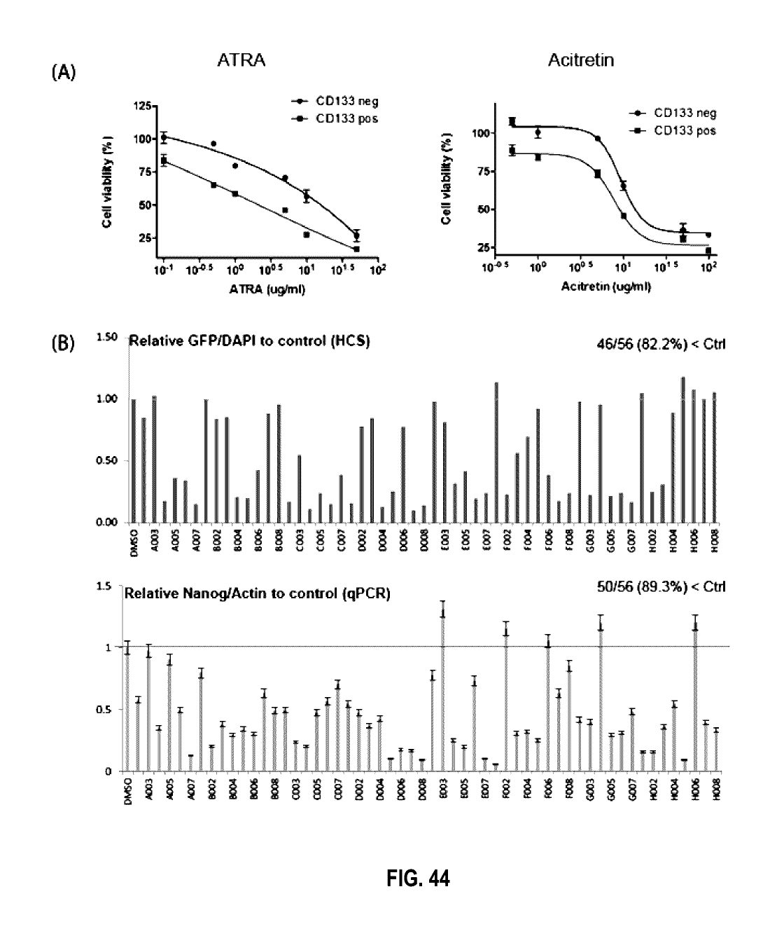

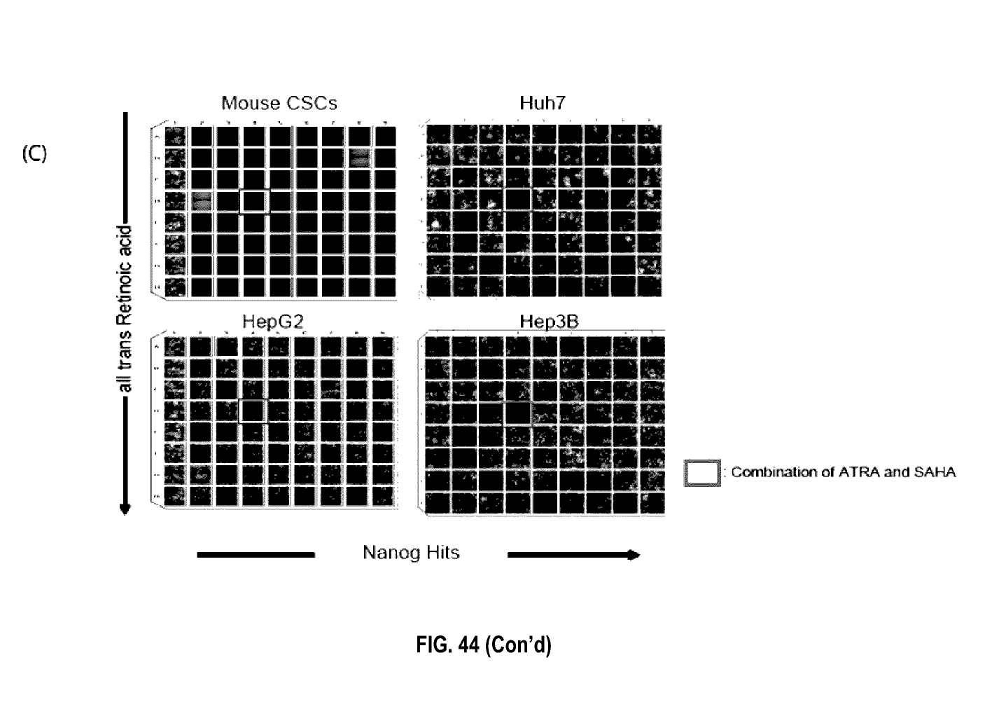

CD133 (+) and CD133 (−) cells freshly sorted by either fluorescence activated cell sorting (FACS) or magnetic associated cell sorting (MACS) were plated into individual wells in a 96-well platform and compounds were assayed in duplicate for cell viability after 48-hours treatment. Among the 640 compounds tested, most exhibited a similar growth inhibitory effect for both CD133 (+) and CD133 (−) cell populations (R²=0.80) (FIG. 37C). However, two compounds showed a selective inhibitory effect on the CD133 (+) population but not on the CD133 (−) cell population. ATRA was inhibitory to cell growth with resulting cell viabilities for CD133 (+) and CD133 (−) cells of 41.4% and 72%, respectively in response to this drug, Another drug was acitretin, a second generation retinoic acid derivative with cell viabilities for CD133 (+) and CD133 (−) cells of 12% and 70.4%, respectively) (FIG. 37D). Next, the IC50s for these two compounds were determined to be 4.06 μg/ml (13.5 μM) for ATRA and 7.37 μg/ml (22.58 μM) for acitretin (see FIG. 44A). In consideration of the lower pharmacologically deliverable dose, ATRA was used for the subsequent study.

Next, to target the TIC population further, a NANOG-green fluorescent protein (GFP) reporter cell line was generated by using TICs derived from mouse liver tumors. The lentivirus NANOG-GFP vector was transduced into TICs and followed by antibiotic selection. To characterize this reporter cell line, the GFP high (top 20%) and low (bottom 20%) populations were sorted by FACS. Virtually 100% of the cells expressed the transduced reporter (FIG. 37E, left). Quantitative real-time PCR (qPCR) data revealed that the Nanog expression in the GFP-high population was 2-fold higher in the GFP-low population (FIG. 37E, Right). Additionally, immuno-fluorescent staining was conducted to confirm that the GFP signal accurately represented the Nanog expression (FIG. 44B). These data suggested that the NANOG-GFP cell line was reliable for drug screening.

Freshly sorted Nanog-GFP(+) cells were plated into individual wells in a 96-well culture plate. Once the cells became adherent, each drug in the aforementioned drug library was added to individual wells at a final concentration 20 μg/ml in duplicate. The cells were fixed and stained with DAPI (4′,6-diamidino-2-phenylindole) after 12 h of incubation, and the GFP and DAPI signals were read using a high-content screening reader. The criterion used for positive drug candidates was a z-score less than −1.0 (the average z-score of vehicle control is 2.00±1.04) (FIG. 37F). With this strategy, 56 hits were selected from the primary screening (not shown). After GFP screening, cells were assayed by qPCR for Nanog gene expression; this analysis confirmed that ninety percent of the hits had a positive correlation between the GFP screening and the qPCR data (FIG. 44B). Using this strategy, 56 hits were selected from the primary screening (FIG. 37F). The subsequent confirmatory GFP screening was performed and Nanog gene expression was further confirmed by qPCR. Ninety percent of the hit patterns matched between the GFP screening and the qPCR results (FIG. 37F). Among these 56 hits, 14% of the drugs were anti-neoplastics, 12.5% of the drugs were anti-inflammatory, and 10.7% of the drugs were anti-hypertensive. More interestingly, based on the mechanisms of these drugs, 25% of the candidates were hormone related (i.e., agonists or antagonists for adrenergic, prostaglandin, or angiotensin receptors), 20% of the hits were neuron signal related (i.e., positive or negative modulators for the N-methyl-D-aspartate receptor, dopamine receptor, or acetylcholine receptors), and 10% of the hits regulated ion-channels or proton pumps.

In order to increase the effectiveness of these drug candidates for elimination of the TIC population, ATRA was combined with 56 candidate compounds from the NANOG screening. Of these drugs combined with ATRA, one drug efficiently eliminated viability of various HCC cell lines and mouse TICs. This hit was the HDAC inhibitor, suberoylanilide hydroxamic acid (SAHA) (FIG. 44C).

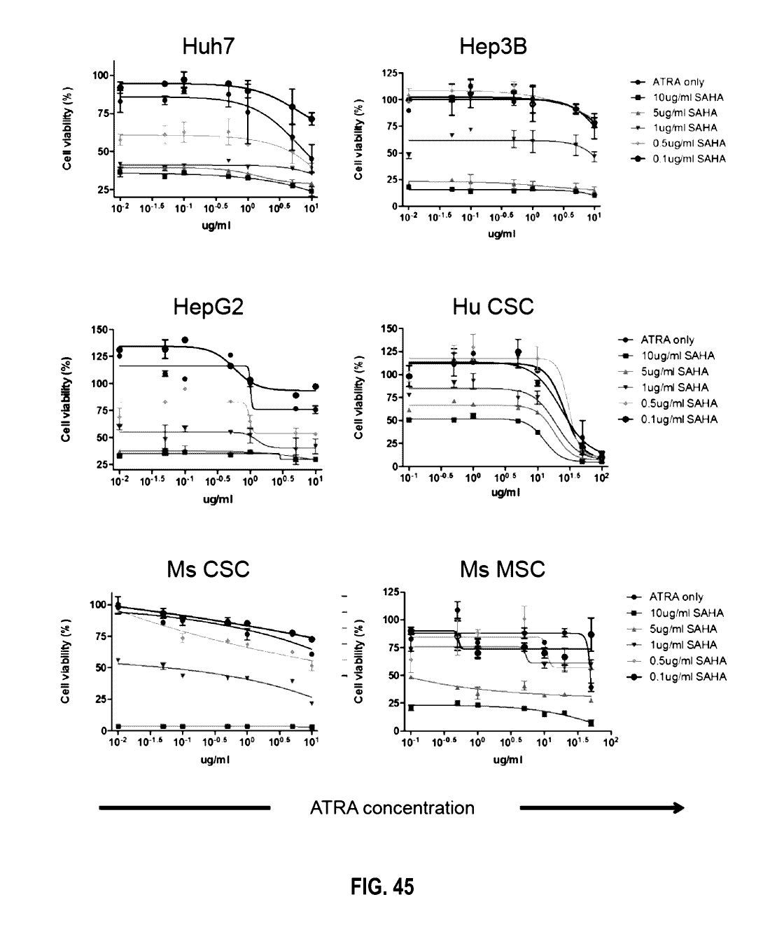

This drug combination was further tested to find the best combined dosage. Various concentrations of SAHA and ATRA were tested holding one constant and varying the other. Furthermore, optimum drug combinations were tested to see if similar inhibitory effects were observed with other different cancer cell lines. These cell lines were human and mouse HCC cell lines, including HepG2, Hep3B, and mouse TICs. The results showed that this combination had a similar dose-response effect on these cancer cell lines (FIG. 45).

To test for specific killing activity toward TICs but not normal stem cells, viability of normal postnatal stem cells (mouse mesenchyme stem cells) were assayed with the combination treatment and it was found that this combination did not demonstrate any toxicity over the concentration ranges tested as observed with TICS (FIG. 45). The cytotoxic effects were only observed at very high combination dosages, indicating that this drug combination showed high specificity for the TIC population but spared the normal stem cell population.

ATRA and SAHA Combination Induces Cell Apoptosis Pathways and Reduces the Self-Renewal Ability of TICs In Vitro:

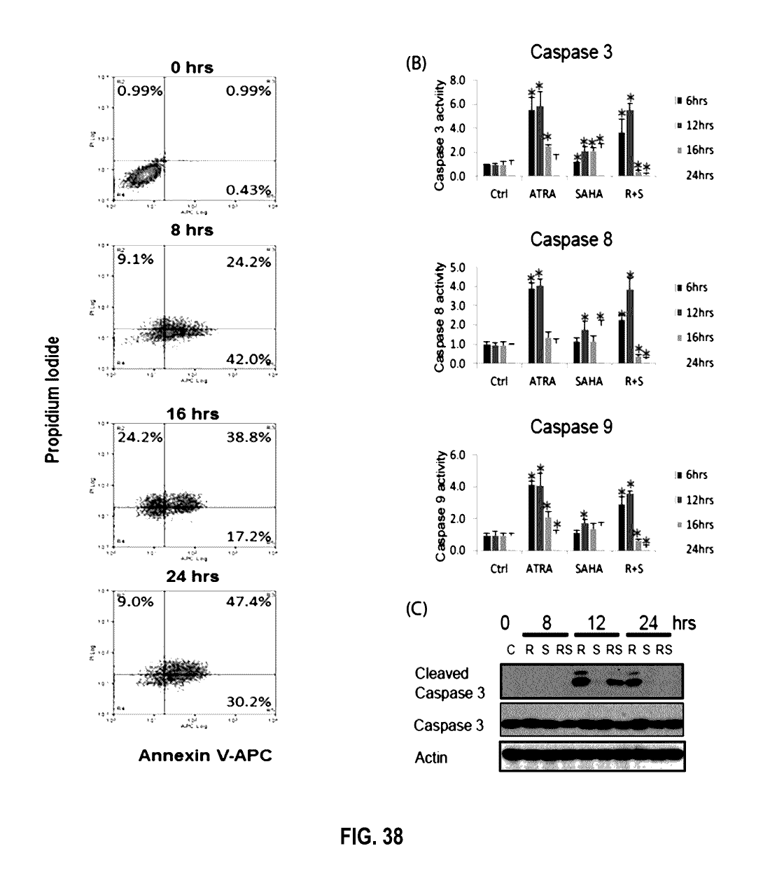

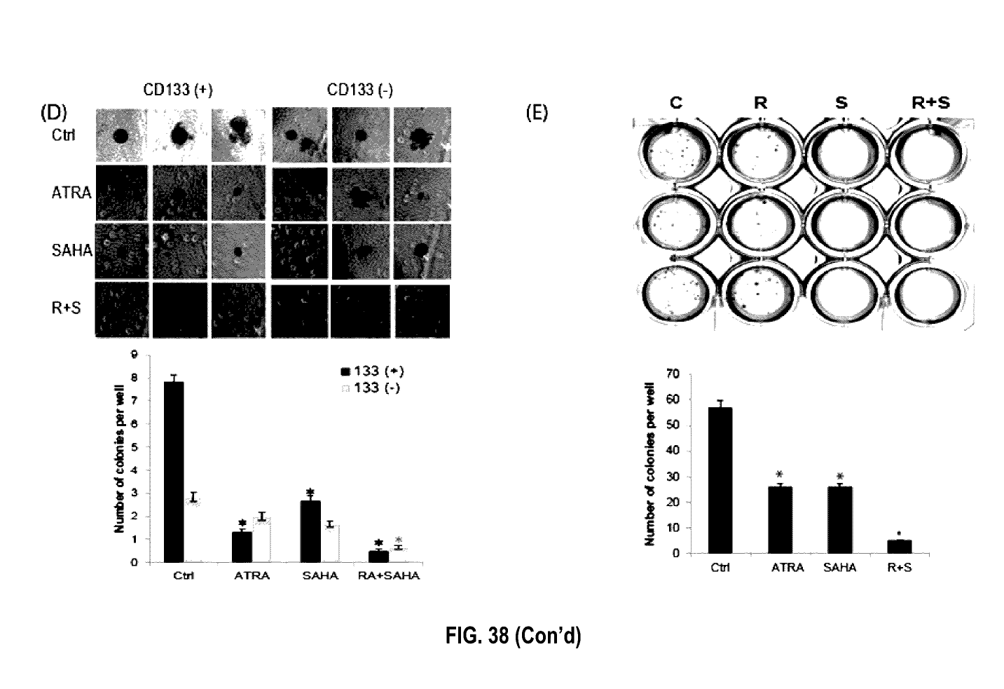

The mechanism of cell killing exhibited by the ATRA-SAHA drug combination was investigated. To test if this drug combination induced TIC apoptosis, the occurrence of apoptosis was first examined by using Annexin V-PI staining. Indeed, the drug combination induced TIC apoptosis following treatment for 8 hours (FIG. 38A). The apoptotic process was further assayed for caspase activity originating from both extrinsic (death receptor) and intrinsic (mitochondrial) pathways. The activities of caspase-3, -8, and -9 were tested at 6, 12, 16, and 24 hour time points. Interestingly, it was found the drug combination not only significantly activated the extrinsic caspase-8 pathway, but also the intrinsic caspase-9 pathways at early times (6 and 12 hours; p<0.05) (FIGS. 38B and 38C). The combination of SAHA did not have any other effects on cell apoptosis, suggesting that in this combination, ATRA plays a major role in the induction of cell apoptosis in the TIC population.

Self-renewal and survival of TICs is the major issue regarding tumor recurrence. Whether this drug combination had an effect on the self-renewal ability of TICs was tested, as assessed by tumor spheroid formation assay. As FIG. 38D shows, the ATRA-only treated group reduced colony numbers by 83% in the CD133 (+) group, and the SAHA-only treated group reduced the colony numbers by 66%. By contrast the combination of ATRA and SAHA significantly reduced the colony numbers by 93.6%. As another assay for anti-tumor properties of these drugs, anchorage independent growth of TICs was examined in soft agar. This assay showed that the ATRA-only treated group only partially reduced colony numbers by 50% as did the SAHA-only treated group; however, the combination of ATRA and SAHA reduced colony numbers by 95% (FIG. 38E). Thus these results indicated that the combination of ATRA and SAHA efficiently inhibited the self-renewal ability of TICs and their tumor forming ability.

Genome-Wide Transcriptome Analysis Reveals the Mechanism for ATRA-SAHA Combination Targeting of TICs.

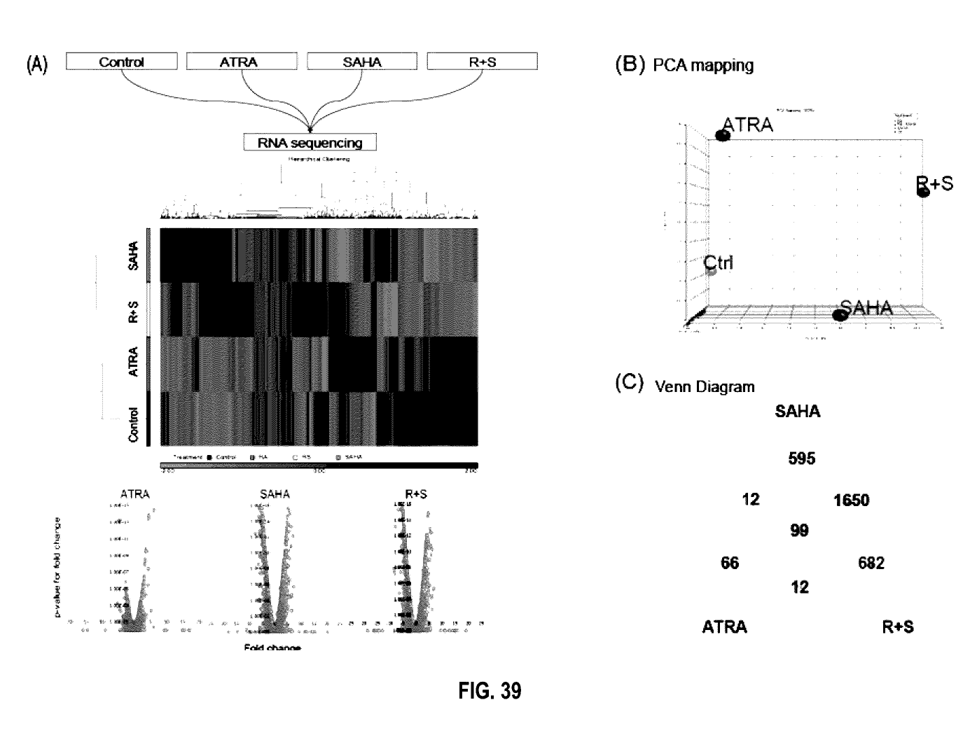

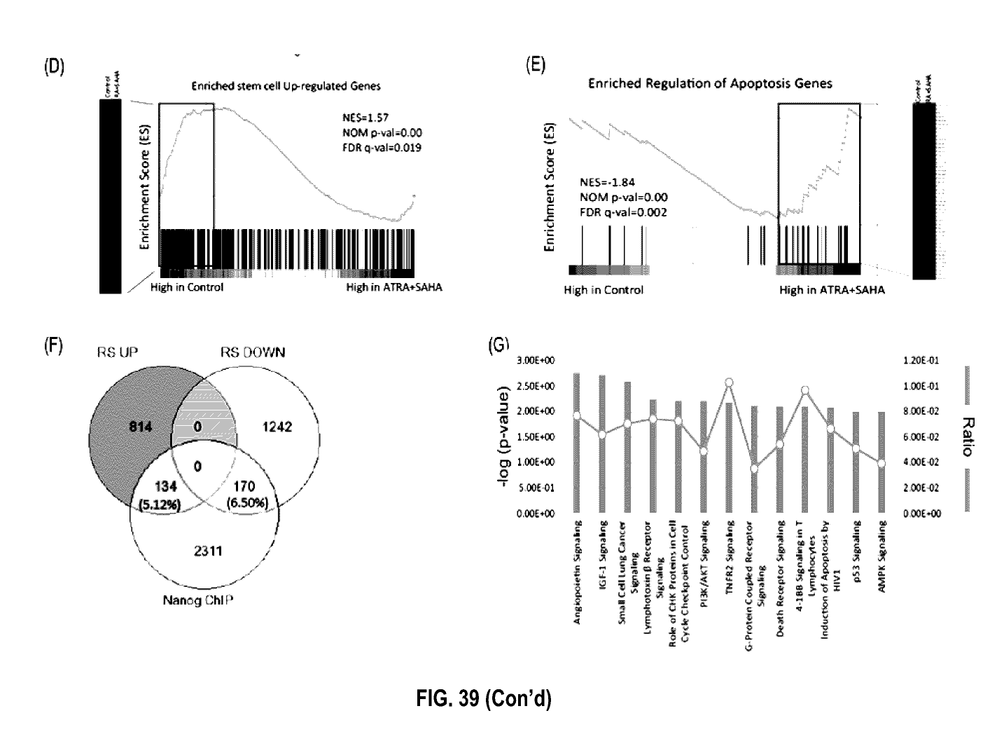

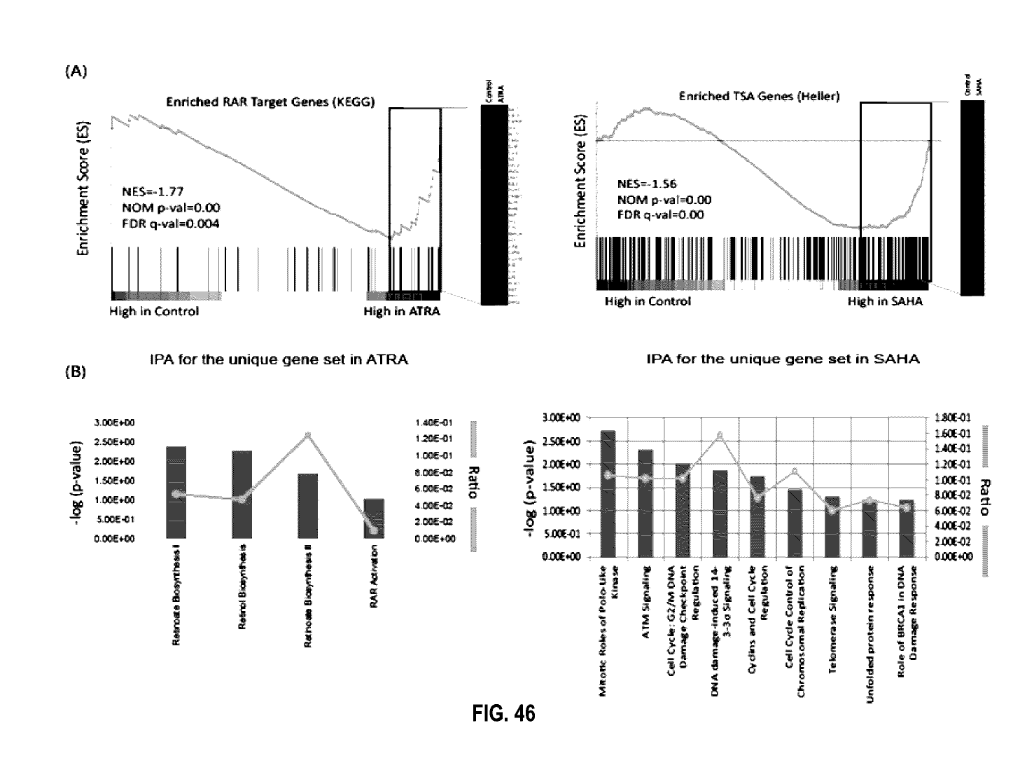

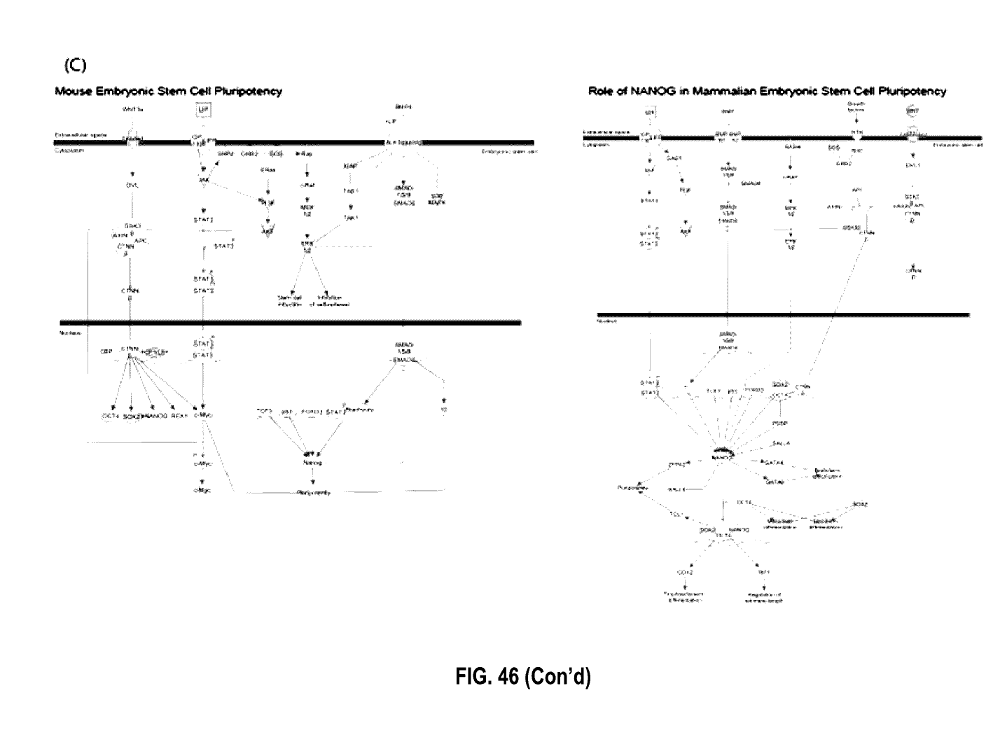

In order to understand the mechanistic basis for the proapoptotic property of the ATRA-SAHA drug combination, whole-transcriptome next-generation sequencing (RNA-seq) was conducted following drug treatments (FIG. 39A). The principal component analysis (PCA) of RNA-seq data showed that the RNA profile of ATRA-treatment was relatively similar to the control group (FIG. 3B). Only 189 genes were differentially expressed from the controls (FIG. 39C). As expected, the subset of affected genes (66 genes) following ATRA treatment was related to retinoid pathways (FIGS. 46A and 46B, left panel) In contrast, the pattern of gene expression of SAHA treatment was quite different from the control group (FIGS. 46A and 46B, right panel). More interestingly, the gene set enrichment analysis (GSEA) shows that the stem cell up-regulated gene set is highly enriched in control group, but not in the drug combination group (FIG. 39D), which is consistent with IPA result (FIG. 46C). In contrast, the apoptosis regulatory gene set, which includes caspase activation, death-association protein kinase (DAPK) and protein ubiquitination pathways, is highly enriched in the drug combination group (FIG. 39E). These results corroborated the cell growth studies that the drug combination inhibited the self-renewal ability and induced apoptosis of TICs.

2617 NANOG target genes were previously identified in TICs via NANOG-ChIP sequencing (Chen et al., 2016). The NANOG-ChIP sequencing data were compared to the RNA sequencing data and discovered that ATRA+SAHA treatment influenced the transcription of 11% of NANOG target genes (FIG. 39F). Furthermore, the IPA showed that the 11% of NANOG target genes effected by ATRA+SAHA treatment plays a vital role in cell survival and cell death pathways (FIG. 39G). These results further substantiated that the drug combination targeted NANOG regulated self-renewal ability and cell survival of TICs.

The ATRA+SAHA Treatment Induces the TIC Growth Arrest and Apoptosis Via the PTEN-FOXO Pathway:

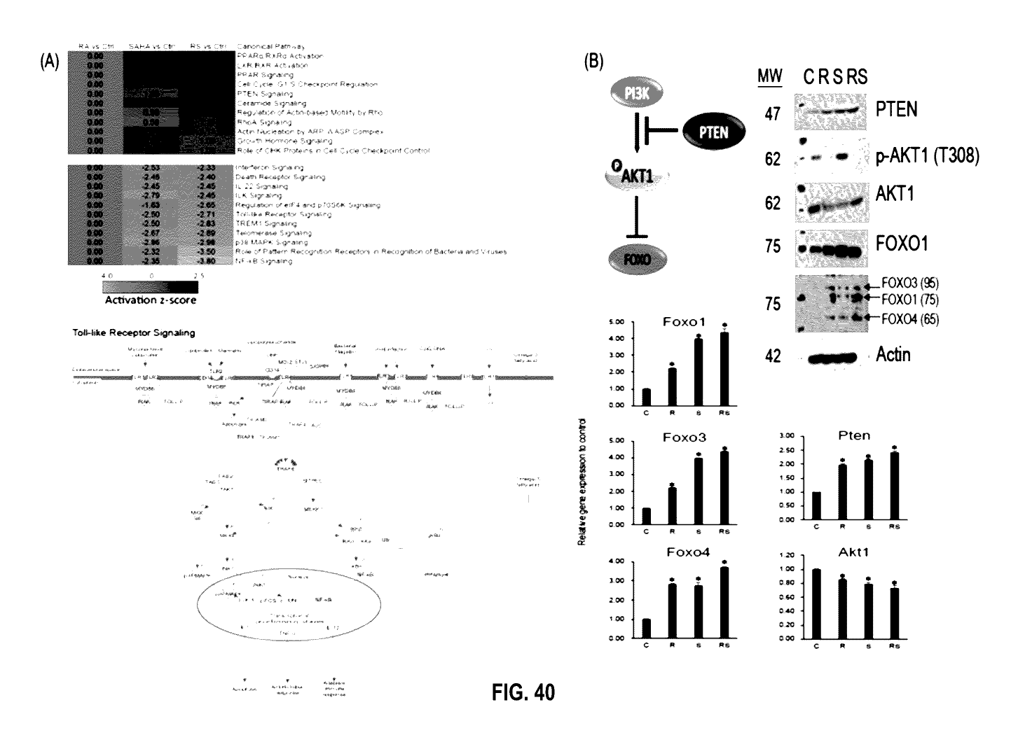

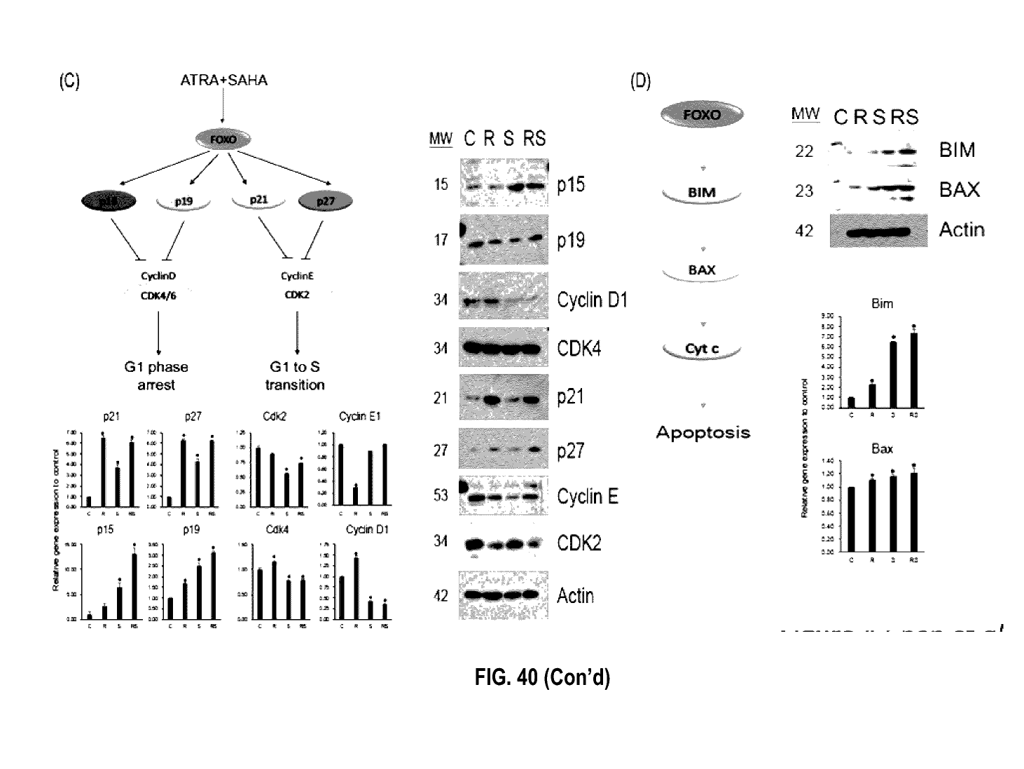

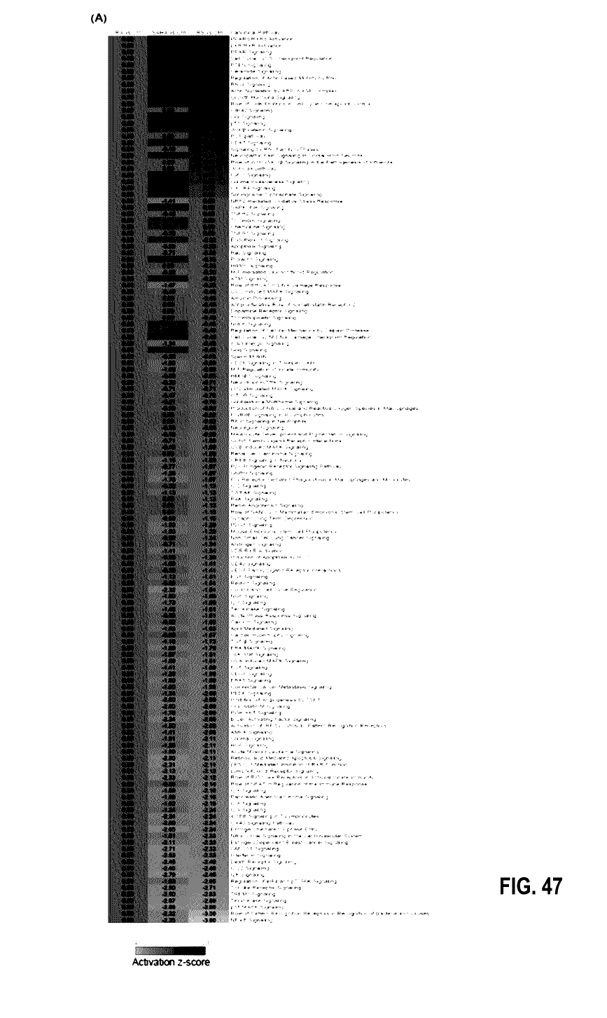

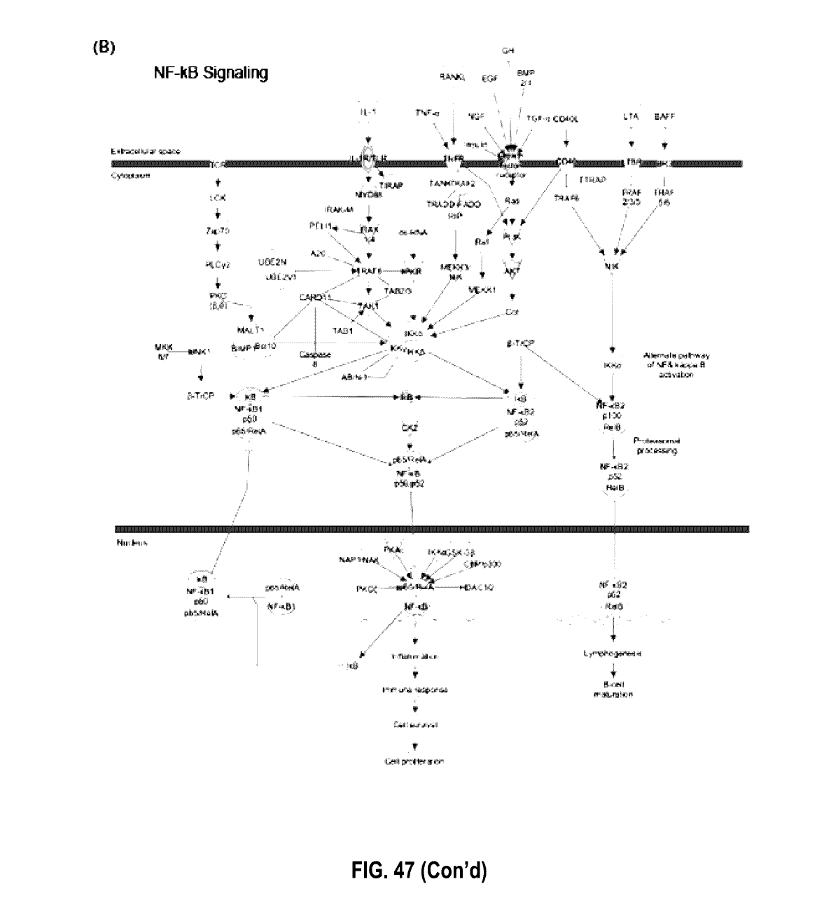

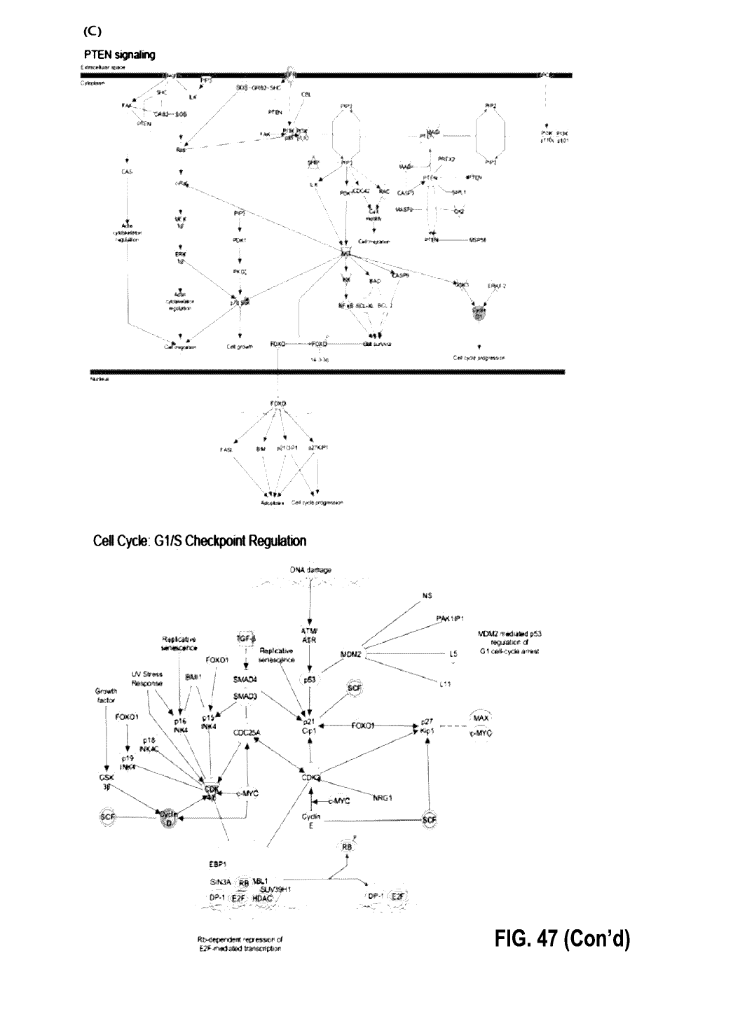

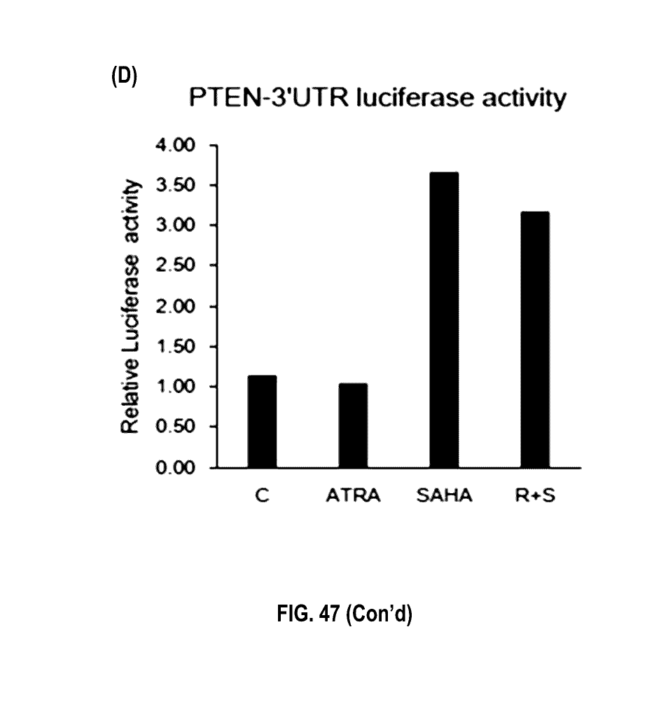

The gene network(s) subject to regulation was examined by drug combination treatment. When comparing the candidate gene pathways among three drug treatment groups and untreated cells (FIG. 40A and FIG. 47A), it was observed that drug combination treatment down-regulated the Toll-like receptor pathway and the NF-κB pathway (FIG. 40A and FIG. 47B). These were previously shown by us to play a critical role for oncogenesis and maintenance of the TIC population (Chen et al., 2013 and Lim et al., 2007). Specifically, it was observed that the drug combination activated PTEN signaling (FIG. 40A and FIG. 47C). PTEN (Phosphatase and tensin homolog deleted on chromosome 10) is a tumor-suppressor gene and regulates cell growth and apoptosis through the PTEN-FOXO pathways (Song et al., 2012). In silico analysis by using Oncomine showed that PTEN is downregulated in two independent HCC libraries (TCGA liver library and Guichard liver library) (data not shown).

An examination of PTEN expression following drug combination treatment was performed with a translation reporter for PTEN. It was observed that luciferase-PTEN 3′-UTR reporter activity increased in response to SAHA treatment but not by ATRA (FIG. 47D). This indicated to us that PTEN was subject to post-transcriptional regulation. The activation of PTEN by drug combination treatment reduced AKT phosphorylation of Thr-308, which led to overexpression of FOXO1/3/4 (FIG. 40B). The FOXO family not only regulates the cell cycle through CDK inhibitors (i.e. p15^{INK4b}, p19^{INK4d}, p21C^{ip1} and p27^{Kip1})(Katayama K et al., 2007), but also activates apoptosis through transactivation of the BIM pathway (Fu Z and Tindall D J, 2008). It was observed that the drug combination treatment induced CDK inhibitors (p15^{INK4b}, p19^{INK4d}, p21^{Cip1} and p27^{Kip1})expression, leading to reduction of cyclins (Cyclin D1 and Cyclin E) and cyclin-dependent kinases (CDK2) (FIG. 40C). The drug combination also induced the expression of BIM, BAX and cytochrome c (FIG. 40D). Thus, these results demonstrated that the drug combination treatment induced TIC growth arrest and apoptosis through the PTEN-FOXO pathway.

ATRA+SAHA Combination Treatment Targets the TIC Population Via Suppression of miR-22

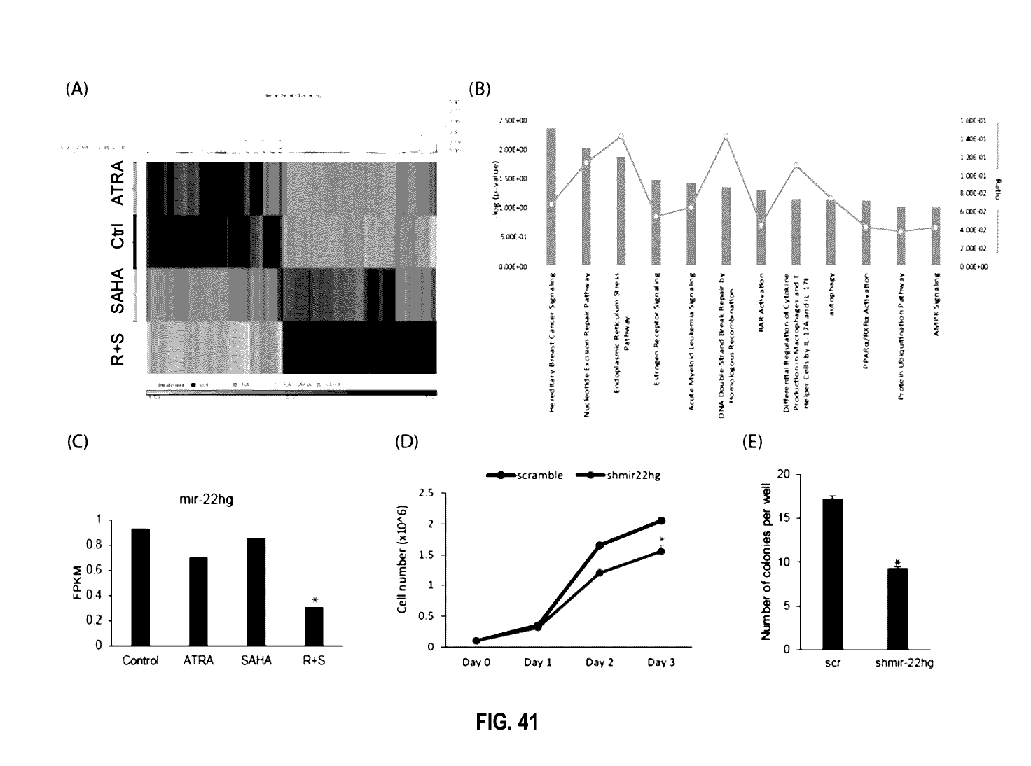

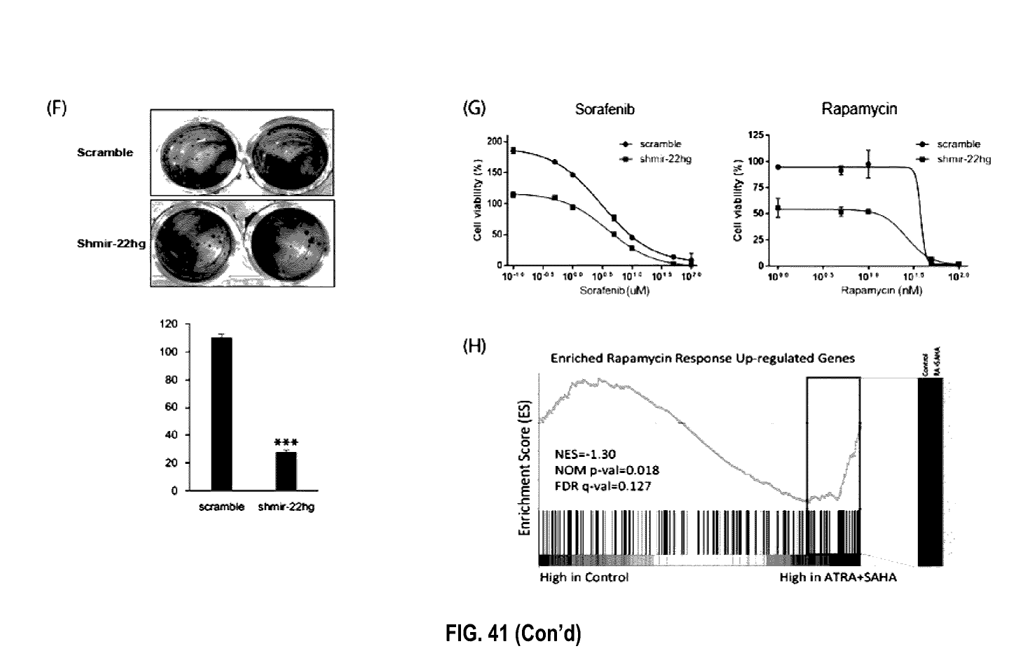

Based on RNA-seq data, a unique set of genes (682 genes) are differentially expressed among the three drug-treatment groups (FIG. 41A). Ingenuity Pathway Analysis indicated a unique subset of genes was associated with solid cancer pathways (such as hereditary breast cancer) and with non-solid cancer pathways (such as acute myeloid leukemia) (FIG. 41B). In addition, this unique subset of genes was related to DNA repair signaling, such as nucleotide excision repair and DNA double strand break repair by homologous recombination. These data suggested the candidate genes in the unique gene subset expressed upon drug combination treatment promoted TIC proliferation.

Notably, few important associations involved DNA repair signaling, such as nucleotide excision repair and DNA double strand break repair by homologous recombination. Recent evidence suggests that microRNAs (miRNAs) play a crucial regulatory role in DNA damage and repair (Tessitore et al., 2014). It was therefore reasoned that any differentially expressed miRNA transcripts in the pool of 595 genes could be a potential candidate to explain the cause behind defective self-renewal in TICs post combination drug treatment. Post-transcriptional regulation by microRNA is another layer of regulation of overall gene expression. The unique set of affected genes identified by RNA sequencing, in the drug combination group included downregulation of non-coding RNA miR-22 host gene (miR-22hg) after drug combination treatment (FIG. 41C). To determine if miR-22hg promoted self-renewal of TICs, miR-22hg was knocked down in TICs and subjected these cells to colony formation assays. Silencing miR-22hg reduced TIC growth (FIG. 41D) and colony numbers (FIGS. 41E and 41F), indicating that down-regulation of miR-22hg was important for suppressing TIC self-renewal. To test if the effects of miR-22hg knockdown were not restricted to ATRA-SAHA treatment but a more general property, TICs were with the conventional chemotherapy drug Sorafenib and Rapamycin. Silencing of miR-22hg rendered TICs susceptible to conventional chemotherapy (FIG. 41G), suggesting that higher expression levels of miR-22hg promoted self-renewal of TICs and drug resistance. Gene Set Enrichment Analysis (GSEA) further confirmed that the rapamycin response up-regulated genes also highly enriched in the ATRA-SAHA combination group (FIG. 41H). This indicated that suppression of miR-22 expression via dual-drug combination increased the susceptibility of TICs to drug treatment.

The ATRA+SAHA Combination Treatment Alters the DNA Methylation Pattern of Nanog Via Regulation of miR-22 and TET2:

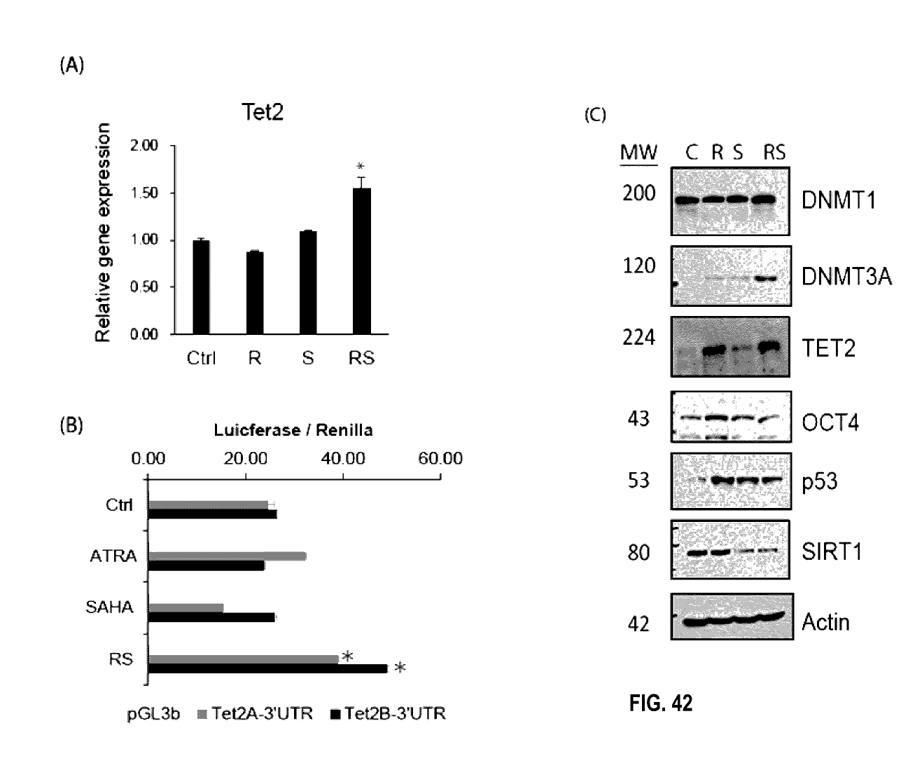

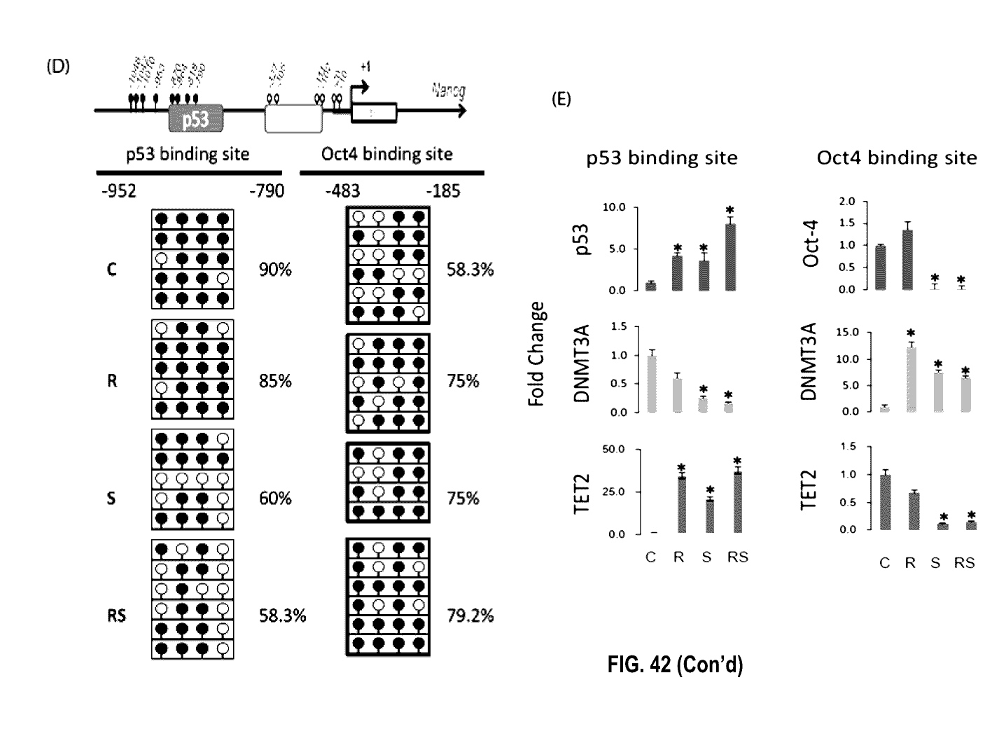

Upregulation of microRNA 22 promotes tumor metastasis by directly down-regulating members of the TET gene family, which are methylcytosine dioxygenases (Song et al., 2013). It was found that Tet2 was up-regulated after treatment with the drug combination (FIG. 42A). This post-transcriptional regulation was confirmed by employing luciferase reporter genes fused to the TET2a or TET2B 3′-UTR. As shown in FIG. 42B luciferase reporter activity was elevated after the drug combination treatment. These data indicated that the dual-drug combination up-regulated TET2 by repressing miR-22 expression.

Changes in TET2 levels could have a consequence on DNA methylation patterns of genes associated with ATRA-SAHA sensitivity. In order to investigate this possibility, DNA bisulfite sequencing was performed of the Nanog promoter/enhancer regions to determine if changes occurred. The Nanog promoter is hypomethylated in the CD133 (+) population of human HCC cell lines (Wang et al., 2013), by contrast it is highly methylated in primary hepatocytes. Similar to CD133 (+) cell lines, primary TICs were found also to be hypomethylated in the Nanog promoter proximal region consistent with the observed higher expression levels of NANOG.

In addition, the luciferase activity of the TET2 3′UTR was activated after the combination treatment (FIG. 42B). These data indicate that the drug combination upregulated TET2 by suppressing miR-22.

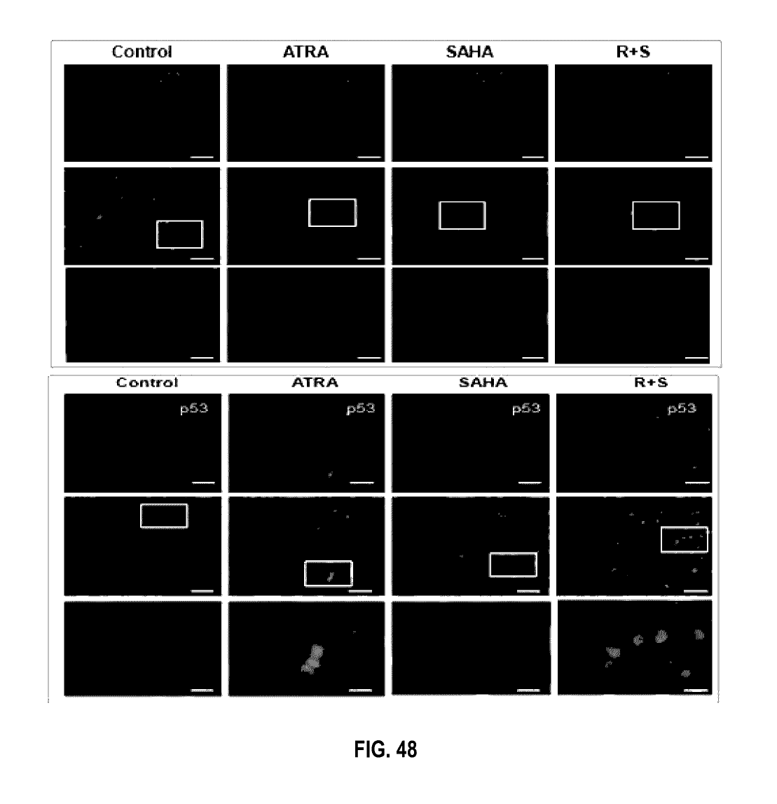

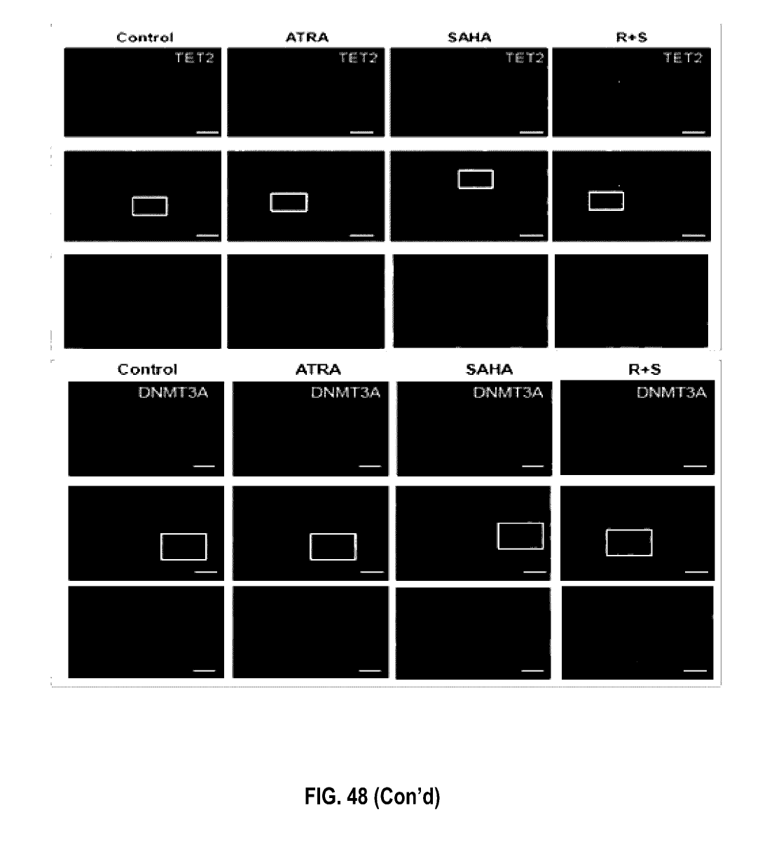

Because members of the TET family are methylcytosine dioxygenases, whether or not the promoter pattern of DNA methylation is altered after drug treatment was further investigated by DNA bisulfite sequencing, especially on the Nanog promoter region. It has been shown that dysregulated hypomethylation of the Nanog promoter was observed in the CD133 (+) population of human HCC cell lines (Wang et al., 2013). It was also observed that the different methylation pattern of Nanog promoter among mouse embryonic stem cells, TICs and normal hepatocytes (FIG. 48A). Two important transcription factor binding sites, Oct4 (−285) and p53 (−790), are located on the Nanog promoter region and regulate Nanog expression in contrasting ways. OCT4 is recruited to the Nanog promoter to activate Nanog (Boyer et al., 2005, Rodda et al., 2005, Lo, 2008, van den Berg et al., 2010), whereas p53 is recruited to the Nanog promoter to suppress Nanog expression (Meletis et al., 2006, Pan et al., 2007 and Han et al., 2008). TICs were examined for changes in OCT4 and p53 levels following drug treatment. The drug combination reduced mRNA levels of OCT4 and SIRT1, the latter is a suppressor of p53 (Li et al., 2012). By contrast, the combination treatment induced TET2, DNMT3A, and p53 upregulation (FIG. 42C and FIG. 48B). As shown in FIG. 42D, the p53 binding site in the Nanog promoter was highly methylated in TICs; however, the OCT4 binding site of the Nanog promoter was less methylated in TICs (58.3% and 90%, respectively). More interestingly, after the drug combination treatment increased DNA methylation in the OCT4 binding site (the control and the combination treatment were 58.3% and 79.2%, respectively), whereas methylation was decreased in the p53 binding site (the control and the combination treatment were 90% and 58.3%, respectively).

As confirmation of the change in DNA binding activity of p53 and Oct4 to the Nanog promoter, chromatin immunoprecipitation-qPCR (ChIP-qPCR) was performed from TICs treated with the drug combination. Under these conditions, it was observed that p53 was recruited to the Nanog promoter region whereas Oct4 was absent (FIG. 42E). Thus the consequence of drug combination treatment appeared to increase recruitment of two major DNA methylation regulators, TETs and DNMTs to the Nanog promoter region with a subsequent effect on transcription factor binding and a corresponding change in Nanog transcription. On the other hand, DNMT3A was removed from the p53 binding site and recruited to the Oct4 binding site after the combination treatment. These results demonstrated that the alteration of the DNA methylation pattern of the Nanog promoter resulted in repression of Nanog expression.

Dual Drug Combination Treatment Attenuates Tumor Growth In Vivo:

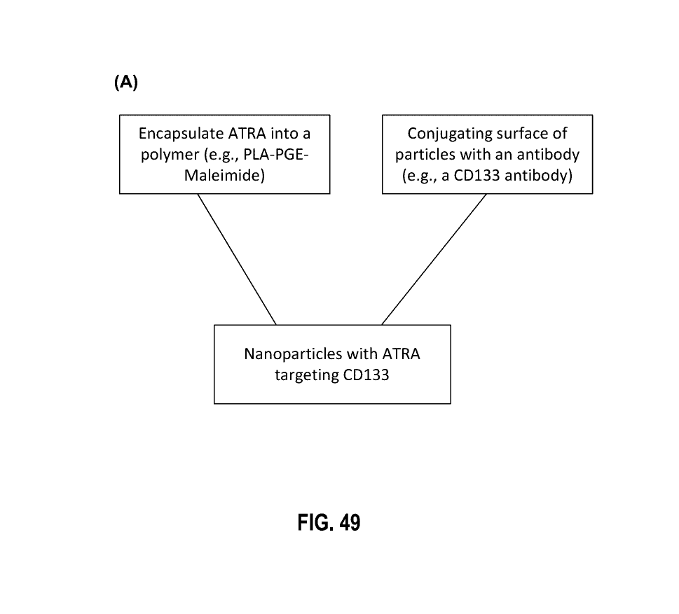

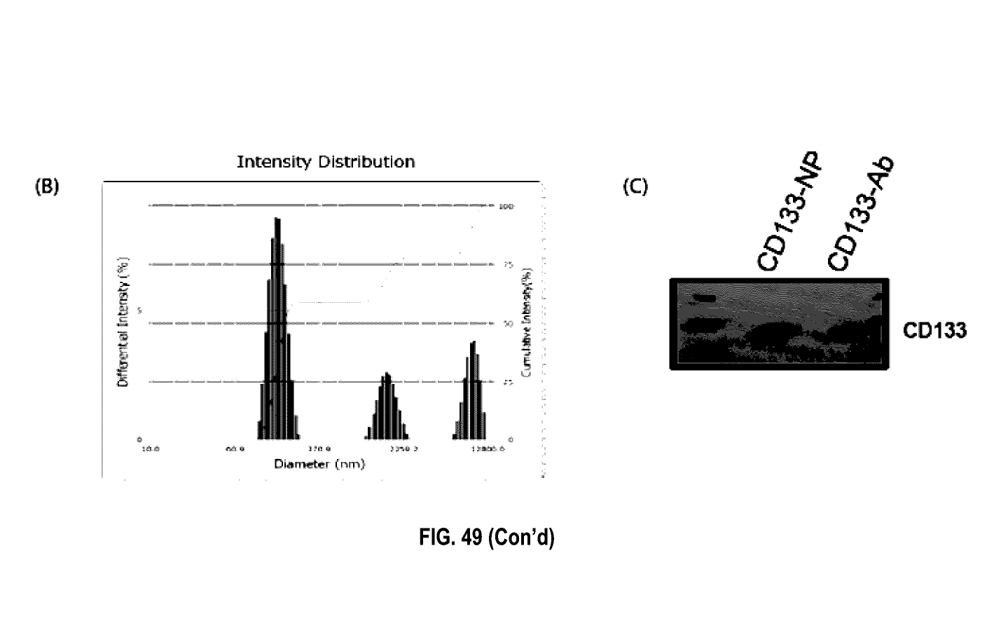

The efficacy of ATRA-SAHA on TIC viability in vitro prompted us to examine if the drug combination inhibited tumor growth in vivo. For these studies, 10⁶ CD133 (+) Huh7 cells were subcutaneously implanted into NOD/Shi-scid/IL-2Rγ^{null} (NOG) mice. In order to specifically target the CD133 (+) population, ATRA was encapsulated into nanoparticles conjugated with CD133 antibody using biodegradable poly(D,L-lactide-co-glycolide) (PLGA) polymer (FIG. 49). Once a tumor size of 100 mm³ was reached, the mice were treated with ATRA only (5 μg/ml), SAHA only (0.5 μg/ml), or the combination with empty nanoparticles as a control for all treatments. It was observed that the single drug treatment groups did not reduce or even promote tumor growth while the combination treatment significantly inhibited tumor growth when compared to the single drug treatments and control groups after 4 days of treatment (FIG. 42A). These data indicated that the drug combination was indispensable for tumor growth inhibition.

The tumor morphology was examined in the control group from hematoxylin and eosin stained tissue sections. Representative tumor tissues sections from ATRA-treated mice were found to have necrotic regions (FIG. 42B). The SAHA-treatment group did not induce cell death of tumor cells, but showed increased vascularization; which may explain why SAH-treated tumors had the largest tumor sizes. The combination treatment induced extensive necrosis, indicating that the dual drug regimen eliminated almost all tumor cells. In order to understand the basis for cell death, TUNEL staining was performed for each of the drug treatment groups. The tumors from the ATRA-treatment group had TUNEL-positive tumor cells, but far less than that observed for tumors in the SAHA-treatment group (FIG. 42C). The drug combination group showed a significant increase of apoptosis, indicating that the combination of ATRA and SAHA effectively inhibited tumor growth.

A comparison of gene expression patterns in liver cancers with overall survival was performed using GSEA analysis. Both the liver cancer recurrence up-regulated gene set (FIG. 42D) and liver cancer survival down-regulation gene set (FIG. 42E) are enriched in control group, indicating that the ATRA-SAHA drug combination effectively suppressed tumor growth and tumor recurrence and improved the overall survival rate.

In conclusion, the results showed the drug combination suppressed miR-22 expression, which in turn was permissive for induction of the PTEN-regulated apoptosis pathway and suppressed Nanog gene expression. The latter occurred through a change in the DNA methylation pattern of the Nanog promoter itself leading to a loss of self-renewal ability and drug susceptibility. These results summarized in the model shown in FIG. 42F.

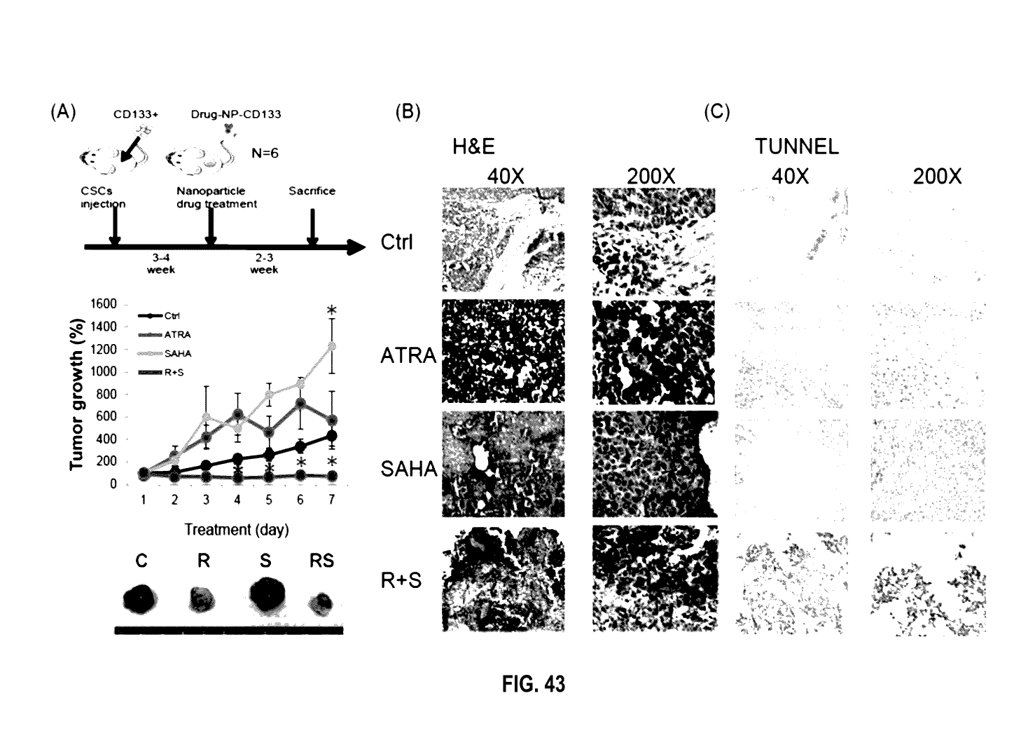

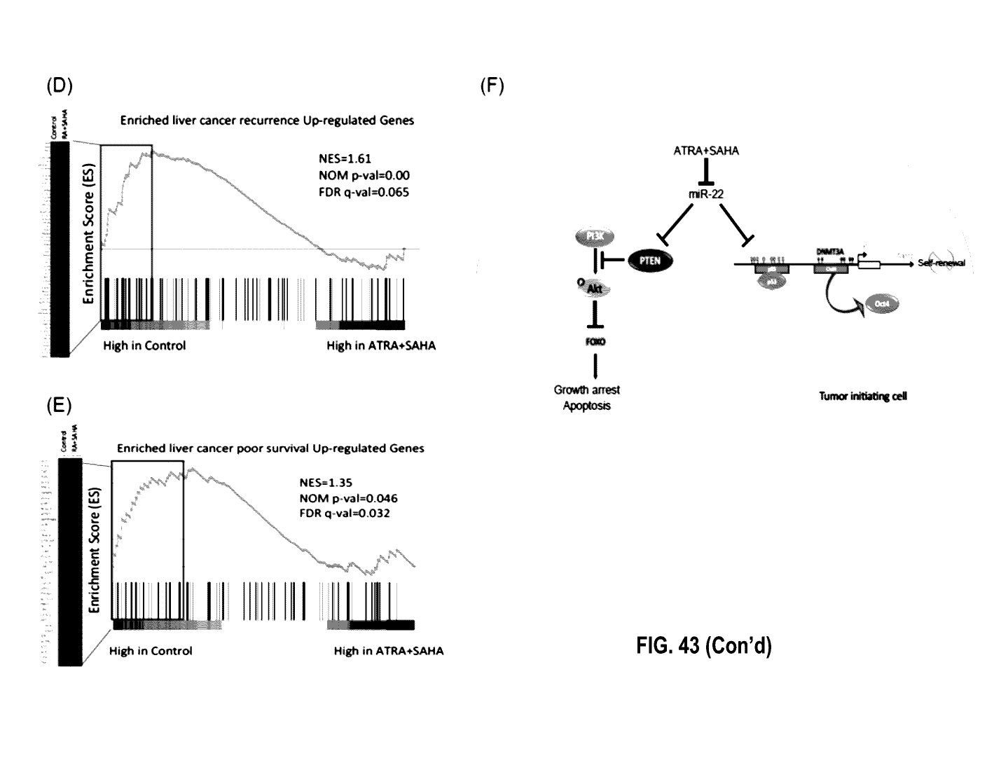

The goal of these studies was to identify drugs that would specifically target the TIC population in HCC. By employing a high throughput, TIC viability screen tested against a library of FDA approved drugs, it was found that a retinoic acid derivative, ATRA, showed the best inhibition of cell growth (FIGS. 43B and 43C). This drug was included in a refined screening approach targeting Nanog expression in a secondary dual drug regimen with the FDA approved drug library. From this secondary drug screening, it was found that the combination of ATRA and SAHA demonstrated the best efficacy for inhibition of TIC growth in vitro and in vivo (FIGS. 43A-43C). The analysis of the mechanism by which these drugs killed tumor cells showed a bipartite process leading to cell death. Nanog expression was suppressed due to increased expression of TET2 leading to a change in promoter methylation and subsequent repression of Nanog transcription. The initiating event for this was found to be repression of miR-22 expression. As a consequence of the latter, PTEN activity increased with a corresponding induction of the apoptosis pathway in TICs leading to cell death. Of particular interest was the observation that knockdown of miR-22 expression also sensitized cells to killing by other chemotherapeutic agents, e.g., rapamycin. Although our favored drug combination is ATRA+SAHA, this general strategy of repressing miR-22 may be useful for sensitizing cancer cells to other therapeutic drugs.

The TIC population plays a major role in tumor recurrence and therapy resistance. Identifying candidates that can specifically target this population should be a final goal for cancer therapy. As such most molecular screenings only focus on one marker when assay against a large molecule library (Gupta et al., 2009); however, the marker may not be efficient in eliminating the target population of malignant cells. In this study, three different kinds of screens were conducted for the TIC population, including a CD133 cell viability screen, a NANOG-GFP high-content screen, and a combination screen. Based on the screening results, it was found that ATRA can specifically inhibited the CD133 (+) TIC population.

Regulation of cell growth via retinoic acid signaling has been widely used to treat various types of cancer, such as breast cancer (Garattini et al., 2007), lung cancer (Dahl et al., 2000), ovarian cancer (Harant et al., 1993), prostate cancer (Zhao et al., 1999), neuroblastoma (Reynolds et al., 2003), renal cell carcinoma (Motzer et al., 2000), pancreatic cancer (Weiss et al., 2009), liver cancer (Meyskens et al., 1998), head and neck cancer (Rubin Grandis et al., 1996), and acute promyelocytic leukemia (Huang et al., 1988). Retinoic acid is also an inducer of embryonic stem cell and hematopoietic stem cell differentiation (Simandi et al., 2010, Rochette-Egly, 2015, Chanda et al., 2013). In HCC, it has been shown that induction and intracellular localization of the nerve growth factor IB (NGFIB aka Nur77 via Fenretinide, a structural analogue of retinoic acid, could induce cell apoptosis through activation of caspase-3/7 (Yang et al., 2010). In the study, it was further demonstrated that retinoic acid not only activated the extrinsic caspase-8 pathway, but also the intrinsic caspase-9 pathway.

The HDAC inhibitors are widely used in treatment of various cancers such as leukemia (Rosato et al., 2003), pancreatic cancer (Kumagai et al., 2007), lung cancer (Komatsu et al., 2006), breast and colon tumors (Butler et al., 2002), ovarian cancer (Strait et al., 2005), and cervical cancer (Li and Wu, 2004). These inhibitors have broad effects on the regulation of the cell cycle, apoptosis, cell differentiation, autophagy, and are anti-angiogenic (Khan & La Thangue, 2012). In addition, the HDAC inhibitors can induce cell cycle arrest through the induction of p21 and downregulation of cyclins (Sabdor et al., 2000). Furthermore, HDAC inhibitor treatments induce accumulation of reactive oxygen species, which results in DNA damage and subsequent apoptosis (Petruccelli et al., 2011). In this study, it was shown that treatment with the HDAC inhibitor (SAHA) alone failed to reduce cell growth in vitro or to reduce tumor growth in vivo, strongly suggesting that the single treatment for conventional cancer therapy was not sufficient. It was shown that only the combination of the HDAC inhibitor with ATRA successfully reprogrammed the TIC population for cell apoptosis and suppress tumor growth (FIG. 43A-43C).

It was found that miR-22hg was downregulated following drug combination treatment. Moreover, TET2, the target of miR22, was upregulated, indicating that epigenetic modification, especially DNA methylation, was a response to the drug combination therapy. It is well known that this combination is widely used in acute myeloid leukemia patients to induce leukemia cell differentiation (Salomini and Pandolfi, 2000). In human malignant melanoma, the combination of 13-cis-retinoic acid with the HDAC inhibitor LAQ824 induces cell growth arrest and apoptosis (Kato et al., 2007). It has also been shown that the HDAC inhibitor DWP0016 suppresses miR-22 via p53-independent PTEN activation and inhibits neuroblastoma cell growth (Jin et al., 2013). In cervical cancer, the combination of retinoic acid with the HDAC inhibitor BML-210 can induce HeLa cell apoptosis through the p53 pathway (Borutinskaite et al., 2006). In HCC, the combination of Fenretinide with TSA, another kind of general HDAC inhibitor, can further induce cell apoptosis via up-regulation of Nur77 (Yang et al., 2010). However, few of these results provided the detailed epigenetic mechanism dependent upon the combination treatment. The data indicated that this drug combination not only induces cell apoptosis but also inhibited the ability of self-renewal via epigenetic regulation.

MicroRNA analogues or antagonist therapies are an emerging anti-cancer strategy; however, the miRNA-based therapies are still in the clinical trial phase, and the therapeutic concerns regarding dosage, stability, and safety still remain unclear. Here, it was demonstrated that the combination of the FDA approved drugs ATRA and SAHA can manipulate microRNA expression with improved safety control.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

1. Ahfeldt, T., Schinzel, R. T., Lee, Y. K., Hendrickson, D., Kaplan, A., Lum, D. H., Camahort, R., Xia, F., Shay, J., Rhee, E. P., et al. (2012). Programming human pluripotent stem cells into white and brown adipocytes. Nat Cell Biol 14, 209-219.
2. Aoyama, T., Souri, M., Ueno, I., Kamijo, T., Yamaguchi, S., Rhead, W. J., Tanaka, K., and Hashimoto, T. (1995). Cloning of human very-long-chain acyl-coenzyme A dehydrogenase and molecular characterization of its deficiency in two patients. Am J Hum Genet 57, 273-283.
3. Bauer, A. K., Dixon, D., DeGraff, L. M., Cho, H. Y., Walker, C. R., Malkinson, A. M., and Kleeberger, S. R. (2005). Toll-like receptor 4 in butylated hydroxytoluene-induced mouse pulmonary inflammation and tumorigenesis. J Natl Cancer Inst 97, 1778-1781.
4. Borutinskaite V V, Navakauskiene R, Magnusson K E. Retinoic acid and histone deacetylase inhibitor BML-210 inhibit proliferation of human cervical cancer HeLa cells. Ann N Y Acad Sci. 2006 December; 1091:346-55.
5. Boyer L A, Lee T I, Cole M F, Johnstone S E, Levine S S, Zucker J P, Guenther M G, Kumar R M, Murray H L, Jenner R G, Gifford D K, Melton D A, Jaenisch R, Young R A. Core transcriptional regulatory circuitry in human embryonic stem cells. Cell. 2005 Sep. 23; 122(6):947-56.
6. Bussolati B, Bruno S, Grange C, Ferrando U, Camussi G. Identification of a tumor-initiating stem cell population in human renal carcinomas. FASEB J. 2008 October; 22(10):3696-705.
7. Butler L M, Zhou X, Xu W S, Scher H I, Rifkind R A, Marks P A, Richon V M. The histone deacetylase inhibitor SAHA arrests cancer cell growth, up-regulates thioredoxin-binding protein-2, and down-regulates thioredoxin. Proc Natl Acad Sci USA. 2002 Sep. 3; 99(18):11700-5.
8. Campanero, M. R., Armstrong, M., and Flemington, E. (1999). Distinct cellular factors regulate the c-myb promoter through its E2F element. Mol Cell Biol 19, 8442-8450.
9. Caro, P., Kishan, A. U., Norberg, E., Stanley, I. A., Chapuy, B., Ficarro, S. B., Polak, K., Tondera, D., Gounarides, J., Yin, H., et al. (2012). Metabolic signatures uncover distinct targets in molecular subsets of diffuse large B cell lymphoma. Cancer Cell 22, 547-560.
10. Chanda B, Ditadi A, Iscove N N, Keller G. Retinoic acid signaling is essential for embryonic hematopoietic stem cell development. Cell. 2013 Sep. 26; 155(1):215-27.
11. Chen C L, Uthaya Kumar D B, Punj V, Xu J, Sher L, Tahara S M, Hess S, Machida K. NANOG metabolically reprograms tumor-initiating stem-like cells through tumorigenic changes in oxidative phosphorylation and fatty acid metabolism. Cell Metabolism 2016 Jan. 12; 23(1):206-219.
12. Chen, C. L., Tsukamoto, H., Liu, J. C., Kashiwabara, C., Feldman, D., Sher, L., Dooley, S., French, S. W., Mishra, L., Petrovic, L., et al. (2013). Reciprocal regulation by TLR4 and TGF-beta in tumor-initiating stem-like cells. The Journal of clinical investigation 123, 2832-2849.
13. Cheng G Z, Zhang W Z, Sun M, et al. Twist is transcriptionally induced by activation of STAT3 and mediates STAT3 oncogenic function. J Biol Chem 2008; 283:14665-73.
14. Chiou S H, Yu C C, Huang C Y, Lin S C, Liu C J, Tsai T H, Chou S H, Chien C S, Ku H H, Liu F, Kong X, Lv L, Gao J. MiR-155 targets TP53INP1 to regulate liver cancer stem cell acquisition and self-renewal. FEBS Lett. 2015 Feb. 13; 589(4):500-6.
15. Chiou S H, Yu C C, Huang C Y, Lin S C, Liu C J, Tsai T H, Chou S H, Chien C S, Ku H H, Lo J F. Positive correlations of Oct-4 and Nanog in oral cancer stem-like cells and high-grade oral squamous cell carcinoma. Clin Cancer Res. 2008 Jul. 1; 14(13):4085-95.
16. Dahl A R, Grossi I M, Houchens D P, Scovell L J, Placke M E, Imondi A R, Stoner G D, De Luca L M, Wang D, Mulshine J L. Inhaled isotretinoin (13-cis retinoic acid) is an effective lung cancer chemopreventive agent in AUJ mice at low doses: a pilot study. Clin Cancer Res. 2000 August; 6(8):3015-24.
17. Dapito, D. H., Mencin, A., Gwak, G. Y., Pradere, J. P., Jong, M. K., Mederacke, I., Caviglia, J. M., Khiabanian, H., Adeyemi, A., Bataller, R., et al. (2012). Promotion of hepatocellular carcinoma by the intestinal microbiota and TLR4. Cancer Cell 21, 504-516.
18. DeBerardinis, R. J., Mancuso, A., Daikhin, E., Nissim, I., Yudkoff, M., Wehrli, S., and Thompson, C. B. (2007). Beyond aerobic glycolysis: transformed cells can engage in glutamine metabolism that exceeds the requirement for protein and nucleotide synthesis. Proceedings of the National Academy of Sciences of the United States of America 104, 19345-19350.
19. Diehl, A. M., and Hoek, J. B. (1999). Mitochondrial uncoupling: role of uncoupling protein anion carriers and relationship to thermogenesis and weight control “the benefits of losing control”. J Bioenerg Biomembr 31, 493-506.
20. Donato F, Gelatti U, Limina R M, et al. Southern Europe as an example of interaction between various environmental factors: a systematic review of the epidemiologic evidence. Oncogene 2006; 25:3756-70.
21. Erridge C, Attina T, Spickett C M, et al. A high-fat meal induces low-grade endotoxemia: evidence of a novel mechanism of postprandial inflammation. Am J Clin Nutr 2007; 86:1286-92.
22. Ezeh U I, Turek P J, Reijo R A, Clark A T. Human embryonic stem cell genes OCT4, NANOG, STELLAR, and GDF3 are expressed in both seminoma and breast carcinoma. Cancer. 2005 Nov. 15; 104(10):2255-65.
23. Fabbri M, Garzon R, Cimmino A, Liu Z, Zanesi N, Callegari E, Liu S, Alder H, Costinean S, Fernandez-Cymering C, Volinia S, Guler G, Morrison C D, Chan K K, Marcucci G, Calin G A, Huebner K, Croce C M. MicroRNA-29 family reverts aberrant methylation in lung cancer by targeting DNA methyltransferases 3A and 3B. Proc Natl Acad Sci USA. 2007 Oct. 2; 104(40):15805-10.
24. Fagan, R., Flint, K. J., and Jones, N. (1994). Phosphorylation of E2F-1 modulates its interaction with the retinoblastoma gene product and the adenoviral E4 19 kDa protein. Cell 78, 799-811.
25. Fan, W., Morinaga, H., Kim, J. J., Bae, E., Spann, N. J., Heinz, S., Glass, C. K., and Olefsky, J. M. (2010). FoxO1 regulates Tlr4 inflammatory pathway signalling in macrophages. EMBO J 29, 4223-4236.
26. Farnie G, Clarke R B. Breast stem cells and cancer. Ernst Schering Found Symp Proc 2006:141-53.
27. Feingold K R, Grunfeld C. Role of cytokines in inducing hyperlipidemia. Diabetes 1992; 41 Suppl 2:97-101.
28. Feldman D E, Chen C, Punj V, et al. Pluripotency factor-mediated expression of the leptin receptor (OB-R) links obesity to oncogenesis through tumor-initiating stem cells. Proc Natl Acad Sci USA; 109:829-34.
29. Ferrick, D. A., Neilson, A., and Beeson, C. (2008). Advances in measuring cellular bioenergetics using extracellular flux. Drug Discov Today 13, 268-274.
30. Fu Z, Tindall D J. FOXOs, cancer and regulation of apoptosis. Oncogene. 2008 Apr. 7; 27(16):2312-9.
31. Fukata, M., Chen, A., Vamadevan, A. S., Cohen, J., Breglio, K., Krishnareddy, S., Hsu, D., Xu, R., Harpaz, N., Dannenberg, A. J., et al. (2007). Toll-like receptor-4 promotes the development of colitis-associated colorectal tumors. Gastroenterology 133, 1869-1881.
32. Gale M J, Jr., Korth M J, Tang N M, et al. Evidence that hepatitis C virus resistance to interferon is mediated through repression of the PKR protein kinase by the nonstructural 5A protein. Virology 1997; 230:217-27.
33. Gan, B., Hu, J., Jiang, S., Liu, Y., Sahin, E., Zhuang, L., Fletcher-Sananikone, E., Colla, S., Wang, Y. A., Chin, L., et al. (2010). Lkbl regulates quiescence and metabolic homeostasis of haematopoietic stem cells. Nature 468, 701-704.
34. Garattini E, Gianni M, Terao M. Retinoids as differentiating agents in oncology: a network of interactions with intracellular pathways as the basis for rational therapeutic combinations. Curr Pharm Des. 2007; 13(13):1375-400.
35. Giordano, A., Calvani, M., Petillo, O., Grippo, P., Tuccillo, F., Melone, M. A., Bonelli, P., Calarco, A., and Peluso, G. (2005). tBid induces alterations of mitochondrial fatty acid oxidation flux by malonyl-CoA-independent inhibition of carnitine palmitoyltransferase-1. Cell Death Differ 12, 603-613.
36. Gripon, P., Rumin, S., Urban, S., Le Seyec, J., Glaise, D., Cannie, I., Guyomard, C., Lucas, J., Trepo, C., and Guguen-Guillouzo, C. (2002). Infection of a human hepatoma cell line by hepatitis B virus. Proceedings of the National Academy of Sciences of the United States of America 99, 15655-15660.
37. Guichard C, Amaddeo G, Imbeaud S, et al. Integrated analysis of somatic mutations and focal copy-number changes identifies key genes and pathways in hepatocellular carcinoma. Nat Genet 2012; 44:694-8.
38. Gupta P B, Onder T T, Jiang G, Tao K, Kuperwasser C, Weinberg R A, Lander E S. Identification of selective inhibitors of cancer stem cells by high-throughput screening. Cell. 2009 Aug. 21; 138(4):645-59.
39. Gurumurthy, S., Xie, S. Z., Alagesan, B., Kim, J., Yusuf, R. Z., Saez, B., Tzatsos, A., Ozsolak, F., Milos, P., Ferrari, F., et al. (2010). The Lkbl metabolic sensor maintains haematopoietic stem cell survival. Nature 468, 659-663.
40. Gutgesell, A., Wen, G., Konig, B., Koch, A., Spielmann, J., Stangl, G. I., Eder, K., and Ringseis, R. (2009). Mouse carnitine-acylcarnitine translocase (CACT) is transcriptionally regulated by PPARalpha and PPARdelta in liver cells. Biochim Biophys Acta 1790, 1206-1216.
41. Haluzik, M., Gavrilova, O., and LeRoith, D. (2004). Peroxisome proliferator-activated receptor-alpha deficiency does not alter insulin sensitivity in mice maintained on regular or high-fat diet: hyperinsulinemic-euglycemic clamp studies. Endocrinology 145, 1662-1667.
42. Han M K, Song E K, Guo Y, Ou X, Mantel C, Broxmeyer H E. SIRT1 regulates apoptosis and Nanog expression in mouse embryonic stem cells by controlling p53 subcellular localization. Cell Stem Cell. 2008 Mar. 6; 2(3):241-51.
43. Harant H, Korschineck I, Krupitza G, Fazeny B, Dittrich C, Grunt T W. Retinoic acid receptors in retinoid responsive ovarian cancer cell lines detected by polymerase chain reaction following reverse transcription. Br J Cancer. 1993 September; 68(3):530-6.
44. Harbour, J. W., and Dean, D. C. (2000). The Rb/E2F pathway: expanding roles and emerging paradigms. Genes & development 14, 2393-2409.
45. Hassan M M, Hwang L Y, Hatten C J, et al. Risk factors for hepatocellular carcinoma: synergism of alcohol with viral hepatitis and diabetes mellitus. Hepatology 2002; 36:1206-13.
46. Hawley, S. A., Davison, M., Woods, A., Davies, S. P., Beri, R. K., Carling, D., and Hardie, D. G. (1996). Characterization of the AMP-activated protein kinase kinase from rat liver and identification of threonine 172 as the major site at which it phosphorylates AMP-activated protein kinase. J Biol Chem 271, 27879-27887.
47. He, N., Park, K., Zhang, Y., Huang, J., Lu, S., and Wang, L. (2008). Epigenetic inhibition of nuclear receptor small heterodimer partner is associated with and regulates hepatocellular carcinoma growth. Gastroenterology 134, 793-802.
48. Hoei-Hansen C E. Application of stem cell markers in search for neoplastic germ cells in dysgenetic gonads, extragonadal tumours, and in semen of infertile men. Cancer Treat Rev. 2008 June; 34(4):348-67.
49. Hritz, I., Mandrekar, P., Velayudham, A., Catalano, D., Dolganiuc, A., Kodys, K., Kurt-Jones, E., and Szabo, G. (2008). The critical role of toll-like receptor (TLR) 4 in alcoholic liver disease is independent of the common TLR adapter MyD88. Hepatology 48, 1224-1231.
50. Huang M E, Ye Y C, Chen S R, Chai J R, Lu J X, Zhoa L, Gu L J, Wang Z Y. Use of all-trans retinoic acid in the treatment of acute promyelocytic leukemia. Blood. 1988 August; 72(2):567-72.
51. Huynh, H., Ngo, V. C., Koong, H. N., Poon, D., Choo, S. P., Thng, C. H., Chow, P., Ong, H. S., Chung, A., and Soo, K. C. (2009). Sorafenib and rapamycin induce growth suppression in mouse models of hepatocellular carcinoma. J Cell Mol Med 13, 2673-2683.
52. Ito, K., Carracedo, A., Weiss, D., Arai, F., Ala, U., Avigan, D. E., Schafer, Z. T., Evans, R. M., Suda, T., Lee, C. H., et al. (2012). A PML-PPAR-delta pathway for fatty acid oxidation regulates hematopoietic stem cell maintenance. Nat Med 18, 1350-1358.
53. Ito, K., Hirao, A., Arai, F., Matsuoka, S., Takubo, K., Hamaguchi, I., Nomiyama, K., Hosokawa, K., Sakurada, K., Nakagata, N., et al. (2004). Regulation of oxidative stress by ATM is required for self-renewal of haematopoietic stem cells. Nature 431, 997-1002.
54. Ito, K., Hirao, A., Arai, F., Takubo, K., Matsuoka, S., Miyamoto, K., Ohmura, M., Naka, K., Hosokawa, K., Ikeda, Y., et al. (2006). Reactive oxygen species act through p38 MAPK to limit the lifespan of hematopoietic stem cells. Nat Med 12, 446-451.
55. Jeter C R, Badeaux M, Choy G, Chandra D, Patrawala L, Liu C, Calhoun-Davis T, Zaehres H, Daley G Q, Tang D G. Functional evidence that the self-renewal gene NANOG regulates human tumor development. Stem Cells. 2009 May; 27(5):993-1005.
56. Jeter C R, Liu B, Liu X, Chen X, Liu C, Calhoun-Davis T, Repass J, Zaehres H, Shen J J, Tang D G. NANOG promotes cancer stem cell characteristics and prostate cancer resistance to androgen deprivation. Oncogene. 2011 Sep. 8; 30(36):3833-45.
57. Jiang R, Deng L, Zhao L, Li X, Zhang F, Xia Y, Gao Y, Wang X, Sun B. miR-22 promotes HBV-related hepatocellular carcinoma development in males. Clin Cancer Res. 2011 Sep. 1; 17(17):5593-603.
58. Jiang, J., Chan, Y. S., Loh, Y. H., Cai, J., Tong, G. Q., Lim, C. A., Robson, P., Zhong, S., and Ng, H. H. (2008). A core Klf circuitry regulates self-renewal of embryonic stem cells. Nat Cell Biol 10, 353-360.
59. Jin H, Liang L, Liu L, Deng W, Liu J. HDAC inhibitor DWP0016 activates p53 transcription and acetylation to inhibit cell growth in U251 glioblastoma cells. J Cell Biochem. 2013 July; 114(7):1498-509.
60. Johnson, D. G., and Schneider-Broussard, R. (1998). Role of E2F in cell cycle control and cancer. Front Biosci 3, d447-448.
61. Kang Y, Massague J. Epithelial-mesenchymal transitions: twist in development and metastasis. Cell 2004; 118:277-9.
62. Katayama K, Nakamura A, Sugimoto Y, Tsuruo T, Fujita N. FOXO transcription factor-dependent p15(INK4b) and p19(INK4d) expression. Oncogene. 2008 Mar. 13; 27(12):1677-86.
63. Kato N, Lan K H, Ono-Nita S K, et al. Hepatitis C virus nonstructural region 5A protein is a potent transcriptional activator. J Virol 1997; 71:8856-9.
64. Kato Y, Salumbides B C, Wang X F, Qian D Z, Williams S, Wei Y, Sanni T B, Atadja P, Pili R. Antitumor effect of the histone deacetylase inhibitor LAQ824 in combination with 13-cis-retinoic acid in human malignant melanoma. Mol Cancer Ther. 2007 January; 6(1):70-81.
65. Kennedy, D. W., and Abkowitz, J. L. (1997). Kinetics of central nervous system microglial and macrophage engraftment: analysis using a transgenic bone marrow transplantation model. Blood 90, 986-993.
66. Kersten, S., Seydoux, J., Peters, J. M., Gonzalez, F. J., Desvergne, B., and Wahli, W. (1999). Peroxisome proliferator-activated receptor alpha mediates the adaptive response to fasting. The Journal of clinical investigation 103, 1489-1498.
67. Khacho, M., Tarabay, M., Patten, D., Khacho, P., MacLaurin, J. G., Guadagno, J., Bergeron, R., Cregan, S. P., Harper, M. E., Park, D. S., et al. (2014). Acidosis overrides oxygen deprivation to maintain mitochondrial function and cell survival. Nat Commun 5, 3550.
68. Khan O, La Thangue N B. HDAC inhibitors in cancer biology: emerging mechanisms and clinical applications. Immunol Cell Biol. 2012 January; 90(1):85-94.
69. Kim, K., Punj, V., Choi, J., Heo, K., Kim, J. M., Laird, P. W., and An, W. (2013). Gene dysregulation by histone variant H2A.Z in bladder cancer. Epigenetics Chromatin 6, 34.
70. Kisseleva, T., Uchinami, H., Feirt, N., Quintana-Bustamante, O., Segovia, J. C., Schwabe, R. F., and Brenner, D. A. (2006). Bone marrow-derived fibrocytes participate in pathogenesis of liver fibrosis. J Hepatol 45, 429-438.
71. Klingenberg, M., and Huang, S. G. (1999). Structure and function of the uncoupling protein from brown adipose tissue. Biochim Biophys Acta 1415, 271-296.
72. Komatsu N, Kawamata N, Takeuchi S, Yin D, Chien W, Miller C W, Koeffler H P. SAHA, a HDAC inhibitor, has profound anti-growth activity against non-small cell lung cancer cells. Oncol Rep. 2006 January; 15(1):187-191.
73. Krauss, S., Zhang, C. Y., and Lowell, B. B. (2005). The mitochondrial uncoupling-protein homologues. Nat Rev Mol Cell Biol 6, 248-261.
74. Kumagai T, Wakimoto N, Yin D, Gery S, Kawamata N, Takai N, Komatsu N, Chumakov A, Imai Y, Koeffler H P. Histone deacetylase inhibitor, suberoylanilide hydroxamic acid (Vorinostat, SAHA) profoundly inhibits the growth of human pancreatic cancer cells. Int J Cancer. 2007 Aug. 1; 121(3):656-665.
75. Kuroda, T., Tada, M., Kubota, H., Kimura, H., Hatano, S. Y., Suemori, H., Nakatsuji, N., and Tada, T. (2005). Octamer and Sox elements are required for transcriptional cis regulation of Nanog gene expression. Mol Cell Biol 25, 2475-2485.
76. Kurtz, D. M., Rinaldo, P., Rhead, W. J., Tian, L., Millington, D. S., Vockley, J., Hamm, D. A., Brix, A. E., Lindsey, J. R., Pinkert, C. A., et al. (1998). Targeted disruption of mouse long-chain acyl-CoA dehydrogenase gene reveals crucial roles for fatty acid oxidation. Proceedings of the National Academy of Sciences of the United States of America 95, 15592-15597.
77. Lai M S, Hsieh M S, Chiu Y H, et al. Type 2 diabetes and hepatocellular carcinoma: A cohort study in high prevalence area of hepatitis virus infection. Hepatology 2006; 43:1295-302.
78. Lamonte, G., Tang, X., Chen, J. L., Wu, J., Ding, C. K., Keenan, M. M., Sangokoya, C., Kung, H. N., Ilkayeva, O., Boros, L. G., et al. (2013). Acidosis induces reprogramming of cellular metabolism to mitigate oxidative stress. Cancer Metab 1, 23.
79. Lee, W. N. (1996). Stable isotopes and mass isotopomer study of fatty acid and cholesterol synthesis. A review of the MIDA approach. Adv Exp Med Biol 399, 95-114.
80. Lee, W. N., Edmond, J., Bassilian, S., and Morrow, J. W. (1996). Mass isotopomer study of glutamine oxidation and synthesis in primary culture of astrocytes. Dev Neurosci 18, 469-477.
81. Li H, Wu X. Histone deacetylase inhibitor, Trichostatin A, activates p21WAF1/CIP1 expression through downregulation of c-myc and release of the repression of c-myc from the promoter in human cervical cancer cells. Biochem Biophys Res Commun. 2004 Nov. 12; 324(2):860-7.
82. Li L, Wang L, Li L, Wang Z, Ho Y, McDonald T, Holyoake T L, Chen W, Bhatia R. Activation of p53 by SIRT1 inhibition enhances elimination of CML leukemia stem cells in combination with imatinib. Cancer Cell. 2012 Feb. 14; 21(2):266-81.
83. Li, H., and Durbin, R. (2009). Fast and accurate short read alignment with Burrows-Wheeler transform. Bioinformatics 25, 1754-1760.
84. Liang, J., Wan, M., Zhang, Y., Gu, P., Xin, H., Jung, S. Y., Qin, J., Wong, J., Cooney, A. J., Liu, D., et al. (2008). Nanog and Oct4 associate with unique transcriptional repression complexes in embryonic stem cells. Nat Cell Biol 10, 731-739.
85. Lim, C. A., Yao, F., Wong, J. J., George, J., Xu, H., Chiu, K. P., Sung, W. K., Lipovich, L., Vega, V. B., Chen, J., et al. (2007). Genome-wide mapping of RELA(p65) binding identifies E2F1 as a transcriptional activator recruited by NF-kappaB upon TLR4 activation. Mol Cell 27, 622-635.
86. Lin, T., Chao, C., Saito, S., Mazur, S. J., Murphy, M. E., Appella, E., and Xu, Y. (2005). p53 induces differentiation of mouse embryonic stem cells by suppressing Nanog expression. Nat Cell Biol 7, 165-171.
87. Llovet, J. M., and Bruix, J. (2008). Molecular targeted therapies in hepatocellular carcinoma. Hepatology 48, 1312-1327.
88. Lo J F. Positive correlations of Oct-4 and Nanog in oral cancer stem-like cells and high-grade oral squamous cell carcinoma. Clin Cancer Res. 2008 Jul. 1; 14(13):4085-95.
89. Lundberg, A. S., and Weinberg, R. A. (1998). Functional inactivation of the retinoblastoma protein requires sequential modification by at least two distinct cyclin-cdk complexes. Mol Cell Biol 18, 753-761.
90. Ma S, Lee T K, Zheng B J, Chan K W, Guan X Y. CD133+HCC cancer stem cells confer chemoresistance by preferential expression of the Akt/PKB survival pathway. Oncogene. 2008 Mar. 13; 27(12):1749-58.
91. Ma S, Tang K H, Chan Y P, Lee T K, Kwan P S, Castilho A, Ng I, Man K, Wong N, To K F, Zheng B J, Lai P B, Lo C M, Chan K W, Guan X Y. miR-130b Promotes CD133(+) liver tumor-initiating cell growth and self-renewal via tumor protein 53-induced nuclear protein 1. Cell Stem Cell. 2010 Dec. 3; 7(6):694-707.
92. Machida K, Cheng K T, Sung V M, et al. Hepatitis C virus induces toll-like receptor 4 expression, leading to enhanced production of beta interferon and interleukin-6. J Virol 2006; 80:866-74.
93. Machida, K., Tsukamoto, H., Liu, J. C., Han, Y. P., Govindarajan, S., Lai, M. M., Akira, S., and Ou, J. H. (2010). c-Jun mediates hepatitis C virus hepatocarcinogenesis through signal transducer and activator of transcription 3 and nitric oxide-dependent impairment of oxidative DNA repair. Hepatology 52, 480-492.
94. Machida, K., Tsukamoto, H., Mkrtchyan, H., Duan, L., Dynnyk, A., Liu, H. M., Asahina, K., Govindarajan, S., Ray, R., Ou, J. H., et al. (2009). Toll-like receptor 4 mediates synergism between alcohol and HCV in hepatic oncogenesis involving stem cell marker Nanog. Proc Natl Acad Sci USA 106, 1548-1553.
95. Majid S, Dar A A, Saini S, Shahryari V, Arora S, Zaman M S, Chang I, Yamamura S, Tanaka Y, Chiyomaru T, Deng G, Dahiya R. miRNA-34b inhibits prostate cancer through demethylation, active chromatin modifications, and AKT pathways. Clin Cancer Res. 2013 Jan. 1; 19(1):73-84.
96. Majumder M, Ghosh A K, Steele R, et al. Hepatitis C virus NS5A protein impairs TNF-mediated hepatic apoptosis, but not by an anti-FAS antibody, in transgenic mice. Virology 2002; 294:94-105.
97. Majumder M, Steele R, Ghosh A K, et al. Expression of hepatitis C virus non-structural 5A protein in the liver of transgenic mice. FEBS Lett 2003; 555:528-32.
98. Mani S A, Guo W, Liao M J, et al. The epithelial-mesenchymal transition generates cells with properties of stem cells. Cell 2008; 133:704-15.
99. Mathurin, P., Deng, Q. G., Keshavarzian, A., Choudhary, S., Holmes, E. W., and Tsukamoto, H. (2000). Exacerbation of alcoholic liver injury by enteral endotoxin in rats. Hepatology 32, 1008-1017.
100. Meletis K, Wirta V, Hede S M, Nistér M, Lundeberg J, Frisén J. p53 suppresses the self-renewal of adult neural stem cells. Development. 2006 January; 133(2):363-9.
101. Meyskens F L Jr, Jacobson J, Nguyen B, Weiss G R, Gandara D R, MacDonald J S. Phase II trial of oral beta-all trans-retinoic acid in hepatocellular carcinoma (SWOG 9157). Invest New Drugs. 1998; 16(2):171-3.
102. Mittal, D., Saccheri, F., Venereau, E., Pusterla, T., Bianchi, M. E., and Rescigno, M. (2010). TLR4-mediated skin carcinogenesis is dependent on immune and radioresistant cells. Embo J 29, 2242-2252.
103. Morel A P, Lievre M, Thomas C, et al. Generation of breast cancer stem cells through epithelial-mesenchymal transition. PLoS One 2008; 3:e2888.
104. Motzer R J, Murphy B A, Bacik J, Schwartz L H, Nanus D M, Mariani T, Loehrer P, Wilding G, Fairclough D L, Cella D, Mazumdar M. Phase III trial of interferon alfa-2a with or without 13-cis-retinoic acid for patients with advanced renal cell carcinoma. J Clin Oncol. 2000 August; 18(16):2972-80.
105. Nakada, D., Saunders, T. L., and Morrison, S. J. (2010). Lkbl regulates cell cycle and energy metabolism in haematopoietic stem cells. Nature 468, 653-658.
106. Nawshad A, Lagamba D, Polad A, et al. Transforming growth factor-beta signaling during epithelial-mesenchymal transformation: implications for embryogenesis and tumor metastasis. Cells Tissues Organs 2005; 179:11-23.
107. Noh K H, Kim B W, Song K H, Cho H, Lee Y H, Kim J H, Chung J Y, Kim J H, Hewitt S M, Seong S Y, Mao C P, Wu T C, Kim T W. Nanog signaling in cancer promotes stem-like phenotype and immune evasion. J Clin Invest. 2012 November; 122(11):4077-93.
108. Okuda, M., Li, K., Beard, M. R., Showalter, L. A., Scholle, F., Lemon, S. M., and Weinman, S. A. (2002). Mitochondrial injury, oxidative stress, and antioxidant gene expression are induced by hepatitis C virus core protein. Gastroenterology 122, 366-375.
109. Onay-Besikci, A. (2006). Regulation of cardiac energy metabolism in newborn. Mol Cell Biochem 287, 1-11.
110. Pan G, Thomson J A. Nanog and transcriptional networks in embryonic stem cell pluripotency. Cell Res. 2007 January; 17(1):42-9.
111. Parent, R., Marion, M. J., Furio, L., Trepo, C., and Petit, M. A. (2004). Origin and characterization of a human bipotent liver progenitor cell line. Gastroenterology 126, 1147-1156.
112. Paumen, M. B., Ishida, Y., Han, H., Muramatsu, M., Eguchi, Y., Tsujimoto, Y., and Honjo, T. (1997). Direct interaction of the mitochondrial membrane protein carnitine palmitoyltransferase I with Bcl-2. Biochem Biophys Res Commun 231, 523-525.
113. Pestereva, E., Kanakasabai, S., and Bright, J. J. (2012). PPARgamma agonists regulate the expression of stemness and differentiation genes in brain tumour stem cells. British journal of cancer 106, 1702-1712.
114. Peters M G, Terrault N A. Alcohol use and hepatitis C. Hepatology 2002; 36:5220-5.
115. Petruccelli L A, Dupéré-Richer D, Pettersson F, Retrouvey H, Skoulikas S, Miller W H Jr. Vorinostat induces reactive oxygen species and DNA damage in acute myeloid leukemia cells. PLoS One. 2011; 6(6):e20987.
116. Quintens, R., Singh, S., Lemaire, K., De Bock, K., Granvik, M., Schraenen, A., Vroegrijk, I. O., Costa, V., Van Noten, P., Lambrechts, D., et al. (2013). Mice deficient in the respiratory chain gene Cox6a2 are protected against high-fat diet-induced obesity and insulin resistance. PLoS One 8, e56719.
117. Reynolds C P, Matthay K K, Villablanca J G, Maurer B J. Retinoid therapy of high-risk neuroblastoma. Cancer Lett. 2003 Jul. 18; 197(1-2):185-92.
118. Ribeiro P S, Cortez-Pinto H, Sola S, et al. Hepatocyte apoptosis, expression of death receptors, and activation of NF-kappaB in the liver of nonalcoholic and alcoholic steatohepatitis patients. Am J Gastroenterol 2004; 99:1708-17.
119. Rochette-Egly C. Retinoic acid signaling and mouse embryonic stem cell differentiation: Cross talk between genomic and non-genomic effects of R A. Biochim Biophys Acta. 2015 January; 1851(1):66-75.
120. Rodda, D. J., Chew, J. L., Lim, L. H., Loh, Y. H., Wang, B., Ng, H. H., and Robson, P. (2005). Transcriptional regulation of nanog by OCT4 and SOX2. J Biol Chem 280, 24731-24737.
121. Rosato R R, Almenara J A, Dai Y, Grant S. Simultaneous activation of the intrinsic and extrinsic pathways by histone deacetylase (HDAC) inhibitors and tumor necrosis factor-related apoptosis-inducing ligand (TRAIL) synergistically induces mitochondrial damage and apoptosis in human leukemia cells. Mol Cancer Ther. 2003 December; 2(12):1273-84.
122. Roses A D. Pharmacogenetics in drug discovery and development: a translational perspective. Nat Rev Drug Discov. 2008 October; 7(10):807-17.
123. Rountree, C. B., Senadheera, S., Mato, J. M., Crooks, G. M., and Lu, S. C. (2008). Expansion of liver cancer stem cells during aging in methionine adenosyltransferase 1A-deficient mice. Hepatology 47, 1288-1297.
124. Rubin Grandis J, Zeng Q, Tweardy D J. Retinoic acid normalizes the increased gene transcription rate of TGF-alpha and EGFR in head and neck cancer cell lines. Nat Med. 1996 February; 2(2):237-40.
125. Saberi, M., Woods, N. B., de Luca, C., Schenk, S., Lu, J. C., Bandyopadhyay, G., Verma, I. M., and Olefsky, J. M. (2009). Hematopoietic cell-specific deletion of toll-like receptor 4 ameliorates hepatic and adipose tissue insulin resistance in high-fat-fed mice. Cell Metab 10, 419-429.
126. Salomoni P, Pandolfi P P. Transcriptional regulation of cellular transformation. Nat Med. 2000 July; 6(7):742-744.
127. Samudio, I., Harmancey, R., Fiegl, M., Kantarjian, H., Konopleva, M., Korchin, B., Kaluarachchi, K., Bornmann, W., Duvvuri, S., Taegtmeyer, H., et al. (2010). Pharmacologic inhibition of fatty acid oxidation sensitizes human leukemia cells to apoptosis induction. The Journal of clinical investigation 120, 142-156.
128. Sandor V, Senderowicz A, Mertins S, Sackett D, Sausville E, Blagosklonny M V, Bates S E. P21-dependent g(1)arrest with downregulation of cyclin D1 and upregulation of cyclin E by the histone deacetylase inhibitor FR901228. Br J Cancer. 2000 September; 83(6):817-25.
129. Sato, A., Okada, M., Shibuya, K., Watanabe, E., Seino, S., Narita, Y., Shibui, S., Kayama, T., and Kitanaka, C. (2014). Pivotal role for ROS activation of p38 MAPK in the control of differentiation and tumor-initiating capacity of glioma-initiating cells. Stem Cell Res 12, 119-131.
130. Seki, E., De Minicis, S., Osterreicher, C. H., Kluwe, J., Osawa, Y., Brenner, D. A., and Schwabe, R. F. (2007). TLR4 enhances TGF-beta signaling and hepatic fibrosis. Nat Med 13, 1324-1332.
131. Shen J J, Tang D G. NANOG promotes cancer stem cell characteristics and prostate cancer resistance to androgen deprivation. Oncogene. 2011 Sep. 8; 30(36):3833-45.
132. Shintani Y, Fujie H, Miyoshi H, et al. Hepatitis C virus infection and diabetes: direct involvement of the virus in the development of insulin resistance. Gastroenterology 2004; 126:840-8.
133. Siddique, H. R., and Saleem, M. (2012). Role of BMI1, a stem cell factor, in cancer recurrence and chemoresistance: preclinical and clinical evidences. Stem Cells 30, 372-378.
134. Simandi Z, Balint B L, Poliska S, Ruhl R, Nagy L. Activation of retinoic acid receptor signaling coordinates lineage commitment of spontaneously differentiating mouse embryonic stem cells in embryoid bodies. FEBS Lett. 2010 Jul. 16; 584(14):3123-30.
135. Singh S, Singh P P, Roberts L R, et al. Chemopreventive strategies in hepatocellular carcinoma. Nat Rev Gastroenterol Hepatol 2014; 11:45-54.
136. Song M S, Salmena L, Pandolfi P P. The functions and regulation of the PTEN tumour suppressor. Nat Rev Mol Cell Biol. 2012 Apr. 4; 13(5):283-296.
137. Song S J, Ito K, Ala U, Kats L, Webster K, Sun S M, Jongen-Lavrencic M, Manova-Todorova K, Teruya-Feldstein J, Avigan D E, Delwel R, Pandolfi P P. The oncogenic microRNA miR-22 targets the TET2 tumor suppressor to promote hematopoietic stem cell self-renewal and transformation. Cell Stem Cell. 2013 Jul. 3; 13(1):87-101.
138. Song S J, Poliseno L, Song M S, Ala U, Webster K, Ng C, Beringer G, Brikbak N J, Yuan X, Cantley L C, Richardson A L, Pandolfi P P. MicroRNA-antagonism regulates breast cancer stemness and metastasis via TET-family-dependent chromatin remodeling. Cell. 2013 Jul. 18; 154(2):311-324.
139. Spender, L. C., and Inman, G. J. (2009). TGF-beta induces growth arrest in Burkitt lymphoma cells via transcriptional repression of E2F-1. J Biol Chem 284, 1435-1442.
140. Storm, M. P., Bone, H. K., Beck, C. G., Bourillot, P. Y., Schreiber, V., Damiano, T., Nelson, A., Savatier, P., and Welham, M. J. (2007). Regulation of Nanog expression by phosphoinositide 3-kinase-dependent signaling in murine embryonic stem cells. J Biol Chem 282, 6265-6273.
141. Strait K A, Warnick C T, Ford C D, Dabbas B, Hammond E H, Ilstrup S J. Histone deacetylase inhibitors induce G2-checkpoint arrest and apoptosis in cisplatinum-resistant ovarian cancer cells associated with overexpression of the Bcl-2-related protein Bad. Mol Cancer Ther. 2005 April; 4(4):603-11.
142. Suetsugu A, Nagaki M, Aoki H, Motohashi T, Kunisada T, Moriwaki H. Characterization of CD133+ hepatocellular carcinoma cells as cancer stem/progenitor cells. Biochem Biophys Res Commun. 2006 Dec. 29; 351(4):820-4.
143. Sun A X, Liu C J, Sun Z Q, Wei Z. NANOG: a promising target for digestive malignant tumors. World J Gastroenterol. 2014 Sep. 28; 20(36):13071-8.
144. Sun T, Zhao N, Zhao X L, et al. Expression and functional significance of Twist1 in hepatocellular carcinoma: its role in vasculogenic mimicry. Hepatology; 51:545-56.
145. Suzuki, A., Raya, A., Kawakami, Y., Morita, M., Matsui, T., Nakashima, K., Gage, F. H., Rodriguez-Esteban, C., and Izpisua Belmonte, J. C. (2006). Nanog binds to Smad1 and blocks bone morphogenetic protein-induced differentiation of embryonic stem cells. Proceedings of the National Academy of Sciences of the United States of America 103, 10294-10299.
146. Swaminathan S K, Roger E, Toti U, Niu L, Ohlfest J R, Panyam J. CD133-targeted paclitaxel delivery inhibits local tumor recurrence in a mouse model of breast cancer. J Control Release. 2013 Nov. 10; 171(3):280-7.
147. Takubo, K., Nagamatsu, G., Kobayashi, C. I., Nakamura-Ishizu, A., Kobayashi, H., Ikeda, E., Goda, N., Rahimi, Y., Johnson, R. S., Soga, T., et al. (2013). Regulation of glycolysis by Pdk functions as a metabolic checkpoint for cell cycle quiescence in hematopoietic stem cells. Cell Stem Cell 12, 49-61.
148. Tessitore A, Cicciarelli G, Del Vecchio F, Gaggiano A, Verzella D, Fischietti M, Vecchiotti D, Capece D, Zazzeroni F, Alesse E. MicroRNAs in the DNA Damage/Repair Network and Cancer. Int J Genomics. 2014; 2014:820248.
149. Thiery J P. Epithelial-mesenchymal transitions in development and pathologies. Curr Opin Cell Biol 2003; 15:740-6.
150. Tikhanovich, I., Kuravi, S., Campbell, R. V., Kharbanda, K. K., Artigues, A., Villar, M. T., and Weinman, S. A. (2014). Regulation of FOXO3 by phosphorylation and methylation in hepatitis C virus infection and alcohol exposure. Hepatology 59, 58-70.
151. Torres, J., and Watt, F. M. (2008). Nanog maintains pluripotency of mouse embryonic stem cells by inhibiting NFkappaB and cooperating with Stat3. Nat Cell Biol 10, 194-201.
152. Tsung, A., Hoffman, R. A., Izuishi, K., Critchlow, N. D., Nakao, A., Chan, M. H., Lotze, M. T., Geller, D. A., and Billiar, T. R. (2005). Hepatic ischemia/reperfusion injury involves functional TLR4 signaling in nonparenchymal cells. Journal of immunology 175, 7661-7668.
153. Valent, P., Bonnet, D., De Maria, R., Lapidot, T., Copland, M., Melo, J. V., Chomienne, C., Ishikawa, F., Schuringa, J. J., Stassi, G., et al. (2012). Cancer stem cell definitions and terminology: the devil is in the details. Nat Rev Cancer 12, 767-775.
154. van den Berg D L, Snoek T, Mullin N P, Yates A, Berstarosti K, Demmers J, Chambers I, Poot R A. An Oct4-centered protein interaction network in embryonic stem cells. Cell Stem Cell. 2010 Apr. 2; 6(4):369-81.
155. Van Heek, M., Compton, D. S., France, C. F., Tedesco, R. P., Fawzi, A. B., Graziano, M. P., Sybertz, E. J., Strader, C. D., and Davis, H. R., Jr. (1997). Diet-induced obese mice develop peripheral, but not central, resistance to leptin. The Journal of clinical investigation 99, 385-390.
156. Van Rooijen, N., and Sanders, A. (1994). Liposome mediated depletion of macrophages: mechanism of action, preparation of liposomes and applications. Journal of immunological methods 174, 83-93.
157. Vickers, A. E. (2009). Characterization of hepatic mitochondrial injury induced by fatty acid oxidation inhibitors. Toxicol Pathol 37, 78-88.
158. Villanueva A, Hernandez-Gea V, Llovet J M. Medical therapies for hepatocellular carcinoma: a critical view of the evidence. Nat Rev Gastroenterol Hepatol 2013; 10:34-42.
159. Villanueva, A., Chiang, D. Y., Newell, P., Peix, J., Thung, S., Alsinet, C., Tovar, V., Roayaie, S., Minguez, B., Sole, M., et al. (2008). Pivotal role of mTOR signaling in hepatocellular carcinoma. Gastroenterology 135, 1972-1983, 1983 e1971-1911.
160. Wan, B., and Moreadith, R. W. (1995). Structural characterization and regulatory element analysis of the heart isoform of cytochrome c oxidase VIa. J Biol Chem 270, 26433-26440.
161. Wang X Q, Ng R K, Ming X, Zhang W, Chen L, Chu A C, Pang R, Lo C M, Tsao S W, Liu X, Poon R T, Fan S T. Epigenetic regulation of pluripotent genes mediates stem cell features in human hepatocellular carcinoma and cancer cell lines. PLoS One. 2013 Sep. 4; 8(9):e72435.
162. Wang, E., Bhattacharyya, S., Szabolcs, A., Rodriguez-Aguayo, C., Jennings, N. B., Lopez-Berestein, G., Mukherjee, P., Sood, A. K., and Bhattacharya, R. (2011). Enhancing chemotherapy response with Bmi-1 silencing in ovarian cancer. PLoS One 6, e17918.
163. Wang, Z. X., Kueh, J. L., Teh, C. H., Rossbach, M., Lim, L., Li, P., Wong, K. Y., Lufkin, T., Robson, P., and Stanton, L. W. (2007). Zfp206 is a transcription factor that controls pluripotency of embryonic stem cells. Stem Cells 25, 2173-2182.
164. Weinberg, F., Hamanaka, R., Wheaton, W. W., Weinberg, S., Joseph, J., Lopez, M., Kalyanaraman, B., Mutlu, G. M., Budinger, G. R., and Chandel, N. S. (2010). Mitochondrial metabolism and ROS generation are essential for Kras-mediated tumorigenicity. Proceedings of the National Academy of Sciences of the United States of America 107, 8788-8793.
165. Weiss F U, Marques L I, Woltering J M, Vlecken D H, Aghdassi A, Partecke L I, Heidecke C D, Lerch M M, Bagowski C P. Retinoic acid receptor antagonists inhibit miR-10a expression and block metastatic behavior of pancreatic cancer. Gastroenterology. 2009 December; 137(6):2136-45.e1-7.
166. Wu da, Y., and Yao, Z. (2005). Isolation and characterization of the murine Nanog gene promoter. Cell Res 15, 317-324.
167. Wu, Q., Chen, X., Zhang, J., Loh, Y. H., Low, T. Y., Zhang, W., Zhang, W., Sze, S. K., Lim, B., and Ng, H. H. (2006). Sal14 interacts with Nanog and co-occupies Nanog genomic sites in embryonic stem cells. J Biol Chem 281, 24090-24094.
168. Wu, Z., Puigserver, P., Andersson, U., Zhang, C., Adelmant, G., Mootha, V., Troy, A., Cinti, S., Lowell, B., Scarpulla, R. C., et al. (1999). Mechanisms controlling mitochondrial biogenesis and respiration through the thermogenic coactivator PGC-1. Cell 98, 115-124.
169. Xu F, Dai C, Zhang R, Zhao Y, Peng S, Jia C. Nanog: a potential biomarker for liver metastasis of colorectal cancer. Dig Dis Sci. 2012 September; 57(9):2340-6.
170. Yamaguchi, S., Kurimoto, K., Yabuta, Y., Sasaki, H., Nakatsuji, N., Saitou, M., and Tada, T. (2009). Conditional knockdown of Nanog induces apoptotic cell death in mouse migrating primordial germ cells. Development 136, 4011-4020.
171. Yang H, Bushue N, Bu P, Wan Y J. Induction and intracellular localization of Nur77 dictate fenretinide-induced apoptosis of human liver cancer cells. Biochem Pharmacol. 2010 Apr. 1; 79(7):948-54.
172. Yang J, Mani S A, Donaher J L, et al. Twist, a master regulator of morphogenesis, plays an essential role in tumor metastasis. Cell 2004; 117:927-39.
173. Ye F, Zhou C, Cheng Q, Shen J, Chen H. Stem-cell-abundant proteins Nanog, Nucleostemin and Musashil are highly expressed in malignant cervical epithelial cells. BMC Cancer. 2008 Apr. 18; 8:108.
174. Yuan, J. M., Govindarajan, S., Arakawa, K., and Yu, M. C. (2004). Synergism of alcohol, diabetes, and viral hepatitis on the risk of hepatocellular carcinoma in blacks and whites in the U. S. Cancer 101, 1009-1017.
175. Zhang J, Luo N, Luo Y, Peng Z, Zhang T, Li S. microRNA-150 inhibits human CD133-positive liver cancer stem cells through negative regulation of the transcription factor c-Myb. Int J Oncol. 2012 March; 40(3):747-56.
176. Zhang S, Balch C, Chan M W, Lai H C, Matei D, Schilder J M, Yan P S, Huang T H, Nephew K P. Identification and characterization of ovarian cancer-initiating cells from primary human tumors. Cancer Res. 2008 Jun. 1; 68(11):4311-4320.
177. Zhang, D., Liu, Z. X., Choi, C. S., Tian, L., Kibbey, R., Dong, J., Cline, G. W., Wood, P. A., and Shulman, G. I. (2007). Mitochondrial dysfunction due to long-chain Acyl-CoA dehydrogenase deficiency causes hepatic steatosis and hepatic insulin resistance. Proceedings of the National Academy of Sciences of the United States of America 104, 17075-17080.
178. Zhang, J., Khvorostov, I., Hong, J. S., Oktay, Y., Vergnes, L., Nuebel, E., Wahjudi, P. N., Setoguchi, K., Wang, G., Do, A., et al. (2011). UCP2 regulates energy metabolism and differentiation potential of human pluripotent stem cells. EMBO J 30, 4860-4873.
179. Zhang, L. F., Ding, J. H., Yang, B. Z., He, G. C., and Roe, C. (2003). Characterization of the bidirectional promoter region between the human genes encoding VLCAD and PSD-95. Genomics 82, 660-668.
180. Zhao X Y, Ly L H, Peehl D M, Feldman D. Induction of androgen receptor by 1alpha,25-dihydroxyvitamin D3 and 9-cis retinoic acid in LNCaP human prostate cancer cells. Endocrinology. 1999 March; 140(3): 1205-12.
181. Zhou B B, Zhang H, Damelin M, Geles K G, Grindley J C, Dirks P B. Tumour-initiating cells: challenges and opportunities for anticancer drug discovery. at Rev Drug Discov. 2009 October; 8(10):806-23.

We claim:

1. A method of identifying subjects with metastatic hepatocellular carcinoma (HCC) for tumor-initiating stem-like cells (TICs) or circulating tumor cells (CTCs) targeted therapy comprising:
obtaining whole blood from a subject;
retrieving CTCs and/or TICs from the whole blood;
performing quantitative reverse transcriptase-PCR (qRT PCR) on retrieved CTCs and/or TICs; and
identifying genes selected from the group consisting of NANOG, TWIST1, LIN28, MSI2, ACADVL, BIRC5, miR-22, LepR, YAP1 and IGF2BP3 that are upregulated and/or genes selected from the group consisting of COX6A2, COX15, TET1, TET2 and PTEN that are downregulated.

2. The method of claim 1, wherein the TICs are CD133+, CD49f+, and CD45−.

3. The method of claim 1, wherein the CTCs are CD45− and cytokeratins negative.

4. The method of claim 1, wherein upon the identification of one or more of the genes that are upregulated and/or one or more of the genes that are downregulated, a targeted therapy is initiated.

5. The method of claim 4, wherein the targeted therapy comprises inhibiting a NANOG pathway.

6. The method of claim 4, wherein the targeted therapy comprises inhibiting a NANOG and Stat3 pathway.

7. The method of claim 4, wherein a chemotherapeutic drug is concurrently administered with the targeted therapy.

8. The method of claim 7, wherein the chemotherapeutic drug is sorafenib.

9. The method of claim 4, wherein the targeted therapy comprises enhancing regeneration of mitochondrial oxidative phosphorylation (OXPHOS) genes or reactive oxygen species (ROS).

10. The method of claim 9, wherein the targeted therapy further comprises concurrently administering a chemotherapeutic drug.

11. The method of claim 10, wherein the chemotherapeutic drug is sorafenib.

12. The method of claim 4, wherein the targeted therapy comprises inhibiting mitochondrial fatty acid oxidation (FAO).

13. The method of claim 12, wherein the targeted therapy further comprises concurrently administering a chemotherapeutic drug.

14. The method of claim 13, wherein the chemotherapeutic drug is sorafenib.

15. A method for epigenetically modifying and eradicating tumor-initiating stem-like cells (TICs) in a subject in need thereof, comprising:
administering, to the subject, an effective amount of suberoylanilide hydroxamic acid (SAHA).

16. The method of claim 13, further comprising:
administering, to the subject, an effective amount of all trans retinoic acid (ATRA).
